(19) United States
(12) Patent Application Publication
(10) Pub. No.: US 2026/0285895 A1
(43) Pub. Date: Sep. 24, 2026

(54) PROCESSES OF PRODUCING VOLIXIBAT AND ANALOGS THEREOF

(71) Applicant: Mirum Pharmaceuticals, Inc., Foster City, CA (US)

(72) Inventors: Florina VOICA, Foster City, CA (US); Angela LEW, Foster City, CA (US); Andrea AMBROSI, Foster City, CA (US); Antal HARSANYI, Forster City, CA (US); Patrick LOOS, Foster City, CA (US); Kai ROSSEN, Forster City, CA (US); Andreas HANS, Foster City, CA (US); Pablo MORA, Foster City, CA (US); Wei DING, Foster City, CA (US); Johannes PLATZEK, Foster City, CA (US)

(73) Assignee: Mirum Pharmaceuticals, Inc., Foster City, CA (US)

(21) Appl. No.: 19/546,125

(22) Filed: Feb. 20, 2026

Related U.S. Application Data
(60) Provisional application No. 63/761,415, filed on Feb. 21, 2025.

(51) Int. Cl. C07H 15/26; C07C 69/614; C07C 69/732; C07C 317/22; C07C 317/24; C07C 323/19; C07C 323/22; C07C 327/28; C07D 337/08; C07H 15/203

(52) U.S. Cl. CPC C07H 15/26; C07C 69/614; C07C 69/732; C07C 317/22; C07C 317/24; C07C 323/19; C07C 323/22; C07C 327/28; C07D 337/08; C07H 15/203

(57) ABSTRACT

Provided herein are processes of producing volixibat and volixibat analogs, or their pharmaceutically acceptable salts, hydrates, or solvates thereof. Further provided herein are compounds of volixibat analogs, intermediates, or their pharmaceutically acceptable salts, hydrates, or solvates thereof. Additionally provided herein are crystalline forms of potassium volixibat salts. Provided herein are pharmaceutical compositions and dosage forms of the compositions comprising the compounds as described herein. Further provided herein are methods and kits of treating liver disease, hyperlipidemia, high cholesterol, arteriosclerosis, or Syndrome X using the compositions comprising the compounds as described herein.

### CROSS-REFERENCE TO RELATED APPLICATION

This application claims priority to U.S. Provisional Application No. 63/761,415, filed Feb. 21, 2025, the contents of which is incorporated by reference herein in its entirety.

### FIELD OF THE INVENTION

Provided herein are processes of producing volixibat and volixibat analogs, as well as their pharmaceutically acceptable salts, hydrates, and solvates thereof. Further provided herein are volixibat analog compounds, intermediates, or their pharmaceutically acceptable salts, hydrates, or solvates thereof. Additionally provided herein are crystalline forms of potassium volixibat salts.

### BACKGROUND

Hypercholemia and cholestatic liver diseases are liver diseases associated with impaired bile secretion (i.e., cholestasis), associated with and often secondary to the intracellular accumulation of bile acids/salts in the hepatocyte. Hypercholemia is characterized by increased serum concentration of bile acid or bile salt. Cholestasis can be categorized clinicopathologically into two principal categories of obstructive, often extrahepatic, cholestasis, and nonobstructive, or intrahepatic, cholestasis. Nonobstructive intrahepatic cholestasis can further be classified into two principal subgroups of primary intrahepatic cholestasis that result from constitutively defective bile secretion, and secondary intrahepatic cholestasis that result from hepatocellular injury. Primary intrahepatic cholestasis includes diseases such as benign recurrent intrahepatic cholestasis, which is predominantly an adult form with similar clinical symptoms, and progressive familial intrahepatic cholestasis (PFIC) types 1, 2, and 3, which are diseases that affect children. Pediatric cholestatic liver diseases affect a small percentage of children, but therapy results in significant healthcare costs each year. Currently, many of the pediatric cholestatic liver diseases require invasive and costly treatments such as liver transplantation and surgery.

Volixibat, also known as SHP626, LUM002, or ((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate), is an ileal bile acid transporter (IBAT) inhibitor to treat hypercholemia and cholestatic liver diseases. There remains a need to discover a more efficient, higher yielding, scalable synthesis of volixibat employing GMP standards. The method for the production of 1,4-benzothiepin-1,1-dioxide derivatives has been described in U.S. Pat. No. 8,461,312. The present disclosure relates to an improved method for the production of volixibat and its analogs.

### SUMMARY OF THE INVENTION

As specified in the Background section above, there is a great need in the art for a more efficient, higher yielding, and scalable synthesis of volixibat. The present application addresses these and other needs.

Provided herein is a process of preparing a compound of Formula (I),

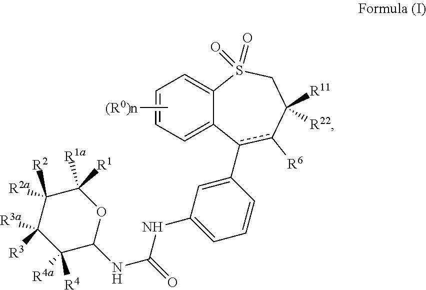

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
one of R¹ and R^{1a} is an optionally substituted (C₁₋₆)alkylene-OSO₃H or —C(O)H and the other of R¹ and R^{1a} is H, (C₁₋₆)alkyl, NH₂, NH(C₁₋₆)alkyl, or N((C₁₋₆)alkyl)₂;
one of R² and R^{2a} is an optionally substituted (C₁₋₆)alkylene-OH, OH, or OSO₃H, and the other of R² and R^{2a} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R⁴ and R^{4a} is an optionally substituted (C₁₋₆)alkylene-OH, OH, or OSO₃H, and the other of R⁴ and R^{4a} is H, (C₁₋₆)alkyl, NH₂, NH(C₁₋₆)alkyl, or N((C₁₋₆)alkyl)₂;
each of R¹¹ and R²² is independently an optionally substituted (C₁₋₆)alkyl;
n is an integer of 0-4;
R⁶ is H or OH;
the process comprising:
contacting the compound of Formula (I-26),

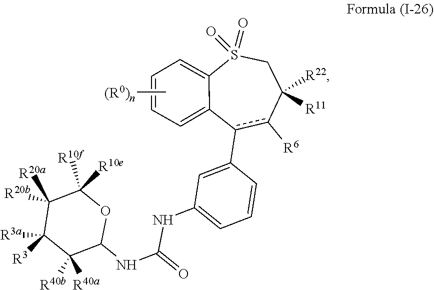

or a pharmaceutically acceptable hydrate or solvate thereof, wherein:
R⁰, R¹¹, R²², R³, R^{3a}, R⁶, and n are defined above;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂;
one of R^{10e} and R^{10f} is C(O)H or an optionally substituted (C₁₋₆)alkylene-OSO₃⁻(pyridine)⁺ and the other of R^{10e} and R^{10f} is H, (C₁₋₆)alkyl, NH₂, NH(C₁₋₆)alkyl, or N((C₁₋₆)alkyl)₂;
with a base in an organic solvent comprising dichloromethane to produce a compound of Formula (I).

Additionally provided herein is a process of preparing a compound of Formula (II-15),

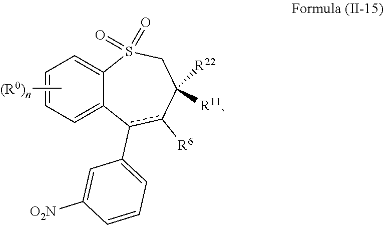

or a pharmaceutically acceptable salt, hydrate, or solvate thereof, the process comprising:
isolating the compound of Formula (II-15) from the mixture represented by Formula (II-15/115),

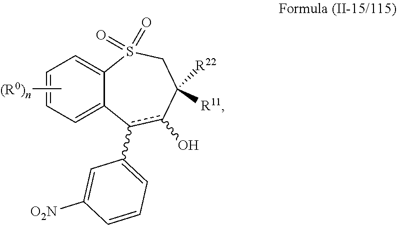

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R²² and R¹¹ is independently an optionally substituted (C₁₋₆)alkyl;
R⁶ is OH; and
n is an integer of 0-4;
and the process further comprising oxidizing and subsequently reducing the compound of Formula (II-15) or the mixture represented by Formula (II-15/115), forming a mixture represented by Formula (II-15/115).

Further provided herein is a process of preparing a compound of Formula (II-12),

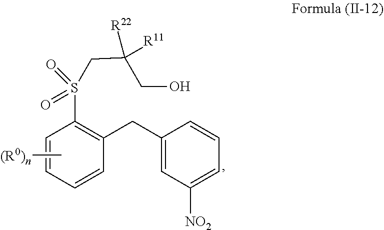

or a pharmaceutically acceptable salt, hydrate, or solvate thereof, the process comprising: contacting the compound of Formula (II-11-int),

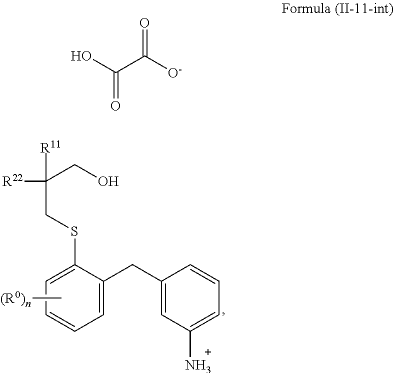

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with an oxidizing reagent in an organic solvent to produce a compound of Formula (II-12).

Additionally provided herein is a process of preparing a compound of Formula (II-11),

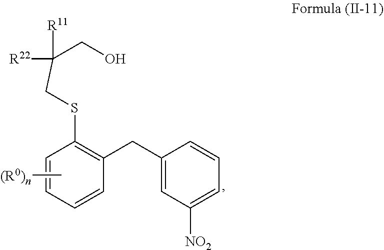

or a pharmaceutically acceptable salt, hydrate, or solvate thereof, the process comprising:
contacting the compound of Formula (II-215),

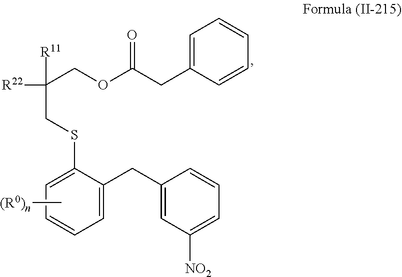

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-11).

Further provided herein is a process of preparing a compound of Formula (II-11),

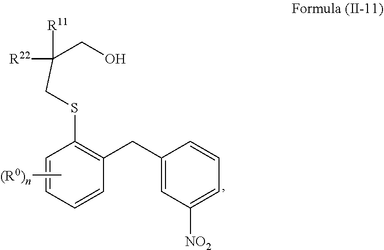

or a pharmaceutically acceptable salt, hydrate, or solvate thereof, the process comprising: contacting the compounds of Formula (II-208),

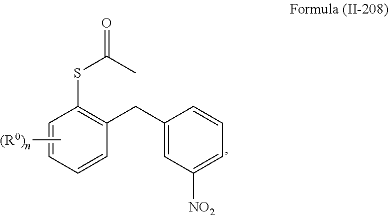

or a pharmaceutically acceptable salt, hydrate, or solvate thereof, and Formula (II-33),

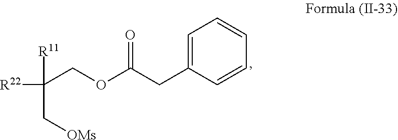

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-11).

Additionally provided herein is a process of preparing a compound of Formula (II-A-7),

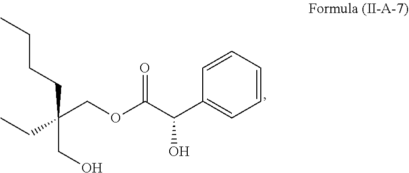 [SMILES: CCCC[C@](CC)(CO)COC(=O)[C@@H](O)c1ccccc1]

or a pharmaceutically acceptable salt, hydrate, or solvate thereof, the process comprising: contacting the compounds of 2-butyl-2-ethylpropane-1,3-diol and ethyl (2S)-2-hydroxy-2-phenyl-acetate, in an organic solvent to produce a compound of Formula (II-A-7).

Further provided herein is a process of preparing a compound of Formula (I-A-65),

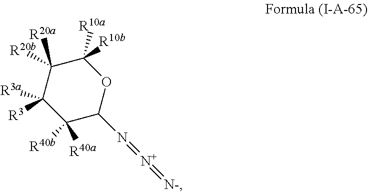

or a pharmaceutically acceptable salt, hydrate, or solvate thereof, the process comprising:
using a flow process to contact the compound of Formula (I-A-63),

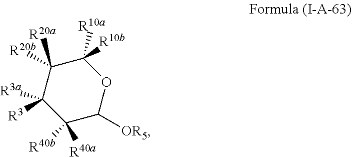

or a pharmaceutically acceptable salt, hydrate, or solvate thereof, wherein:
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; and
R⁵ is a fifth alcohol protecting group;
with trimethylsilyl azide in the presence of a Lewis acid to produce a compound of Formula (I-A-65).

Additionally provided herein is a compound of Formula (I),

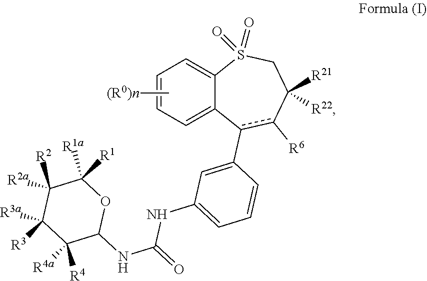

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
one of R¹ and R^{1a} is an optionally substituted (C₁₋₆)alkylene-OSO₃H, (C₁₋₆)alkylene-OH, (C₁₋₆)alkylene-OC(O)R⁰⁰, or C(O)H and the other of R¹ and R^{1a} is H, (C₁₋₆)alkyl, NH₂, NH(C₁₋₆)alkyl, or N((C₁₋₆)alkyl)₂;
one of R² and R^{2a} is an optionally substituted (C₁₋₆)alkylene-OH, OH, OSO₃H, or OC(O)R⁰⁰ and the other of R² and R^{2a} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R⁴ and R^{4a} is an optionally substituted (C₁₋₆)alkylene-OH, OH, OSO₃H, or OC(O)R⁰⁰ and the other of R⁴ and R^{4a} is H, (C₁₋₆)alkyl, NH₂, NH(C₁₋₆)alkyl, or N((C₁₋₆)alkyl)₂;
each of R¹¹ and R²² is independently an optionally substituted (C₁₋₆)alkyl;
n is an integer of 0-4;
R⁶ is H or OH;
wherein the compound of Formula (I) does not have the structure of Formula (III-27),

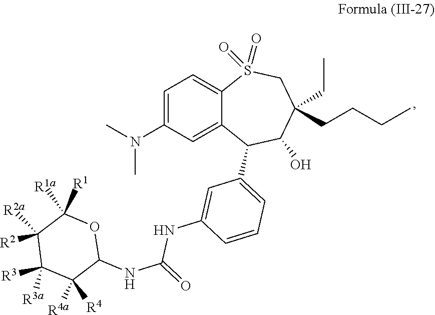

or a pharmaceutically acceptable salt, hydrate, or solvate thereof, wherein:
one of R¹ and R^{1a} is an optionally substituted (C₁₋₆)alkylene-OSO₃H and the other of R¹ and R^{1a} is H or (C₁₋₆)alkyl;
one of R² and R^{2a} is an optionally substituted (CH₂)—OH or OH and the other of R² and R^{2a} is H or (C₁₋₆)alkyl;
one of R³ and R^{3a} is an optionally substituted (C₁₋₆)alkylene-O—R^{1P} or —O—R^{1P}, and the other of R³ and R^{3a} is H or (C₁₋₆)alkyl;
wherein R^{1P} is (C₁-C₆)-alkyl optionally substituted by fluorine, phenyl, —(CH₂)₁₋₆-phenyl, O-phenyl, O—(CH₂)₁₋₆-phenyl, —(CH₂)—O—(CH₂)₁₋₆-phenyl, where the phenyl ring is optionally mono- to trisubstituted with F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, or CONH₂; and
one of R⁴ and R^{4a} is an optionally substituted (CH₂)—OH or OH and the other of R⁴ and R^{4a} is H or (C₁₋₆)alkyl.

Further provided herein is a compound of Formula (I-26)

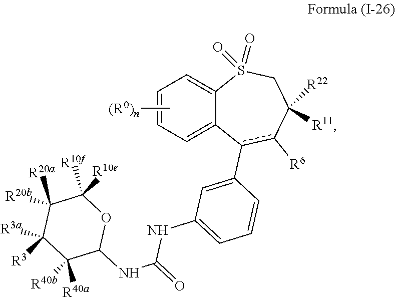

or a pharmaceutically acceptable hydrate or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
   each of R¹¹ and R²² is independently an optionally substituted (C₁₋₆)alkyl;
   n is an integer of 0-4;
   R⁶ is H or OH;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{10e} and R^{10f} is C(O)H or an optionally substituted (C₁₋₆)alkylene-OSO₃⁻(pyridine)⁺ and the other of R^{10e} and R^{10f} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P} or O—R^{2P}, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P} or O—R^{3P}, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; wherein the compound of Formula (I-26) does not have the structure of Formula (II-A-26),

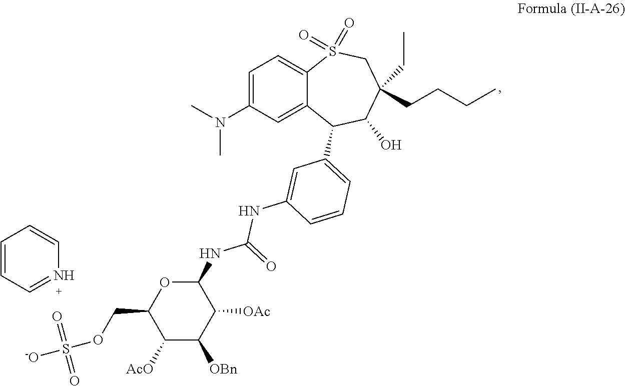

or a pharmaceutically acceptable hydrate or solvate thereof.

Additionally provided herein is a compound of Formula (I-25),

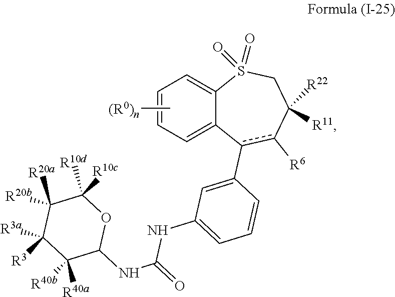

or a pharmaceutically acceptable hydrate or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰—S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R¹¹ and R²² is independently an optionally substituted (C₁₋₆)alkyl;
n is an integer of 0-4;
R⁶ is H or OH;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{10d} and R^{10e} is C(O)H or an optionally substituted (C₁₋₆)alkylene-OH or —OH, and the other of R^{10d} and R^{10e} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P} or O—R^{2P}, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P} or O—R^{3P}, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; wherein the compound of Formula (I-25) does not have the structure of Formula (II-A-25),

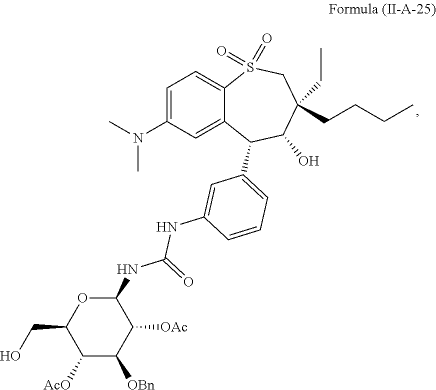 [SMILES: CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

or a pharmaceutically acceptable hydrate or solvate thereof.

Additionally provided herein is a compound of Formula (I-20),

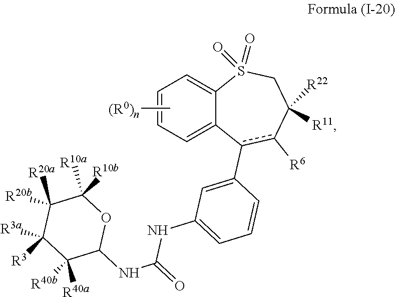

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R¹¹ and R²² is independently an optionally substituted (C₁₋₆)alkyl;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂;
n is an integer of 0-4;
R⁶ is H or OH; and
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂; wherein the compound of Formula (I-20) does not have the structure of Formula (II-A-20),

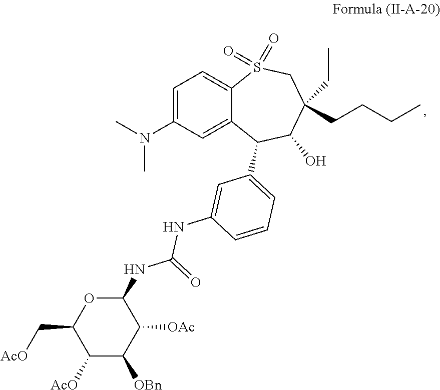 [SMILES: CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

or a pharmaceutically acceptable salt, hydrate, or solvate thereof.

Further provided herein is a compound of Formula (II-17),

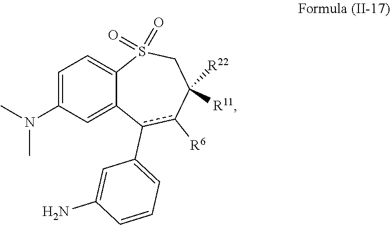

or a pharmaceutically acceptable salt, hydrate, or solvate thereof, wherein:
each of R¹¹ and R²² is independently an optionally substituted (C₁₋₆)alkyl; and
R⁶ is H or OH, wherein the compound of Formula (II-17) does not have the structure of Formula (II-A-17),

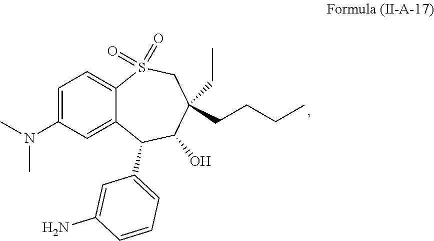 [SMILES: CCCC[C@]1(CC)CS(=O)(=O)c2ccc(N(C)C)cc2[C@@H](c2cccc(N)c2)[C@H]1O]

or a pharmaceutically acceptable salt, hydrate, or solvate thereof.

Additionally provided herein is a compound of Formula (II-16),

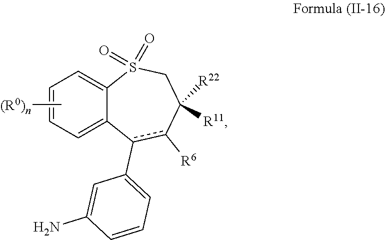

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl; and
R⁶ is H or OH, wherein the compound of Formula (II-16) does not have the structure of Formula (II-E-16),

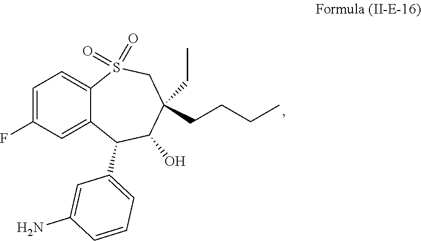 [SMILES: CCCC[C@]1(CC)CS(=O)(=O)c2ccc(F)cc2[C@@H](c2cccc(N)c2)[C@H]1O]

or a pharmaceutically acceptable salt, hydrate, or solvate thereof.

Further provided herein is a compound of Formula (II-15),

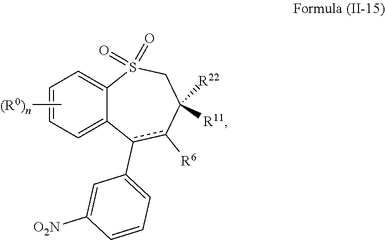

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl; and
R⁶ is H or OH, wherein the compound of Formula (II-15) does not have the structure of Formula (II-E-15),

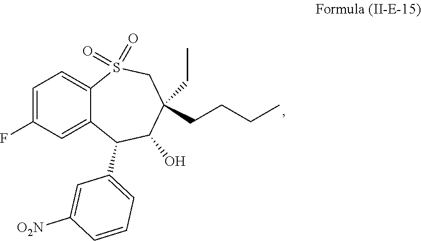 [SMILES: CCCC[C@]1(CC)CS(=O)(=O)c2ccc(F)cc2[C@@H](c2cccc([N+](=O)[O-])c2)[C@H]1O]

or a pharmaceutically acceptable salt, hydrate, or solvate thereof.

Additionally provided herein is a compound of Formula (II-C-150),

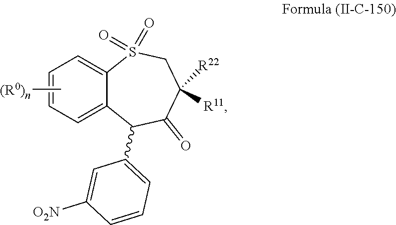

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-13),

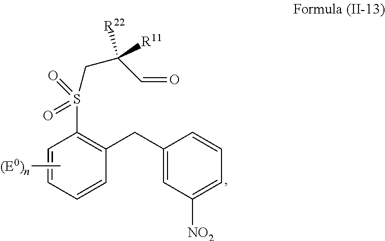

or a pharmaceutically acceptable salt, hydrate, or solvate thereof, wherein:
each of R¹¹ and R²² is independently an optionally substituted (C₁₋₆)alkyl;
n is an integer of 0-4; and each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-13) does not have the structure of Formula (II-A-13),

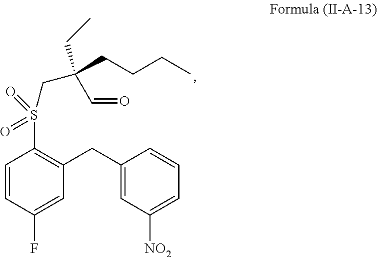 [SMILES: CCCC[C@](C=O)(CC)CS(=O)(=O)c1ccc(F)cc1Cc1cccc([N+](=O)[O-])c1]

or a pharmaceutically acceptable salt, hydrate, or solvate thereof.

Additionally provided herein is a compound of Formula (II-12),

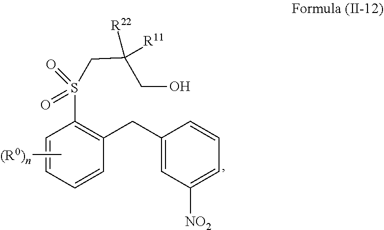

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-12) does not have the structure of Formula (II-A-12),

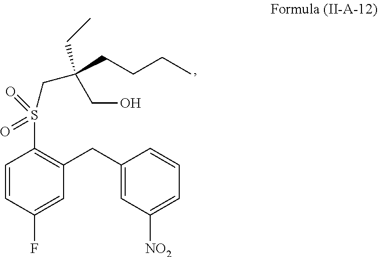 [SMILES: CCCC[C@@](CC)(CO)CS(=O)(=O)c1ccc(F)cc1Cc1cccc([N+](=O)[O-])c1]

or a pharmaceutically acceptable salt, hydrate, or solvate thereof.

Further provided herein is a compound of Formula (II-11-int),

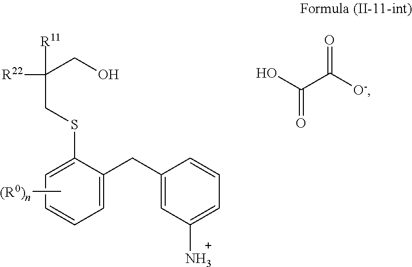

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-11),

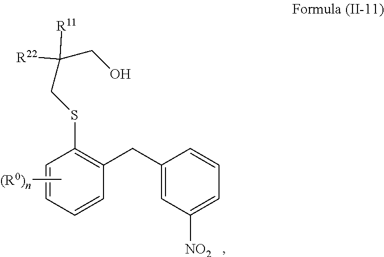

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-11) does not have the structure of Formula (II-A-11),

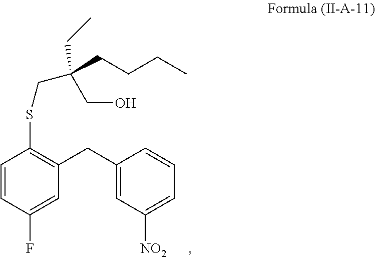 [SMILES: CCCC[C@@](CC)(CO)CSc1ccc(F)cc1Cc1cccc([N+](=O)[O-])c1]

or a pharmaceutically acceptable salt, hydrate, or solvate thereof.

Additionally provided herein is a compound of Formula (II-10),

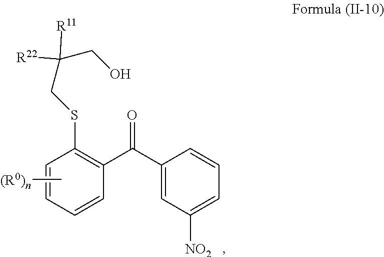

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-10) does not have the structure of Formula (II-A-10),

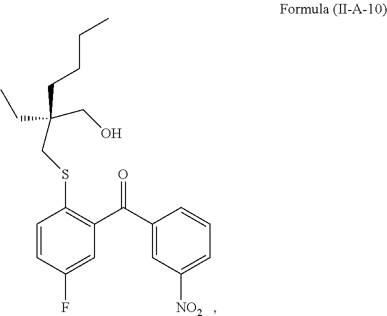 [SMILES: CCCC[C@@](CC)(CO)CSc1ccc(F)cc1C(=O)c1cccc([N+](=O)[O-])c1]

or a pharmaceutically acceptable salt, hydrate, or solvate thereof.

Further provided herein is a compound of Formula (II-215),

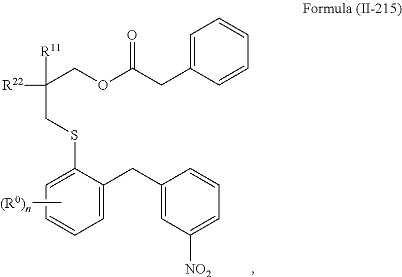

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Additionally provided herein is a compound of Formula (II-210),

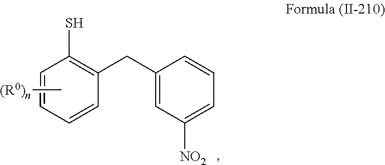

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-214),

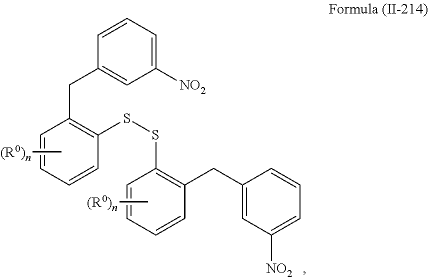

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Additionally provided herein is a compound of Formula (II-205),

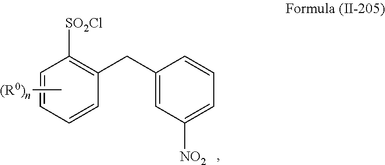

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-209),

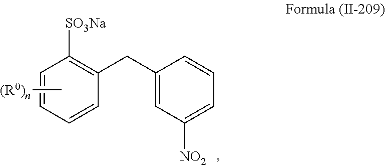

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Additionally provided herein is a compound of Formula (II-204),

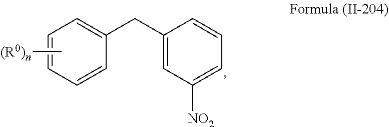

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-203),

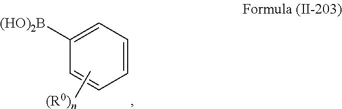

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Additionally provided herein is a compound of Formula (II-202),

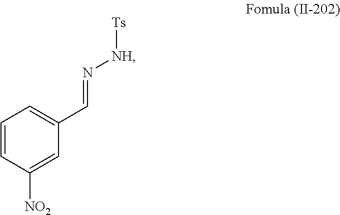 [SMILES: Cc1ccc(S(=O)(=O)N/N=C/c2cccc([N+](=O)[O-])c2)cc1]

or a pharmaceutically acceptable salt, hydrate, or solvate thereof.

Further provided herein is a compound of Formula (II-208),

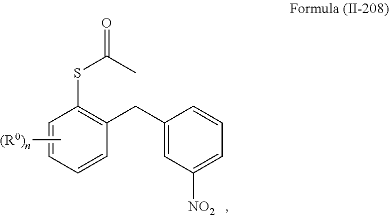

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Additionally provided herein is a compound of Formula (II-207),

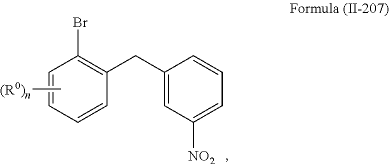

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-206),

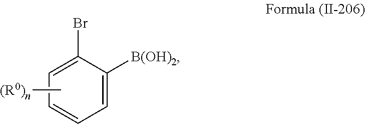

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

Further provided herein is a compound of Formula (II-B-7),

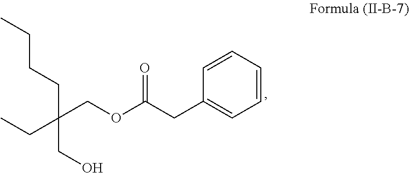 [SMILES: CCCCC(CC)(CO)COC(=O)Cc1ccccc1]

or a pharmaceutically acceptable salt, hydrate, or solvate thereof.

Additionally provided herein is a compound of Formula (II-C-9),

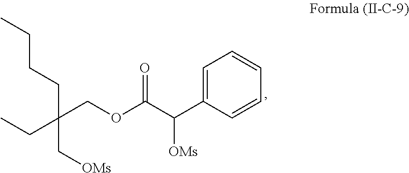 [SMILES: CCCCC(CC)(COC(=O)C(OS(C)(=O)=O)c1ccccc1)COS(C)(=O)=O]

or a pharmaceutically acceptable salt, hydrate, or solvate thereof.

Further provided herein is a compound of Formula (II-B-9),

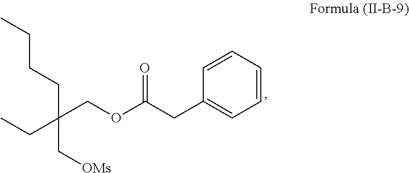 [SMILES: CCCCC(CC)(COC(=O)Cc1ccccc1)COS(C)(=O)=O]

or a pharmaceutically acceptable salt, hydrate, or solvate thereof.

Additionally provided herein is a compound of Formula (II-B-44),

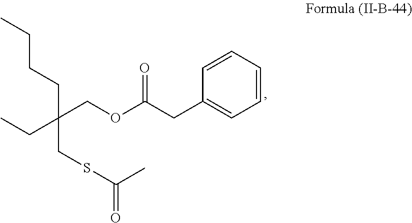 [SMILES: CCCCC(CC)(COC(=O)Cc1ccccc1)CSC(C)=O]

or a pharmaceutically acceptable salt, hydrate, or solvate thereof.

Further provided herein is a compound of Formula (I-A-66),

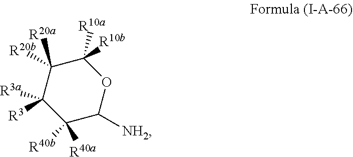

or a pharmaceutically acceptable salt, hydrate, or solvate thereof, wherein:
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂.

Further provided herein is a compound prepared by the processes as described herein.

Additionally provided herein is a pharmaceutical composition comprising a therapeutically effective amount of any of the compounds as described herein.

Further provided herein is a pharmaceutical dosage form comprising any of the compounds or the pharmaceutical compositions as described herein.

Additionally provided herein is a method of treating a cholestatic liver disease in a subject in need thereof, comprising administering to the subject a therapeutically effective amount of any of the compounds, the pharmaceutical compositions, or the pharmaceutical dosage forms as described herein.

Further provided herein is a method of treating hyperlipidemia in a subject in need thereof, wherein the method comprises administering to the subject a therapeutically effective amount of any of the compounds, the pharmaceutical compositions, or the pharmaceutical dosage forms as described herein.

Additionally provided herein is a method of lowering the serum cholesterol level in a subject in need thereof, wherein the method comprises administering to the subject a therapeutically effective amount of any of the compounds, the pharmaceutical compositions, or the pharmaceutical dosage forms as described herein.

Further provided herein is a method of treating arteriosclerosis in a subject in need thereof, wherein the method comprises administering to the subject a therapeutically effective amount of any of the compounds, the pharmaceutical compositions, or the pharmaceutical dosage forms as described herein.

Additionally provided herein is a method of treating Syndrome X in a subject in need thereof, wherein the method comprises administering to the subject a therapeutically effective amount of any of the compounds, the pharmaceutical compositions, or the pharmaceutical dosage forms as described herein.

Further provided herein is a pharmaceutical composition comprising a therapeutically effective amount of the compound of Formula (I) and one or more excipients selected from the group consisting of:
(i) a diluent or a filler,
(ii) a disintegrant,
(iii) a channeling agent,
(iv) a glidant, and
(v) a lubricant.

Additionally provided herein is a kit for treating cholestatic liver disease in a subject in need thereof, wherein the kit comprises at least one unit dosage of a therapeutically effective amount of any of the compounds, the pharmaceutical compositions, or the pharmaceutical dosage forms as described herein.

Further provided herein is a kit for treating hyperlipidemia in a subject in need thereof, wherein the kit comprises a therapeutically effective amount of any of the compounds, the pharmaceutical compositions, or the pharmaceutical dosage forms as described herein.

Additionally provided herein is a kit for lowering the serum cholesterol level in a subject in need thereof, wherein the kit comprises a therapeutically effective amount of any of the compounds, the pharmaceutical compositions, or the pharmaceutical dosage forms as described herein.

Further provided herein is a kit for treating arteriosclerosis in a subject in need thereof, wherein the kit comprises a therapeutically effective amount of any of the compounds, the pharmaceutical compositions, or the pharmaceutical dosage forms as described herein.

Additionally provided herein is a kit for treating Syndrome X in a subject in need thereof, wherein the kit comprises a therapeutically effective amount of any of the compounds, the pharmaceutical compositions, or the pharmaceutical dosage forms as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the scheme to synthesize compound 17 (Formula (II-A-17)).

FIG. 1B shows the scheme to synthesize compound 27 (Formula (II-A-27)).

FIG. 1C shows the scheme to synthesize compound 66 (Formula (II-A-66)).

FIG. 2 shows NMR of compound 7 (Formula (II-A-7)).

FIG. 3 shows NMR of compound 33 (Formula (II-A-33)).

FIG. 4 shows NMR of compound 44 (Formula (II-A-44)).

FIG. 5A shows NMR of compound 10 (Formula (II-A-10)).

FIG. 5B shows NMR of compound 11 (Formula (II-A-11)).

FIG. 5C shows NMR of compound 12 (Formula (II-A-12)).

FIG. 5D shows NMR of compound 13 (Formula (II-A-13)).

FIG. 6A shows NMR of compound 15 (Formula (II-E-15)).

FIG. 6B shows NMR of compound 15A (Formula (II-F-15)).

FIG. 6C shows NMR of compound 16 (Formula (II-E-16)).

FIG. 6D shows NMR of compound 17 (Formula (II-A-17)).

FIG. 7A shows NMR of compound 65 (Formula (II-A-65)).

FIG. 7B shows NMR of compound 66 (Formula (II-A-66)).

FIG. 8A shows NMR of compound 20 (Formula (II-A-20)).

FIG. 8B shows NMR of compound 25 (Formula (II-A-25)).

FIG. 8C shows NMR of compound 26 (Formula (II-A-26)).

FIG. 8D shows NMR of compound 27 (Formula (II-A-27)).

FIG. 9 shows ¹H-NMR (Nuclear Magnetic Resonance) overlay of volixibat potassium salt Form I.

FIG. 10 shows XRPD pattern of volixibat potassium salt Form I.

FIG. 11 shows DSC (Differential Scanning Calorimetric) thermogram of volixibat potassium salt Form I.

FIG. 12 shows TGA (Thermal Gravimetric Analysis) thermogram of volixibat potassium salt Form I.

FIG. 13 shows PLM (Polarized Light Microscope) photograph of volixibat potassium salt Form I.

FIG. 14 shows XRPD (X-ray Powder Diffractometer) overlay of polymorphs.

FIG. 15 shows XRPD pattern of volixibat potassium salt Pattern A.

FIG. 16 shows XRPD overlay of conversion between volixibat potassium salt Pattern A and Form II.

FIG. 17 shows XRPD overlay of samples obtained from volixibat potassium salt Pattern A at different humidities.

FIG. 18 shows XRPD overlay of samples obtained from volixibat potassium salt Pattern A at different humidities.

FIG. 19 shows XRPD pattern of volixibat potassium salt Form I.

FIG. 20 shows SEM (Scanning Electronic Microscope) microphoto of volixibat potassium salt Form I.

FIG. 21 shows DSC thermogram of volixibat potassium salt Form I.

FIG. 22 shows TGA thermogram of volixibat potassium salt Form I.

FIG. 23 shows ¹H-NMR overlay of volixibat potassium salt Form I.

FIG. 24 shows XRPD overlay of samples obtained by stressing volixibat potassium salt Form I.

FIG. 25 shows DVS (Dynamic Vapor Sorption) isotherm plot of volixibat potassium salt Form I at 25° C.

FIG. 26 shows DVS isotherm plot of volixibat potassium salt Form I at 25° C.

FIG. 27 shows XRPD overlay of volixibat potassium salt Form I before and after DVS test.

FIG. 28 shows XRPD overlay of volixibat potassium salt Form I before and after DVS test.

FIG. 29 shows DVS isotherm plot of volixibat potassium salt Form I at 25° C.

FIG. 30 shows DVS isotherm plot of volixibat potassium salt Form I at 25° C.

FIG. 31 shows XRPD overlay of volixibat potassium salt Form I before and after DVS test.

FIG. 32 shows XRPD overlay of volixibat potassium salt Form I before and after DVS test.

FIG. 33 shows XRPD overlay of samples obtained by compressing volixibat potassium salt Form I.

FIG. 34 shows XRPD overlay of samples obtained by dry grinding volixibat potassium salt Form I.

FIG. 35 shows PLM photograph of volixibat potassium salt Form I after ball milling.

FIG. 36 shows XRPD overlay of samples obtained by ball milling volixibat potassium salt Form I.

FIG. 37 shows XRPD overlay of samples obtained by wet grinding volixibat potassium salt Form I.

FIG. 38 shows ORTEP structure of volixibat potassium salt Form I.

FIGS. 39A-39C show packing diagrams of volixibat potassium salt Form I. FIG. 39A shows the packing diagram on the a axis, FIG. 39B shows the packing diagram on the b axis, and FIG. 39C shows the packing diagram on the c axis.

FIG. 40 shows crystals of volixibat potassium salt Form I.

FIG. 41 shows XRPD pattern of volixibat potassium salt Form II.

FIG. 42 shows ¹H-NMR overlay of volixibat potassium salt Form II.

FIG. 43 shows DSC thermogram of volixibat potassium salt Form II.

FIG. 44 shows TGA thermogram of volixibat potassium salt Form II.

FIG. 45 shows DVS isotherm plot of volixibat potassium salt Form II at 25° C.

FIG. 46 shows XRPD overlay of volixibat potassium salt Form II before and after DVS test.

FIG. 47 shows DVS isotherm plot of volixibat potassium salt Form II at 25° C.

FIG. 48 shows XRPD overlay of volixibat potassium salt Form II before and after DVS test.

FIG. 49 shows XRPD pattern of volixibat potassium salt Form III.

FIG. 50 shows XRPD overlay of conversion between volixibat potassium salt Form III and Form I.

FIG. 51 shows XRPD overlay of samples obtained from volixibat potassium salt Form III at different humidities.

FIG. 52 shows XRPD overlay of samples obtained from volixibat potassium salt Form III at different humidities.

FIG. 53 shows XRPD overlay of samples obtained by compressing volixibat potassium salt Form III.

FIG. 54 shows XRPD overlay of samples obtained by ball milling volixibat potassium salt Form III.

FIG. 55 shows XRPD overlay of samples obtained by dry grinding volixibat potassium salt Form III.

FIG. 56 shows ORTEP structure of volixibat potassium salt Form III.

FIGS. 57A-57C show packing diagrams of volixibat potassium salt Form III. FIG. 57A shows the packing diagram on the a axis, FIG. 57B shows the packing diagram on the b axis, and FIG. 57C shows the packing diagram on the c axis.

FIG. 58 shows crystals of volixibat potassium salt Form III.

FIG. 59 shows XRPD overlay of samples obtained from volixibat potassium salt Pattern A in 0% and 22.5% RH at 25° C.

FIG. 60 shows XRPD overlay of samples obtained from heating volixibat potassium salt Form I at 115° C. and 175° C.

FIG. 61 shows ¹H-NMR overlay of samples obtained from heating volixibat potassium salt Form I at 115° C. and 175° C.

FIG. 62 shows XRPD pattern of volixibat potassium salt amorphous form.

FIG. 63 shows ¹H-NMR overlay of samples obtained from heating volixibat potassium salt Form I at 150° C. for 1 hour.

FIG. 64 shows XRPD overlay of samples obtained from competitive equilibration experiments at 25° C.

FIG. 65 shows XRPD overlay of samples obtained from competitive equilibration experiments at 25° C.

FIG. 66 shows XRPD overlay of samples obtained from competitive equilibration experiments at 25° C.

FIG. 67 shows XRPD overlay of samples obtained from competitive equilibration experiments at 25° C. and at 15° C.

FIG. 68 shows XRPD overlay of samples obtained from competitive equilibration experiments at 5° C.

FIG. 69 shows XRPD overlay of samples obtained from equilibration experiments in different water activities at 5° C.

FIG. 70 shows XRPD overlay of samples obtained from equilibration experiments in different water activities at 5° C.

FIG. 71 shows XRPD overlay of samples obtained from equilibration experiments in different water activities at 25° C.

FIG. 72 shows XRPD overlay of samples obtained from equilibration experiments in different water activities at 25° C.

FIG. 73 shows XRPD overlay of samples obtained from Pattern A and Form III at 25° C. & 60% RH.

FIG. 74 shows XRPD overlay of samples obtained from equilibration experiments in different water activities at 50° C.

FIG. 75 shows XRPD overlay of samples obtained from equilibration experiments in different water activities at 50° C.

FIG. 76 shows XRPD overlay of samples obtained from cross seeding experiments at 25° C.

FIG. 77 shows XRPD overlay of samples obtained from cross seeding experiments at 25° C.

FIG. 78 shows XRPD overlay of samples obtained from cross seeding experiments at 25° C.

FIG. 79 shows XRPD overlay of samples obtained from cross seeding experiments at 25° C.

FIG. 80 shows a schematic diagram of the interconversion relationship of polymorphs.

FIG. 81 shows a schematic phase diagram of volixibat potassium salt.

### DETAILED DESCRIPTION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

Volixibat is known as SHP626, LUM002, or ((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate). The structure of volixibat potassium is shown below:

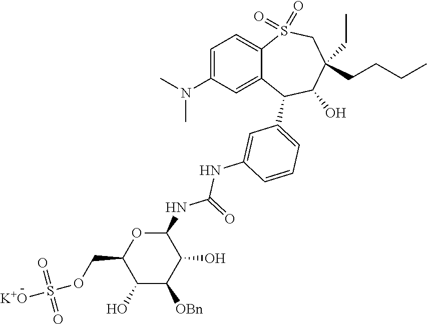

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH₂—).

As used herein, the term “composition” generally refers to a composition of two or more components, usually one or more drugs (e.g., volixibat) and one or more pharmaceutical excipients.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system.

As used herein, a “disintegrant” is an excipient that hydrates a pharmaceutical composition and aids in tablet dispersion.

As used herein, a “diluent” or “filler” is an excipient that adds bulkiness to a pharmaceutical composition.

As used herein, an “excipient” includes functional and non-functional ingredients in a pharmaceutical composition.

As used herein, a “glidant” is an excipient that imparts a pharmaceutical compositions with enhanced flow properties.

As used herein, the term “halo” means halogen groups including, but not limited to fluoro, chloro, bromo, and iodo.

As used herein, the term “inhibitor” means a substance that reduces measurable amount the activity of an enzyme or receptor. The term “inhibit” means reducing measurable amount the activity of an enzyme or receptor.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, a “lubricant” is an excipient that is added to pharmaceutical compositions that are pressed into tablets. The lubricant aids in compaction of granules into tablets and ejection of a tablet of a pharmaceutical composition from a die press.

As used herein, the term “percent” or “%” means the weight percentage of the total weight of the composition (i.e., by weight of the total composition).

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “a pharmaceutically acceptable salt, hydrate or solvate thereof” refers to “a pharmaceutically acceptable salt”, “a pharmaceutically acceptable hydrate”, or “a pharmaceutically acceptable solvate. As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments include pharmaceutically acceptable salt of the compounds described herein. The present embodiments also include quaternary ammonium salts of the compounds described herein, where the compounds have one or more tertiary amine moiety. As used herein, the phrase “pharmaceutically acceptable solvate,” includes, but is not limited to, the solvate formed by a solvent that is not listed by the U.S. FDA as Class 1 solvent in the “Q3C—Tables and List Guidance for Industry” (solvents that should not be employed in the manufacture of drug substances, excipients, and drug products because of their unacceptable toxicity or their deleterious environmental effect). Examples of pharmaceutically acceptable solvates” are, but not limited to, acetic acid, heptane, acetone, isobutyl acetate, anisole, isopropyl acetate, 1-butanol, methyl acetate, 2-butanol, 3-methyl-1-butanol, butyl acetate, methylethyl ketone, tert-butylmethyl ether, 2-methyl-1-propanol, dimethyl sulfoxide, pentane, ethanol, 1-pentanol, ethyl acetate, 1-propanol, ethyl ether, 2-propanol, ethyl formate, propyl acetate, formic acid, triethylamine, “1,1-diethoxypropane, methylisopropyl ketone, 1,1-dimethoxymethane, methyltetrahydrofuran, 2,2-dimethoxypropane, petroleum ether, isooctane, trichloroacetic acid, isopropyl ether, and trifluoroacetic acid. As used herein, “a pharmaceutically acceptable hydrate” refers to a form of a pharmaceutical compound that includes water molecules as part of its structure. The number of water molecules vary and do not have to be of an integer number.

As used herein, the term “phenyl” means —C₆H₅. A phenyl group can be unsubstituted or substituted with one, two, or three suitable substituents.

As used herein, the term “purified” means that when isolated, the isolate contains at least 90%, at least 95%, at least 98%, or at least 99% of a compound described herein by weight of the isolate.

As used herein, the term “solid dosage form” generally refers to a pharmaceutical composition, which when used in an oral mode of administration include capsules, tablets, pills, powders, and granules. In such solid dosage forms, the active compound is mixed with at least one inert, pharmaceutically acceptable excipient or carrier.

As used herein, the term “subject” means any animal, including mammals, such as mice, rats, other rodents, rabbits, dogs, cats, swine, cattle, sheep, horses, or primates, such as humans.

As used herein, “suitable substituent”, “substituent”, “optional substituent”, or the substituent for any optionally substituted group means a group that does not nullify the synthetic or pharmaceutical utility of the compounds described herein or the intermediates useful for preparing them. Examples of “suitable substituent”, “substituent”, “optional substituent”, or the substituent for any optionally substituted group includes, but are not limited to: C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(phenyl), —N(phenyl)₂, —CHO, —CO(C₁-C₆alkyl), —CO(phenyl), —CO₂(C₁-C₆alkyl), and —CO₂(phenyl). In some embodiments, one “suitable substituent”, “substituent”, “optional substituent”, or the substituent for any optionally substituted group is further substituted by one or more groups selected from C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(phenyl), —N(phenyl)₂, —CHO, —CO(C₁-C₆alkyl), —CO(phenyl), —CO₂(C₁-C₆alkyl), or —CO₂(phenyl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor, or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, elimination or amelioration of a disorder, or side-effects. The amount needed to elicit the therapeutic response can be determined based on the age, health, size, and sex of the subject. Optimal amounts can also be determined based on monitoring of the subject's response to treatment.

As used herein, the terms “treat,” “treated,”, “treatment”, or “treating” mean both therapeutic treatment wherein the object is to slow down (lessen) an undesired physiological condition, disorder, or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment.

At various places in the present specification, substituents of compounds may be disclosed in groups or in ranges. It is specifically intended that embodiments include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

As used herein, the half-dashed double bond, i.e., [symbol] , represents that there is at a minimum a single bond at that location and that further there may or may not be a double bond at that location. The half-dashed double bond can signify a single bond or a double bond between atoms.

As used herein, the wavy single bond, i.e., [symbol] , represents an unspecified stereochemistry, an unknown stereochemistry, and/or a mixture of isomers. It may represent a straight bond, a solid wedge bond, or a hashed wedge bond. The wavy single bond represents an unspecified stereochemistry, an unknown stereochemistry, and/or a mixture of isomers at the stereocenter, i.e., the chiral center, to which it is attached.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

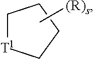

then it is understood that substituent R can occur s number of times on the ring, and R can be a different moiety at each occurrence. Further, in the above example, where the variable T¹ is defined to include hydrogens, such as when T¹ is CH₂, NIT, etc., any H can be replaced with a substituent.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompass the use, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present embodiments. Cis and trans geometric isomers of the compounds are also included within the scope of the embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Resolution Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound of the embodiments. Substantial separation can include compositions containing at least about 50%, at least about 60%, at least about 70%, at least about 80%, at least about 90%, at least about 95%, at least about 97%, or at least about 99% by weight of the compound of the embodiments, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid, or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

Embodiments of various compounds and salts thereof are provided. Where a variable is not specifically recited, the variable can be any option described herein, except as otherwise noted or dictated by context.

### Processes of Preparing Volixibat and its Analogs

Provided herein is a process of preparing a compound of Formula (I),

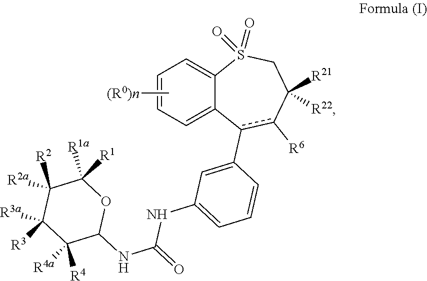

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
one of R¹ and R^{1a} is an optionally substituted (C₁₋₆)alkylene-OSO₃H or —C(O)H and the other of R¹ and R^{1a} is H, (C₁₋₆)alkyl, NH₂, NH(C₁₋₆)alkyl, or N((C₁₋₆)alkyl)₂;
one of R² and R^{2a} is an optionally substituted (C₁₋₆)alkylene-OH, OH, or OSO₃H, and the other of R² and R^{2a} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R⁴ and R^{4a} is an optionally substituted (C₁₋₆)alkylene-OH, OH, or OSO₃H, and the other of R⁴ and R^{4a} is H, (C₁₋₆)alkyl, NH₂, NH(C₁₋₆)alkyl, or N((C₁₋₆)alkyl)₂;
each of R¹¹ and R²² is independently an optionally substituted (C₁₋₆)alkyl;
n is an integer of 0-4;
R⁶ is H or OH;
the process comprising:
contacting the compound of Formula (I-26),

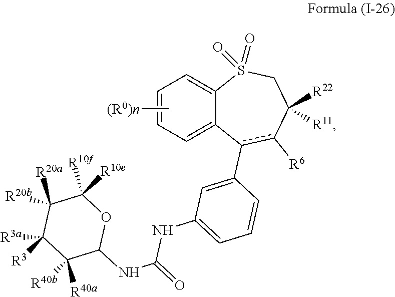

or a pharmaceutically acceptable hydrate or solvate thereof, wherein:
R⁰, R¹¹, R²², R³, R^{3a}, R⁶, and n are defined above;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂;
one of R^{10e} and R^{10f} is C(O)H or an optionally substituted (C₁₋₆)alkylene-OSO₃⁻(pyridine)⁺ and the other of R^{10e} and R^{10f} is H, (C₁₋₆)alkyl, NH₂, NH(C₁₋₆)alkyl, or N((C₁₋₆)alkyl)₂;
with a base in an organic solvent comprising dichloromethane to produce a compound of Formula (I).

In some embodiments, the base is potassium methoxide or sodium methoxide.

In another embodiment,

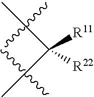

has the structure selected from the group consisting of

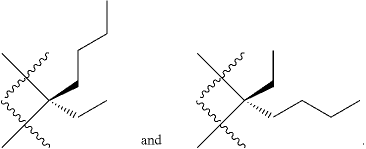

In some embodiments, R¹ is optionally substituted (C₁₋₆)alkylene —OSO₃⁻K⁺, (C₁₋₆)alkylene —OSO₃⁻Na⁺, or (C₁₋₆)alkylene —OSO₃⁻NH₄⁺ in the pharmaceutically acceptable salt of Formula (I).

In an additional embodiment, R¹ is optionally substituted —CH₂—OSO₃⁻K⁺, —CH₂—OSO₃⁻Na⁺, or —CH₂—OSO₃⁻NH₄⁺ in the pharmaceutically acceptable salt of Formula (I).

In some embodiments, R¹ is —CH₂—OSO₃⁻K⁺ in the pharmaceutically acceptable salt of Formula (I).

In another embodiment, each of R^{1a}, R², R^{3a}, and R⁴ is H.

In some embodiments, R³ is O-Bn.

In another embodiment, R^{2a} and R^{4a} are both —OH.

In some embodiments, R^{20b} and R^{40b} are both —O—Ac.

In an additional embodiment, R^{10e} is —CH₂—OSO₃⁻(pyridine)⁺.

In an additional embodiment, the organic solvent comprises dichloromethane in combination with methanol.

In some embodiments, the contacting the compound of Formula (I-26) with the base to produce a compound of Formula (I) occurs at a reaction temperature of about 0° C.

In some embodiments, the process further comprises isolating the compound of Formula (I) or a pharmaceutically acceptable salt, hydrate, or solvate thereof.

In some embodiments, the isolation of the compound of Formula (I) comprises recrystallizing the compound of Formula (I) from water and ethanol.

In another embodiment, the compound of Formula (I-26) has the structure selected from the group consisting of Formula (I-AA-26), Formula (I-AB-26), Formula (I-BA-26), Formula (I-BB-26), Formula (I-CA-26), Formula (I-CB-26), Formula (I-DA-26), and Formula (I-DB-26),

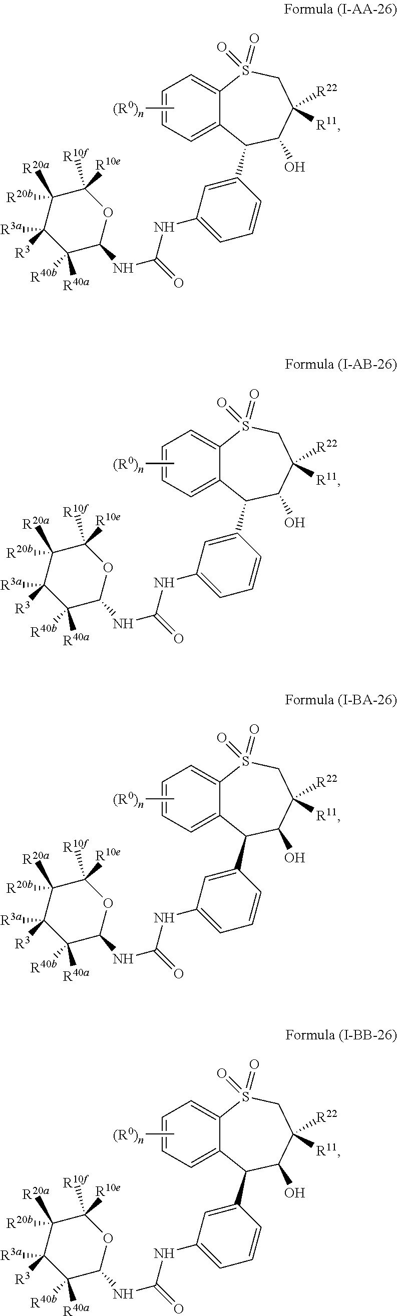

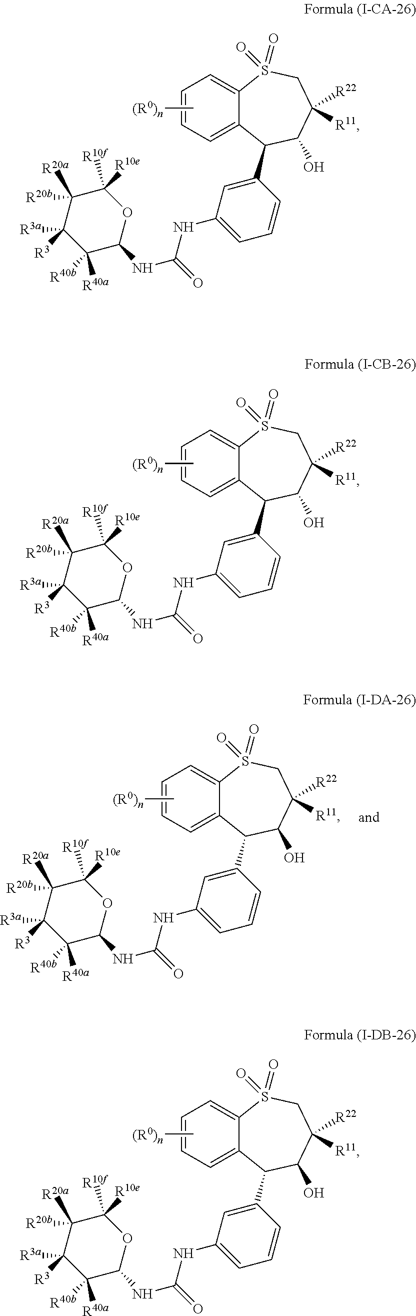

or a pharmaceutically acceptable hydrate or solvate thereof.

In some embodiments, the compound of Formula (I-26) has the structure of Formula (I-A-26),

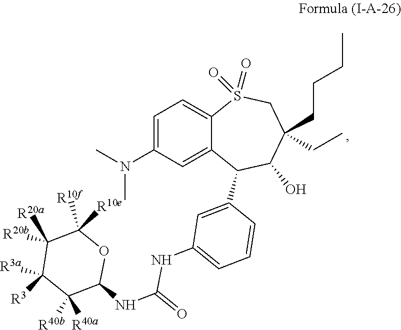

or a pharmaceutically acceptable hydrate or solvate thereof, wherein:
R^{10e} is (C₁₋₆)alkylene-OSO₃⁻(pyridine)⁺;
each of R^{10f}, R^{20a}, R^{3a}, and R^{40a} is independently H, (C₁₋₆)alkyl, NH₂, NH(C₁₋₆)alkyl, or N((C₁₋₆)alkyl)₂;
R^{20b} is an optionally substituted (C₁₋₆)alkylene-O—R^{2P} or O—R^{2P}, wherein R^{2P} is the second alcohol protecting group;
R^{40b} is an optionally substituted (C₁₋₆)alkylene-O—R^{3P} or O—R^{3P}, wherein R^{3P} is the third alcohol protecting group; and
R³ is an optionally substituted (C₁₋₆)alkylene-O—R^{1P} or —O—R^{1P}, wherein R^{1P} is the first alcohol protecting group.

In an additional embodiment, the compound of Formula (I-26) has the structure of Formula (II-A-26), Formula (II-A-126), Formula (II-A-137), Formula (II-A-94), Formula (II-A-133), Formula (II-A-75), Formula (II-A-98), Formula (II-A-174), Formula (II-A-178),

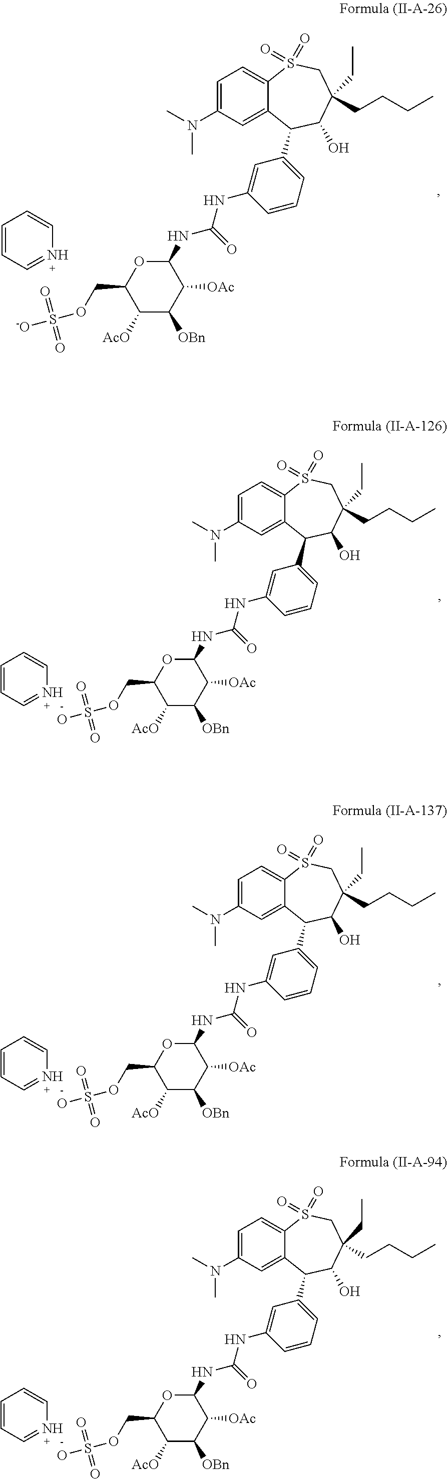

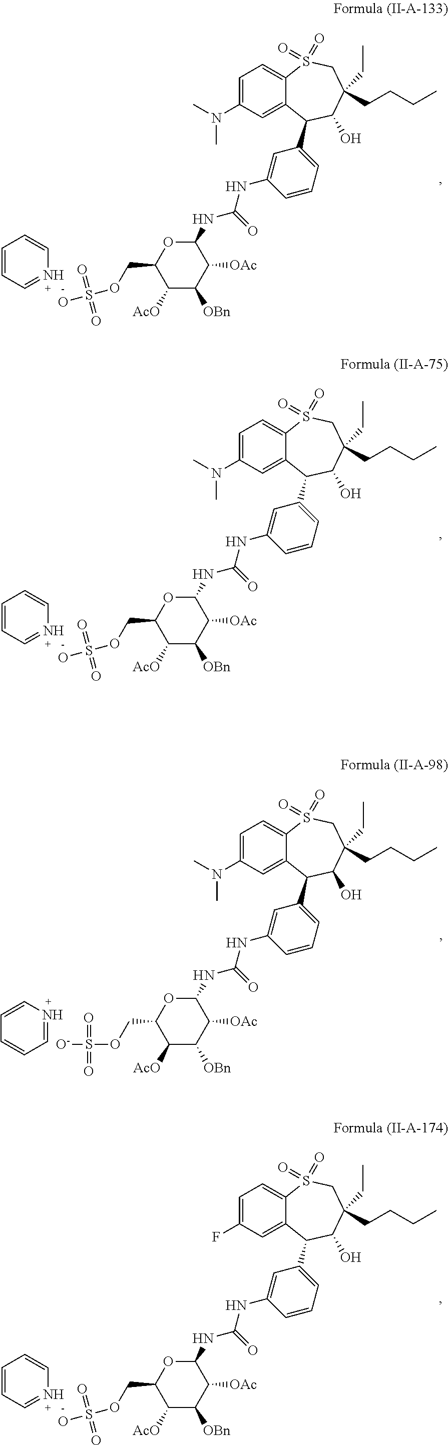

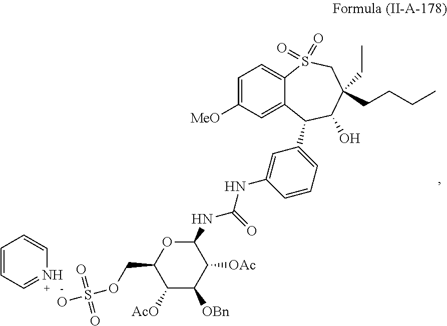

or a pharmaceutically acceptable hydrate or solvate thereof.

In some embodiments, the compound of Formula (I) has the structure selected from the group consisting of Formula (I-AA), Formula (I-AB), Formula (I-BA), Formula (I-BB), Formula (I-CA), Formula (I-CB), Formula (I-DA), and Formula (I-DB),

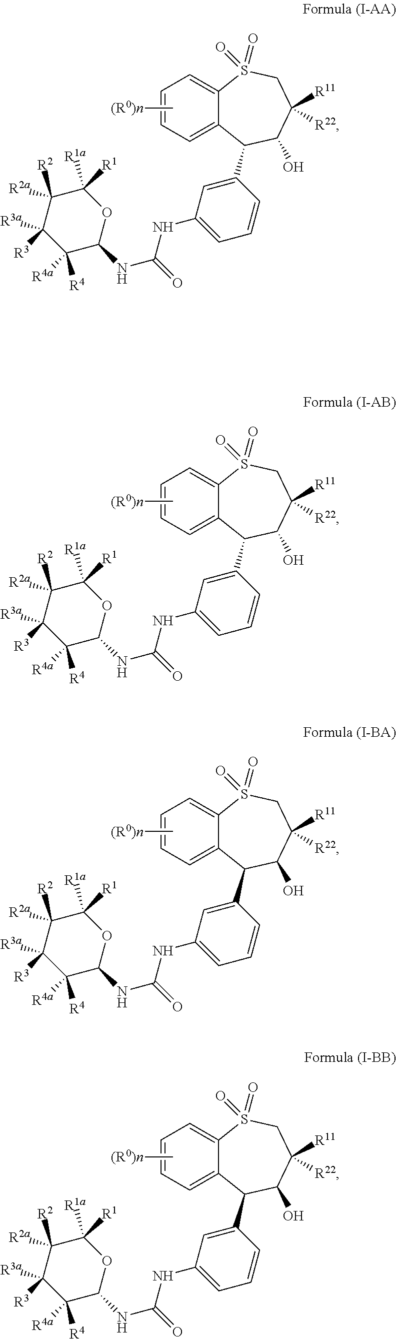

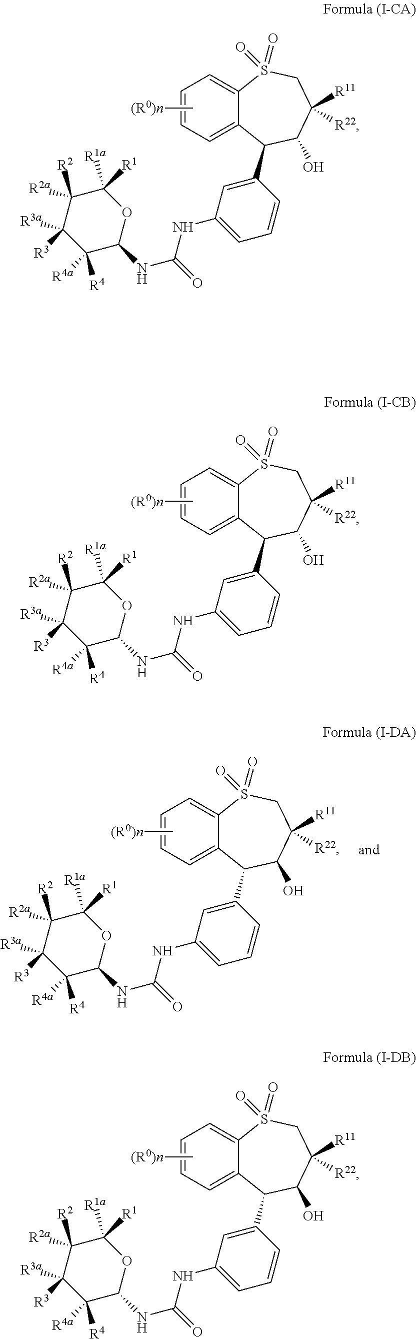

or a pharmaceutically acceptable salt, hydrate, or solvate thereof.

In some embodiments, the compound of Formula (I) has the structure of Formula (I-A),

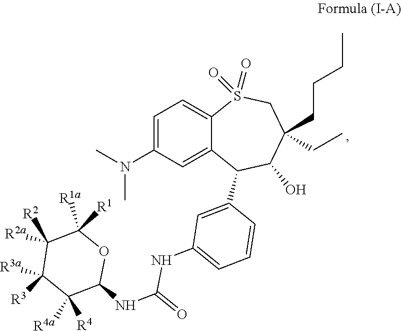

or a pharmaceutically acceptable salt, hydrate, or solvate thereof, wherein:
R¹ is an optionally substituted (C₁₋₆)alkylene-OSO₃H;
each of R^{1a}, R², R^{3a}, and R⁴ is independently H, (C₁₋₆)alkyl, NH₂, NH(C₁₋₆)alkyl, or N((C₁₋₆)alkyl)₂;
each of R^{2a} and R^{4a} is independently an optionally substituted (C₁₋₆)alkylene-OH or OH; and
R³ is an optionally substituted (C₁₋₆)alkylene-O—R^{1P} or —O—R^{1P}, wherein R^{1P} is the first alcohol protecting group.

In some embodiments, the compound of Formula (I) has the structure of Formula (II-A-27),

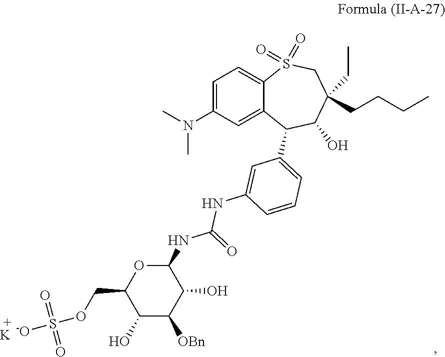

or a pharmaceutically acceptable salt, hydrate, or solvate thereof.

In some embodiments, the compound of Formula (I) has the structure selected from the group consisting of Formula (II-A-123), Formula (II-B-175), Formula (II-C-138), Formula (II-D-95), Formula (II-E-179), Formula (II-F-134), Formula (II-G-76), and Formula (II-H-99),

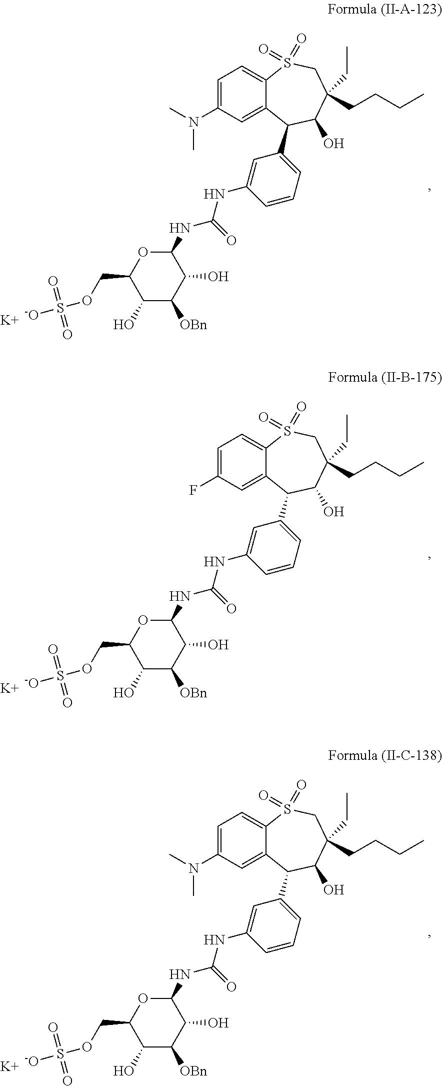

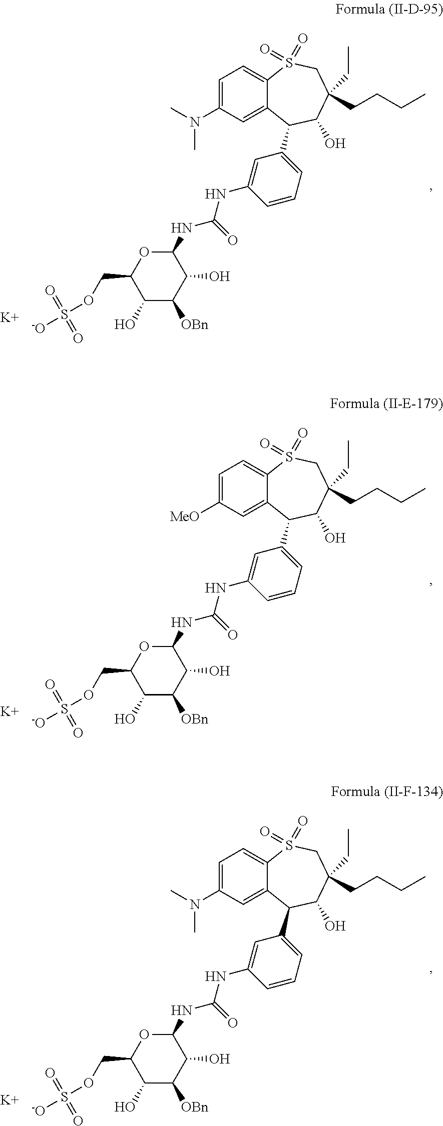

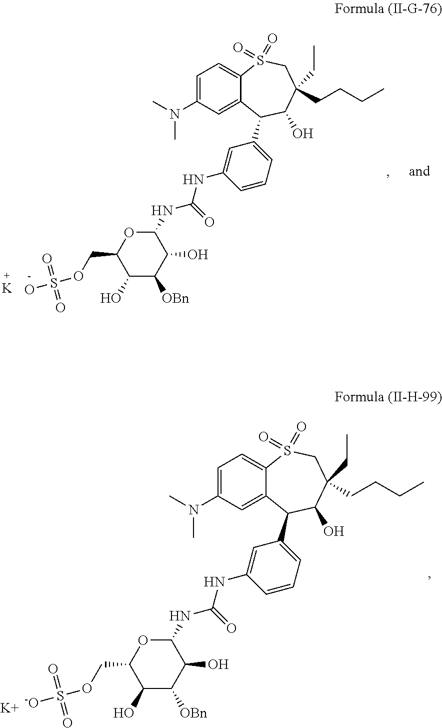

or a pharmaceutically acceptable salt, hydrate, or solvate thereof.

In some embodiments, the compound of Formula (I) has the structure of Formula (II-A-186),

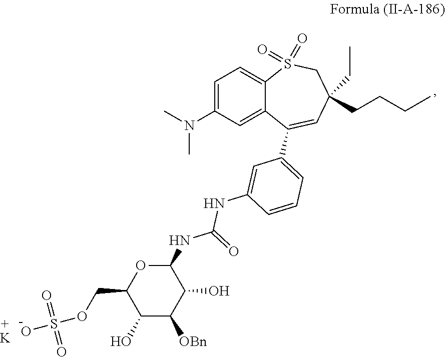

or a pharmaceutically acceptable salt, hydrate, or solvate thereof

In some embodiments, the process further comprises the step of preparing a compound of Formula (I-26),

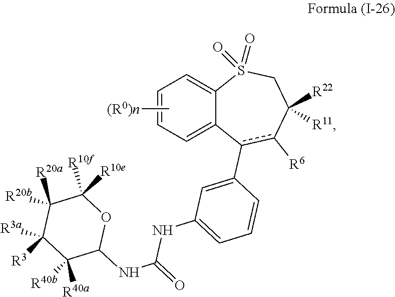

or a pharmaceutically acceptable hydrate or solvate thereof, the process comprising: contacting the compound of Formula (I-25),

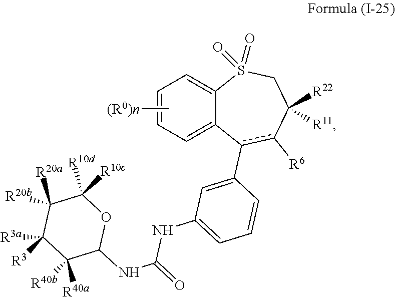

or a pharmaceutically acceptable hydrate or solvate thereof, wherein:
R⁰, R¹¹, R²², R^{10e}, R^{10f}, R^{20a}, R^{20b}, R³, R^{3a}, R^{40a}, R^{40b}, R⁶, and n are defined above;
one of R^{10e} and R^{10d} is C(O)H or an optionally substituted (C₁₋₆)alkylene-OH or —OH and the other of R^{10e} and R^{10d} is H, (C₁₋₆)alkyl, NH₂, NH(C₁₋₆)alkyl, or N((C₁₋₆)alkyl)₂;
with a sulfur trioxide-pyridine complex to produce a compound of Formula (I-26).

In some embodiments, the contacting the compound of Formula (I-25) with the sulfur trioxide-pyridine complex to produce a compound of Formula (I-26) occurs with about two equivalents of the sulfur trioxide-pyridine complex.

In some embodiments, the contacting the compound of Formula (I-25) with the sulfur trioxide-pyridine complex to produce a compound of Formula (I-26) occurs at a reaction temperature of about −10° C.

In some embodiments, the contacting the compound of Formula (I-25) with the sulfur trioxide-pyridine complex to produce a compound of Formula (I-26) occurs in an organic solvent.

In another embodiment, the organic solvent is a polar aprotic solvent.

In an additional embodiment, the polar aprotic solvent is pyridine.

In some embodiments, the process further comprises reaction quenching at a temperature of about −10° C.

In some embodiments, the process further comprises isolating the compound of Formula (I-26).

In some embodiments, isolating the compound of Formula (I-26) comprises recrystallizing the compound of Formula (I-26) from isopropanol, water, isopropyl acetate, or any combination thereof.

In some embodiments, the recrystallizing the compound of Formula (I-26) occurs at a temperature of about 0° C.

In some embodiments, the process further comprises creating a slurry of a wet cake product comprising the compound of Formula (I-26) after recrystallization.

In some embodiments, the creating the slurry of the wet cake product is performed with isopropanol, isopropyl acetate, or a combination thereof.

In another embodiment, R¹⁰ is —CH₂—OH.

In some embodiments, the compound of Formula (I-25) has the structure selected from the group consisting of Formula (I-AA-25), Formula (I-AB-25), Formula (I-BA-25), Formula (I-BB-25), Formula (I-CA-25), Formula (I-CB-25), Formula (I-DA-25), and Formula (I-DB-25),

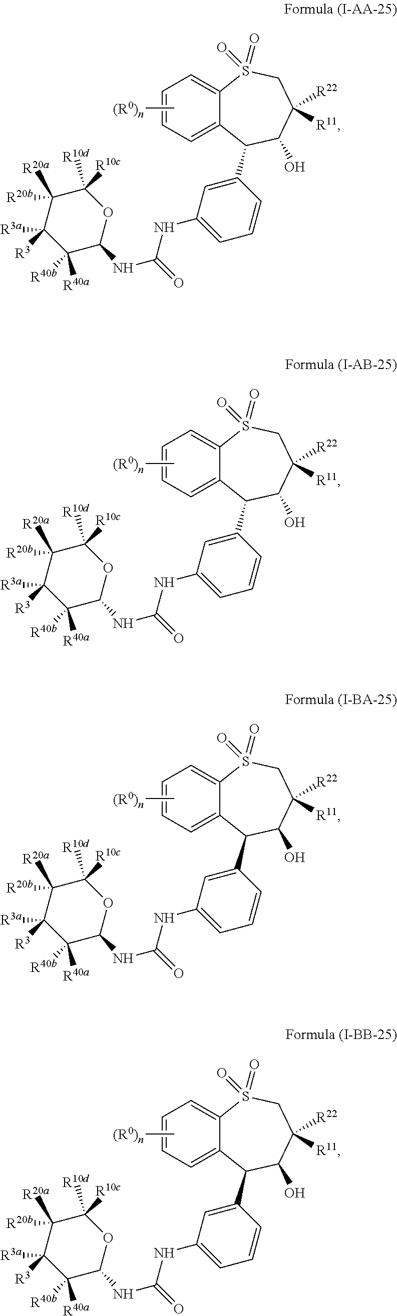

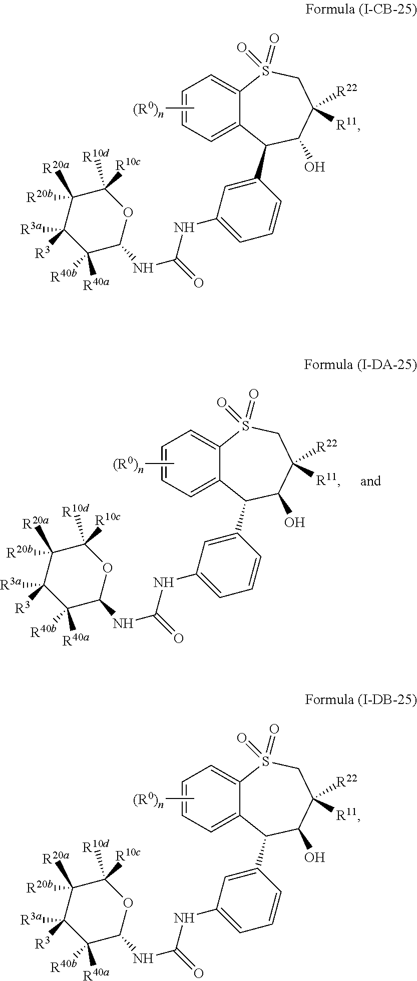

or a pharmaceutically acceptable hydrate or solvate thereof.

In some embodiments, the compound of Formula (I-25) has the structure of Formula (I-A-25),

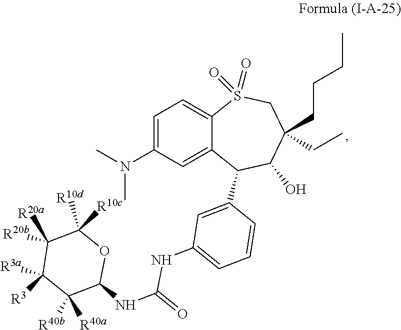

or a pharmaceutically acceptable hydrate or solvate thereof, wherein:
R^{10e} is (C₁₋₆)alkylene-OH or —OH;
each of R^{10d}, R^{20a}, R^{3a}, and R^{40a} is independently H, (C₁₋₆)alkyl, NH₂, NH(C₁₋₆)alkyl, or N((C₁₋₆)alkyl)₂;
R^{20b} is an optionally substituted (C₁₋₆)alkylene-O—R^{2P} or O—R^{2P}, wherein R^{2P} is the second alcohol protecting group;
R^{40b} is an optionally substituted (C₁₋₆)alkylene-O—R^{3P} or O—R^{3P}, wherein R^{3P} is the third alcohol protecting group; and
R³ is an optionally substituted (C₁₋₆)alkylene-O—R^{1P} or —O—R^{1P}, wherein R^{1P} is the first alcohol protecting group.

In some embodiments, the compound of Formula (I-25) has the structure of Formula (II-A-25), Formula (II-A-125), Formula (II-A-136), Formula (II-A-93), Formula (II-A-132), Formula (II-A-74), Formula (II-A-97), Formula (II-A-53), Formula (II-A-177),

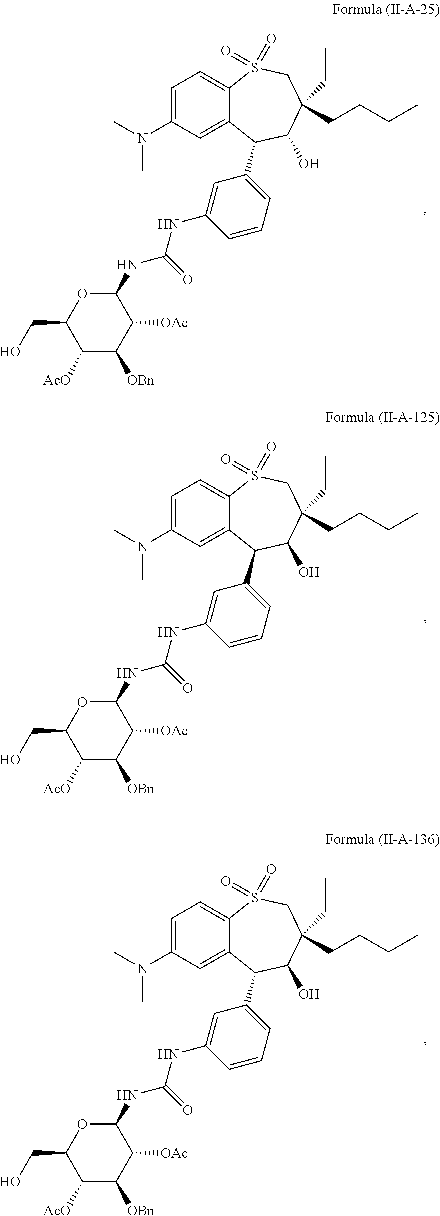 [SMILES: CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@@H]1O]

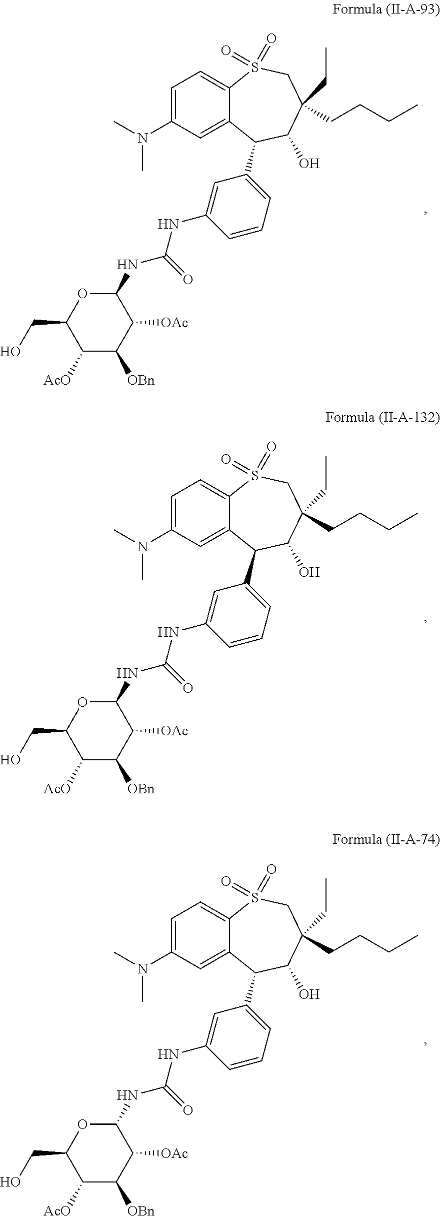 [SMILES: CCCC[C@@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](CO)[C@@H](OC(C)=O)[C@H](OCc4ccccc4)[C@H]3OC(C)=O)c2)[C@H]1O]

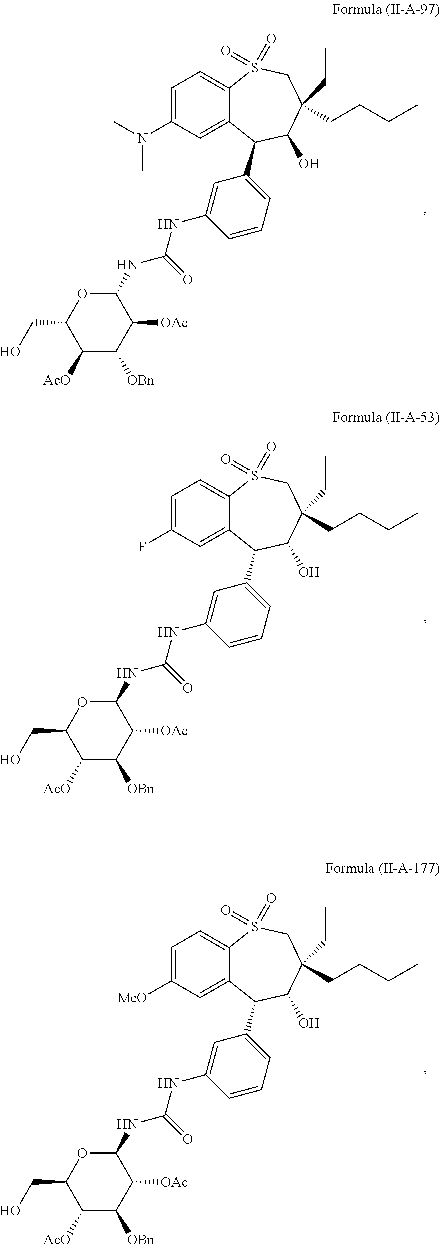 [SMILES: CCCC[C@@]1(CC)CS(=O)(=O)c2ccc(N(C)C)cc2[C@H](c2cccc(NC(=O)N[C@H]3O[C@@H](CO)[C@H](OC(C)=O)[C@@H](OCc4ccccc4)[C@@H]3OC(C)=O)c2)[C@@H]1O ; CCCC[C@]1(CC)CS(=O)(=O)c2ccc(F)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(OC)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

or a pharmaceutically acceptable hydrate or solvate thereof.

In some embodiments, the process further comprises the step of preparing a compound of Formula (I-25), or a pharmaceutically acceptable hydrate or solvate thereof, the process comprising: contacting the compound of Formula (I-20),

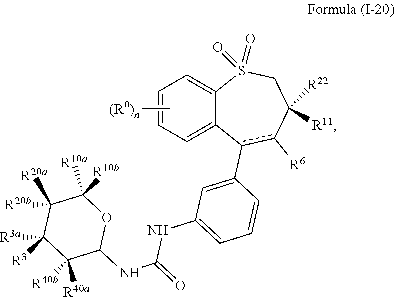

or a pharmaceutically acceptable salt, hydrate, or solvate thereof, wherein:
R⁰, R¹¹, R²², R^{20a}, R^{20b}, R³, R^{3a}, R^{40a}, R^{40b}, R⁶, and n are defined above;
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂;
with a Sn-based catalyst to produce a compound of Formula (I-25).

In some embodiments, the Sn-based catalyst is [tert-Bu₂SnOH(Cl)]₂.

In another embodiment, the contacting the compound of Formula (I-20) with the Sn-based catalyst to produce a compound of Formula (I-25) occurs in the presence of an organic solvent.

In an additional embodiment, the organic solvent is an alcohol, an ether, or a combination thereof.

In another embodiment, the alcohol is methanol.

In some embodiments, the process further comprises reaction quenching with acetic acid.

In some embodiments, the process further comprises performance of a reaction workup by filtration through CUNO.

In some embodiments, the process further comprises isolating the compound of Formula (I-25).

In some embodiments, the isolation of the compound of Formula (I-25) comprises recrystallizing the compound of Formula (I-25) from a second organic solvent.

In another embodiment, the second organic solvent is toluene.

In another embodiment, R^{10b} is —CH₂—O—Ac.

In some embodiments, the compound of Formula (I-20) has the structure selected from the group consisting of Formula (I-AA-20), Formula (I-AB-20), Formula (I-BA-20), Formula (I-BB-20), Formula (I-CA-20), Formula (I-CB-20), Formula (I-DA-20), and Formula (I-DB-20),

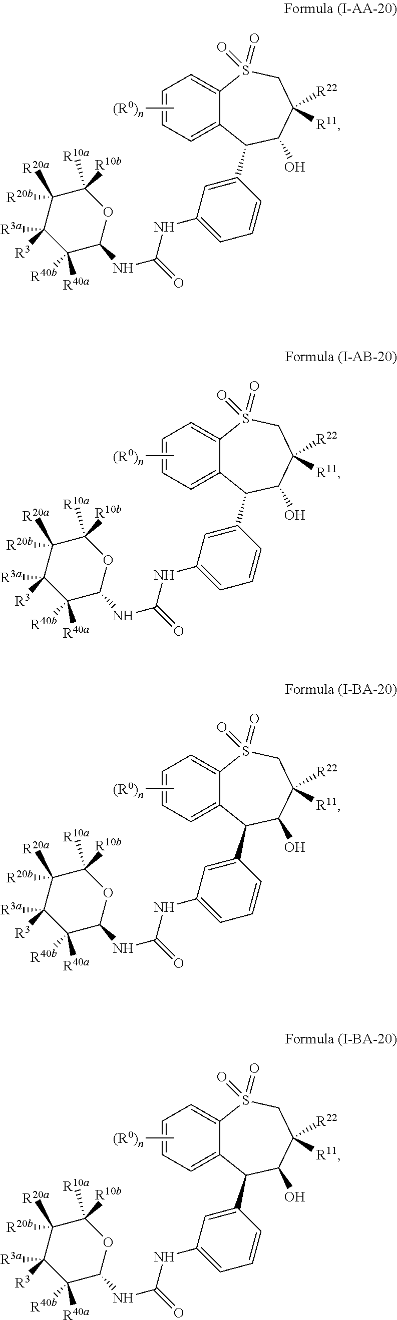

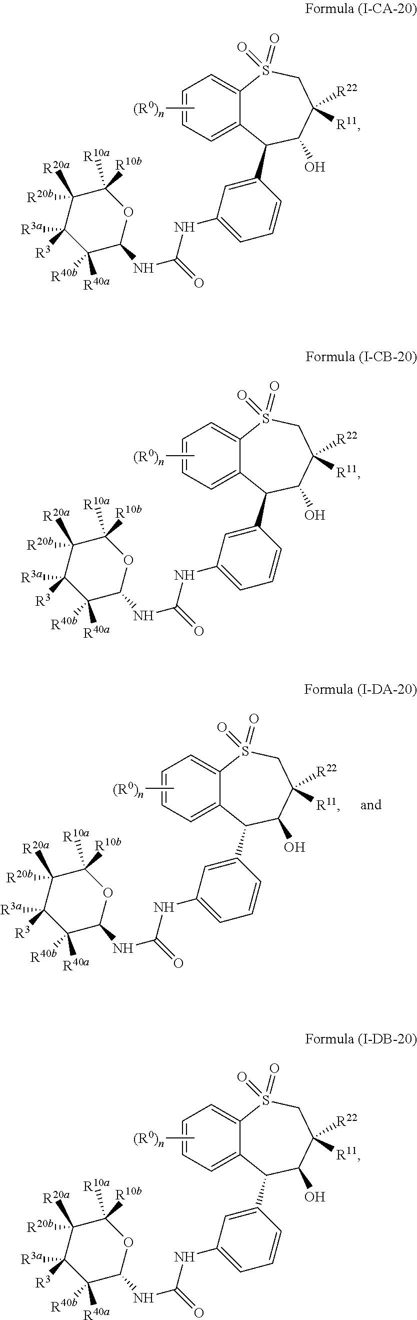

or a pharmaceutically acceptable salt, hydrate, or solvate thereof.

In some embodiments, the compound of Formula (I-20) has the structure of Formula (I-A-20),

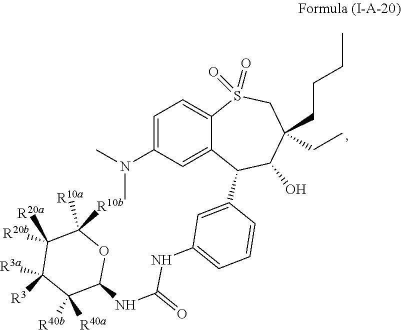

or a pharmaceutically acceptable salt, hydrate, or solvate thereof, wherein:
each of R^{10a}, R^{20a}, R^{3a}, and R^{40a} is independently H, (C₁₋₆)alkyl, NH₂, NH(C₁₋₆)alkyl, or N((C₁₋₆)alkyl)₂;
R³ is an optionally substituted (C₁₋₆)alkylene-O—R^{1P} or —O—R^{1P}, wherein R^{1P} is the first alcohol protecting group;
R^{10b} is an optionally substituted (C₁₋₆)alkylene-O—R^{4P} or O—R^{4P}, wherein R^{4P} is the fourth alcohol protecting group;
R^{20b} is an optionally substituted (C₁₋₆)alkylene-O—R^{2P} or O—R^{2P}, wherein R^{2P} is the second alcohol protecting group;
R^{40b} is an optionally substituted (C₁₋₆)alkylene-O—R^{3P} or O—R^{3P}, wherein R^{3P} is the third alcohol protecting group.

In some embodiments, the compound of Formula (I-20) has the structure of Formula (II-A-20), Formula (II-A-120), Formula (II-A-135), Formula (II-A-92), Formula (II-A-131), Formula (II-A-49), Formula (II-A-96), Formula (II-A-51), Formula (II-A-176),

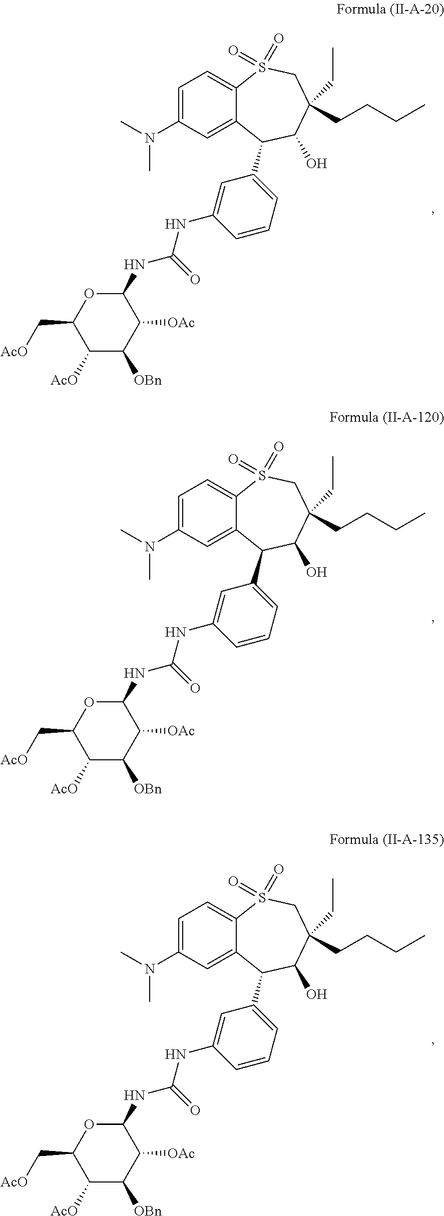 [SMILES: CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O]

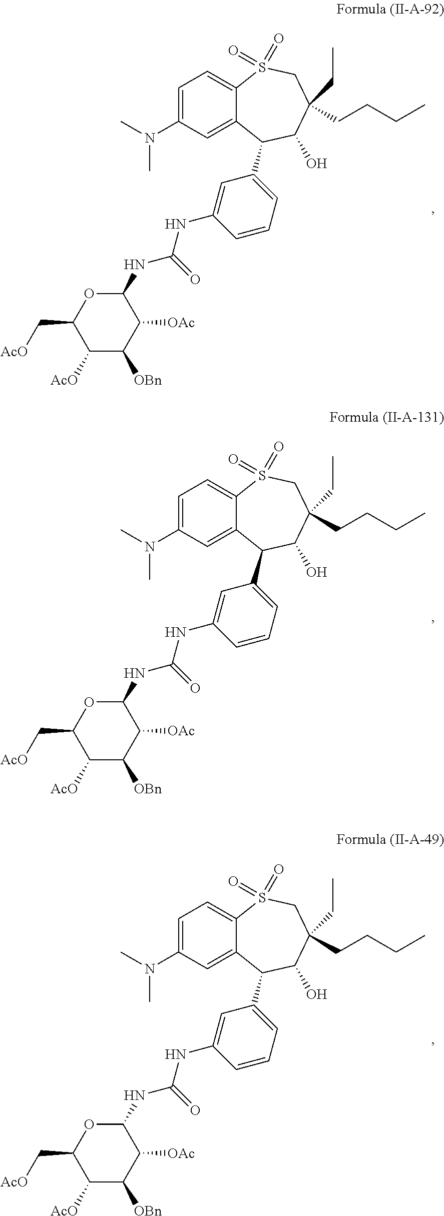 [SMILES: CCCC[C@@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](COC(C)=O)[C@@H](OC(C)=O)[C@H](OCc4ccccc4)[C@H]3OC(C)=O)c2)[C@H]1O]

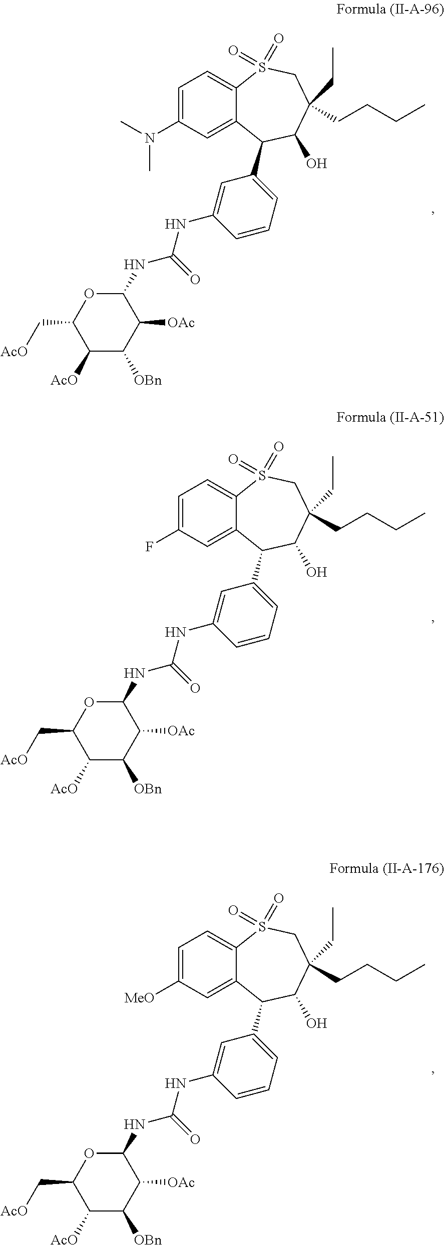 [SMILES: CCCC[C@@]1(CC)CS(=O)(=O)c2ccc(N(C)C)cc2[C@H](c2cccc(NC(=O)N[C@H]3O[C@@H](COC(C)=O)[C@H](OC(C)=O)[C@@H](OCc4ccccc4)[C@@H]3OC(C)=O)c2)[C@@H]1O ; CCCC[C@]1(CC)CS(=O)(=O)c2ccc(F)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(OC)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

or a pharmaceutically acceptable salt, hydrate, or solvate thereof.

Further provided herein is a process of preparing a compound of Formula (II-15),

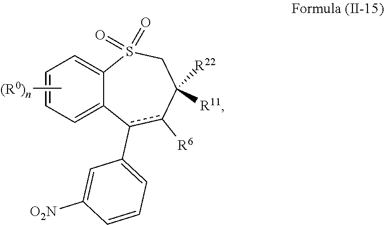

or a pharmaceutically acceptable salt, hydrate, or solvate thereof, the process comprising:
isolating the compound of Formula (II-15) from the mixture represented by Formula (II-15/115),

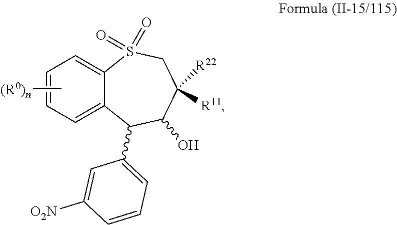

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R²² and R¹¹ is independently an optionally substituted (C₁₋₆)alkyl;
R⁶ is OH; and
n is an integer of 0-4;
and the process further comprising oxidizing and subsequently reducing the compound of Formula (II-15) or the mixture represented by Formula (II-15/115), forming a mixture represented by Formula (II-15/115).

In some embodiments, isolating the compound of Formula (II-15) from the mixture comprises purification via supercritical fluid chromatography (SFC).

In some embodiments, the compound of Formula (II-15) has the structure selected from the group consisting of:

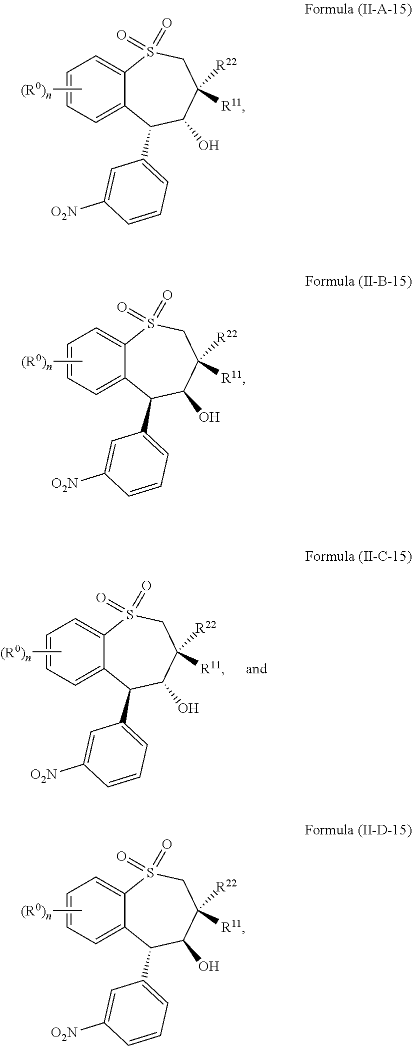

or a pharmaceutically acceptable salt, hydrate, or solvate thereof.

In some embodiments, the compound of Formula (II-15) has the structure selected from the group consisting of:

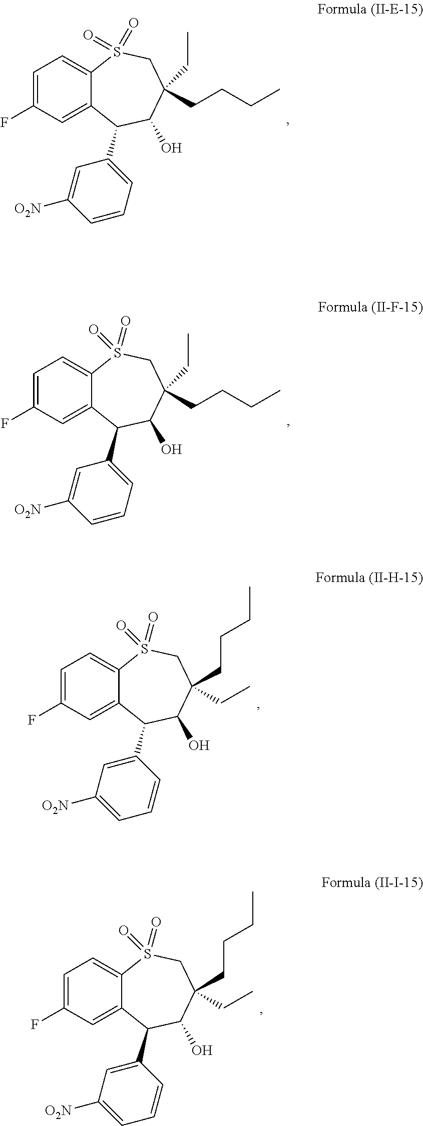 [SMILES: CCCC[C@]1(CC)CS(=O)(=O)c2ccc(F)cc2[C@@H](c2cccc([N+](=O)[O-])c2)[C@H]1O ; CCCC[C@]1(CC)CS(=O)(=O)c2ccc(F)cc2[C@H](c2cccc([N+](=O)[O-])c2)[C@@H]1O ; CCCC[C@]1(CC)CS(=O)(=O)c2ccc(F)cc2[C@@H](c2cccc([N+](=O)[O-])c2)[C@@H]1O ; CCCC[C@]1(CC)CS(=O)(=O)c2ccc(F)cc2[C@H](c2cccc([N+](=O)[O-])c2)[C@H]1O]

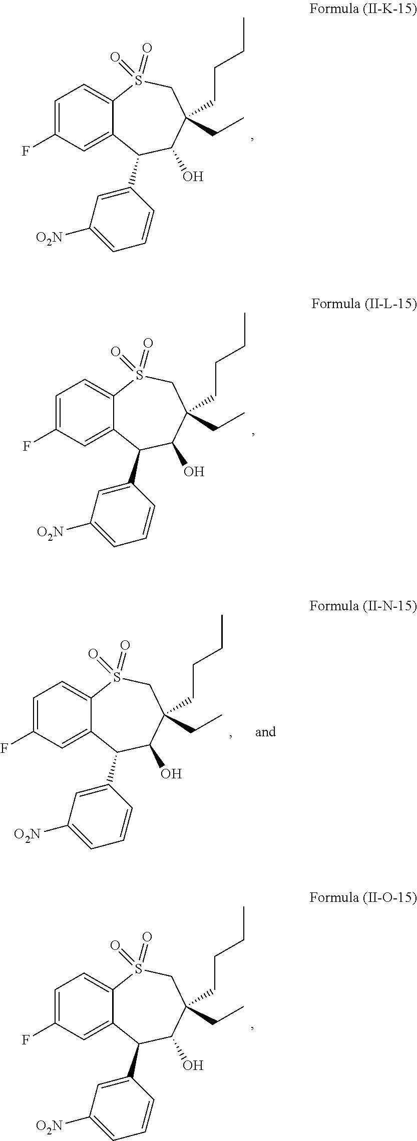 [SMILES: CCCC[C@@]1(CC)CS(=O)(=O)c2ccc(F)cc2[C@@H](c2cccc([N+](=O)[O-])c2)[C@H]1O ; CCCC[C@@]1(CC)CS(=O)(=O)c2ccc(F)cc2[C@H](c2cccc([N+](=O)[O-])c2)[C@@H]1O ; CCCC[C@@]1(CC)CS(=O)(=O)c2ccc(F)cc2[C@@H](c2cccc([N+](=O)[O-])c2)[C@@H]1O ; CCCC[C@@]1(CC)CS(=O)(=O)c2ccc(F)cc2[C@H](c2cccc([N+](=O)[O-])c2)[C@H]1O]

or a pharmaceutically acceptable salt, hydrate, or solvate thereof.

In some embodiments, the mixture is represented by

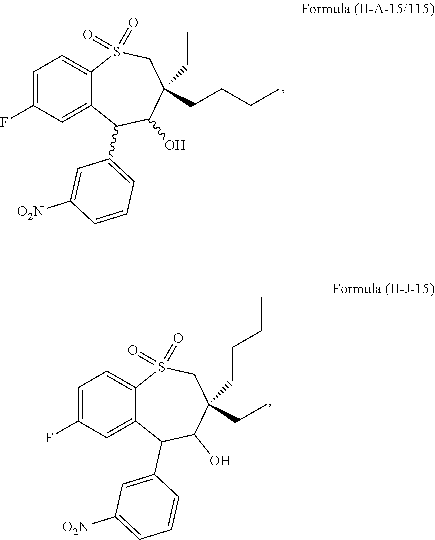 [SMILES: CCCC[C@]1(CC)CS(=O)(=O)c2ccc(F)cc2C(c2cccc([N+](=O)[O-])c2)C1O ; CCCC[C@@]1(CC)CS(=O)(=O)c2ccc(F)cc2C(c2cccc([N+](=O)[O-])c2)C1O]

or a pharmaceutically acceptable salt, hydrate, or solvate thereof.

In some embodiments, the oxidizing occurs with Dess-Martin periodinane.

In some embodiments, the oxidizing occurs in an organic solvent.

In some embodiments, the organic solvent is dichloromethane.

In some embodiments, the oxidizing produces a compound of Formula (II-C-150),

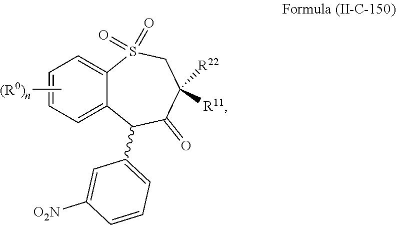

or a pharmaceutically acceptable salt, hydrate, or solvate thereof, wherein:
R⁰, R²², R¹¹, and n are defined above.

In some embodiments, the compound of Formula (II-C-150) has the structure of Formula (II-A-150), Formula (II-B-150),

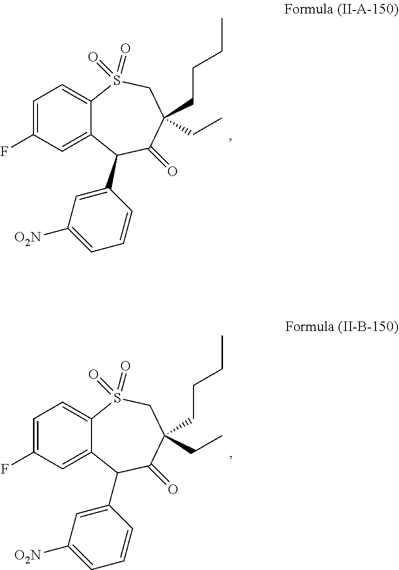 [SMILES: CCCC[C@]1(CC)CS(=O)(=O)c2ccc(F)cc2[C@H](c2cccc([N+](=O)[O-])c2)C1=O ; CCCC[C@@]1(CC)CS(=O)(=O)c2ccc(F)cc2C(c2cccc([N+](=O)[O-])c2)C1=O]

or a pharmaceutically acceptable salt, hydrate, or solvate thereof.

In some embodiments, the reducing occurs with sodium borohydride.

In some embodiments, the reducing occurs in an organic solvent.

In some embodiments, the organic solvent is an ether.

In some embodiments, the ether is tetrahydrofuran.

Further provided herein is a process of preparing a compound of Formula (II-12),

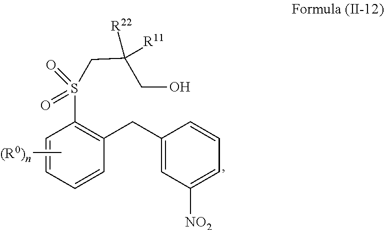

or a pharmaceutically acceptable salt, hydrate, or solvate thereof, the process comprising: contacting the compound of Formula (II-11-int),

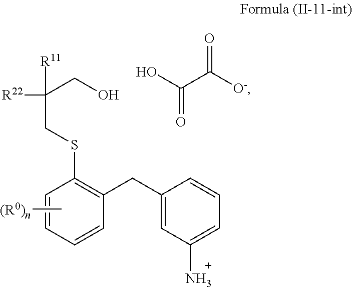

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with an oxidizing reagent in an organic solvent to produce a compound of Formula (II-12).

In some embodiments, the oxidizing reagent is mCPBA.

In some embodiments, the organic solvent is a nonpolar aprotic solvent.

In some embodiments, the nonpolar aprotic solvent is toluene.

In some embodiments, the contacting the compound of Formula (II-11-int) with the oxidizing reagent to produce a compound of Formula (II-12) occurs at a reaction temperature of about 30° C.

In some embodiments, the method further comprises isolating the compound of Formula (II-12) or a pharmaceutically acceptable salt, hydrate, or solvate thereof.

In some embodiments, the isolation of the compound of Formula (II-12) comprises crystallizing the compound of Formula (II-12).

In some embodiments, the compound of Formula (II-12) has the structure of Formula (II-A-12),

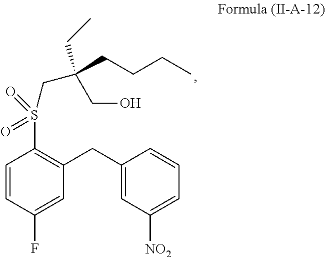 [SMILES: CCCC[C@@](CC)(CO)CS(=O)(=O)c1ccc(F)cc1Cc1cccc([N+](=O)[O-])c1]

or a pharmaceutically acceptable salt, hydrate, or solvate thereof.

In some embodiments, the compound of Formula (II-11-int) has the structure of Formula (II-A-11-int),

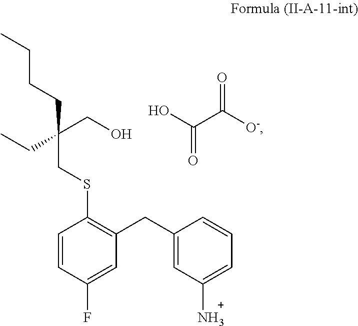

or a pharmaceutically acceptable salt, hydrate, or solvate thereof.

In some embodiments, the method further comprises the step of preparing a compound of Formula (II-11-int),

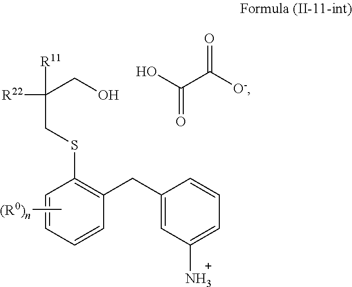

or a pharmaceutically acceptable salt, hydrate, or solvate thereof, the process comprising: contacting the compound of Formula (II-11),

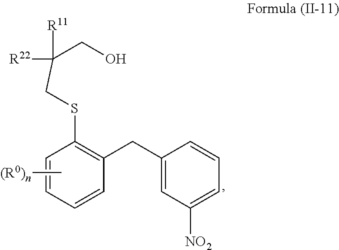

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a reducing agent and oxalic acid in an organic solvent to produce a compound of Formula (II-11-int).

In some embodiments, the reducing agent is Fe/NH₄Cl, H₂ with Pd/C, H₂ with Raney Ni, SnCl₂/NH₄Cl, Zn/AcOH, or Na₂S₂O₄.

In some embodiments, the organic solvent is an alcohol.

In some embodiments, the alcohol is ethanol.

In some embodiments, the organic solvent is a nonpolar aprotic solvent.

In some embodiments, the nonpolar aprotic solvent is toluene.

In some embodiments, the organic solvent is an ether.

In some embodiments, the ether is MTBE.

In some embodiments, the contacting the compound of Formula (II-11) with the reducing agent and oxalic acid to produce a compound of Formula (II-11-int) occurs at a reaction temperature of about 80° C.

In some embodiments, the compound of Formula (II-11-int) has the structure of Formula (II-A-11-int),

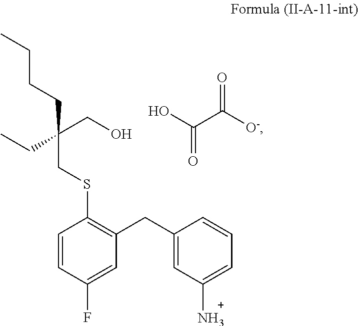

or a pharmaceutically acceptable salt, hydrate, or solvate thereof.

In some embodiments, the compound of Formula (II-11) has the structure of Formula (II-A-11),

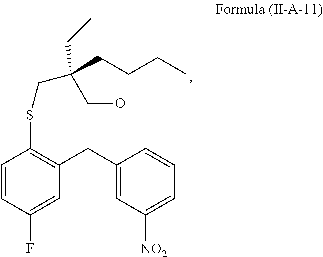 [SMILES: CCCC[C@@](CC)(CO)CSc1ccc(F)cc1Cc1cccc([N+](=O)[O-])c1]

or a pharmaceutically acceptable salt, hydrate, or solvate thereof.

Additionally provided herein is a process of preparing a compound of Formula (II-11),

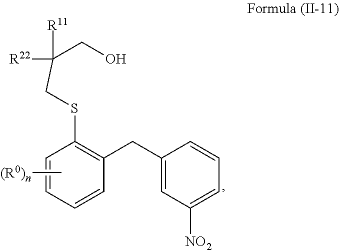

or a pharmaceutically acceptable salt, hydrate, or solvate thereof, the process comprising:
contacting the compound of Formula (II-215),

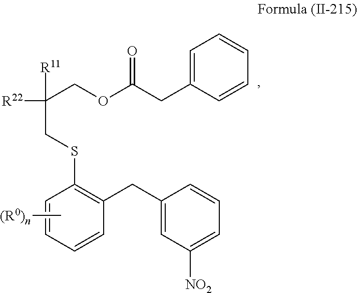

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-11).

In some embodiments, the base is LiOH, NaOH, or KOH.

In some embodiments, the organic solvent is a polar aprotic solvent.

In some embodiments, the organic solvent is an ether.

In some embodiments, the ether is THF.

In some embodiments, the contacting the compound of Formula (II-215) with the base to produce a compound of Formula (II-11) occurs at a reaction temperature of about 80° C.

In some embodiments, the method further comprises isolating the compound of Formula (II-11) or a pharmaceutically acceptable salt, hydrate, or solvate thereof.

In some embodiments, the compound of Formula (II-11) has the structure of Formula (II-A-11),

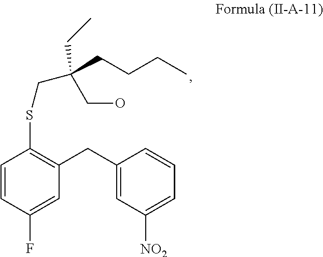 [SMILES: CCCC[C@@](CC)(CO)CSc1ccc(F)cc1Cc1cccc([N+](=O)[O-])c1]

or a pharmaceutically acceptable salt, hydrate, or solvate thereof.

In some embodiments, the compound of Formula (II-215) has the structure of Formula (II-A-215),

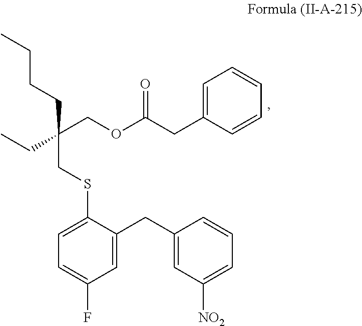 [SMILES: CCCC[C@@](CC)(COC(=O)Cc1ccccc1)CSc1ccc(F)cc1Cc1cccc([N+](=O)[O-])c1]

or a pharmaceutically acceptable salt, hydrate, or solvate thereof.

In some embodiments, the method further comprises the step of preparing a compound of Formula (II-215),

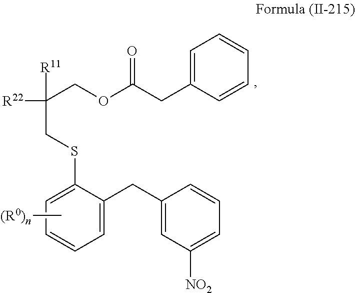

or a pharmaceutically acceptable salt, hydrate, or solvate thereof, the process comprising: contacting the compounds of Formula (II-210),

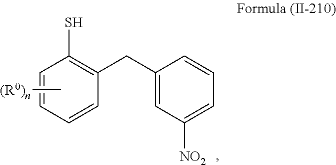

or a pharmaceutically acceptable salt, hydrate, or solvate thereof, and Formula (II-33),

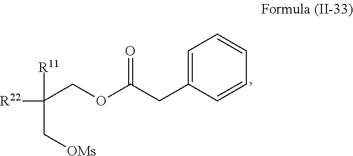

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-215).

In some embodiments, the base is NaH, LDA, NaHMDS, or KHMDS.

In some embodiments, the organic solvent is a polar aprotic solvent.

In some embodiments, the polar aprotic solvent is NMP.

In some embodiments, the contacting the compounds of Formula (II-210) and Formula (II-33) with the base to produce a compound of Formula (II-215) occurs at a reaction temperature of about 60° C.

In some embodiments, the method further comprises isolating the compound of Formula (II-215) or a pharmaceutically acceptable salt, hydrate, or solvate thereof.

In some embodiments, the compound of Formula (II-215) has the structure of Formula (II-A-215),

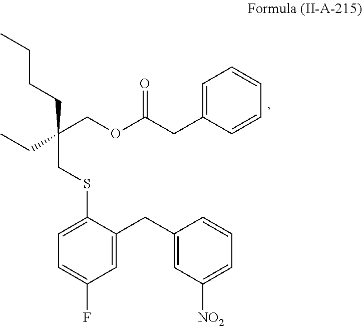 [SMILES: CCCC[C@@](CC)(COC(=O)Cc1ccccc1)CSc1ccc(F)cc1Cc1cccc([N+](=O)[O-])c1]

or a pharmaceutically acceptable salt, hydrate, or solvate thereof.

In some embodiments, the compound of Formula (II-210) has the structure of Formula (II-A-210),

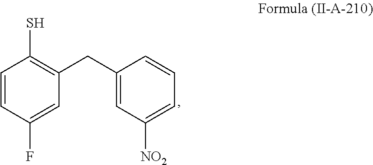 [SMILES: O=[N+]([O-])c1cccc(Cc2cc(F)ccc2S)c1]

or a pharmaceutically acceptable salt, hydrate, or solvate thereof.

In some embodiments, the compound of Formula (II-33) has the structure of Formula (II-A-33),

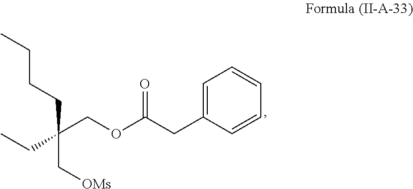 [SMILES: CCCC[C@@](CC)(COC(=O)Cc1ccccc1)COS(C)(=O)=O]

or a pharmaceutically acceptable salt, hydrate, or solvate thereof.

In some embodiments, the method further comprises the step of preparing a compound of Formula (II-210),

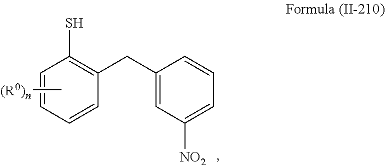

or a pharmaceutically acceptable salt, hydrate, or solvate thereof, the process comprising: contacting the compound of Formula (II-214),

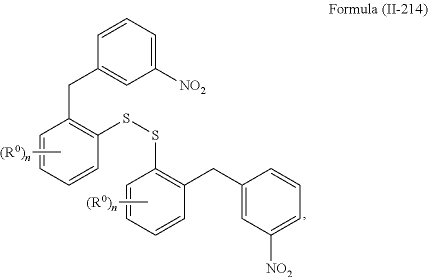

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a reducing agent in an organic solvent to produce a compound of Formula (II-210).

In some embodiments, the reducing agent is NaBH₄.

In some embodiments, the organic solvent is an ether.

In some embodiments, the ether is THF.

In some embodiments, the contacting the compound of Formula (II-214) with the reducing agent to produce a compound of Formula (II-210) occurs at a reaction temperature of about 25° C.

In some embodiments, the compound of Formula (II-210) has the structure of Formula (II-A-210),

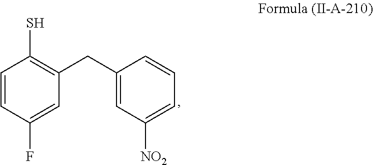 [SMILES: O=[N+]([O-])c1cccc(Cc2cc(F)ccc2S)c1]

or a pharmaceutically acceptable salt, hydrate, or solvate thereof.

In some embodiments, the compound of Formula (II-214) has the structure of Formula (II-A-214),

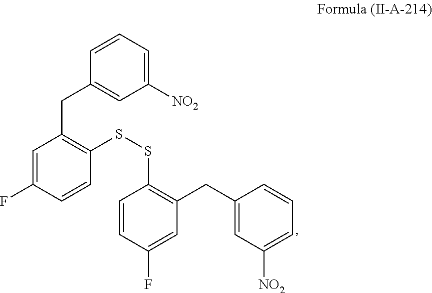 [SMILES: O=[N+]([O-])c1cccc(Cc2cc(F)ccc2SSc2ccc(F)cc2Cc2cccc([N+](=O)[O-])c2)c1]

or a pharmaceutically acceptable salt, hydrate, or solvate thereof.

In some embodiments, the method further comprises the step of preparing a compound of Formula (II-210),

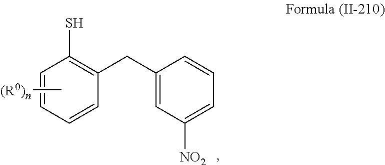

or a pharmaceutically acceptable salt, hydrate, or solvate thereof, the process comprising: contacting the compound of Formula (II-205),

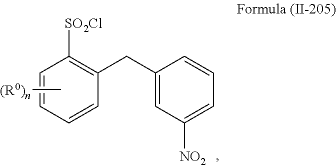

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with an organophosphorus compound in an organic solvent to produce a compound of Formula (II-210).

In some embodiments, the organophosphorus compound is PPh₃.

In some embodiments, the organic solvent is a nonpolar aprotic solvent.

In some embodiments, the nonpolar aprotic solvent is toluene.

In some embodiments, the contacting the compound of Formula (II-205) with the organophosphorus compound to produce a compound of Formula (II-210) occurs at a reaction temperature of about 60° C.

In some embodiments, the compound of Formula (II-210) has the structure of Formula (II-A-210),

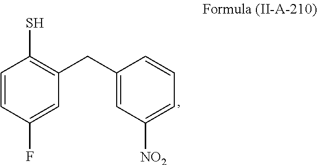 [SMILES: O=[N+]([O-])c1cccc(Cc2cc(F)ccc2S)c1]

or a pharmaceutically acceptable salt, hydrate, or solvate thereof.

In some embodiments, the compound of Formula (II-205) has the structure of Formula (II-A-205),

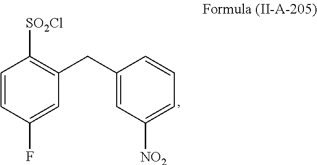 [SMILES: O=[N+]([O-])c1cccc(Cc2cc(F)ccc2S(=O)(=O)Cl)c1]

or a pharmaceutically acceptable salt, hydrate, or solvate thereof.

In some embodiments, the method further comprises the step of preparing a compound of Formula (II-214),

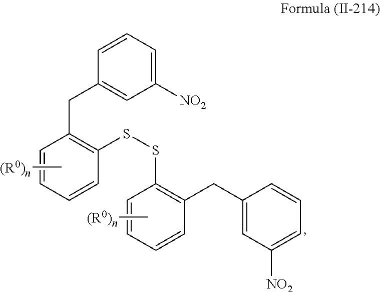

or a pharmaceutically acceptable salt, hydrate, or solvate thereof, the process comprising: contacting the compound of Formula (II-204),

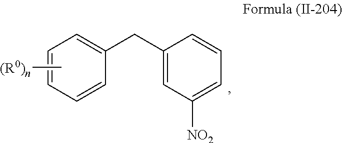

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a sulfurizing agent and a Lewis acid in an organic solvent to produce a compound of Formula (II-214).

In some embodiments, the sulfurizing agent is S₂Cl₂.

In some embodiments, the Lewis acid is FeCl₃.

In some embodiments, the organic solvent is DCM.

In some embodiments, the contacting the compound of Formula (II-204) with the sulfurizing agent to produce a compound of Formula (II-214) occurs at a reaction temperature of about 25° C.

In some embodiments, the compound of Formula (II-214) has the structure of Formula (II-A-214),

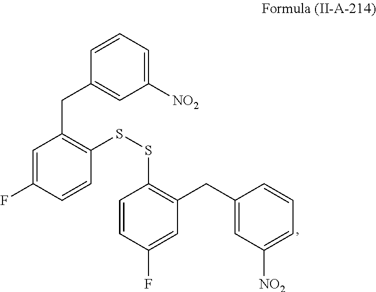 [SMILES: O=[N+]([O-])c1cccc(Cc2cc(F)ccc2SSc2ccc(F)cc2Cc2cccc([N+](=O)[O-])c2)c1]

or a pharmaceutically acceptable salt, hydrate, or solvate thereof.

In some embodiments, the compound of Formula (II-204) has the structure of Formula (II-A-204),

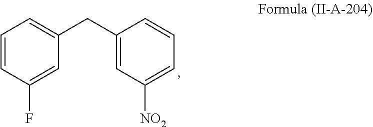 [SMILES: O=[N+]([O-])c1cccc(Cc2cccc(F)c2)c1]

or a pharmaceutically acceptable salt, hydrate, or solvate thereof.

In some embodiments, the method further comprises the step of preparing a compound of Formula (II-214),

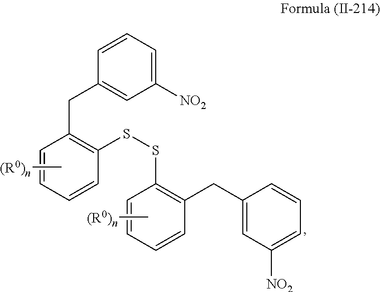

or a pharmaceutically acceptable salt, hydrate, or solvate thereof, the process comprising: contacting the compound of Formula (II-205),

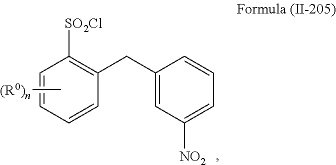

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a sulfite to produce a compound of Formula (II-214).

In some embodiments, the sulfite is Na₂S₂O₅ or Na₂SO₃.

In some embodiments, the method further comprises addition of KI.

In some embodiments, the method further comprises addition of HOAc.

In some embodiments, the contacting the compound of Formula (II-205) with the sulfite to produce a compound of Formula (II-214) occurs at a reaction temperature of about 105° C.

In some embodiments, the method further comprises isolating the compound of Formula (II-214) or a pharmaceutically acceptable salt, hydrate, or solvate thereof.

In some embodiments, the isolation of the compound of Formula (II-214) comprises recrystallizing the compound of Formula (II-214) from toluene and n-heptane.

In some embodiments, the compound of Formula (II-214) has the structure of Formula (II-A-214),

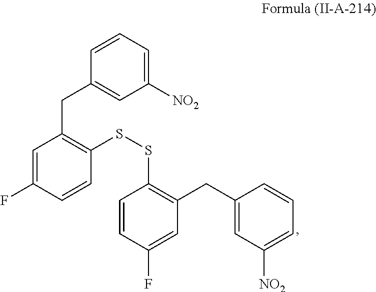 [SMILES: O=[N+]([O-])c1cccc(Cc2cc(F)ccc2SSc2ccc(F)cc2Cc2cccc([N+](=O)[O-])c2)c1]

or a pharmaceutically acceptable salt, hydrate, or solvate thereof.

In some embodiments, the compound of Formula (II-205) has the structure of Formula (II-A-205),

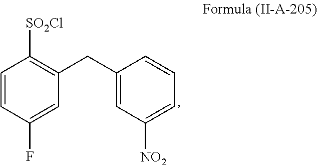 [SMILES: O=[N+]([O-])c1cccc(Cc2cc(F)ccc2S(=O)(=O)Cl)c1]

or a pharmaceutically acceptable salt, hydrate, or solvate thereof.

In some embodiments, the method further comprises the step of preparing a compound of Formula (II-205),

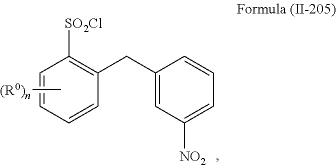

or a pharmaceutically acceptable salt, hydrate, or solvate thereof, the process comprising: contacting the compound of Formula (II-204),

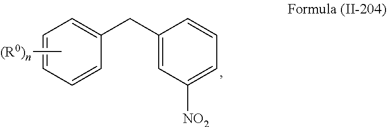

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a sulfonic acid in an organic solvent to produce a compound of Formula (II-205).

In some embodiments, the sulfonic acid is chlorosulfonic acid.

In some embodiments, the organic solvent is DCM.

In some embodiments, the contacting the compound of Formula (II-204) with the sulfonic acid to produce a compound of Formula (II-205) occurs at a reaction temperature of about 25° C. or about 45° C.

In some embodiments, the compound of Formula (II-205) has the structure of Formula (II-A-205),

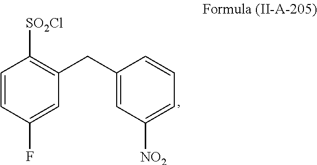 [SMILES: O=[N+]([O-])c1cccc(Cc2cc(F)ccc2S(=O)(=O)Cl)c1]

or a pharmaceutically acceptable salt, hydrate, or solvate thereof.

In some embodiments, the compound of Formula (II-204) has the structure of Formula (II-A-204),

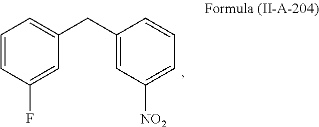 [SMILES: O=[N+]([O-])c1cccc(Cc2cccc(F)c2)c1]

or a pharmaceutically acceptable salt, hydrate, or solvate thereof.

In some embodiments, the method further comprises the step of preparing a compound of Formula (II-205),

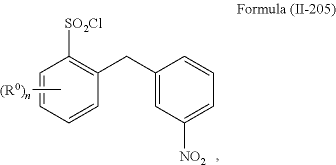

or a pharmaceutically acceptable salt, hydrate, or solvate thereof, the process comprising: contacting the compound of Formula (II-209),

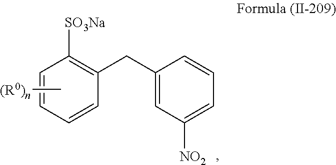

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a chlorinating agent in an organic solvent to produce a compound of Formula (II-205).

In some embodiments, the chlorinating agent is SOCl₂, POCl₃, or PCl₅.

In some embodiments, the organic solvent is DMF.

In some embodiments, the contacting the compound of Formula (II-209) with the chlorinating agent to produce a compound of Formula (II-205) occurs at a reaction temperature of about 60° C.

In some embodiments, the compound of Formula (II-205) has the structure of Formula (II-A-205),

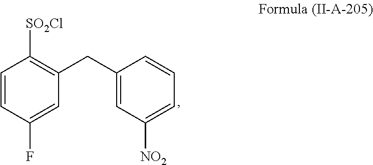 [SMILES: O=[N+]([O-])c1cccc(Cc2cc(F)ccc2S(=O)(=O)Cl)c1]

or a pharmaceutically acceptable salt, hydrate, or solvate thereof.

In some embodiments, the compound of Formula (II-209) has the structure of Formula (II-A-209),

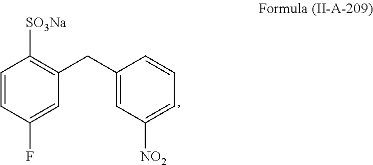

or a pharmaceutically acceptable salt, hydrate, or solvate thereof.

In some embodiments, the method further comprises the step of preparing a compound of Formula (II-209),

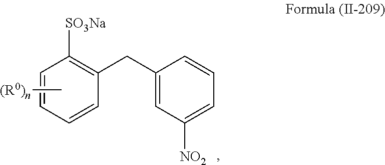

or a pharmaceutically acceptable salt, hydrate, or solvate thereof, the process comprising: contacting the compound of Formula (II-204),

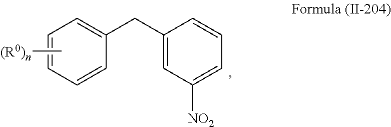

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with sulfuric acid to produce a compound of Formula (II-209).

In some embodiments, the sulfuric acid is concentrated sulfuric acid and fuming sulfuric acid.

In some embodiments, the method further comprises isolating the compound of Formula (II-209) or a pharmaceutically acceptable salt, hydrate, or solvate thereof.

In some embodiments, the isolation of the compound of Formula (II-209) comprises crystallizing the compound of Formula (II-209).

In some embodiments, the contacting the compound of Formula (II-204) with the sulfuric acid to produce a compound of Formula (II-209) occurs at a reaction temperature of about 20° C. or about 40° C.

In some embodiments, the compound of Formula (II-209) has the structure of Formula (II-A-209),

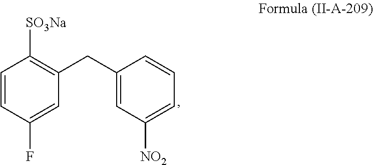

or a pharmaceutically acceptable salt, hydrate, or solvate thereof.

In some embodiments, the compound of Formula (II-204) has the structure of Formula (II-A-204),

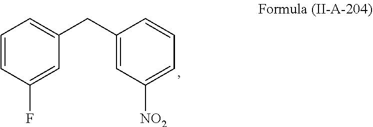 [SMILES: O=[N+]([O-])c1cccc(Cc2cccc(F)c2)c1]

or a pharmaceutically acceptable salt, hydrate, or solvate thereof.

In some embodiments, the method further comprises the step of preparing a compound of Formula (II-204),

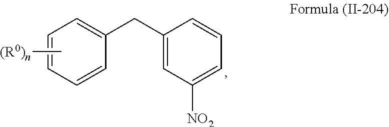

or a pharmaceutically acceptable salt, hydrate, or solvate thereof, the process comprising: contacting the compounds of Formula (II-203),

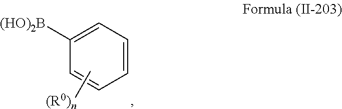

or a pharmaceutically acceptable salt, hydrate, or solvate thereof, and Formula (II-202),

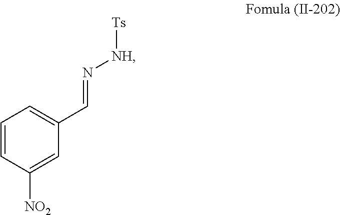 [SMILES: Cc1ccc(S(=O)(=O)N/N=C/c2cccc([N+](=O)[O-])c2)cc1]

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-204).

In some embodiments, the base is K₂CO₃ or NaOH.

In some embodiments, the organic solvent is MeTHF, CPME, toluene, anisole, or 1,4-dioxane.

In some embodiments, the contacting the compound of Formula (II-203) and Formula (II-202) with the base to produce a compound of Formula (II-204) occurs at a reaction temperature of about 85° C.

In some embodiments, the compound of Formula (II-204) has the structure of Formula (II-A-204),

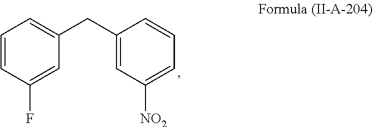 [SMILES: O=[N+]([O-])c1cccc(Cc2cccc(F)c2)c1]

or a pharmaceutically acceptable salt, hydrate, or solvate thereof.

In some embodiments, the compound of Formula (II-203) has the structure of Formula (II-A-203),

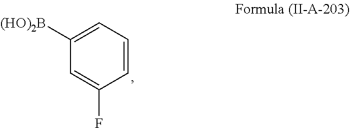

or a pharmaceutically acceptable salt, hydrate, or solvate thereof.

In some embodiments, the method further comprises the step of preparing a compound of Formula (II-202),

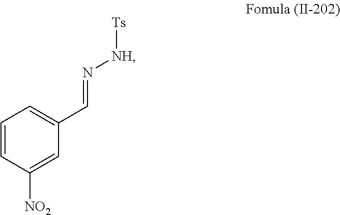 [SMILES: Cc1ccc(S(=O)(=O)N/N=C/c2cccc([N+](=O)[O-])c2)cc1]

or a pharmaceutically acceptable salt, hydrate, or solvate thereof, the process comprising: contacting the compound of Formula (II-201),

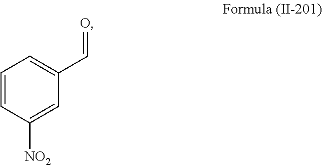 [SMILES: O=Cc1cccc([N+](=O)[O-])c1]

or a pharmaceutically acceptable salt, hydrate, or solvate thereof, with a hydrazide in an organic solvent to produce a compound of Formula (II-202).

In some embodiments, the hydrazide is tosyl hydrazide.

In some embodiments, the organic solvent is MeTHF.

In some embodiments, the contacting the compound of Formula (II-201) with the hydrazide to produce a compound of Formula (II-202) occurs at a reaction temperature of about 60° C.

In some embodiments, the method further comprises the step of preparing a compound of Formula (II-204),

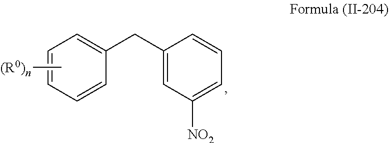

or a pharmaceutically acceptable salt, hydrate, or solvate thereof, the process comprising: contacting the compounds of Formula (II-203),

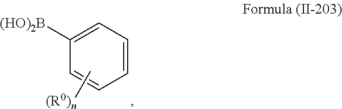

or a pharmaceutically acceptable salt, hydrate, or solvate thereof, and Formula (II-229),

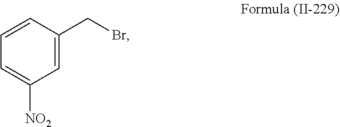 [SMILES: O=[N+]([O-])c1cccc(CBr)c1]

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a palladium catalyst, a ligand, and a base in a solvent to produce a compound of Formula (II-204).

In some embodiments, the palladium catalyst is Pd(OAc)₂.

In some embodiments, the ligand is PCy₃, PPh₃, dppf, Brettphos, XantPhos, SPhos, RuPhos, XPhos, P(tBu)₃, or tBuBrettPhos.

In some embodiments, the base is K₃PO₄, K₂CO₃, NaOH, NaHCO₃, Et₃N, or DIPEA.

In some embodiments, the solvent is THF, toluene, 2-MeTHF, MIBK, IPA, n-Butanol, water, or any mixture thereof.

In some embodiments, the contacting the compounds of Formula (II-203) and Formula (II-229) with the palladium catalyst and the ligand to produce a compound of Formula (II-204) occurs at a reaction temperature of about 65° C.

In some embodiments, the compound of Formula (II-204) has the structure of Formula (II-A-204),

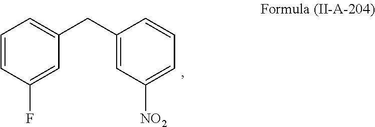 [SMILES: O=[N+]([O-])c1cccc(Cc2cccc(F)c2)c1]

or a pharmaceutically acceptable salt, hydrate, or solvate thereof.

In some embodiments, the compound of Formula (II-203) has the structure of Formula (II-A-203),

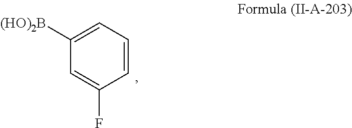

or a pharmaceutically acceptable salt, hydrate, or solvate thereof.

Further provided herein is a process of preparing a compound of Formula (II-11),

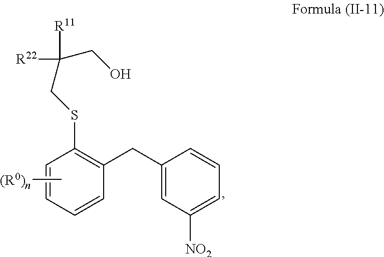

or a pharmaceutically acceptable salt, hydrate, or solvate thereof, the process comprising: contacting the compounds of Formula (II-208),

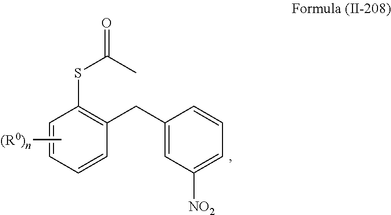

or a pharmaceutically acceptable salt, hydrate, or solvate thereof, and Formula (II-33),

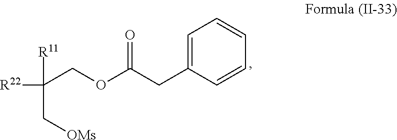

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a base in an organic solvent to produce a compound of Formula (II-11).

In some embodiments, the base is K₂CO₃, Cs₂CO₃, NaOMe, K₃PO₄, or NH₄OH.

In some embodiments, the organic solvent is a polar aprotic solvent.

In some embodiments, the polar aprotic solvent is NMP.

In some embodiments, the organic solvent is an alcohol.

In some embodiments, the alcohol is methanol.

In some embodiments, the organic solvent is a mixture of a polar aprotic solvent and an alcohol.

In some embodiments, the contacting the compounds of Formula (II-208) and Formula (II-33) with the base to produce a compound of Formula (II-11) occurs at a reaction temperature of about 85° C.

In some embodiments, the method further comprises isolating the compound of Formula (II-11) or a pharmaceutically acceptable salt, hydrate, or solvate thereof.

In some embodiments, the compound of Formula (II-11) has the structure of Formula (II-A-11),

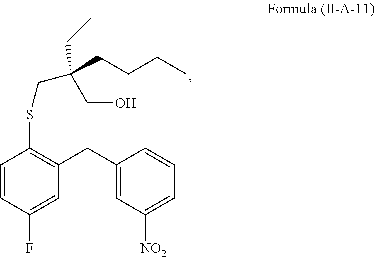 [SMILES: CCCC[C@@](CC)(CO)CSc1ccc(F)cc1Cc1cccc([N+](=O)[O-])c1]

or a pharmaceutically acceptable salt, hydrate, or solvate thereof.

In some embodiments, the compound of Formula (II-33) has the structure of Formula (II-A-33),

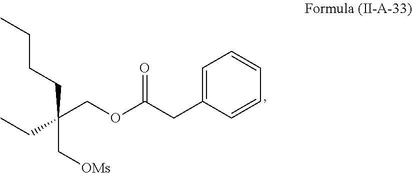 [SMILES: CCCC[C@@](CC)(COC(=O)Cc1ccccc1)COS(C)(=O)=O]

or a pharmaceutically acceptable salt, hydrate, or solvate thereof.

In some embodiments, the compound of Formula (II-208) has the structure of Formula (II-A-208),

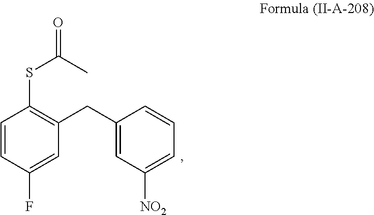 [SMILES: CC(=O)Sc1ccc(F)cc1Cc1cccc([N+](=O)[O-])c1]

or a pharmaceutically acceptable salt, hydrate, or solvate thereof.

In some embodiments, the method further comprises the step of preparing a compound of Formula (II-208),

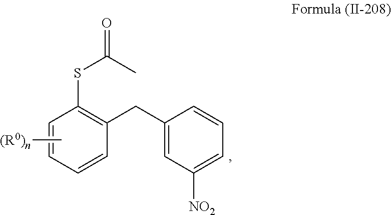

or a pharmaceutically acceptable salt, hydrate, or solvate thereof, the process comprising: contacting the compound of Formula (II-207),

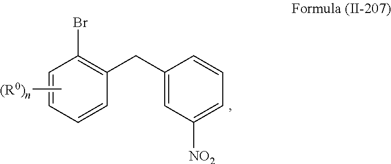

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a palladium catalyst, a ligand, and a thioacetate in an organic solvent to produce a compound of Formula (II-208).

In some embodiments, the palladium catalyst is Pd₂(dba)₃.

In some embodiments, the ligand is XPhos, CyPFtBu, XantPhos, DavePhos, SPhos, tBuXPhos, JohnPhos, or RuPhos.

In some embodiments, the thioacetate is KSAc.

In some embodiments, the organic solvent is a nonpolar aprotic solvent.

In some embodiments, the nonpolar aprotic solvent is toluene.

In some embodiments, the contacting the compound of Formula (II-207) with the palladium catalyst and the ligand to produce a compound of Formula (II-208) occurs at a reaction temperature of about 110° C.

In some embodiments, the method further comprises isolating the compound of Formula (II-208) or a pharmaceutically acceptable salt, hydrate, or solvate thereof.

In some embodiments, the compound of Formula (II-208) has the structure of Formula (II-A-208),

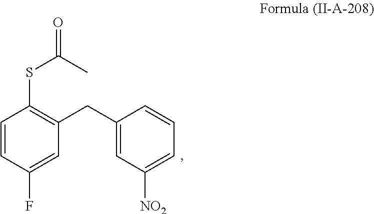 [SMILES: CC(=O)Sc1ccc(F)cc1Cc1cccc([N+](=O)[O-])c1]

or a pharmaceutically acceptable salt, hydrate, or solvate thereof.

In some embodiments, the compound of Formula (II-207) has the structure of Formula (II-A-207),

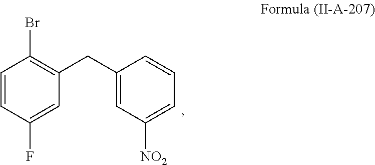 [SMILES: O=[N+]([O-])c1cccc(Cc2cc(F)ccc2Br)c1]

or a pharmaceutically acceptable salt, hydrate, or solvate thereof.

In some embodiments, the method further comprises the step of preparing a compound of Formula (II-207),

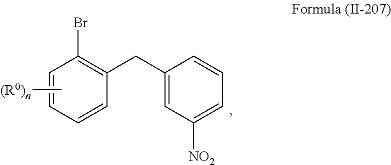

or a pharmaceutically acceptable salt, hydrate, or solvate thereof, the process comprising: contacting the compounds of Formula (II-206),

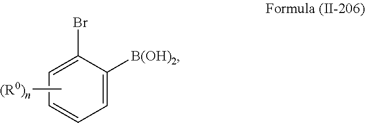

or a pharmaceutically acceptable salt, hydrate, or solvate thereof, and Formula (II-229),

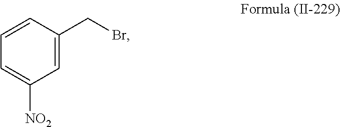 [SMILES: O=[N+]([O-])c1cccc(CBr)c1]

or a pharmaceutically acceptable salt, hydrate, or solvate thereof, wherein:
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
with a palladium catalyst, a ligand, and a base in a solvent to produce a compound of Formula (II-207).

In some embodiments, the palladium catalyst is PdCl₂.

In some embodiments, the ligand is dppf, PPh₃, Brettphos, XantPhos, SPhos, RuPhos, XPhos, P(tBu)₃, or tBuBrettPhos.

In some embodiments, the base is K₃PO₄, K₂CO₃, NaOH, NaHCO₃, Et₃N, or DIPEA.

In some embodiments, the solvent is 1,4-dioxane, THF, toluene, 2-MeTHF, MIBK, IPA, n-Butanol, water, or any mixture thereof.

In some embodiments, the contacting the compounds of Formula (II-206) and Formula (II-229) with the palladium catalyst and the ligand to produce a compound of Formula (II-207) occurs at a reaction temperature of about 90° C.

In some embodiments, the compound of Formula (II-207) has the structure of Formula (II-A-207),

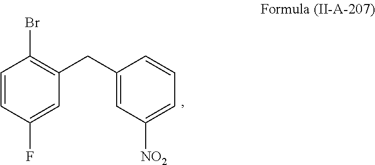 [SMILES: O=[N+]([O-])c1cccc(Cc2cc(F)ccc2Br)c1]

or a pharmaceutically acceptable salt, hydrate, or solvate thereof.

In some embodiments, the compound of Formula (II-206) has the structure of Formula (II-A-206),

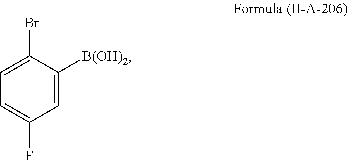 [SMILES: OB(O)c1cc(F)ccc1Br]

or a pharmaceutically acceptable salt, hydrate, or solvate thereof.

Additionally provided herein is a process of preparing a compound of Formula (II-A-7),

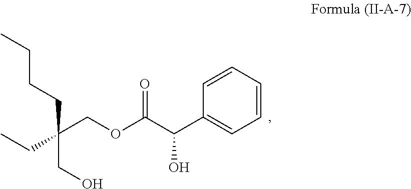 [SMILES: CCCC[C@](CC)(CO)COC(=O)[C@@H](O)c1ccccc1]

or a pharmaceutically acceptable salt, hydrate, or solvate thereof, the process comprising: contacting the compounds of 2-butyl-2-ethylpropane-1,3-diol and ethyl (2S)-2-hydroxy-2-phenyl-acetate, in an organic solvent to produce a compound of Formula (II-A-7).

In some embodiments, the method further comprises addition of an acid.

In some embodiments, the acid is DBSA, TfOH, pTsOH, MsOH, H₂SO₄, HCl, H₃PO₄, or TFA.

In some embodiments, the method further comprises addition of a base.

In some embodiments, the base is a hydroxide base.

In some embodiments, the hydroxide base is LiOH, NaOH, or KOH.

In some embodiments, the base is an alkoxide base.

In some embodiments, the alkoxide base is LiOMe, NaOMe, KOMe, LiOEt, NaOEt, or KOEt.

In some embodiments, the base is a carbonate.

In some embodiments, the carbonate is Li₂CO₃, Na₂CO₃, K₂CO₃, or Cs₂CO₃.

In some embodiments, the method further comprises addition of a metal oxide.

In some embodiments, the metal oxide is CaO or MgO.

In some embodiments, the method further comprises addition of a Lewis acid.

In some embodiments, the Lewis acid is AlCl₃, AlBr₃, FeCl₃, FeBr₃, ZnCl₂, Ti(OiPr)₄, or Al(OEt)₃.

In some embodiments, the method further comprises addition of a heterogeneous acid catalyst.

In some embodiments, the heterogeneous acid catalyst is an ion exchange resin or zeolite.

In some embodiments, the organic solvent is an alkane.

In some embodiments, the alkane is a pentane, a hexane, a heptane, an octane, cyclopentane, cyclohexane, or methyl cyclohexane.

In some embodiments, the organic solvent is an aromatic solvent.

In some embodiments, the aromatic solvent is benzene, toluene, ethyl benzene, or chlorobenzene.

In some embodiments, the organic solvent is an ether.

In some embodiments, the ether is diethyl ether, di-n-butyl ether, diisopropyl ether, methyl tert-butyl ether, ethyl tert-butyl ether, cyclopropyl methyl ether, tert-amyl methyl ether, THF, methyl THF, or dioxane.

In some embodiments, the contacting the compounds of 2-butyl-2-ethylpropane-1,3-diol and ethyl (2S)-2-hydroxy-2-phenyl-acetate to produce a compound of Formula (II-A-7) occurs at a reaction temperature of about 50° C.

In some embodiments, the method further comprises isolating the compound of Formula (II-A-7) or a pharmaceutically acceptable salt, hydrate, or solvate thereof.

In some embodiments, the isolation of the compound of Formula (II-A-7) comprises crystallizing the compound of Formula (II-A-7).

Further provided herein is a process of preparing a compound of Formula (I-A-65),

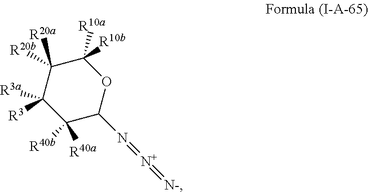

or a pharmaceutically acceptable salt, hydrate, or solvate thereof, the process comprising:
using a flow process to contact the compound of Formula (I-A-63),

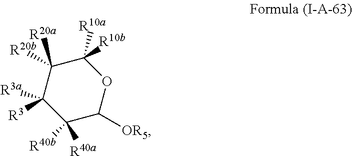

or a pharmaceutically acceptable salt, hydrate, or solvate thereof, wherein:
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; and
R⁵ is a fifth alcohol protecting group;
with trimethylsilyl azide in the presence of a Lewis acid to produce a compound of Formula (I-A-65).

In some embodiments, the Lewis acid is SnCl₄.

In some embodiments, the compound of Formula (I-A-63) has the structure of Formula (II-A-63),

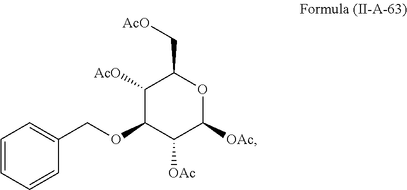 [SMILES: CC(=O)OC[C@H]1O[C@@H](OC(C)=O)[C@H](OC(C)=O)[C@@H](OCc2ccccc2)[C@@H]1OC(C)=O]

or a pharmaceutically acceptable salt, hydrate, or solvate thereof.

In some embodiments, each of the first, second, and third alcohol protecting groups is independently selected from the group consisting of acetyl (Ac), benzoyl, benzyl (Bn), methoxyethoxymethyl, dimethoxytrityl, methoxymethyl, methoxytrityl, p-methoxybenzyl, p-methoxyphenyl, ethylthiomethyl, pivaloyl, tert-butyl, tetrahydropyranyl, tetrahydrofuran, trityl, silyl, methyl, ethoxyethyl, and —C(O)R⁰⁰.

In some embodiments, the fourth alcohol protecting group is selected from the group consisting of acetyl (Ac), benzoyl, benzyl (Bn), methoxyethoxymethyl, dimethoxytrityl, methoxymethyl, methoxytrityl, p-methoxybenzyl, p-methoxyphenyl, ethylthiomethyl, pivaloyl, tert-butyl, tetrahydropyranyl, tetrahydrofuran, trityl, silyl, methyl, ethoxyethyl, and —C(O)R⁰⁰.

In some embodiments, at least one of the second, third, and fourth alcohol protecting groups is —C(O)R⁰⁰.

In some embodiments, each one of the second, third, and fourth alcohol protecting groups is —C(O)R⁰⁰.

In some embodiments, the first alcohol protecting group is different from at least one of the second, third, and fourth alcohol protecting groups.

In some embodiments, the first alcohol protecting group is different from each one of the second, third, and fourth alcohol protecting groups.

In some embodiments, the fourth and the third alcohol protecting groups are the same.

In some embodiments, the second, third, and fourth alcohol protecting groups are the same.

In some embodiments, the first alcohol protecting group is benzyl (Bn).

In some embodiments, at least one of the second, third, and fourth alcohol protecting groups is acetyl (Ac).

In some embodiments, each one of the second, third, and fourth alcohol protecting groups is acetyl (Ac).

In some embodiments, the fifth alcohol protecting group is selected from the group consisting of acetyl (Ac), benzoyl, benzyl (Bn), methoxyethoxymethyl, dimethoxytrityl, methoxymethyl, methoxytrityl, p-methoxybenzyl, p-methoxyphenyl, ethylthiomethyl, pivaloyl, tert-butyl, tetrahydropyranyl, tetrahydrofuran, trityl, silyl, methyl, and ethoxyethyl.

In some embodiments, the fifth alcohol protecting group is acetyl (Ac).

In some embodiments, each of R^{10a}, R^{20a}, R^{3a}, and R^{40a} is H.

In some embodiments, R³ is O-Bn.

In some embodiments, R^{20b} and R^{40b} are both —O—Ac.

In some embodiments, R⁵ is Ac.

In some embodiments, R^{10b} is —CH₂—O—Ac.

### Compounds of Volixibat Analogs and Intermediates

Additionally provided herein is a compound of Formula (I),

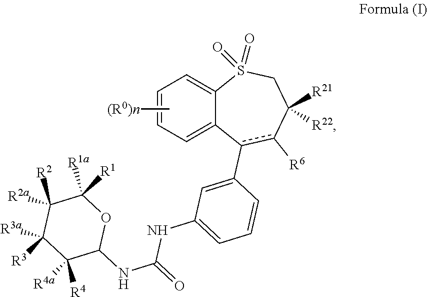

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
one of R¹ and R^{1a} is an optionally substituted (C₁₋₆)alkylene-OSO₃H, (C₁₋₆)alkylene-OH, (C₁₋₆)alkylene-OC(O)R⁰⁰, or C(O)H and the other of R¹ and R^{1a} is H, (C₁₋₆)alkyl, NH₂, NH(C₁₋₆)alkyl, or N((C₁₋₆)alkyl)₂;
one of R² and R^{2a} is an optionally substituted (C₁₋₆)alkylene-OH, OH, OSO₃H, or OC(O)R⁰⁰ and the other of R² and R^{2a} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R⁴ and R^{4a} is an optionally substituted (C₁₋₆)alkylene-OH, OH, OSO₃H, or OC(O)R⁰⁰ and the other of R⁴ and R^{4a} is H, (C₁₋₆)alkyl, NH₂, NH(C₁₋₆)alkyl, or N((C₁₋₆)alkyl)₂;
each of R¹¹ and R²² is independently an optionally substituted (C₁₋₆)alkyl;
n is an integer of 0-4;
R⁶ is H or OH;
wherein the compound of Formula (I) does not have the structure of Formula (III-27),

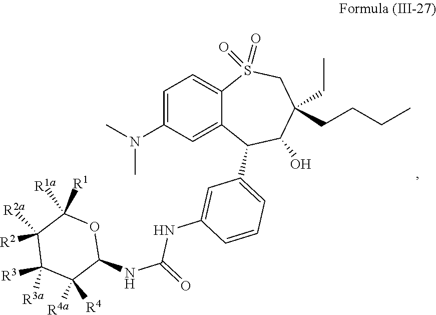

or a pharmaceutically acceptable salt, hydrate, or solvate thereof, wherein:
one of R¹ and R^{1a} is an optionally substituted (C₁₋₆)alkylene-OSO₃H and the other of R¹ and R^{1a} is H or (C₁₋₆)alkyl;
one of R² and R^{2a} is an optionally substituted (CH₂)—OH or OH and the other of R² and R^{2a} is H or (C₁₋₆)alkyl;
one of R³ and R^{3a} is an optionally substituted (C₁₋₆)alkylene-O—R^{1P} or —O—R^{1P}, and the other of R³ and R^{3a} is H or (C₁₋₆)alkyl;
wherein R^{1P} is (C₁-C₆)-alkyl optionally substituted by fluorine, phenyl, —(CH₂)₁₋₆-phenyl, O-phenyl, O—(CH₂)₁₋₆-phenyl, —(CH₂)—O—(CH₂)₁₋₆-phenyl, where the phenyl ring is optionally mono- to trisubstituted with F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, or CONH₂; and one of R⁴ and R^{4a} is an optionally substituted (CH₂)—OH or OH and the other of R⁴ and R^{4a} is H or (C₁₋₆)alkyl.

In some embodiments, the compound of Formula (I) has the structure selected from the group consisting of Formula (I-AA), Formula (I-AB), Formula (I-BA), Formula (I-BB), Formula (I-CA), Formula (I-CB), Formula (I-DA), and Formula (I-DB),

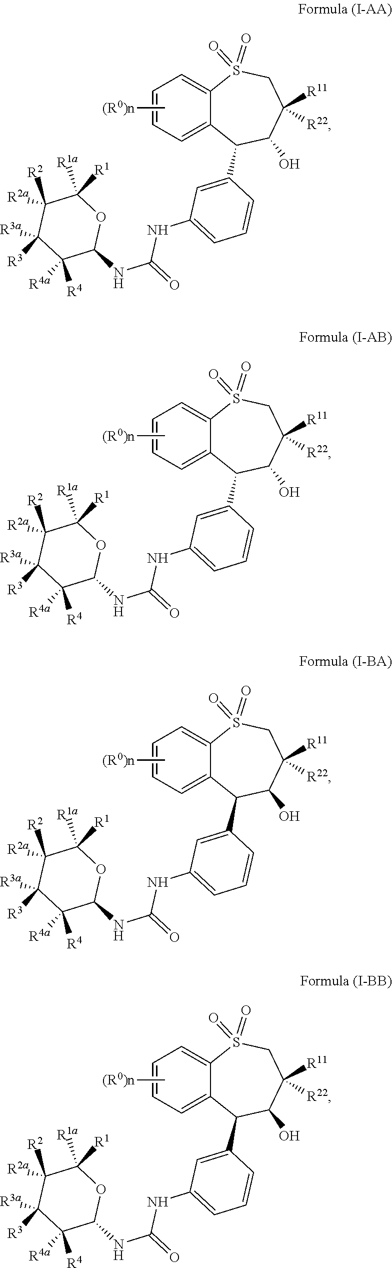

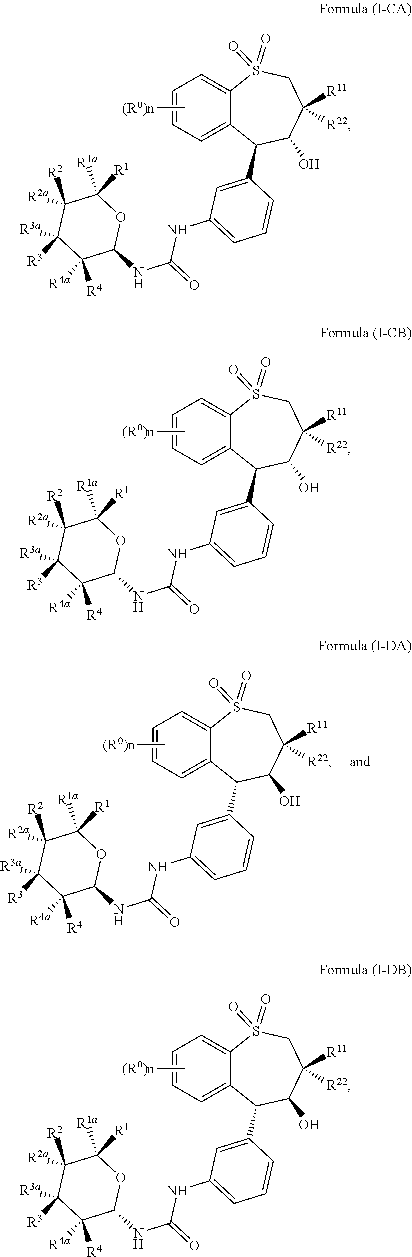

or a pharmaceutically acceptable salt, hydrate, or solvate thereof, wherein:
Formula (I-AA) is not Formula (II-A-27).

In some embodiments, the compound of Formula (I) has the structure selected from the group consisting of Formula (II-A-123), Formula (II-B-175), Formula (II-C-138), Formula (II-D-95), Formula (II-E-179), Formula (II-F-134), Formula (II-G-76), and Formula (II-H-99),

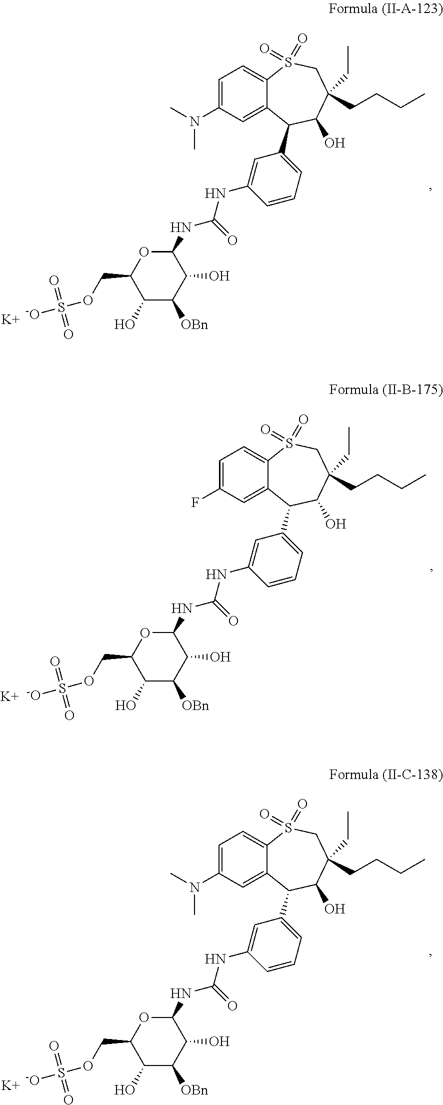

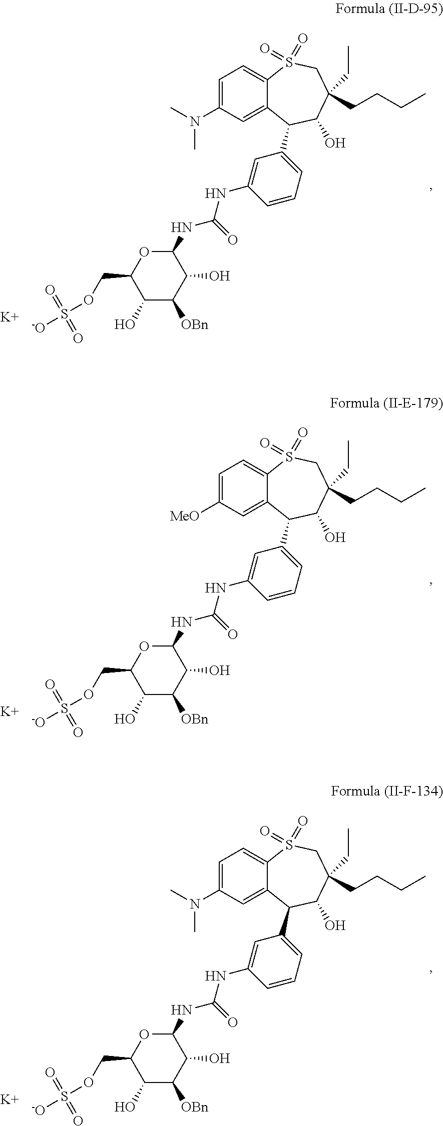

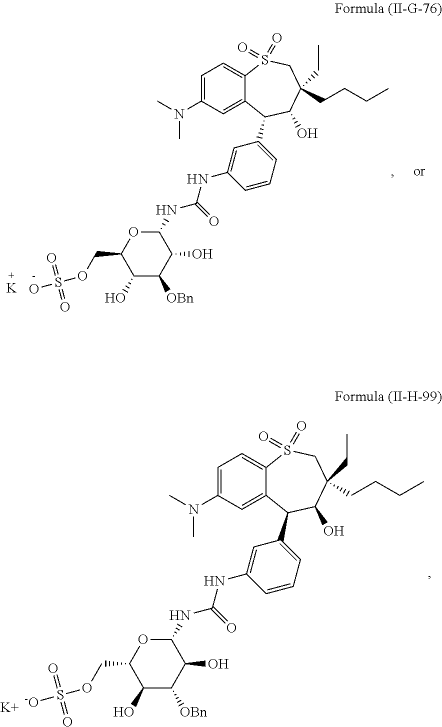

or a pharmaceutically acceptable salt, hydrate, or solvate thereof.

In some embodiments, the compound of Formula (I) has the structure selected from the group consisting of Formula (II-A-186), Formula (II-B-186), and Formula (II-C-186),

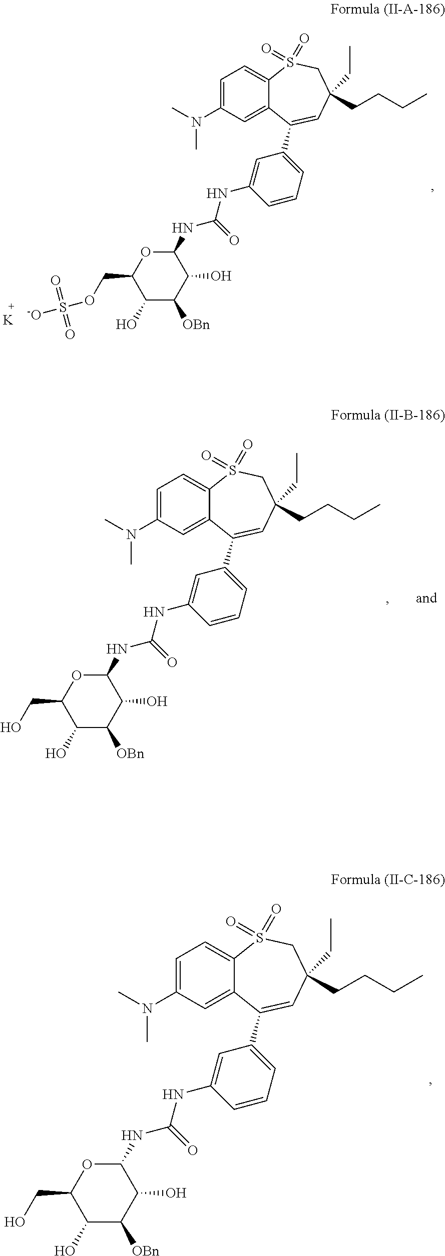

or a pharmaceutically acceptable salt, hydrate, or solvate thereof.

Further provided herein is a compound of Formula (I-26)

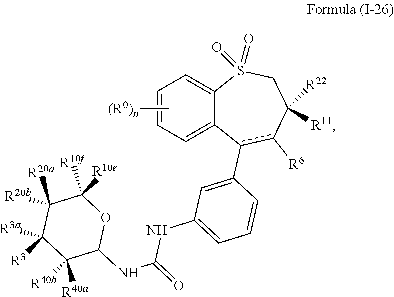

or a pharmaceutically acceptable hydrate or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
   each of R¹¹ and R²² is independently an optionally substituted (C₁₋₆)alkyl;
   n is an integer of 0-4;
   R⁶ is H or OH;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{10e} and R^{10f} is C(O)H or an optionally substituted (C₁₋₆)alkylene-OSO₃⁻(pyridine)⁺ and the other of R^{10e} and R^{10f} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P} or O—R^{2P}, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P} or O—R^{3P}, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; wherein the compound of Formula (I-26) does not have the structure of Formula (II-A-26),

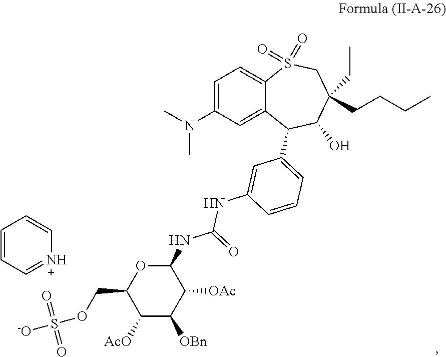

or a pharmaceutically acceptable hydrate or solvate thereof.

In some embodiments, the compound of Formula (I-26) has the structure selected from the group consisting of Formula (I-AA-26), Formula (I-AB-26), Formula (I-BA-26), Formula (I-BB-26), Formula (I-CA-26), Formula (I-CB-26), Formula (I-DA-26), and Formula (I-DB-26),

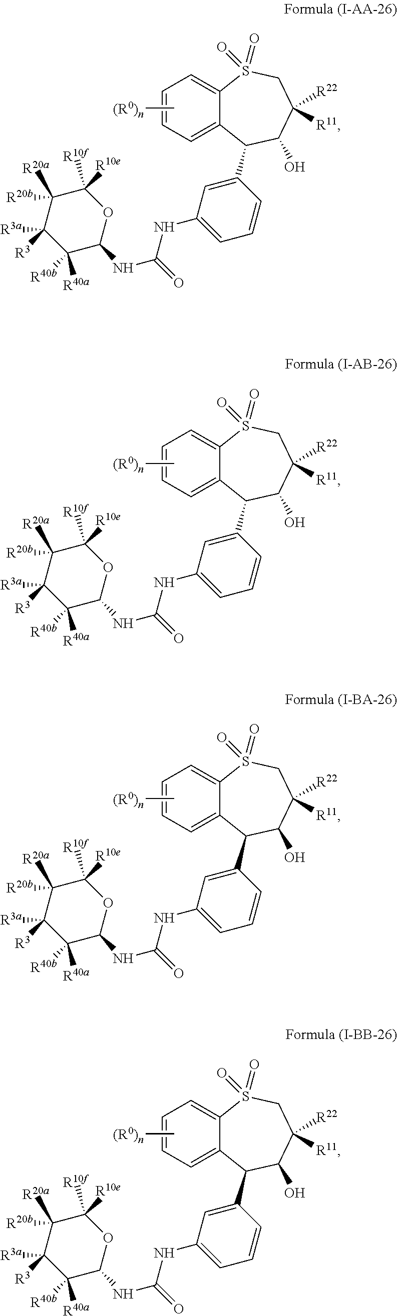

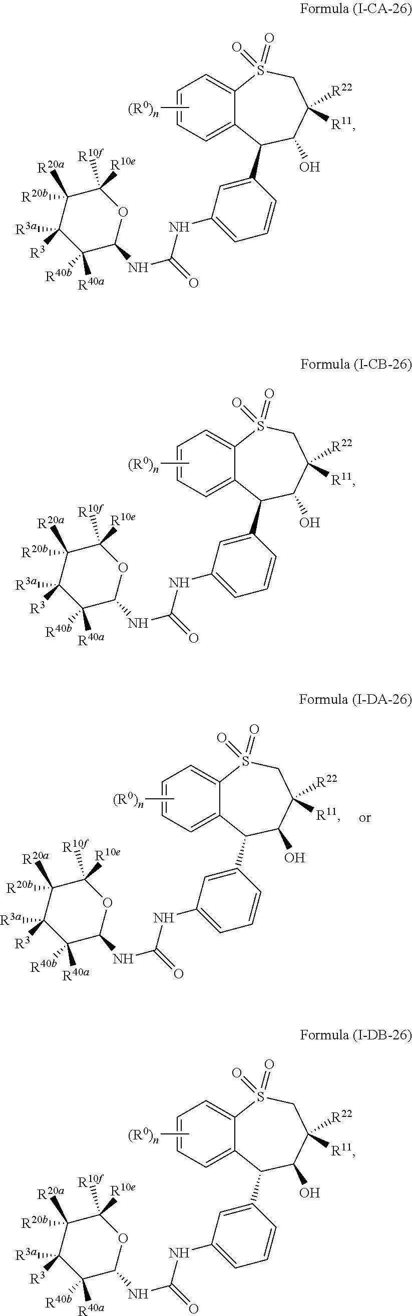

or a pharmaceutically acceptable hydrate or solvate thereof, wherein Formula (I-AA-26) is not Formula (II-A-26).

In some embodiments, the compound of Formula (I-26) has the structure of Formula (I-A-26),

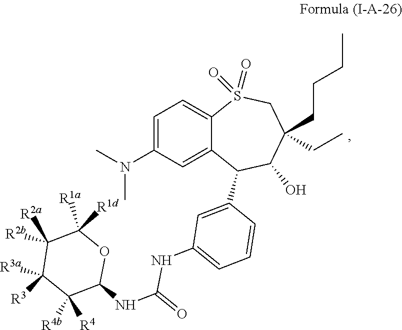

or a pharmaceutically acceptable hydrate or solvate thereof, wherein:
each of R^{1a}, R², R^{3a}, and R⁴ is independently H, (C₁₋₆)alkyl, NH₂, NH(C₁₋₆)alkyl, or N((C₁₋₆)alkyl)₂;
R^{1d} is (C₁₋₆)alkylene-OSO₃⁻(pyridine)⁺;
R^{2b} is an optionally substituted (C₁₋₆)alkylene-O—R^{2P} or O—R^{2P}, wherein R^{2P} is the second alcohol protecting group;
R³ is an optionally substituted (C₁₋₆)alkylene-O—R^{1P} or —O—R^{1P}, wherein R^{1P} is the first alcohol protecting group; and
R^{4b} is an optionally substituted (C₁₋₆)alkylene-O—R^{3P} or O—R^{3P}, wherein R^{3P} is the third alcohol protecting group, wherein Formula (I-A-26) is not Formula (II-A-26).

In some embodiments, the compound of Formula (I-26) has the structure of Formula (II-A-126), Formula (II-A-137), Formula (II-A-94), Formula (II-A-133), Formula (II-A-75), Formula (II-A-98), Formula (II-A-174), Formula (II-A-178),

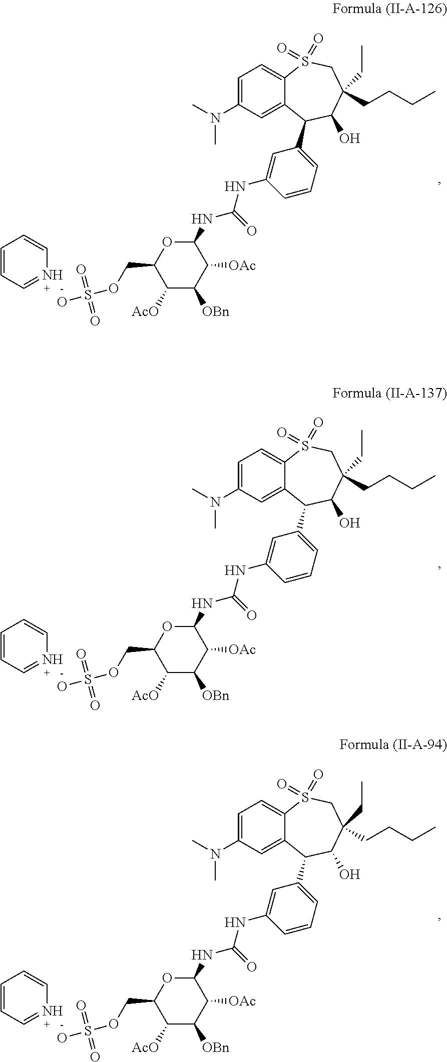

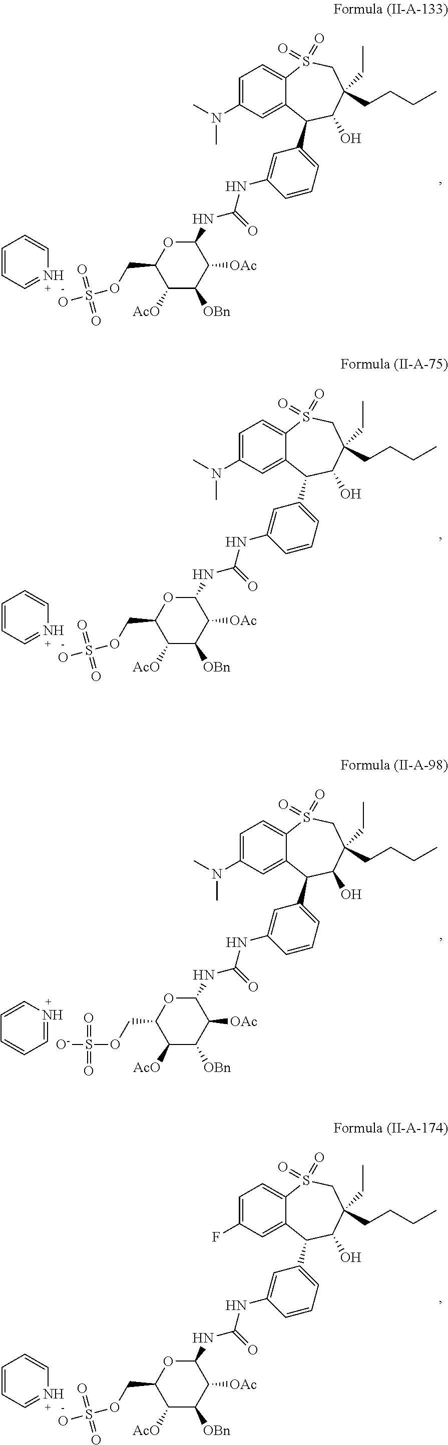

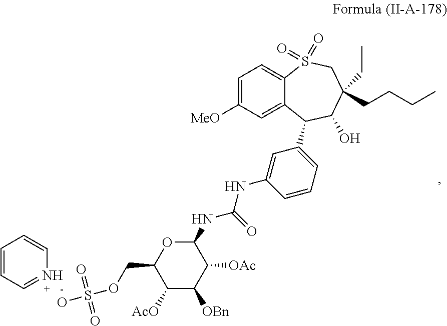

or a pharmaceutically acceptable hydrate or solvate thereof.

Additionally provided herein is a compound of Formula (I-25),

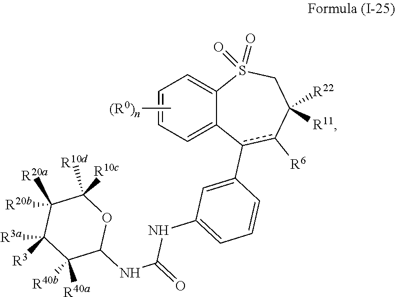

or a pharmaceutically acceptable hydrate or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R¹¹ and R²² is independently an optionally substituted (C₁₋₆)alkyl;
n is an integer of 0-4;
R⁶ is H or OH;
one of R³ and R^{3a} is an optionally substituted (C₁₋₆)alkylene-O—R^{1P} or —O—R^{1P}, wherein R^{1P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{10d} and R^{10e} is C(O)H or an optionally substituted (C₁₋₆)alkylene-OH or —OH, and the other of R^{10d} and R^{10e} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P} or O—R^{2P}, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P} or O—R^{3P}, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂; wherein the compound of Formula (I-25) does not have the structure of Formula (II-A-25),

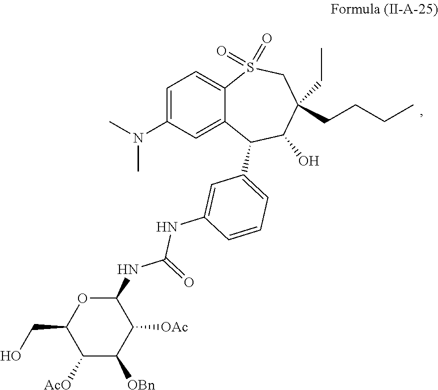 [SMILES: CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

or a pharmaceutically acceptable hydrate or solvate thereof.

In some embodiments, the compound of Formula (I-25) has the structure selected from the group consisting of Formula (I-AA-25), Formula (I-AB-25), Formula (I-BA-25), Formula (I-BB-25), Formula (I-CA-25), Formula (I-CB-25), Formula (I-DA-25), and Formula (I-DB-25),

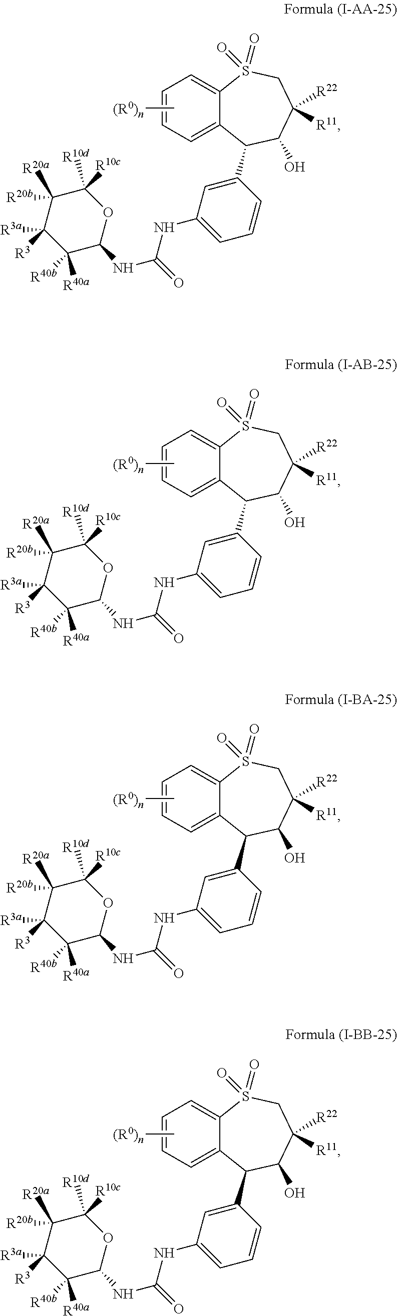

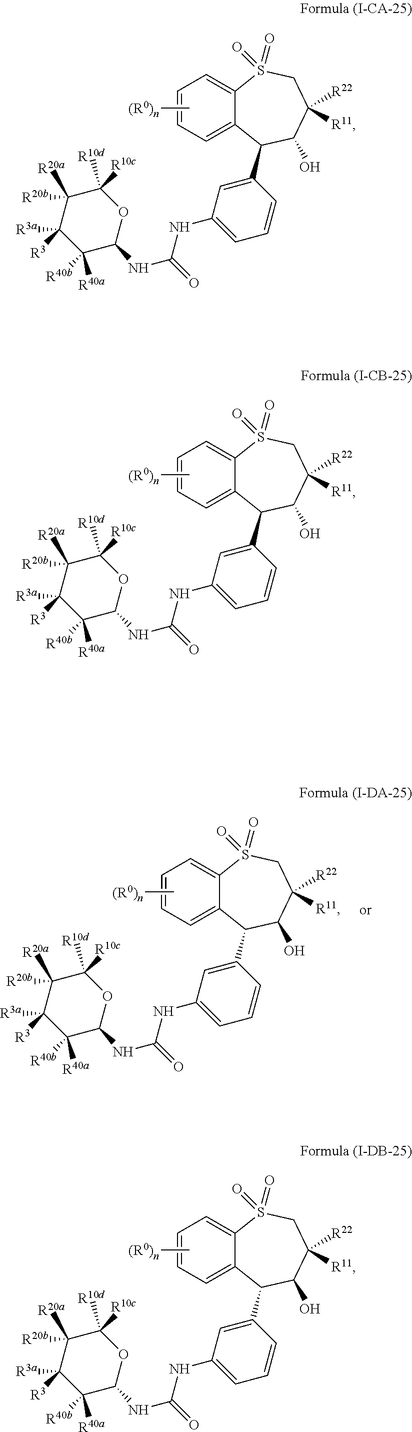

or a pharmaceutically acceptable hydrate or solvate thereof, wherein Formula (I-AA-25) is not Formula (II-A-25).

In some embodiments, the compound of Formula (I-25) has the structure of Formula (I-A-25),

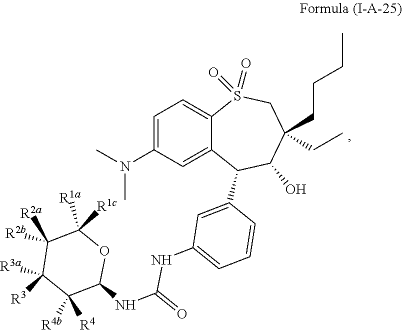

or a pharmaceutically acceptable hydrate or solvate thereof, wherein:
each of R^{1a}, R², R^{3a}, and R⁴ is independently H, (C₁₋₆)alkyl, NH₂, NH(C₁₋₆)alkyl, or N((C₁₋₆)alkyl)₂;
R^{2b} is an optionally substituted (C₁₋₆)alkylene-O—R^{2P} or O—R^{2P}, wherein R^{2P} is the second alcohol protecting group;
R³ is an optionally substituted (C₁₋₆)alkylene-O—R^{1P} or —O—R^{1P}, wherein R^{1P} is the first alcohol protecting group;
R^{4b} is an optionally substituted (C₁₋₆)alkylene-O—R^{3P} or O—R^{3P}, wherein R^{3P} is the third alcohol protecting group; and
R^{1c} is (C₁₋₆)alkylene-OH or —OH, wherein Formula (I-A-25) is not Formula (II-A-25).

In some embodiments, the compound of Formula (I-25) has the structure of Formula (II-A-125), Formula (II-A-136), Formula (II-A-93), Formula (II-A-132), Formula (II-A-74), Formula (II-A-97), Formula (II-A-53), Formula (II-A-177),

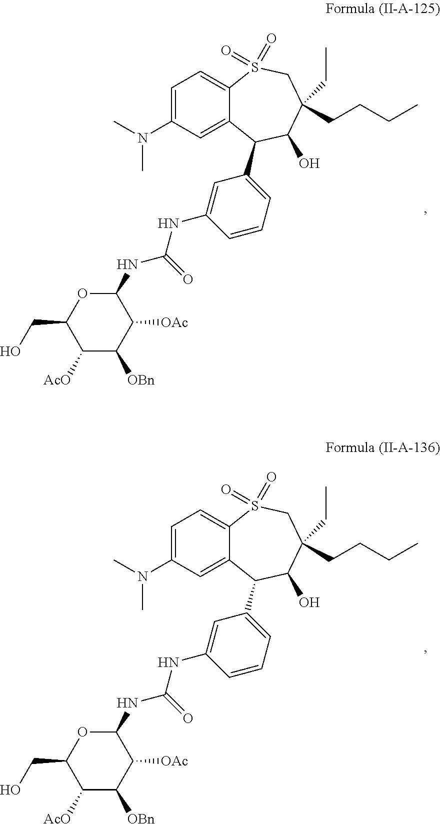 [SMILES: CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@@H]1O]

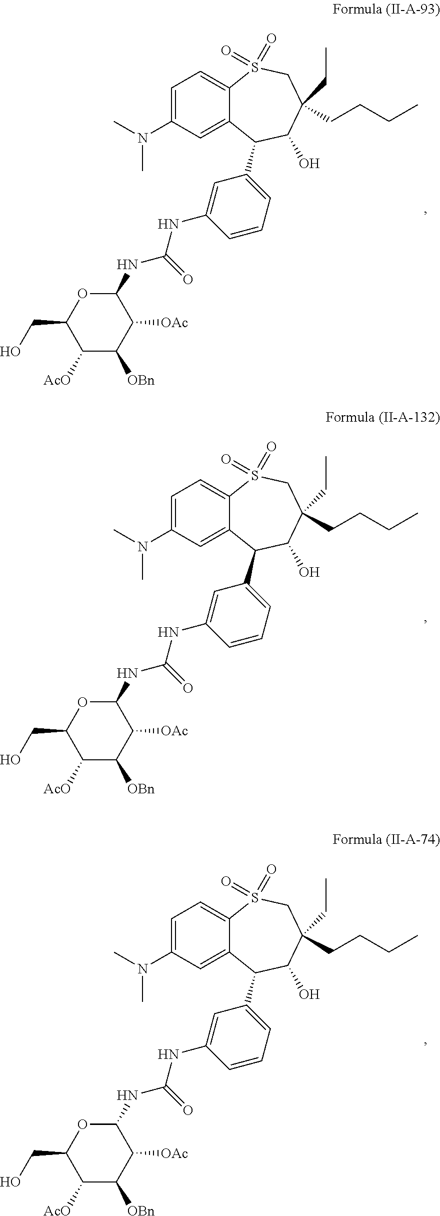 [SMILES: CCCC[C@@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](CO)[C@@H](OC(C)=O)[C@H](OCc4ccccc4)[C@H]3OC(C)=O)c2)[C@H]1O]

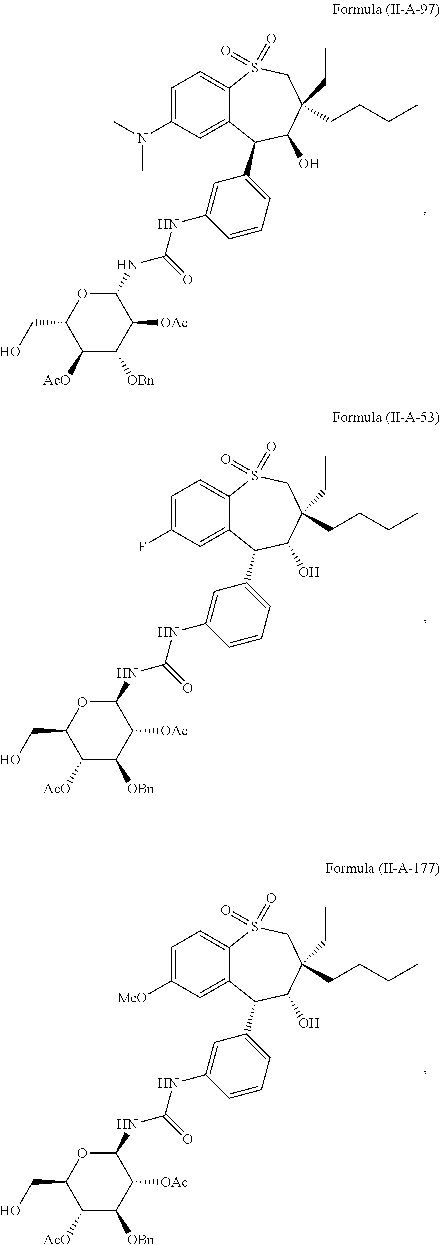 [SMILES: CCCC[C@@]1(CC)CS(=O)(=O)c2ccc(N(C)C)cc2[C@H](c2cccc(NC(=O)N[C@H]3O[C@@H](CO)[C@H](OC(C)=O)[C@@H](OCc4ccccc4)[C@@H]3OC(C)=O)c2)[C@@H]1O ; CCCC[C@]1(CC)CS(=O)(=O)c2ccc(F)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(OC)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

or a pharmaceutically acceptable hydrate or solvate thereof.

Further provided herein is a compound of Formula (I-20),

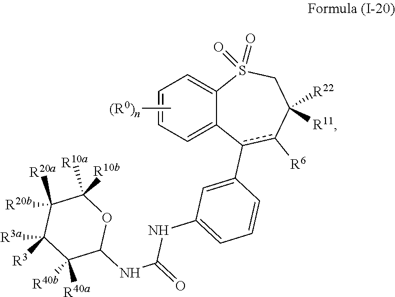

or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R¹¹ and R²² is independently an optionally substituted (C₁₋₆)alkyl;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂;
n is an integer of 0-4;
R⁶ is H or OH; and
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂; wherein the compound of Formula (I-20) does not have the structure of Formula (II-A-20),

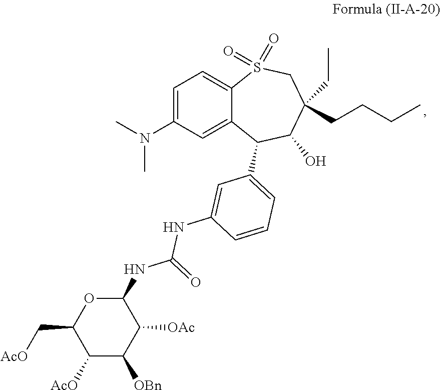 [SMILES: CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

or a pharmaceutically acceptable salt, hydrate, or solvate thereof.

In some embodiments, the compound of Formula (I-20) has the structure selected from the group consisting of Formula (I-AA-20), Formula (I-AB-20), Formula (I-BA-20), Formula (I-BB-20), Formula (I-CA-20), Formula (I-CB-20), Formula (I-DA-20), and Formula (I-DB-20),

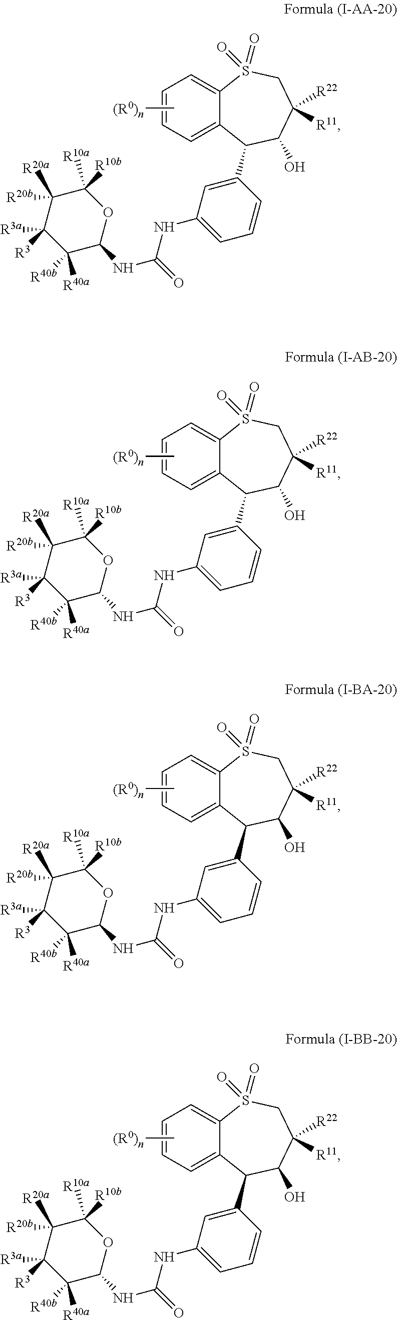

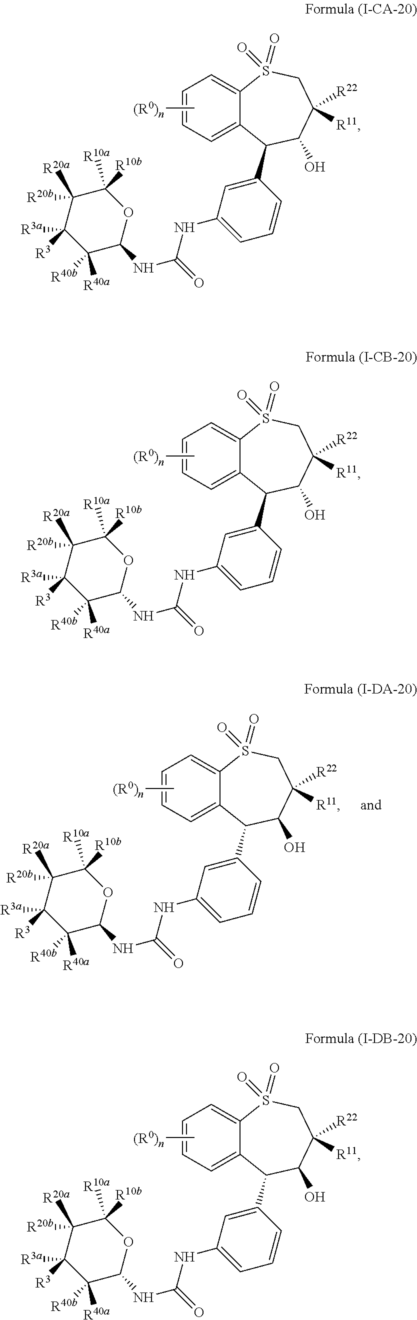

or a pharmaceutically acceptable salt, hydrate, or solvate thereof, wherein Formula (I-AA-20) is not Formula (II-A-20).

In some embodiments, the compound of Formula (I-20) has the structure of Formula (I-A-20),

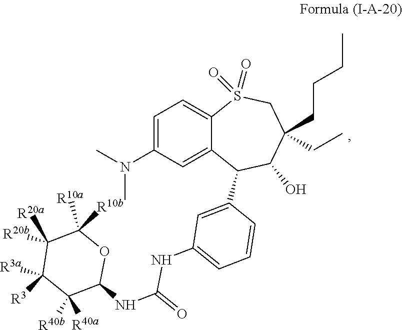

or a pharmaceutically acceptable salt, hydrate, or solvate thereof, wherein:
each of R¹⁰, R^{20a}, R^{3a}, and R^{40a} is independently H, (C₁₋₆)alkyl, NH₂, NH(C₁₋₆)alkyl, or N((C₁₋₆)alkyl)₂;
R³ is an optionally substituted (C₁₋₆)alkylene-O—R^{1P} or —O—R^{1P}, wherein R^{1P} is the first alcohol protecting group;
R^{10b} is an optionally substituted (C₁₋₆)alkylene-O—R^{4P} or O—R^{4P}, wherein R^{4′} is the fourth alcohol protecting group;
R^{20b} is an optionally substituted (C₁₋₆)alkylene-O—R^{2P} or O—R^{2P}, wherein R^{2P} is the second alcohol protecting group;
R^{40b} is an optionally substituted (C₁₋₆)alkylene-O—R^{3P} or O—R^{3P}, wherein R^{3P} is the third alcohol protecting group, wherein Formula (I-A-20) is not Formula (II-A-20).

In some embodiments, the compound of Formula (I-20) has the structure of Formula (II-A-120), Formula (II-A-135), Formula (II-A-92), Formula (II-A-131), Formula (II-A-49), Formula (II-A-96), Formula (II-A-51), Formula (II-A-176),

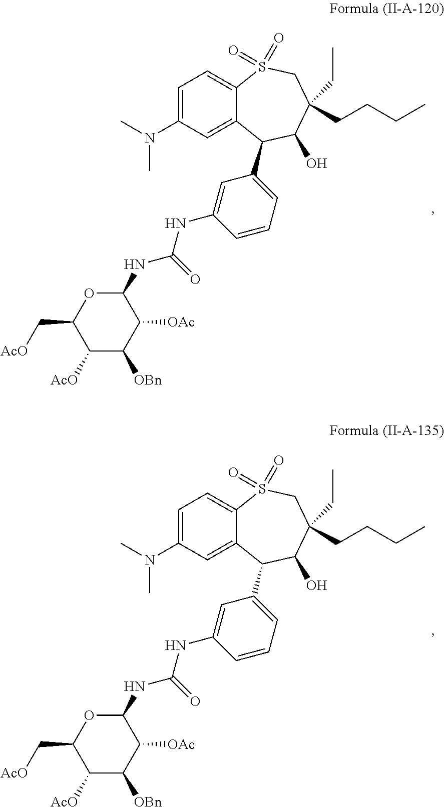 [SMILES: CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O]

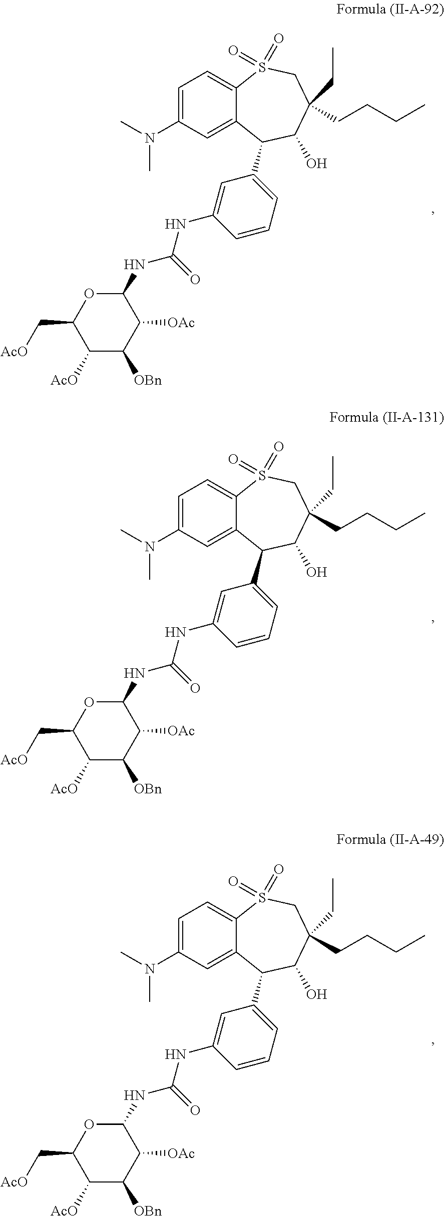 [SMILES: CCCC[C@@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](COC(C)=O)[C@@H](OC(C)=O)[C@H](OCc4ccccc4)[C@H]3OC(C)=O)c2)[C@H]1O]

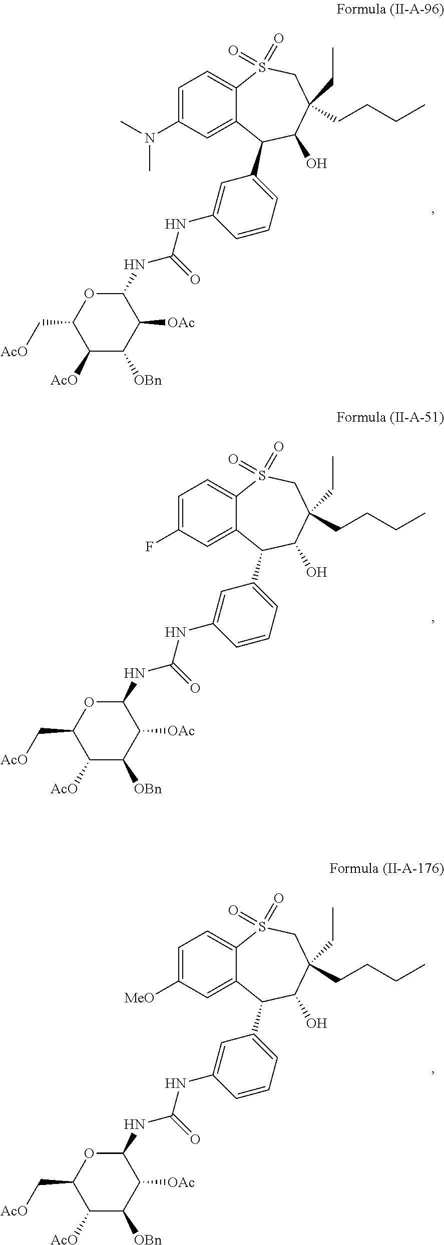 [SMILES: CCCC[C@@]1(CC)CS(=O)(=O)c2ccc(N(C)C)cc2[C@H](c2cccc(NC(=O)N[C@H]3O[C@@H](COC(C)=O)[C@H](OC(C)=O)[C@@H](OCc4ccccc4)[C@@H]3OC(C)=O)c2)[C@@H]1O ; CCCC[C@]1(CC)CS(=O)(=O)c2ccc(F)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(OC)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

or a pharmaceutically acceptable salt, hydrate, or solvate thereof.

Further provided herein is a compound of Formula (II-17),

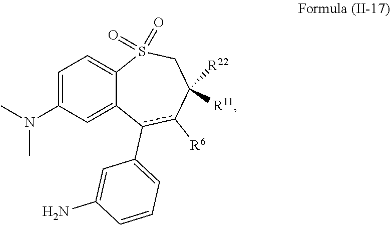

or a pharmaceutically acceptable salt, hydrate, or solvate thereof, wherein:
each of R¹¹ and R²² is independently an optionally substituted (C₁₋₆)alkyl; and
R⁶ is H or OH, wherein the compound of Formula (II-17) does not have the structure of Formula (II-A-17),

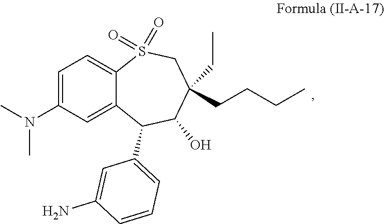 [SMILES: CCCC[C@]1(CC)CS(=O)(=O)c2ccc(N(C)C)cc2[C@@H](c2cccc(N)c2)[C@H]1O]

or a pharmaceutically acceptable salt, hydrate, or solvate thereof.

In some embodiments, the compound of Formula (II-17) has the structure selected from the group consisting of:

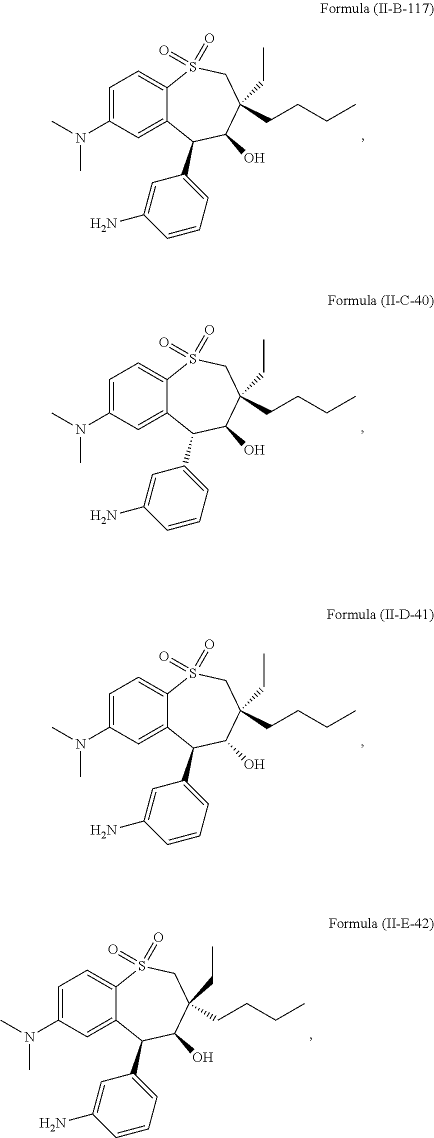

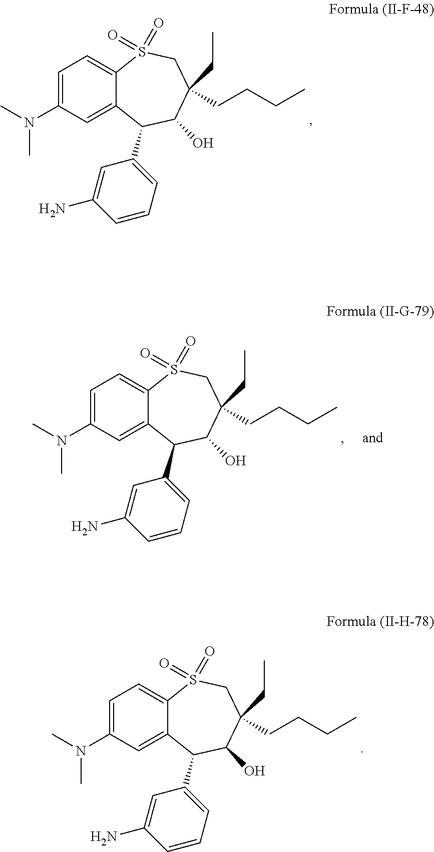

Further provided herein is a compound of Formula (II-16),

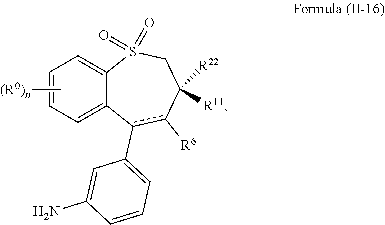

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl; and
R⁶ is H or OH, wherein the compound of Formula (II-16) does not have the structure of Formula (II-E-16),

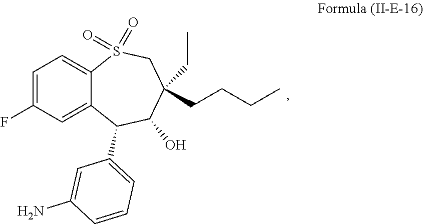 [SMILES: CCCC[C@]1(CC)CS(=O)(=O)c2ccc(F)cc2[C@@H](c2cccc(N)c2)[C@H]1O]

or a pharmaceutically acceptable salt, hydrate, or solvate thereof.

In some embodiments, the compound of Formula (II-16) has the structure selected from the group consisting of:

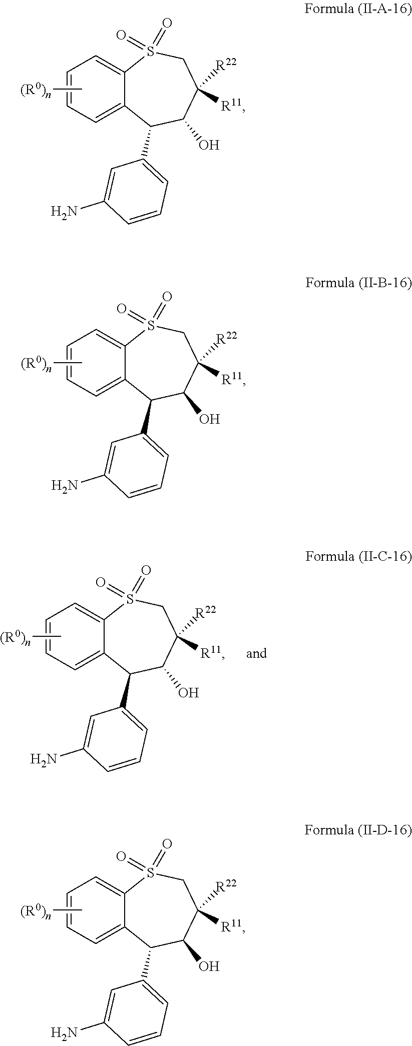

or a pharmaceutically acceptable salt, hydrate, or solvate thereof, wherein Formula (II-A-16) is not Formula (II-E-16).

In some embodiments, the compound of Formula (II-16) has the structure selected from the group consisting of:

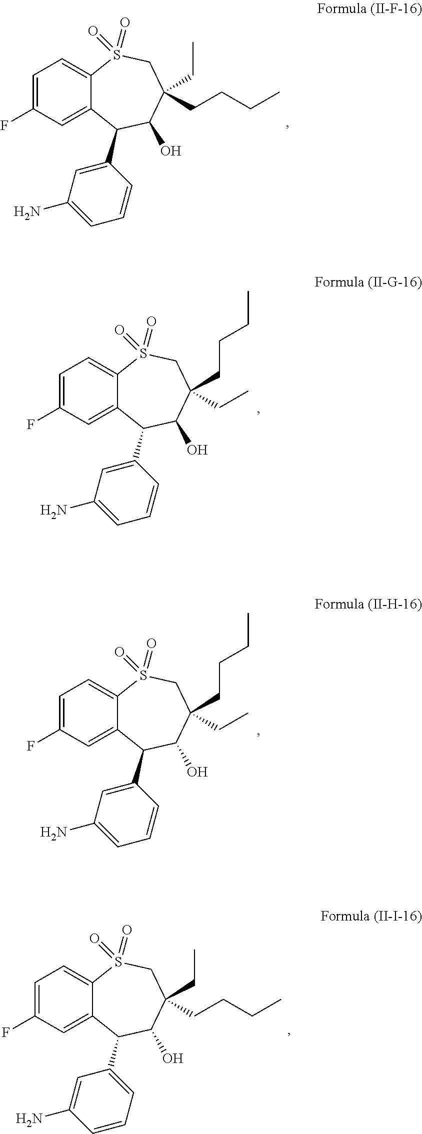 [SMILES: CCCC[C@]1(CC)CS(=O)(=O)c2ccc(F)cc2[C@H](c2cccc(N)c2)[C@@H]1O ; CCCC[C@]1(CC)CS(=O)(=O)c2ccc(F)cc2[C@@H](c2cccc(N)c2)[C@@H]1O ; CCCC[C@]1(CC)CS(=O)(=O)c2ccc(F)cc2[C@H](c2cccc(N)c2)[C@H]1O ; CCCC[C@@]1(CC)CS(=O)(=O)c2ccc(F)cc2[C@@H](c2cccc(N)c2)[C@H]1O]

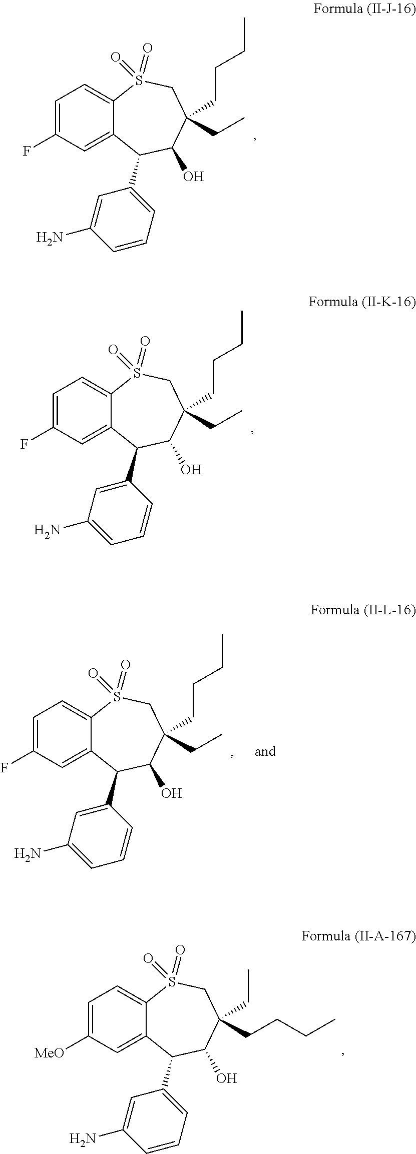 [SMILES: CCCC[C@@]1(CC)CS(=O)(=O)c2ccc(F)cc2[C@@H](c2cccc(N)c2)[C@@H]1O ; CCCC[C@@]1(CC)CS(=O)(=O)c2ccc(F)cc2[C@H](c2cccc(N)c2)[C@H]1O ; CCCC[C@@]1(CC)CS(=O)(=O)c2ccc(F)cc2[C@H](c2cccc(N)c2)[C@@H]1O ; CCCC[C@]1(CC)CS(=O)(=O)c2ccc(OC)cc2[C@@H](c2cccc(N)c2)[C@H]1O]

or a pharmaceutically acceptable salt, hydrate, or solvate thereof.

Further provided herein is a compound of Formula (II-15),

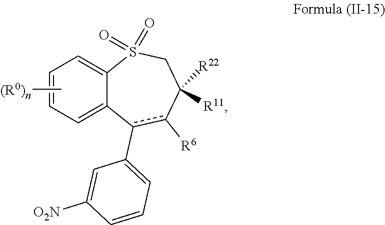

or a pharmaceutically acceptable salt, hydrate, or solvate thereof, wherein:
each of R¹¹ and R²² is independently an optionally substituted (C₁₋₆)alkyl;
n is an integer of 0-4;
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl; and
R⁶ is H or OH, wherein the compound of Formula (II-15) does not have the structure of Formula (II-E-15),

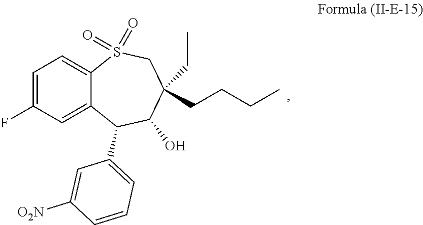 [SMILES: CCCC[C@]1(CC)CS(=O)(=O)c2ccc(F)cc2[C@@H](c2cccc([N+](=O)[O-])c2)[C@H]1O]

or a pharmaceutically acceptable salt, hydrate, or solvate thereof.

In some embodiments, the compound of Formula (II-15) has the structure selected from the group consisting of:

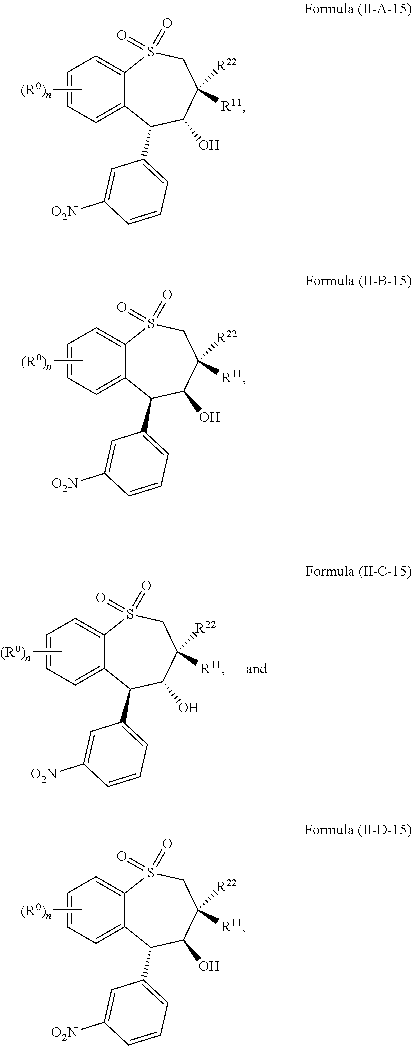

or a pharmaceutically acceptable salt, hydrate, or solvate thereof, wherein Formula (II-A-15) is not Formula (II-E-15).

In some embodiments, the compound of Formula (II-15) has the structure selected from the group consisting of:

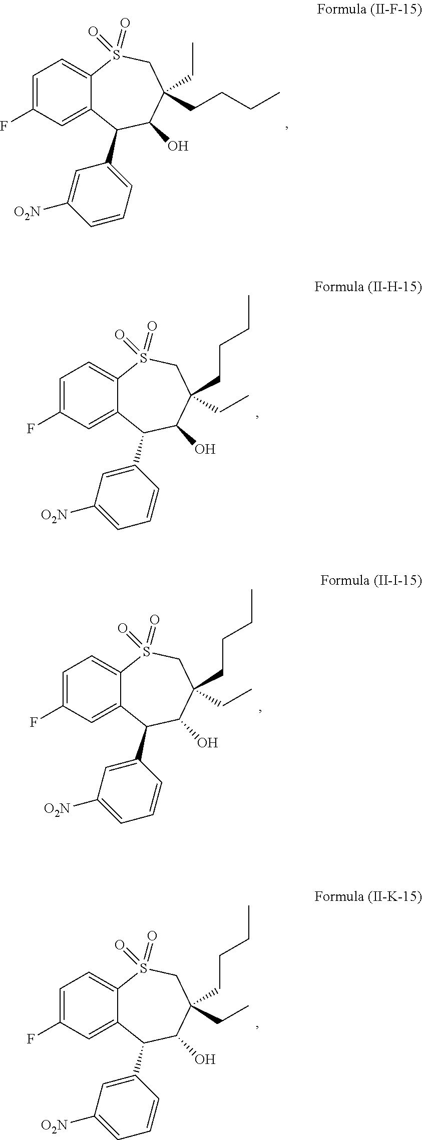 [SMILES: CCCC[C@]1(CC)CS(=O)(=O)c2ccc(F)cc2[C@H](c2cccc([N+](=O)[O-])c2)[C@@H]1O ; CCCC[C@]1(CC)CS(=O)(=O)c2ccc(F)cc2[C@@H](c2cccc([N+](=O)[O-])c2)[C@@H]1O ; CCCC[C@]1(CC)CS(=O)(=O)c2ccc(F)cc2[C@H](c2cccc([N+](=O)[O-])c2)[C@H]1O ; CCCC[C@@]1(CC)CS(=O)(=O)c2ccc(F)cc2[C@@H](c2cccc([N+](=O)[O-])c2)[C@H]1O]

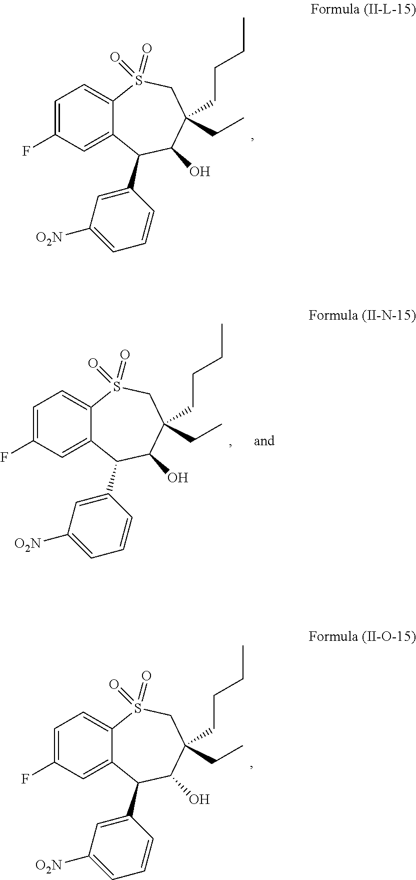 [SMILES: CCCC[C@@]1(CC)CS(=O)(=O)c2ccc(F)cc2[C@H](c2cccc([N+](=O)[O-])c2)[C@@H]1O ; CCCC[C@@]1(CC)CS(=O)(=O)c2ccc(F)cc2[C@@H](c2cccc([N+](=O)[O-])c2)[C@@H]1O ; CCCC[C@@]1(CC)CS(=O)(=O)c2ccc(F)cc2[C@H](c2cccc([N+](=O)[O-])c2)[C@H]1O]

or a pharmaceutically acceptable salt, hydrate, or solvate thereof.

Further provided herein is a compound of Formula (II-C-150),

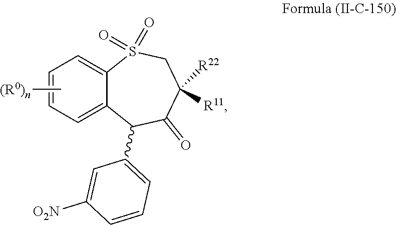

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-C-150) has the structure of Formula (II-A-150), Formula (II-B-150),

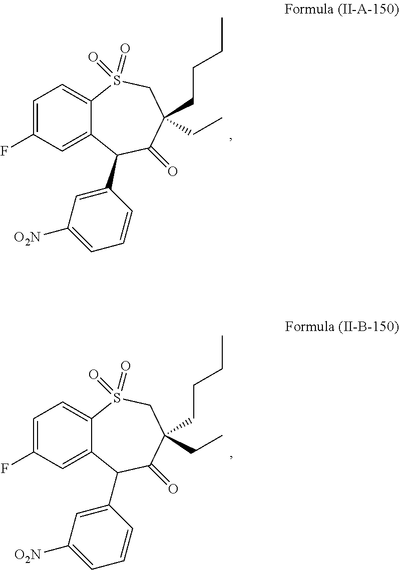 [SMILES: CCCC[C@]1(CC)CS(=O)(=O)c2ccc(F)cc2[C@H](c2cccc([N+](=O)[O-])c2)C1=O ; CCCC[C@@]1(CC)CS(=O)(=O)c2ccc(F)cc2C(c2cccc([N+](=O)[O-])c2)C1=O]

or a pharmaceutically acceptable salt, hydrate, or solvate thereof.

Further provided herein is a compound of Formula (II-13),

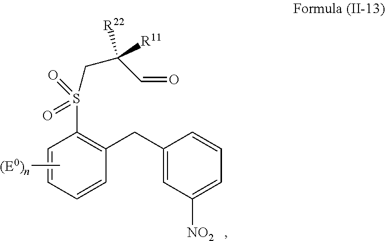

or a pharmaceutically acceptable salt, hydrate, or solvate thereof, wherein:
each of R¹¹ and R²² is independently an optionally substituted (C₁₋₆)alkyl;
n is an integer of 0-4; and each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-13) does not have the structure of Formula (II-A-13),

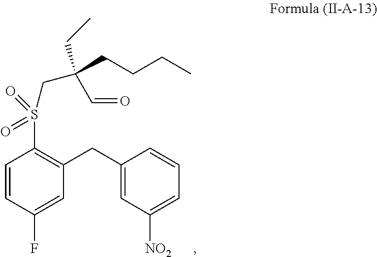 [SMILES: CCCC[C@](C=O)(CC)CS(=O)(=O)c1ccc(F)cc1Cc1cccc([N+](=O)[O-])c1]

or a pharmaceutically acceptable salt, hydrate, or solvate thereof.

In some embodiments, the compound of Formula (II-13) has the structure of Formula (II-B-13), Formula (II-C-13),

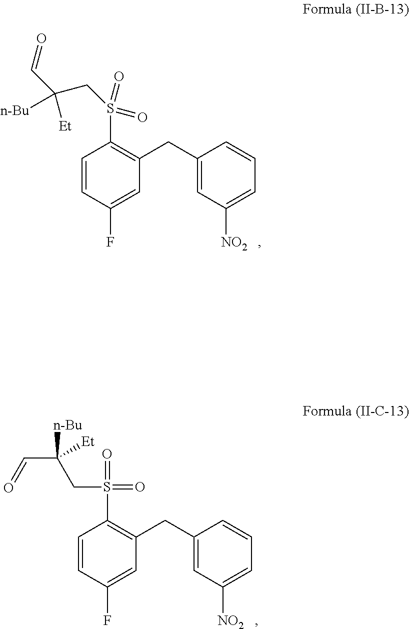

or a pharmaceutically acceptable salt, hydrate, or solvate thereof.

Further provided herein is a compound of Formula (II-12),

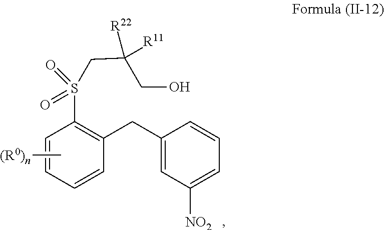

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-12) does not have the structure of Formula (II-A-12),

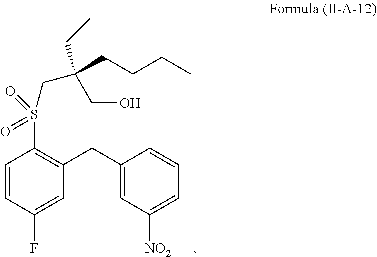 [SMILES: CCCC[C@@](CC)(CO)CS(=O)(=O)c1ccc(F)cc1Cc1cccc([N+](=O)[O-])c1]

or a pharmaceutically acceptable salt, hydrate, or solvate thereof.

In some embodiments, the compound of Formula (II-12) has the structure of Formula (II-B-12),

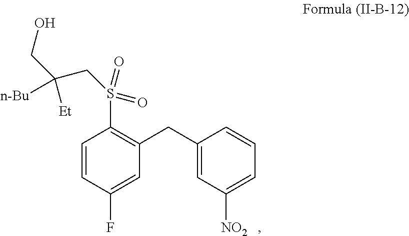

or a pharmaceutically acceptable salt, hydrate, or solvate thereof.

Further provided herein is a compound of Formula (II-11-int),

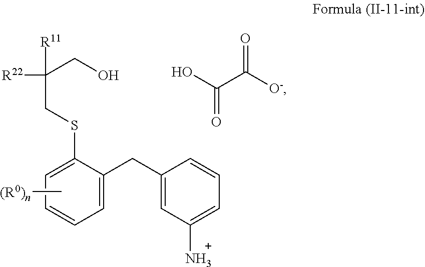

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-11-int) has the structure of Formula (II-A-11-int),

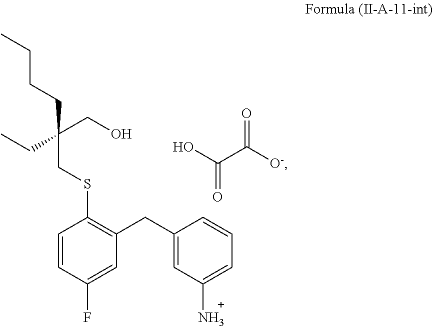

or a pharmaceutically acceptable salt, hydrate, or solvate thereof.

Further provided herein is a compound of Formula (II-11),

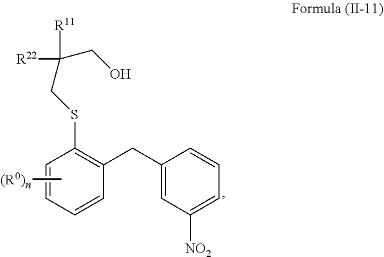

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-11) does not have the structure of Formula (II-A-11),

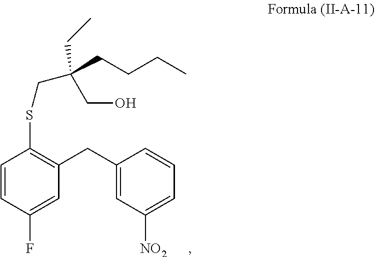 [SMILES: CCCC[C@@](CC)(CO)CSc1ccc(F)cc1Cc1cccc([N+](=O)[O-])c1]

or a pharmaceutically acceptable salt, hydrate, or solvate thereof.

In some embodiments, the compound of Formula (II-11) has the structure of Formula (II-B-11),

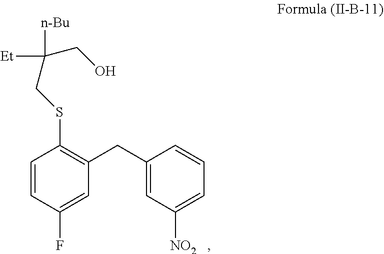

or a pharmaceutically acceptable salt, hydrate, or solvate thereof.

Further provided herein is a compound of Formula (II-10),

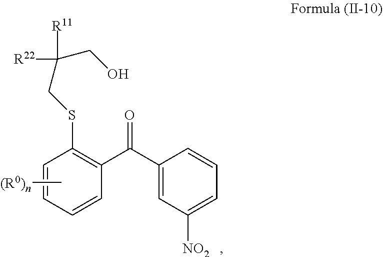

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰, —(C₁₋₆)alkylene-OH, —(C₁₋₆)alkylene-SH, —(C₁₋₆)alkylene-NH₂, wherein each R⁰⁰ is independently (C₁₋₆)alkyl, wherein the compound of Formula (II-10) does not have the structure of Formula (II-A-10),

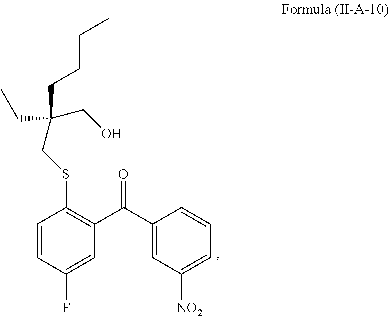 [SMILES: CCCC[C@@](CC)(CO)CSc1ccc(F)cc1C(=O)c1cccc([N+](=O)[O-])c1]

Formula (II-A-10), or a pharmaceutically acceptable salt, hydrate, or solvate thereof.

In some embodiments, the compound of Formula (II-10) has the structure of Formula (II-B-10),

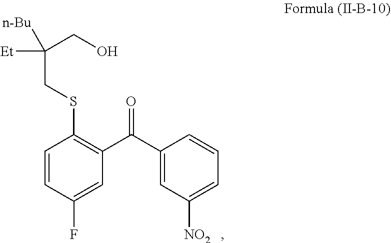

or a pharmaceutically acceptable salt, hydrate, or solvate thereof.

Further provided herein is a compound of Formula (II-215),

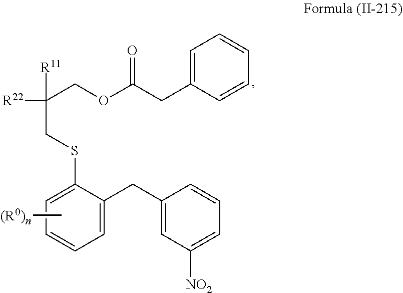

or a pharmaceutically acceptable salt, hydrate, or solvate thereof, wherein:
each of R¹¹ and R²² is independently an optionally substituted (C₁₋₆)alkyl;
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-215) has the structure of Formula (II-A-215),

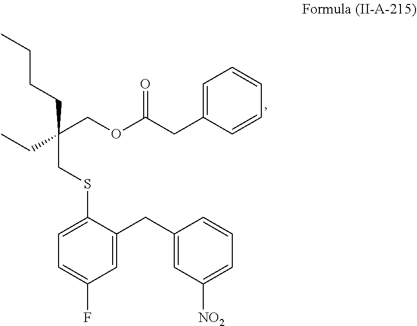 [SMILES: CCCC[C@@](CC)(COC(=O)Cc1ccccc1)CSc1ccc(F)cc1Cc1cccc([N+](=O)[O-])c1]

or a pharmaceutically acceptable salt, hydrate, or solvate thereof.

Additionally provided herein is a compound of Formula (II-210),

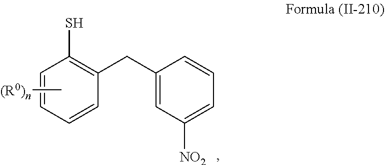

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-210) has the structure of Formula (II-A-210),

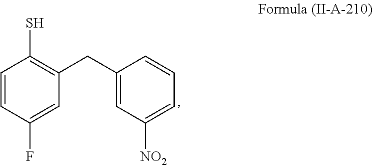 [SMILES: O=[N+]([O-])c1cccc(Cc2cc(F)ccc2S)c1]

or a pharmaceutically acceptable salt, hydrate, or solvate thereof.

Further provided herein is a compound of Formula (II-214),

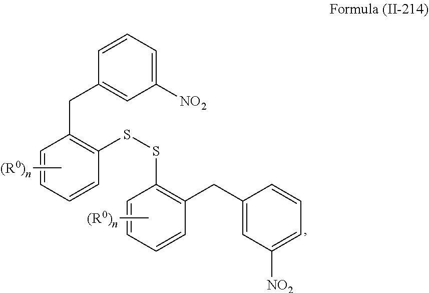

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-214) has the structure of Formula (II-A-214),

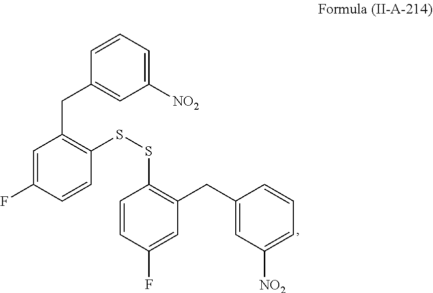 [SMILES: O=[N+]([O-])c1cccc(Cc2cc(F)ccc2SSc2ccc(F)cc2Cc2cccc([N+](=O)[O-])c2)c1]

or a pharmaceutically acceptable salt, hydrate, or solvate thereof.

Additionally provided herein is a compound of Formula (II-205),

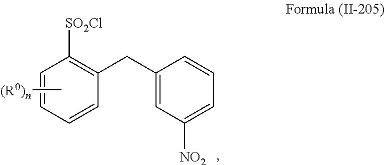

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-205) has the structure of Formula (II-A-205),

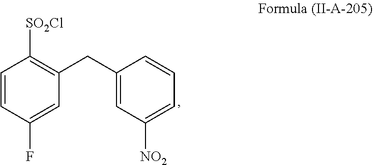 [SMILES: O=[N+]([O-])c1cccc(Cc2cc(F)ccc2S(=O)(=O)Cl)c1]

or a pharmaceutically acceptable salt, hydrate, or solvate thereof.

Further provided herein is a compound of Formula (II-209),

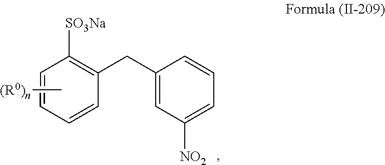

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-209) has the structure of Formula (II-A-209),

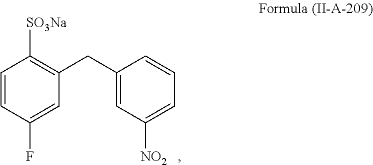

or a pharmaceutically acceptable salt, hydrate, or solvate thereof.

Additionally provided herein is a compound of Formula (II-204),

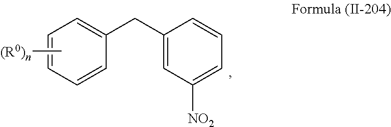

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-204) has the structure of Formula (II-A-204),

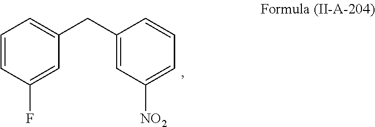 [SMILES: O=[N+]([O-])c1cccc(Cc2cccc(F)c2)c1]

or a pharmaceutically acceptable salt, hydrate, or solvate thereof.

Further provided herein is a compound of Formula (II-203),

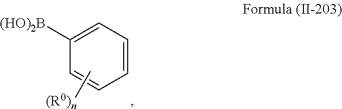

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-203) has the structure of Formula (II-A-203),

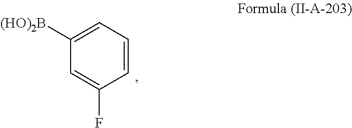

or a pharmaceutically acceptable salt, hydrate, or solvate thereof.

Additionally provided herein is a compound of Formula (II-202),

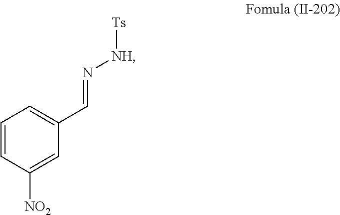 [SMILES: Cc1ccc(S(=O)(=O)N/N=C/c2cccc([N+](=O)[O-])c2)cc1]

or a pharmaceutically acceptable salt, hydrate, or solvate thereof.

Further provided herein is a compound of Formula (II-208),

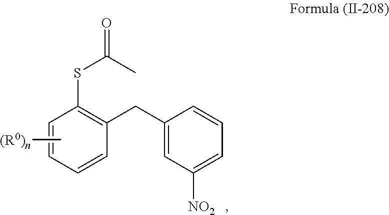

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-208) has the structure of Formula (II-A-208),

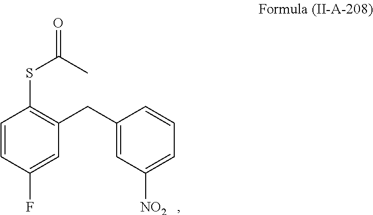 [SMILES: CC(=O)Sc1ccc(F)cc1Cc1cccc([N+](=O)[O-])c1]

or a pharmaceutically acceptable salt, hydrate, or solvate thereof.

Additionally provided herein is a compound of Formula (II-207),

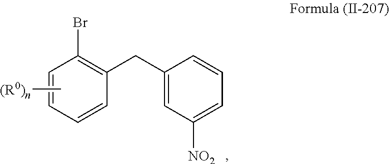

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-207) has the structure of Formula (II-A-207),

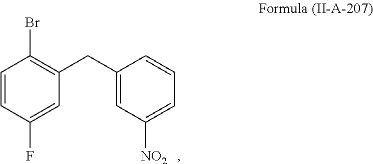 [SMILES: O=[N+]([O-])c1cccc(Cc2cc(F)ccc2Br)c1]

or a pharmaceutically acceptable salt, hydrate, or solvate thereof.

Further provided herein is a compound of Formula (II-206),

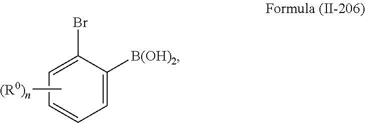

or a pharmaceutically acceptable salt, hydrate, or solvate thereof, wherein:
n is an integer of 0-4; and
each R⁰ is independently halo, —OH, —SH, —CN, —OH, —SH, —COOH, —O—R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl.

In some embodiments, the compound of Formula (II-206) has the structure of Formula (II-A-206),

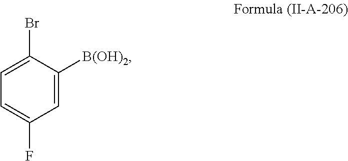 [SMILES: OB(O)c1cc(F)ccc1Br]

or a pharmaceutically acceptable salt, hydrate, or solvate thereof.

Further provided herein is a compound of Formula (II-B-7),

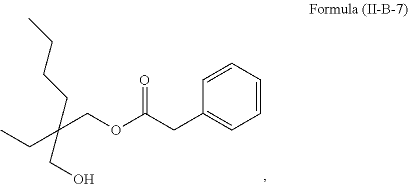 [SMILES: CCCCC(CC)(CO)COC(=O)Cc1ccccc1]

or a pharmaceutically acceptable salt, hydrate, or solvate thereof.

Additionally provided herein is a compound of Formula (II-C-9)

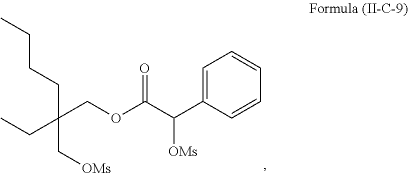 [SMILES: CCCCC(CC)(COC(=O)C(OS(C)(=O)=O)c1ccccc1)COS(C)(=O)=O]

or a pharmaceutically acceptable salt, hydrate, or solvate thereof.

Further provided herein is a compound of Formula (II-B-9),

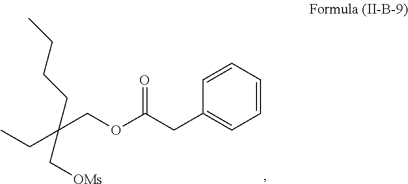 [SMILES: CCCCC(CC)(COC(=O)Cc1ccccc1)COS(C)(=O)=O]

or a pharmaceutically acceptable salt, hydrate, or solvate thereof.

Additionally provided herein is a compound of Formula (II-B-44),

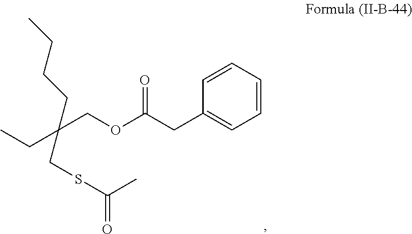 [SMILES: CCCCC(CC)(COC(=O)Cc1ccccc1)CSC(C)=O]

or a pharmaceutically acceptable salt, hydrate, or solvate thereof.

Further provided herein is a compound of Formula (I-A-66),

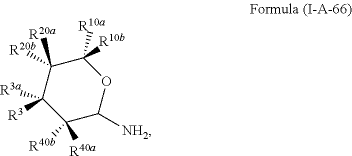

or a pharmaceutically acceptable salt, hydrate, or solvate thereof, wherein:
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁₋₆)alkyl, NH₂, NH(C₁₋₆)alkyl, or N((C₁₋₆)alkyl)₂;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂; and
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂.

In some embodiments, the compound of Formula (I-A-66) has the structure of Formula (II-A-166), Formula (II-A-77),

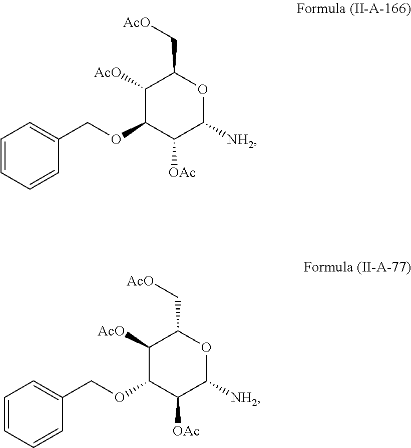 [SMILES: CC(=O)OC[C@H]1O[C@H](N)[C@H](OC(C)=O)[C@@H](OCc2ccccc2)[C@@H]1OC(C)=O ; CC(=O)OC[C@@H]1O[C@H](N)[C@@H](OC(C)=O)[C@H](OCc2ccccc2)[C@H]1OC(C)=O]

or a pharmaceutically acceptable salt, hydrate, or solvate thereof.

Further provided herein is a compound prepared by the processes as described herein.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Additionally provided herein is a pharmaceutical composition comprising a therapeutically effective amount of any of the compounds as described herein.

Generally, the compound as described herein are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

The pharmaceutical compositions of the compound as described herein can be administered by a variety of routes including oral, rectal, intraocular, transdermal, subcutaneous, intravenous, intramuscular, intraperitoneal, intradermal, directly into cerebrospinal fluid, intratracheal, and intranasal. Depending on the intended route of delivery, the compounds as described herein are preferably formulated as oral compositions.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, or the like in the case of solid compositions. In such compositions, the active compound is usually a minor component with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors, and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions containing the compounds as described herein can be prepared in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Provided herein are embodiments directed to a pharmaceutical composition comprising any of the compounds as described herein, or a pharmaceutically acceptable salt, hydrate, or solvate thereof, that is in an amount of less than about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, 5 mg, 3 mg, 2 mg, or 1 mg. In some embodiments, any of the compounds as described herein, or a pharmaceutically acceptable salt, hydrate, or solvate thereof, is in an amount of about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 12.5 mg, 10 mg, 7.5 mg, 5 mg, 3 mg, 2 mg, or 1 mg. In some embodiments, any of the compounds as described herein, or a pharmaceutically acceptable salt, hydrate, or solvate thereof, is in an amount of about 1 mg-150 mg, 3 mg-150 mg, 5 mg-150 mg, 7.5 mg-150 mg, 10 mg-150 mg, 12.5 mg-150 mg, 15 mg-150 mg, 20 mg-150 mg, 25 mg-150 mg, 30 mg-150 mg, 35 mg-150 mg, 40 mg-150 mg, 1 mg-120 mg, 3 mg-120 mg, 5 mg-120 mg, 7.5 mg-120 mg, 10 mg-120 mg, 12.5 mg-120 mg, 15 mg-120 mg, 20 mg-120 mg, 25 mg-120 mg, 30 mg-120 mg, 35 mg-120 mg, 40 mg-120 mg, 1 mg-100 mg, 3 mg-100 mg, 5 mg-100 mg, 7.5 mg-100 mg, 10 mg-100 mg, 12.5 mg-100 mg, 15 mg-100 mg, 20 mg-100 mg, 25 mg-100 mg, 30 mg-100 mg, 35 mg-100 mg, 40 mg-100 mg, 1 mg-80 mg, 3 mg-80 mg, 5 mg-80 mg, 7.5 mg-80 mg, 10 mg-80 mg, 12.5 mg-80 mg, 15 mg-80 mg, 20 mg-80 mg, 25 mg-80 mg, 30 mg-80 mg, 35 mg-80 mg, 40 mg-80 mg, 1 mg-60 mg, 3 mg-60 mg, 5 mg-60 mg, 7.5 mg-60 mg, 10 mg-60 mg, 12.5 mg-60 mg, 15 mg-60 mg, 20 mg-60 mg, 25 mg-60 mg, 30 mg-60 mg, 35 mg-60 mg, 40 mg-60 mg, 1 mg-50 mg, 3 mg-50 mg, 5 mg-50 mg, 7.5 mg-50 mg, 10 mg-50 mg, 12.5 mg-50 mg, 15 mg-50 mg, 20 mg-50 mg, 25 mg-50 mg, 30 mg-50 mg, 35 mg-50 mg, 40 mg-50 mg, 1 mg-40 mg, 3 mg-40 mg, 5 mg-40 mg, 7.5 mg-40 mg, 10 mg-40 mg, 12.5 mg-40 mg, 15 mg-40 mg, 20 mg-40 mg, 25 mg-40 mg, 30 mg-40 mg, 35 mg-40 mg, 1 mg-30 mg, 3 mg-30 mg, 5 mg-30 mg, 7.5 mg-30 mg, 10 mg-30 mg, 12.5 mg-30 mg, 15 mg-30 mg, 20 mg-30 mg, 25 mg-30 mg, 1 mg-20 mg, 3 mg-20 mg, 5 mg-20 mg, 7.5 mg-20 mg, 10 mg-20 mg, 12.5 mg-20 mg, 15 mg-20 mg, 1 mg-15 mg, 3 mg-15 mg, 5 mg-15 mg, 7.5 mg-15 mg, 10 mg-15 mg, 12.5 mg-15 mg, 1 mg-10 mg, 3 mg-10 mg, 5 mg-10 mg, 7.5 mg-10 mg, 1 mg-7.5 mg, 3 mg-7.5 mg, 5 mg-7.5 mg, 1 mg-5 mg, or 3 mg-5 mg.

Provided herein are embodiments directed to a pharmaceutical composition comprising any of the compounds as described herein, or a pharmaceutically acceptable salt, hydrate, or solvate thereof, that is in an amount of less than about 80 mg, 75 mg, 70 mg, 65 mg, 60 mg, 55 mg, 50 mg, 45 mg, 40 mg, 35 mg, 30 mg, 25 mg, or 20 mg. In some embodiments, any of the compounds as described herein, or a pharmaceutically acceptable salt, hydrate, or solvate thereof, is in an amount of about 80 mg, 75 mg, 70 mg, 65 mg, 60 mg, 55 mg, 50 mg, 45 mg, 40 mg, 35 mg, 30 mg, 25 mg, or 20 mg. In some embodiments, any of the compounds as described herein, or a pharmaceutically acceptable salt, hydrate, or solvate thereof, is in an amount of about 20 mg-80 mg, 25 mg-80 mg, 30 mg-80 mg, 35 mg-80 mg, 40 mg-80 mg, 20 mg-60 mg, 25 mg-60 mg, 30 mg-60 mg, 35 mg-60 mg, 40 mg-60 mg, 20 mg-50 mg, 25 mg-50 mg, 30 mg-50 mg, 35 mg-50 mg, 40 mg-50 mg, 20 mg-40 mg, 25 mg-40 mg, 30 mg-40 mg, 35 mg-40 mg, 20 mg-30 mg, or 25 mg-30 mg.

In some embodiments, the compound is present in the composition in an amount of less than about 100 mg.

In some embodiments, the compound is present in the composition in an amount of less than about 80 mg.

In some embodiments, the compound is present in the composition in an amount of less than about 50 mg.

In some embodiments, the compound is present in the composition in an amount of about 20 mg to about 80 mg.

In some embodiments, the compound is present in the composition in an amount of about 20 mg.

In some embodiments, the compound is present in the composition in an amount of about 80 mg.

In some embodiments, the compound is present in the composition in an amount of less than about 20 mg, about 20 mg to 80 mg, or more than about 80 mg.

In some embodiments, the compound is present in the composition in an amount of less than about 30 mg, about 30 mg to 70 mg, or more than about 70 mg.

In some embodiments, the pharmaceutical composition comprising any of the compounds as described herein, or a pharmaceutically acceptable salt, hydrate, or solvate thereof, is dosed at a frequency of once daily or twice daily.

In one embodiment, the pharmaceutical composition comprising about 20 mg of the compound as described herein is administered twice daily.

In one embodiment, the pharmaceutical composition comprising about 80 mg of the compound as described herein is administered twice daily.

### Pharmaceutical Dosage Forms

Further provided herein is a pharmaceutical dosage form comprising any of the compounds or the pharmaceutical compositions as described herein.

### Methods of Treatment

Additionally provided herein is a method of treating a cholestatic liver disease in a subject in need thereof, comprising administering to the subject a therapeutically effective amount of any of the compounds, the pharmaceutical compositions, or the pharmaceutical dosage forms as described herein.

In some embodiments, the cholestatic liver disease is a pediatric cholestatic liver disease.

In some embodiments, the cholestatic liver disease is an adult cholestatic liver disease.

In some embodiments, the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, PFIC type 4, PFIC type 5, PFIC type 6, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

In some embodiments, the cholestatic liver disease is selected from the group consisting of ALGS, PFIC, BA, ICP, BRIC, PSC, and PBC.

In some embodiments, the cholestatic liver disease is PSC.

In some embodiments, the cholestatic liver disease is PBC.

In some embodiments, the cholestatic liver disease is ICP.

In some embodiments, the cholestatic liver disease is characterized by pruritus.

In some embodiments, the subject has elevated total serum bile acids (sBA) prior to the administration of the pharmaceutical composition.

In some embodiments, the compound of Formula (I) is an ileal bile acid transporter (IBAT) inhibitor.

In some embodiments, the compound of Formula (I) has the structure of the compound of Formula (II-A-27), that is an ileal bile acid transporter (IBAT) inhibitor.

Further provided herein is a method of treating hyperlipidemia in a subject in need thereof, wherein the method comprises administering to the subject a therapeutically effective amount of any of the compounds, the pharmaceutical compositions, or the pharmaceutical dosage forms as described herein.

Additionally provided herein is a method of lowering the serum cholesterol level in a subject in need thereof, wherein the method comprises administering to the subject a therapeutically effective amount of any of the compounds, the pharmaceutical compositions, or the pharmaceutical dosage forms as described herein.

Further provided herein is a method of treating arteriosclerosis in a subject in need thereof, wherein the method comprises administering to the subject a therapeutically effective amount of any of the compounds, the pharmaceutical compositions, or the pharmaceutical dosage forms as described herein.

Additionally provided herein is a method of treating Syndrome X in a subject in need thereof, wherein the method comprises administering to the subject a therapeutically effective amount of any of the compounds, the pharmaceutical compositions, or the pharmaceutical dosage forms as described herein.

In some embodiments, the compound, the pharmaceutical composition, or the pharmaceutical dosage is administered to the subject orally.

The pharmaceutical compositions of this invention can be administered by a variety of routes including oral, rectal, intraocular, transdermal, subcutaneous, intravenous, intramuscular, intraperitoneal, intradermal, directly into cerebrospinal fluid, intratracheal, and intranasal. In some embodiments, the composition is administered to the subject orally.

### Pharmaceutical Compositions and Kits

Further provided herein is a pharmaceutical composition comprising a therapeutically effective amount of the compound of Formula (I) and one or more excipients selected from the group consisting of:
(i) a diluent or a filler,
(ii) a disintegrant,
(iii) a channeling agent,
(iv) a glidant, and
(v) a lubricant.

Additionally provided herein is a kit for treating cholestatic liver disease in a subject in need thereof, wherein the kit comprises at least one unit dosage of a therapeutically effective amount of any of the compounds, the pharmaceutical compositions, or the pharmaceutical dosage forms as described herein.

In some embodiments, the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, PFIC type 4, PFIC type 5, PFIC type 6, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, RP2 deficiency syndrome, or neonatal sclerosing cholangitis.

In some embodiments, the cholestatic liver disease is ALGS, PFIC, BA, ICP, BRIC, PSC, or PBC.

In some embodiments, the cholestatic liver disease is PSC.

In some embodiments, the cholestatic liver disease is PBC.

In some embodiments, the cholestatic liver disease is ICP.

Further provided herein is a kit for treating hyperlipidemia in a subject in need thereof, wherein the kit comprises a therapeutically effective amount of any of the compounds, the pharmaceutical compositions, or the pharmaceutical dosage forms as described herein.

Additionally provided herein is a kit for lowering the serum cholesterol level in a subject in need thereof, wherein the kit comprises a therapeutically effective amount of any of the compounds, the pharmaceutical compositions, or the pharmaceutical dosage forms as described herein.

Further provided herein is a kit for treating arteriosclerosis in a subject in need thereof, wherein the kit comprises a therapeutically effective amount of any of the compounds, the pharmaceutical compositions, or the pharmaceutical dosage forms as described herein.

Additionally provided herein is a kit for treating Syndrome X in a subject in need thereof, wherein the kit comprises a therapeutically effective amount of any of the compounds, the pharmaceutical compositions, or the pharmaceutical dosage forms as described herein.

### EXAMPLES

The following examples illustrate the invention. These examples are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

### General Synthetic Procedures

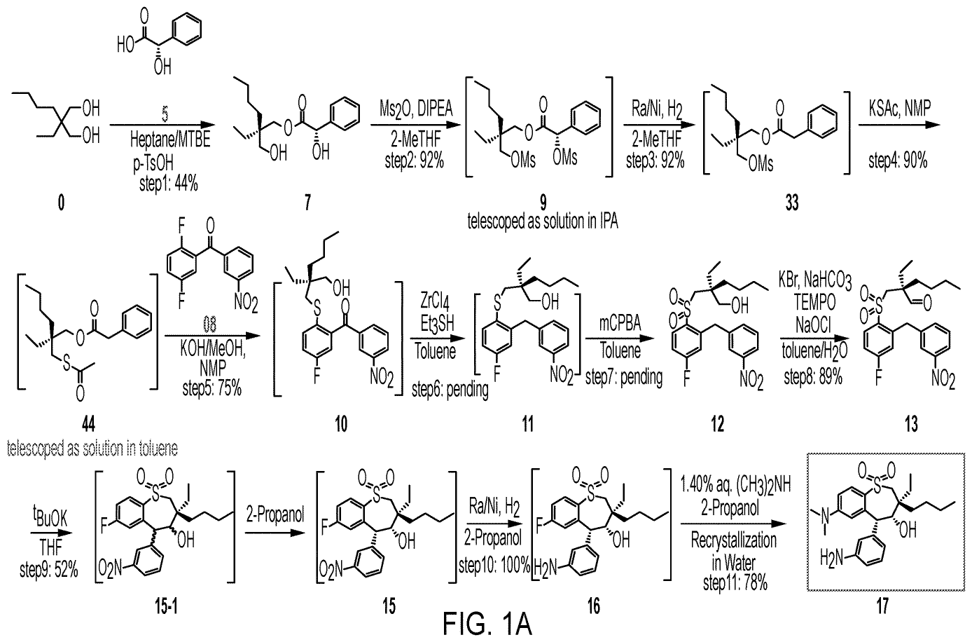

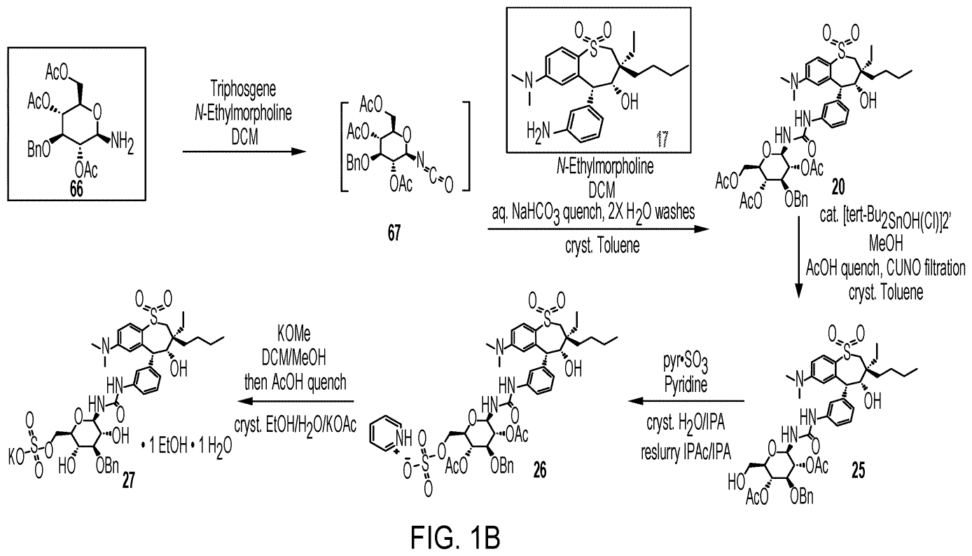

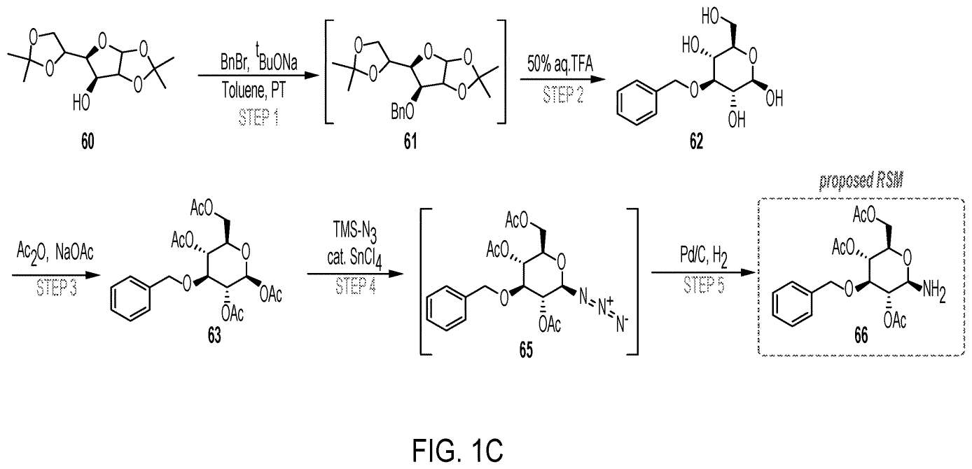

Compound 17 (Formula (II-A-17)) was synthesized by the scheme shown in FIG. 1A. The synthesis as described herein to synthesize compound 10 did not rely on chiral resolution with quinine to set the quaternary stereocenter as previously described. Thus, the synthesis as described herein was higher yielding and required fewer functional group manipulations than previous work. Compound 27 (Formula (II-A-27)) was synthesized by the scheme shown in FIG. 1B. The synthesis as described herein to synthesize compound 27 used a selective Sn-based catalyst for a more streamlined, higher yielding synthesis compared to a non-selective deacetylation and salt/cation exchange process as shown in previous work. Compound 66 (Formula (II-A-66)) was synthesized by the scheme shown in FIG. 1C. The detailed steps are described below.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

### Example 1. Synthesis of Compound 7 (Formula (II-A-7)) and Analog

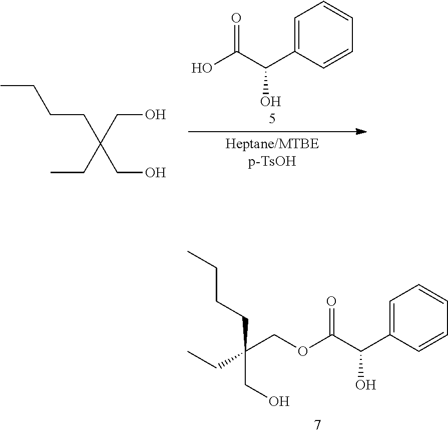 [SMILES: CCCCC(CC)(CO)CO ; O=C(O)[C@@H](O)c1ccccc1 ; CCCC[C@](CC)(CO)COC(=O)[C@@H](O)c1ccccc1]

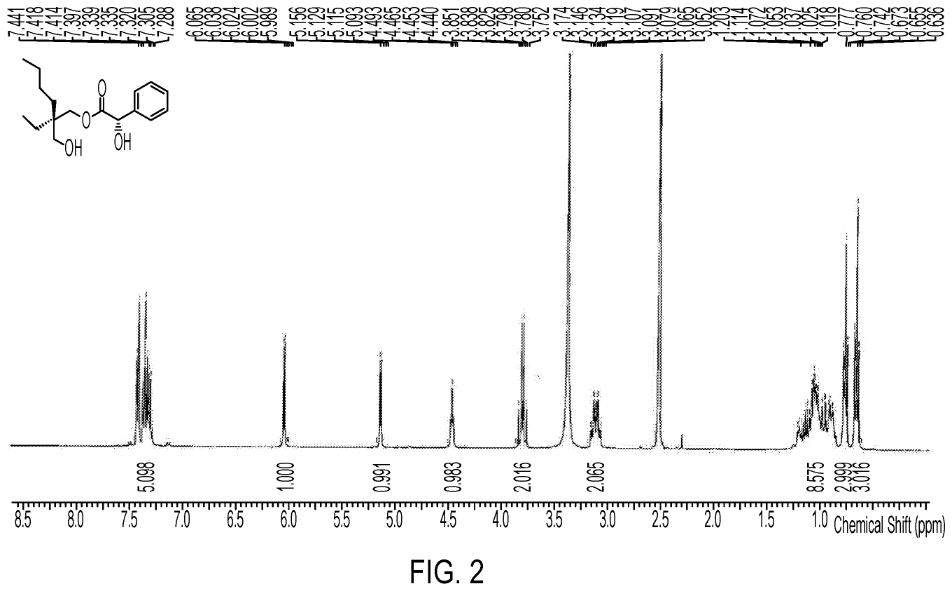

2-Butyl-2-Ethyl-1,3-Propanediol (71.0 kg, 444 mol) and (S)-mandelic acid (67.4 kg, 443 mol) were dissolved in mixture of MTBE (355 L) and n-heptane (1765 L) at 20° C. Then pTSA monohydrate (1.7 kg, 8.9 mol) was added and the mixture was stirred for 2 h at 40-42° C. Distillation was then performed to distillate out total 500 L mixed solvents seven times (each time 71 L with inner temperature maintained between 38-43° C. and stirred at same temperature range for 2 h). After the 7^{th} distillation, the ratio of (7 (Formula (II-A-7))+Bis-mandelate)/5 (Formula (II-A-5)) was checked by HPLC (High Performance Liquid Chromatography). If the ratio was less than 10, the previous distillation process was continued until the ratio met the specification. If the ratio was more than 10, the solvent composition (w/w) of MTBE/n-heptane was checked by GC. If the MTBE/n-heptane ratio was between 8-12 w/w %, the process was moved forward to crystallization. If the MTBE/n-heptane ratio was out of the range, MTBE and n-heptane were added to tune the ratio based on testing results. Then the mixture was cooled to 30° C. in 3 h and seed (0.27 kg, 0.4%) was added and aged at same temperature for 0.5 h. The batch was further cooled to 20° C. and aged for another 24 h. After filtration, 133.45 kg wet cake was obtained. The resulting wet cake was further purified by slurry in mixture of MTBE (177 L) and n-heptane (883 L) twice for 12 h at 20° C. with DIPEA (2.27 kg, 22 mol) added in the 1^{st} slurry to neutralize the residual PTSA. The final wet cake was dried at 35-45° C. for 24 h to yield the product as an off-white solid (7, 55.47 kg, 42.5%). Subsequent iterations of the synthesis of compound 7 yielded compound 7 in 52% yield and >99:1 dr. NMR of compound 7 (Formula (II-A-7)) is shown in FIG. 2. ¹H-NMR (400 MHz, DMSO-d₆): δ0.64 (t, J=7.6 Hz, 3H), δ0.74 (t, J=7.2 Hz, 3H), δ1.02 (m, 8H), δ3.05 (m, 2H), δ3.75 (m, 2H), δ4.44 (q, J=5.2 Hz, 1H), δ5.09 (q, J=8.8 Hz, 1H), δ5.99 (q, J=8.8 Hz, 1H), δ7.29 (m, 5H).

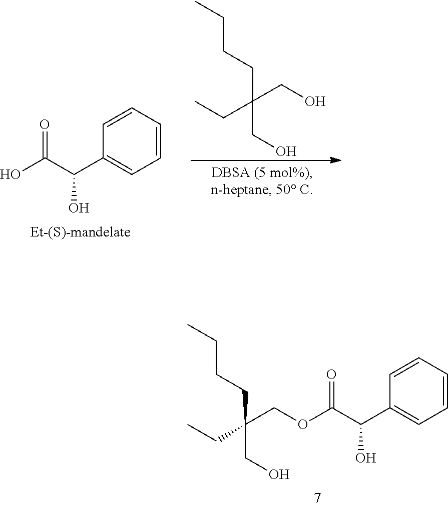

Alternatively, compound 7 was synthesized with Et-(S)-mandelate. A mixture of 2-butyl-2-ethylpropane-1,3-diol (24.0 g, 150 mmol, 1.50 equiv.), ethyl (2S)-2-hydroxy-2-phenyl-acetate (18.0 g, 99.9 mmol, 1.00 equiv.), and n-heptane (36 mL, 2 vol.) was stirred at 50° C. until a solution was obtained. 4-Dodecylbenzenesulfonic acid (DBSA, 1.63 g, 1.54 mL, 4.99 mmol, 5 mol %) was dissolved in n-heptane (2 mL, 0.1 vol.) and added to the reaction mixture. The addition funnel was rinsed with n-heptane (34 mL, 1.9 vol.). The resulting solution was stirred at 50° C., and the n-heptane was evaporated at 150 mbar. The mixture was then stirred under vacuum (100 mbar) with a nitrogen bleed for 20 h. n-Heptane (144 mL, 8 vol.) was added and the mixture was stirred at 50° C. and ambient pressure until a clear solution was obtained. The mixture was cooled to 31° C. and methyl cyclohexane (36 mL, 2 vol.) was added. The solution was treated with compound 7 seeds (882 mg, 3 mol %). The resulting suspension was cooled to 25° C. over two hours, stirred at this temperature for 3.5 hours, cooled to 10° C. over one hour, and stirred at this temperature for 18 hours. The suspension was filtered, and the colorless solid was washed with n-heptane (36 mL, 2 vol.). The solids were then dried in vacuo for 12 h to yield compound 7 as a colorless solid (20.59 g, 67% corrected yield). NMR of compound 7 (Formula (II-A-7)) is as follows: ¹H-NMR (400 MHz, CDCl₃); δ [ppm]=7.44-7.28 (m, 5H), 5.16 (s, 1H), 4.09 (dd, J=11.1, 13.4 Hz, 1H), 3.92 (dd, J=8.6, 11.1 Hz, 1H), 3.19 (dd, J=8.0, 11.5 Hz, 1H), 3.09 (dd, J=11.4, 16.4 Hz, 1H), 2.92 (s, 2H), 1.25-0.89 (m, 8H), 0.83 (dt, J=7.1, 11.6 Hz, 3H), 0.76-0.65 (m, 3H).

Alternative conditions for this reaction include, but are not limited to, other acids (e.g., TfOH, pTsOH, MsOH, H₂SO₄, HCl, H₃PO₄, TFA), the presence of a base (e.g., hydroxide bases such as LiOH, NaOH, KOH; alkoxide bases such as LiOMe, NaOMe, KOMe, LiOEt, NaOEt, KOEt, etc.; or carbonates such as Li₂CO₃, Na₂CO₃, K₂CO₃, Cs₂CO₃, etc.), use of a metal oxide (e.g., CaO, MgO), the presence of a Lewis acid (e.g., AlCl₃, AlBr₃, FeCl₃, FeBr₃, ZnCl₂, Ti(OiPr)₄, Al(OEt)₃, etc.), the presence of a heterogeneous acid catalyst (e.g., ion exchange resins or zeolites), and other solvents (e.g., alkanes such as pentanes, hexanes, octanes, cyclopentane, cyclohexane, methyl cyclohexane, etc.; aromatic solvents such as benzene, toluene, ethyl benzene, chlorobenzene, etc.; or ethers such as diethyl ether, di-n-butyl ether, diisopropyl ether, methyl tert-butyl ether, ethyl tert-butyl ether, cyclopropyl methyl ether, tert-amyl methyl ether, THF, methyl THF, dioxane, etc.).

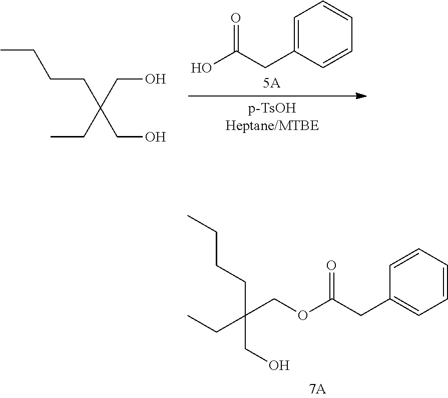 [SMILES: CCCCC(CC)(CO)CO ; O=C(O)Cc1ccccc1 ; CCCCC(CC)(CO)COC(=O)Cc1ccccc1]

Analog of compound 7, compound 7A, was also synthesized. Compound 5A (Formula (II-B-5)) (84.9 g, 624 mmol, 78.6 mL, 1 eq.) was added to a mixture of 2-Butyl-2-Ethyl-1,3-Propanediol (100 g, 624 mmol, 1 eq.) in MTBE (200 mL) and heptane (1000 mL) at 20° C. p-TsOH (2.00 g, 11.6 mmol, 0.0186 eq.) was added and the mixture was stirred at 45° C. for 12 hours. The reaction mixture was concentrated under reduced pressure and a residue was obtained. The residue was purified by column chromatography (Petroleum ether:Ethyl acetate 3:1) and compound 7A (Formula (II-B-7)) was obtained as a colorless oil.

### Example 2. Synthesis of Compound 9 (Formula (II-A-9)) and Analog

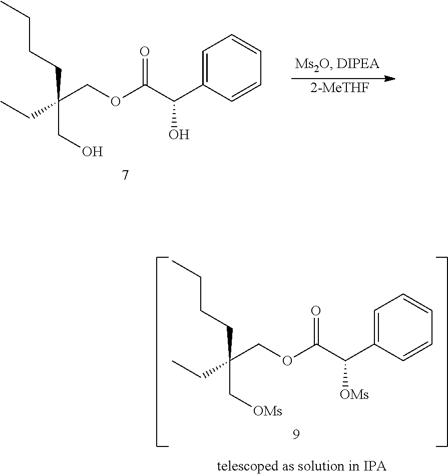

In the first reactor, DIPEA (193 kg, 1493 mol) was dissolved in 2-MeTHF (525 L) at 25° C., then 7 (Formula (II-A-7)) (105 kg, 357 mol) was added to form a clear solution. In the second reactor, Ms₂O (181 kg, 1040 mol) was dissolved in 2-MeTHF (1050 L) to form a clear solution and then transferred into the first reactor portion-wise for 10.5 h with inner temperature at 10° C. The mixture in the first reactor was stirred for total 4 h and monitored by HPLC before being quenched with purified water (630 L). The separated organic phase was washed with HCl aq. solution (1N, 840 L), NaHCO₃ aq. solution (5 wt %, 525 L) and Na₂SO₄ aq. solution (5 wt %, 525 L) in turn and filtered through CUNO to yield a 2-MeTHF solution of 9 (Formula (II-A-9)) (1338.2 kg solution, 100%, telescoped into next step directly).

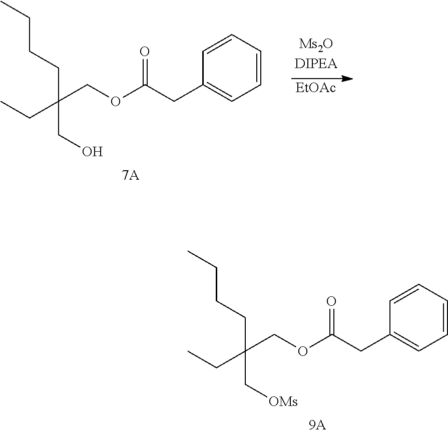 [SMILES: "EtOAc" ; CCCCC(CC)(CO)COC(=O)Cc1ccccc1 ; CCCCC(CC)(COC(=O)Cc1ccccc1)COS(C)(=O)=O]

Also prepared was compound 9A. Compound 7A (Formula (II-B-7)) (200 g, 719.15 mmol, 1 eq.) was combined with Ms₂O (137.80 g, 791.06 mmol, 1.1 eq.) in EtOAc (2000 mL) at 20° C. DIPEA (120 g, 934 mmol, 1.3 eq.) was added and the reaction was agitated at 20° C. for 12 h. LCMS confirmed compound 7A (Formula (II-B-7)) was fully consumed and compound 9A (Formula (II-B-9)) was obtained. The reaction mixture was concentrated under reduced pressure and a residue was obtained. The residue was purified by column chromatography (Petroleum ether:ethyl acetate 5:1) and compound 9A (Formula (II-B-9)) was obtained as a brown oil (136.8 g).

### Example 3. Synthesis of Compound 33 (Formula (II-A-33))

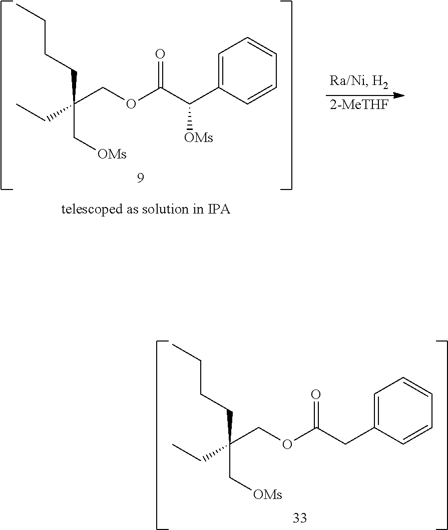

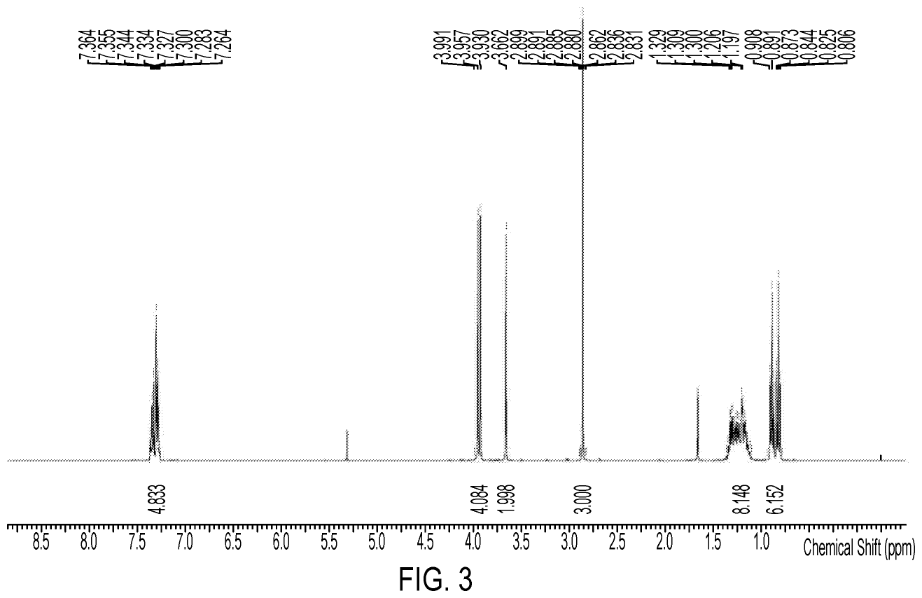

2-MeTHF solution of 9 (Formula (II-A-9)) (1334 kg solution) and Raney Ni (32 kg) were added into a hydrogenation reactor made inert with N₂ three times. The reactor was then swapped with H₂ three times and the final H₂ pressure was filled to 2-3 bar. The mixture was stirred for total 108 h at 35-45° C. and monitored by HPLC. The mixture was filtered and washed with NaHCO₃ aq. solution (5 wt %, 820 L) and Na₂SO₄ aq. solution (5 wt %, 830 L) in turn. After concentration, 755.2 kg NMP solution of 33 (Formula (II-A-33)) was obtained (755.2 kg solution, 111 kg active, 87% over two steps). Subsequent iterations of the synthesis of compound 33 yielded compound 33 in 98% yield over two steps. NMR of compound 33 (Formula (II-A-33)) is shown in FIG. 3. ¹H-NMR (400 MHz, CDCl₃): δ0.81 (t, J=7.6 Hz, 3H), δ0.87 (t, J=7.2 Hz, 3H), δ1.20 (m, 8H), δ2.88 (s, 3H), δ3.66 (s, 2H), δ3.93 (s, 2H), δ3.96 (s, 2H), δ7.26 (m, 5H).

### Example 4. Synthesis of Compound 44 (Formula (II-A-44)) and Enantiomeric Mixture

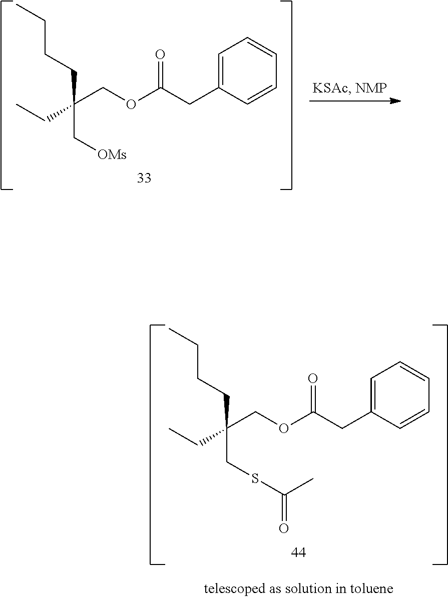

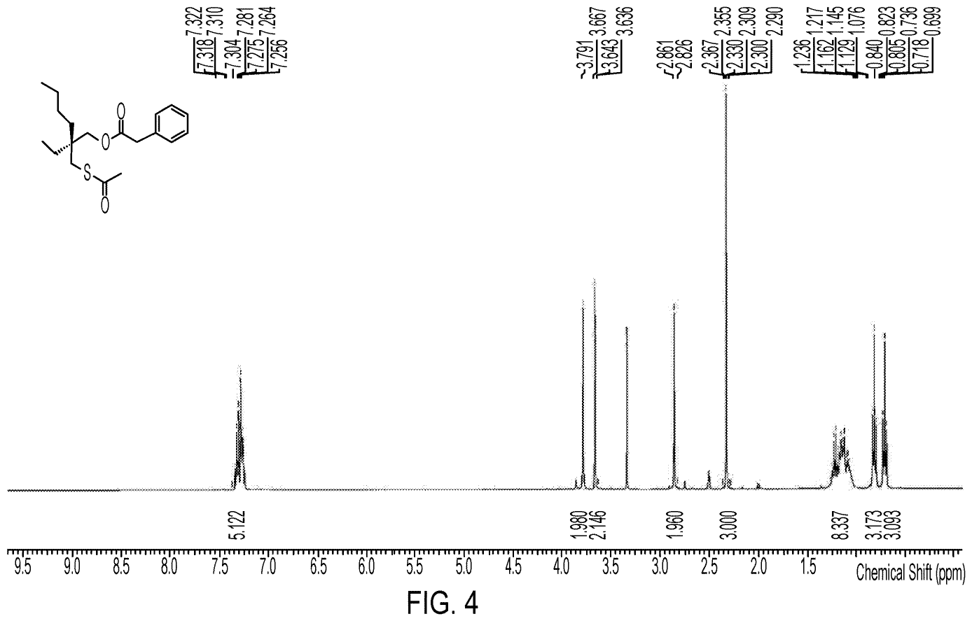

NMP solution of 33 (Formula (II-A-33)) (755 kg solution, 111.0 kg active, 311 mol) was diluted with NMP (577 kg) at 25° C. KSAc (53 kg, 465 mol) was then added as solid and the mixture was stirred for 8.0 h at 100-115° C. until 33 (Formula (II-A-33)) was all consumed. The temperature was cooled to 25° C. and water (888 L) was added slowly for quenching. Toluene (888 L+655 L) was used to extract the product from aqueous phase twice and the resulting organic phase was combined and washed with Na₂SO₄ aq. solution (5 wt %, 444 L). The solution was concentrated to control the water content and yielded the final product as a toluene solution (333.6 kg solution, 102.4 kg active, 97.8%). NMR of compound 44 (Formula (II-A-44)) is shown in FIG. 4. ¹H-NMR (400 MHz, DMSO-d₆): δ0.70 (t, J=7.6 Hz, 3H), δ0.81 (t, J=7.2 Hz, 3H), δ1.08 (m, 8H), δ2.33 (s, 3H), δ2.86 (s, 2H), δ3.67 (s, 2H), δ3.79 (s, 2H), δ7.26 (m, 5H).

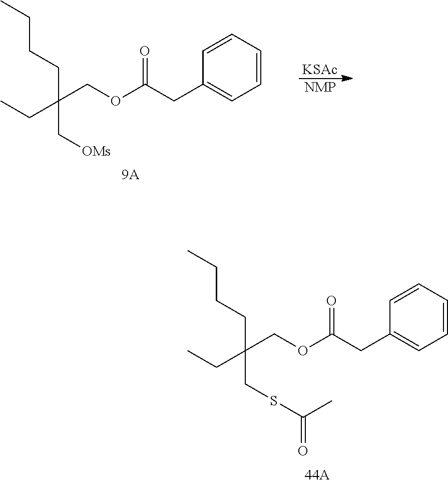

Also prepared was enantiomeric mixture 44A. To a mixture of compound 9A (Formula (II-B-9)) (113 g, 316 mmol, 1 eq.) in NMP (1100 mL) was added KSAc (55.7 g, 487 mmol, 1.54 eq.) in portions at 20° C. The reaction mixture was agitated at 105° C. for 24 h and TLC indicated full consumption of compound 9A (Formula (II-B-9)). The reaction mixture was transferred into cooled water (400 mL) and agitated. The aqueous phase was extracted with EtOAc (1000 mL×2), and the combined organic phase was washed with brine (1000 mL), dried with anhydrous Na₂SO₄, filtered, and concentrated under vacuum. The resulting residue was purified by column chromatography (Petroleum ether:Ethyl acetate 10:1) and compound 44A (Formula (II-B-44)) (80.4 g, 239 mmol) was obtained as a brown oil. NMR of compound 44A (Formula (II-B-44)) is as follows: ¹H-NMR (400 MHz, CDCl₃) δ ppm 7.35-7.28 (m, 4H), 7.26-7.22 (m, 1H), 3.85 (s, 2H), 3.66-3.63 (m, 2H), 2.92 (s, 2H), 2.33 (s, 3H), 1.32-1.05 (m, 8H), 0.89-0.83 (m, 3H), 0.80-0.73 (m, 3H).

### Example 5. Synthesis of Compound 10 (Formula (IT-A-10)) and Enantiomeric Mixture

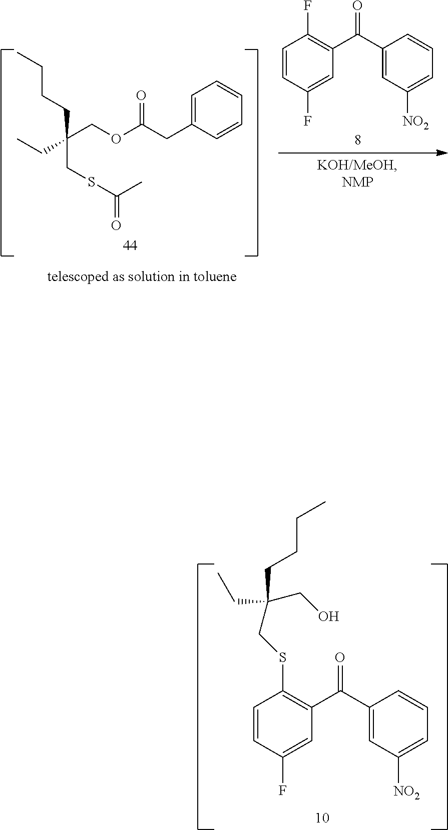

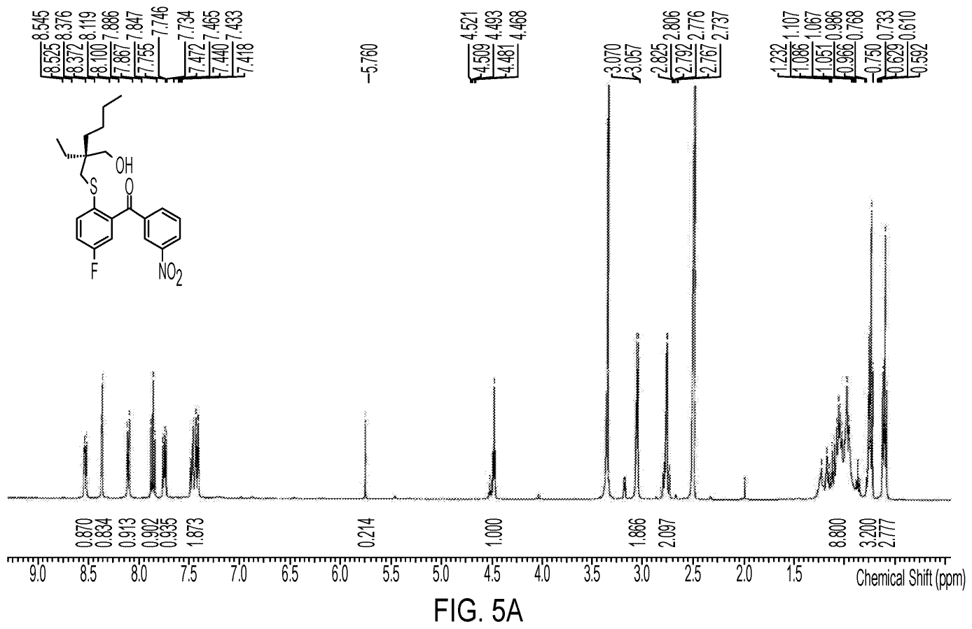

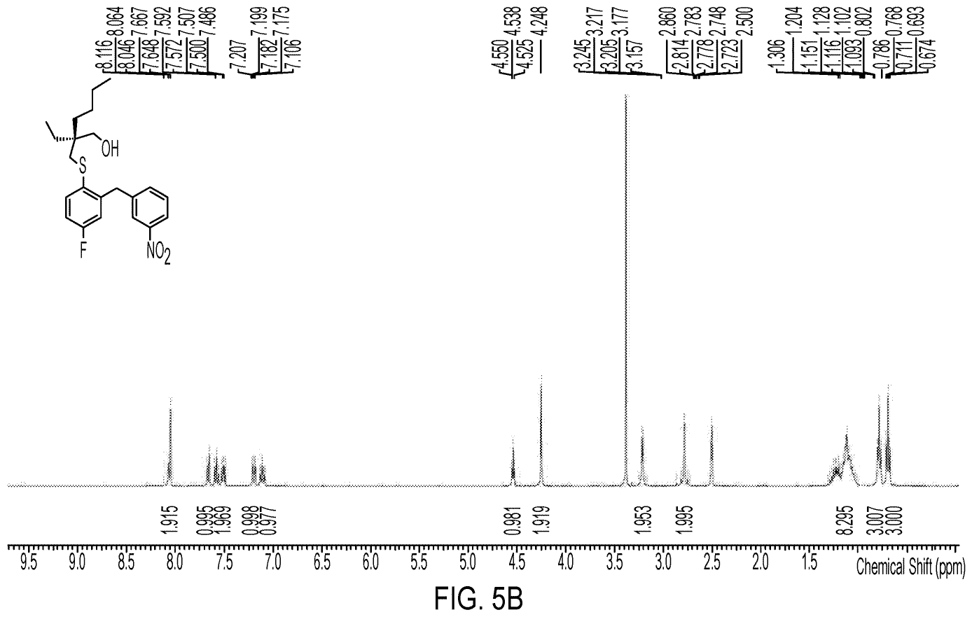

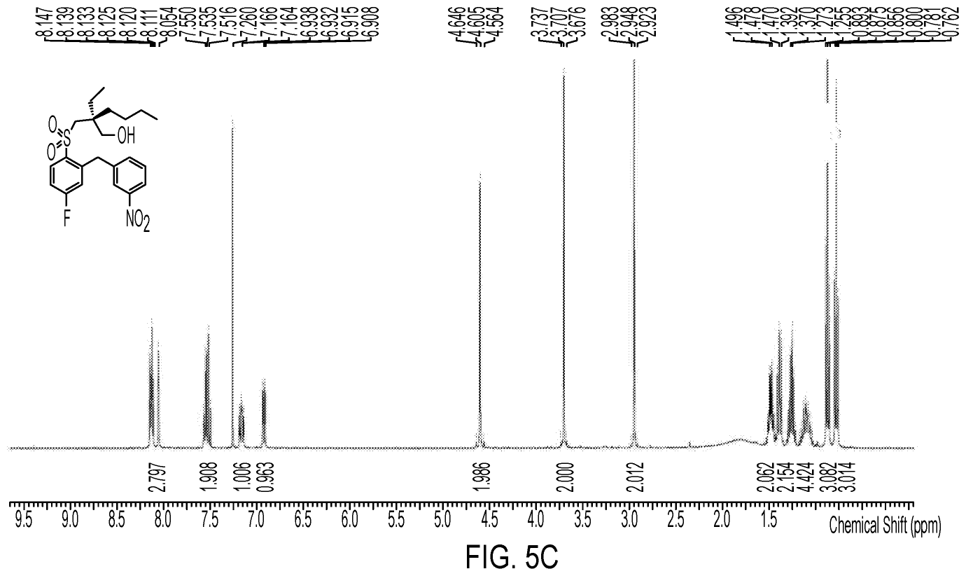

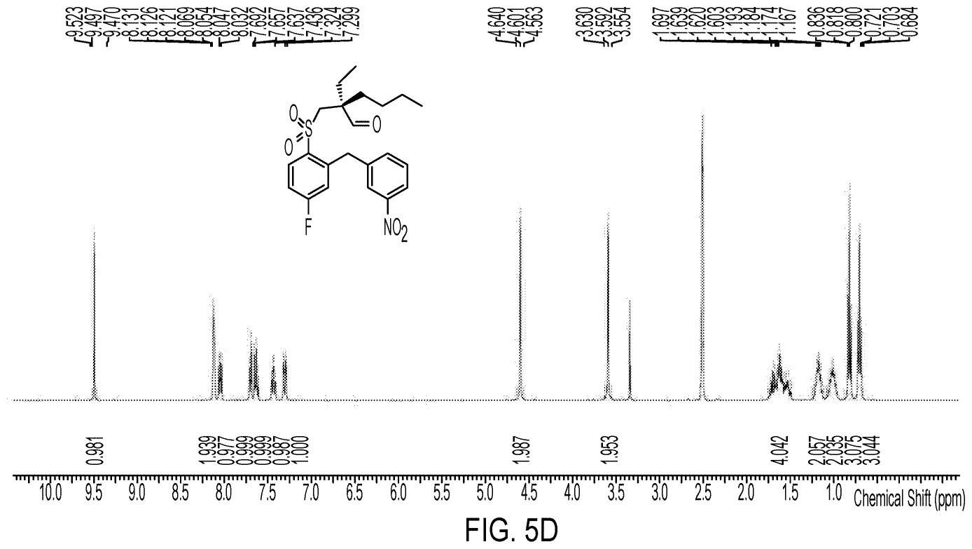

44 (Formula (II-A-44)) solution (active 77.0 kg, 229 mol) from last step was diluted with NMP (900 L) and 8 (Formula (II-A-8)) (53.0 kg, 201 mol) was then added as a solid and stirred until dissolved. The temperature was adjusted to 80-100° C. (e.g., 85° C.) and KOH/MeOH solution (15 wt %, 227 kg, 608 mol KOH) was added drop-wise over 2.0 h and stirred for 1 h at same temperature. The mixture was cooled to 25° C. and then toluene (493 L) and water (800 L) were added for extraction. The organic phase was separated and washed with NaOH solution (0.5N, 265 L), HCl solution (2 wt %, 265 L) and Na₂SO₄ aq. solution (5%, 265 L) in turn. After concentration to remove water, a toluene solution of 10 (Formula (II-A-10)) (238.2 kg solution, active 67.88 kg, 80.4%) was obtained. NMR of compound 10 (Formula (II-A-10)) is shown in FIG. 5A. ¹H-NMR (400 MHz, DMSO-d₆): δ0.59 (t, J=7.2 Hz, 3H), δ0.73 (t, J=6.8 Hz, 3H), δ0.97 (m, 8H), δ2.77 (d, J=3.6 Hz, 2H), δ3.06 (d, J=5.2 Hz, 2H), δ4.47 (t, J=5.2, 1H), δ7.42 (m, 2H), δ7.73 (dd, J=8.4 Hz, 1H), δ7.85 (t, J=8.0 Hz, 1H), δ8.10 (t, J=7.6 Hz, 1H), δ8.37 (t, J=1.6 Hz, 1H), δ8.53 (dd, J=8.0 Hz, 1H).

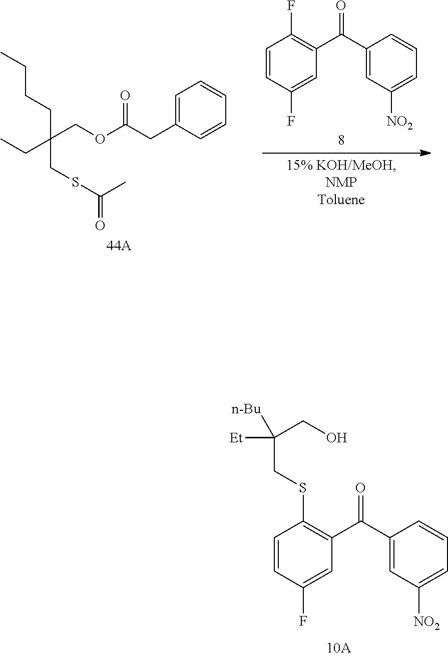

Also prepared was enantiomeric mixture 10A. To a mixture of compound 44A (Formula (II-B-44)) (50 g, 148 mmol, 1.12 eq.) in toluene (135 mL) was added NMP (349 mL) at 20° C. Compound 8 (Formula (II-A-8)) (34.9 g, 132 mmol, 1 eq.) was charged and the reaction was agitated at 105° C. followed by addition of KOH (22.3 g, 398 mmol, 3 eq.) in MeOH (230 mL). TLC indicated compound 44A (Formula (II-B-44)) was fully consumed and compound 10A (Formula (II-B-10)) had formed. The reaction mixture was transferred to cooled water (50 mL) and the aqueous phase was extracted with EtOAc (50 mL×2). The combined organic phase was washed with brine (50 mL), dried with anhydrous Na₂SO₄, filtered, and concentrated in vacuum and a residue was obtained. The residue was purified by column chromatography (petroleum ether:ethyl acetate, 5:1) and compound 10A (Formula (II-B-10)) (82 g) was obtained as a brown oil. NMR of compound 10A (Formula (II-B-10)) is as follows: ¹H-NMR (400 MHz, CDCl₃) δ ppm 8.59-8.55 (m, 1H), 8.50-8.38 (m, 1H), 8.17-8.08 (m, 1H), 7.73-7.60 (m, 2H), 7.24-7.19 (m, 1H), 7.07 (dd, J=2.7, 8.1 Hz, 1H), 3.36 (s, 2H), 2.86-2.83 (m, 2H), 1.29-1.13 (m, 8H), 0.88-0.84 (m, 3H), 0.74 (t, J=7.5 Hz, 3H).

### Example 6. Synthesis of Compound 11 (Formula (II-A-11)) and Enantiomeric Mixture

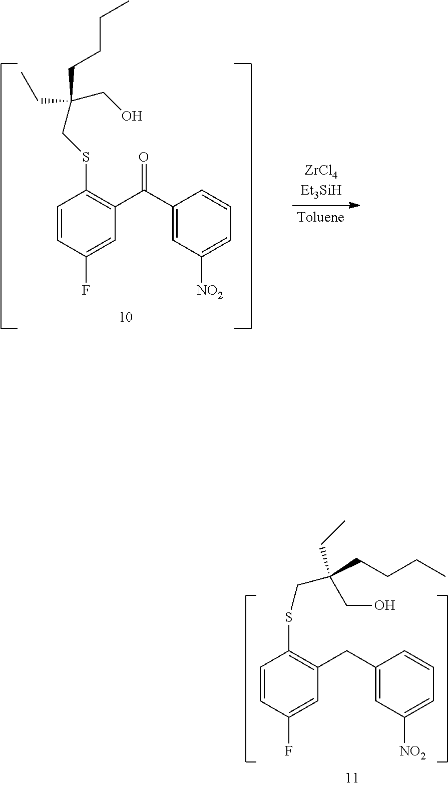

The solution of 10 (Formula (II-A-10)) (69.0 kg active, 164 mol) from the last step, ZrCl₄ (115 kg, 494 mol) and additional toluene (655 L) were charged in turn into reactor. The mixture was stirred at 25° C. for 30 min and heated to 40° C. Then TES (96 kg, 826 mol) was added portion-wise by maintaining the temperature below 50° C. and the mixture was stirred for another 10.0 h at 40° C. and monitored by HPLC before quenching. The temperature was cooled to 0° C. and water (600 L) was added slowly by maintaining the inner temperature below 20° C. The organic phase was separated and aqueous phase was extracted with toluene (120 L). The two toluene solutions were combined and washed with HCl aq. solution (2.5 wt %, 253 L) and water (690 L) in turn at 25° C.

The toluene solution was concentrated to 130 L under vacuum and dissolved in MeOH (720 L) containing conc. HCl aqueous solution (36 L). The mixture was first stirred at 25° C. for 1.5 h then n-heptane (360 L) was added and stirred for another 1 h. The MeOH phase was separated and swapped with DCM (400 L) twice. The DCM solution was washed with NaHCO₃ aq. solution (3%, 207 L) and water (272 L) in turn to remove the acid. The resulting DCM solution was concentrated to 200 L and purified through silica gel (345 kg) column with pure DCM as eluent. The eluent with purity more than 80% were collected and combined to afford 11 (Formula (II-A-11)) (27.5 kg active, 41%). Subsequent iterations of the synthesis of compound 11 yielded compound 11 in 53% yield.

Unexpectedly, it was discovered that column chromatography could be avoided in the purification of compound 11 and that a compound 11 telescoped solution could be used directly to synthesize compound 12. The telescoped synthesis of compound 11 yielded compound 11 in 82% yield, compared to a 40-55% yield with column chromatography purification. The telescoped synthesis of compound 11 was performed at a 100 g scale and comprised the dissolving in MeOH containing conc. HCl aqueous solution as described above. The n-heptane wash of the methanolic layer as described above was found to increase the yield of the telescoped compound 11 from 74% to 82%.

NMR of compound 11 (Formula (II-A-11)) is shown in FIG. 5B. ¹H-NMR (400 MHz, DMSO-d₆): δ0.67 (t, J=7.6 Hz, 3H), δ0.77 (t, J=7.2 Hz, 3H), δ1.12 (m, 8H), δ2.78 (d, J=1.8 Hz, 2H), δ3.20 (d, J=4.8 Hz, 2H), δ4.25 (s, 2H), δ4.53 (t, J=45.2 Hz, 1H), δ7.08 (td, J=8.4 Hz, 1H), δ7.17 (dd, J=9.8 Hz, 1H), δ7.49 (dd, J=8.4 Hz, 1H), δ7.55 (m, 1H), δ7.65 (m, 1H), δ8.05 (m, 2H).

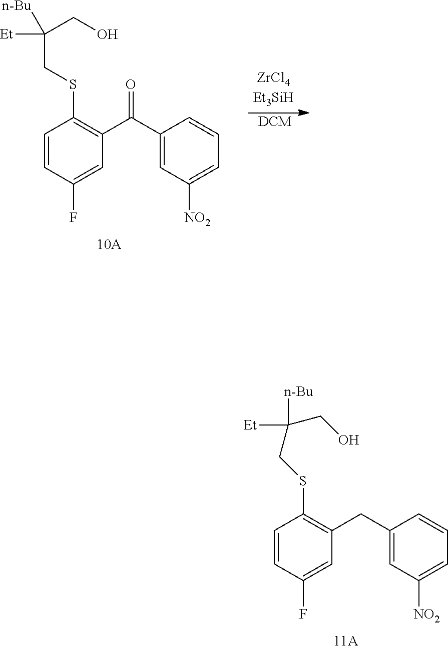

Also prepared was enantiomeric mixture 11A. ZrCl₄ (100 g, 431 mmol, 35.8 mL, 3 eq.) and Et₃SiH (103 g, 890 mmol, 142 mL, 6.2 eq.) were combined in DCM (305 mL) and the mixture was agitated at 45° C. Compound 10A (Formula (II-B-10)) (60.2 g, 143 mmol, 1 eq.) was added and the reaction was agitated at 45° C. for 12 h. LCMS indicated compound 10A (Formula (II-B-10)) was completely consumed and the reaction mixture was transferred into water (200 mL). The aqueous phase was extracted with DCM (100 mL×2). The combined organic phase was washed with brine (100 mL×2), dried with anhydrous Na₂SO₄, filtered, and concentrated under vacuum. The residue was purified by column chromatography (petroleum ether:ethyl acetate 5:1) and compound 11A (Formula (II-B-11)) was obtained as a brown oil. NMR of compound 11A (Formula (II-B-11)) is as follows: ¹H-NMR (400 MHz, CDCl₃) δ ppm 8.15-8.05 (m, 2H), 7.51-7.42 (m, 2H), 7.09-6.93 (m, 2H), 6.85 (dd, J=2.7, 9.3 Hz, 1H), 4.27 (s, 2H), 3.48 (s, 2H), 2.85 (s, 2H), 1.37-1.21 (m, 8H), 0.92-0.88 (m, 3H), 0.84-0.79 (m, 3H).

### Example 7. Additional Syntheses of Compound 11 (Formula (II-A-11))

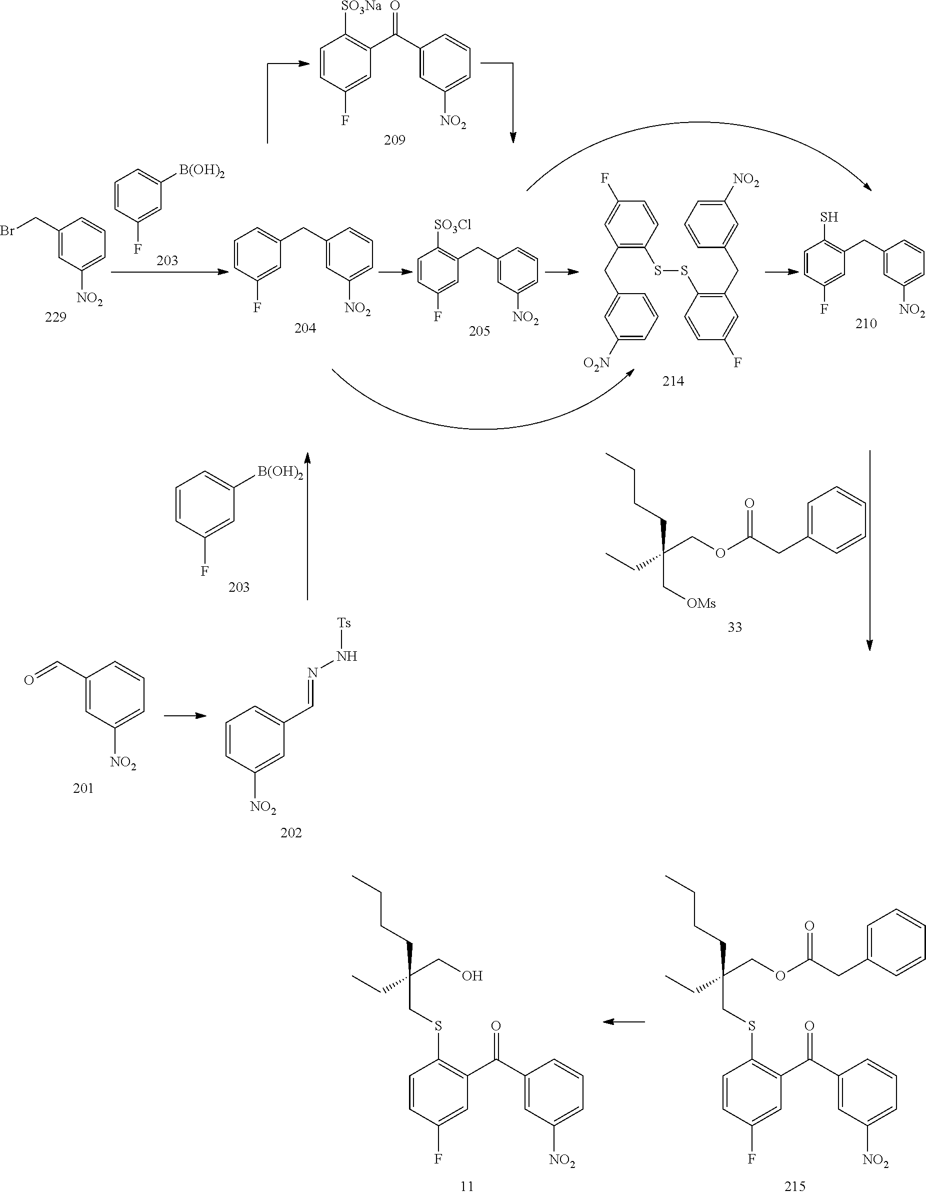

Unexpectedly, additional syntheses of compound 11 (Formula (II-A-11)) were discovered.

### Synthesis of Compound 204

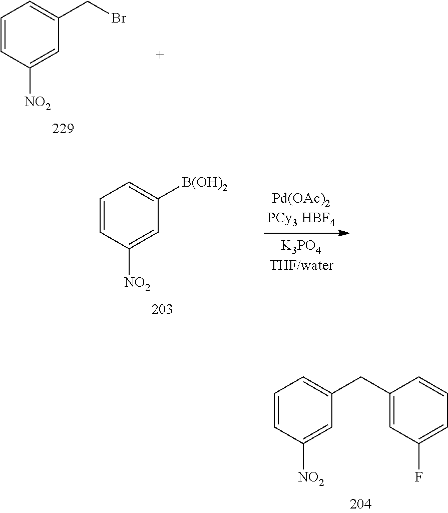 [SMILES: O=[N+]([O-])c1cccc(CBr)c1 ; O=[N+]([O-])c1cccc(B(O)O)c1 ; O=[N+]([O-])c1cccc(Cc2cccc(F)c2)c1]

A mixture of 1-(bromomethyl)-3-nitro-benzene (100 g, 462.2 mmol, 1.00 equiv., 229), (3-fluorophenyl) boronic acid (64.77 g, 462.9 mmol, 1.00 equiv., 203), Pd(OAc)₂ (259 mg, 1.15 mmol, 0.25 mol %), PCy₃·HBF₄ (1.7 g, 4.6 mmol, 1.00 mol %) was stirred in THF (600 mL, 6 vol.) at 25° C. for 10 min. The mixture was degassed by three N₂/vacuum cycles and K₃PO₄ (196.5 g, 925.8 mmol, 2.00 equiv.) and degassed water (200 mL, 2 vol.) were added. The reaction mixture was heated to 65° C. for 18 h. The mixture was cooled to 25° C., and it was washed with a 5M NaOH solution (200 mL, 2 vol.). THF was distilled off in vacuo and the reaction mixture was extracted with cyclohexane (300 mL, 3 vol.). The organic layer was concentrated to provide 204 as a dark oil (110.5 g, 85% corr. yield). NMR of compound 204 is as follows: ¹H-NMR (400 MHz, CDCl₃) δ ppm=8.09 (d, J=7.8, 1H), 8.05 (s, 1H), 7.50 (d, J=7.6, 1H), 7.47 (t, J=7.7, 1H), 7.32-7.25 (m, 1H), 6.99-6.91 (m, 2H), 6.87 (d, J=9.7, 1H), 4.07 (s, 2H). ¹⁹F NMR (175 MHz, CDCl₃) δ ppm=−112.6.

Alternative conditions for this reaction include, but are not limited to, performance with the following Pd precatalysts or the corresponding combination of ligand/Pd(0) or Pd(II) source: PPh₃, dppf, Brettphos, XantPhos, SPhos, RuPhos, XPhos, P(tBu)₃, and tBuBrettPhos; other bases: K₂CO₃, NaOH, NaHCO₃, K₃PO₄, Et₃N, and DIPEA; and other solvents: toluene, toluene/water mixtures, 2-MeTHF/water mixtures, MIBK/water mixtures, IPA/water mixtures and n-Butanol/water mixtures.

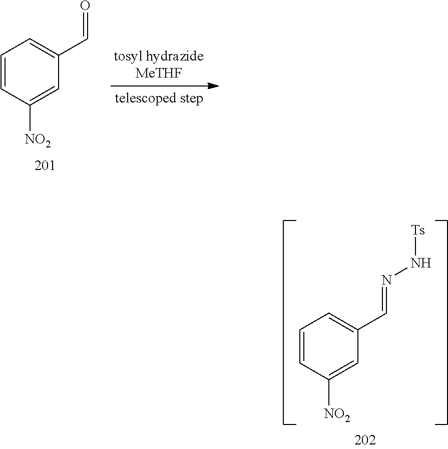

Alternatively, compound 204 may be synthesized starting with compound 201. A reactor was charged with tosyl hydrazide (30.8 g, 165.4 mmol, 1.00 equiv.) suspended in MeTHF (200 mL, 8 vol.) and heated to 60° C. Next, compound 201 (25.0 g, 165.4 mmol, 1.00 equiv.) was added dropwise as a solution in MeTHF (70 mL, 2.8 vol.) over 18 min. By LC it was confirmed that the reaction was concluded after full addition of compound 201 and the solution was cooled to 20° C. and used without further processing.

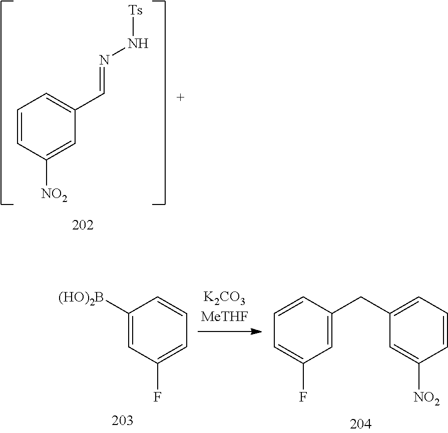

A reactor was charged with compound 203 (34.7 g, 248.1 mmol, 1.50 equiv.) and K₂CO₃ (34.3 g, 248.1 mmol, 1.50 equiv.) and the solids were suspended in MeTHF (125 mL, 3.5 vol.). The suspension was then heated to 85° C. Next, the solution of compound 202 (1.00 equiv. in 14 vol. Me-THF) that had been prepared in the previous step was added dropwise over 1 h. The mixture was stirred for one additional hour and then cooled to 20° C. Next, the suspension was filtered through a pad of celite, and the filter cake was washed with TBME (200 mL, 5.7 vol.). The filtrate was washed with aqueous 20 wt % K₂CO₃ solution (2×100 mL, 2.9 vol.) and water (100 mL, 2.9 vol.), then concentrated in vacuo. The residue was purified by flash chromatography and isolated as a yellow oil (27.9 g, 120.8 mmol, 73% corrected yield). NMR of compound 204 Formula (II-A-204) is as follows: ¹H-NMR (400 MHz, CDCl₃) δ ppm=8.09 (d, J=7.8, 1H), 8.05 (s, 1H), 7.50 (d, J=7.6, 1H), 7.47 (t, J=7.7, 1H), 7.27-7.32 (m, 1H), 6.91-7.01 (m, 2H), 6.87 (d, J=9.7, 1H), 4.07 (s, 2H). ¹⁹F NMR (175 MHz, CDCl₃) δ ppm=−112.6 (m).

Alternative conditions for this reaction include, but are not limited to, use of isolated compound 202, use of another base (e.g., NaOH), and use of another solvent (e.g., CPME, toluene, anisole, and 1,4-dioxane).

### Synthesis of Compound 209

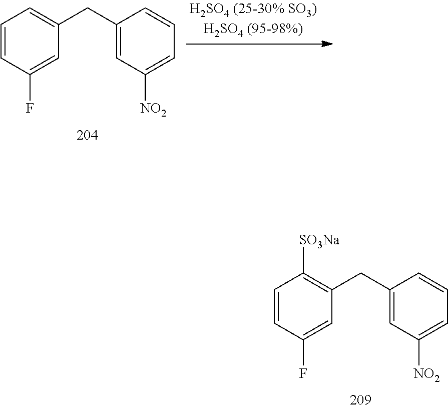

The vessel was charged with compound 204 Formula (II-A-204) (23.60 g, 102.1 mmol, 1.00 equiv.) then conc. sulfuric acid (95-98 wt %, 23.6 mL, 1 vol.) was added over 3 min. The temperature was kept below 30° C. Next fuming sulfuric acid (25-30% SO₃, 27.0 g, 2.70 equiv.) was added over 7 min. while the temperature was maintained below 30° C. The reaction was subsequently stirred at 20° C. for 2 h then heated to 40° C. for 1 h. Next, the reaction mixture was cooled to 20° C. and added dropwise over 10 min. to aqueous 10 wt % NaCl solution (14 vol.). The temperature was kept below 25° C. The product precipitated as an off-white crystalline solid. Afterwards the solid was dissolved by heating the mixture to 50° C. Slow cooling to 20° C. led to the crystallization of the product. Successive filtration followed by washing with aqueous 5 wt % NaCl solution (2 vol.) and aqueous 10 wt % NaCl solution (4 vol.) yielded compound 209 as an off-white crystalline solid (28.33 g, 88% assay, 74.8 mmol, corr. yield 73%). NMR of compound 209 Formula (II-A-209) is as follows: ¹H-NMR (400 MHz, DMSO-d₆) δ[ppm]=8.24 (s, 1H), 8.03 (dd, J=8.3, 2.2 Hz, 1H), 7.90-7.81 (m, 2H), 7.57 (t, J=7.9 Hz, 1H), 6.99 (td, J=8.57, 2.2 Hz, 1H), 6.88 (dd, J=10.2, 2.1 Hz, 1H), 4.57 (s, 2H). ¹⁹F NMR (175 MHz, DMSO-d₆) δ[ppm]=−113.2 (m).

### Synthesis of Compound 205

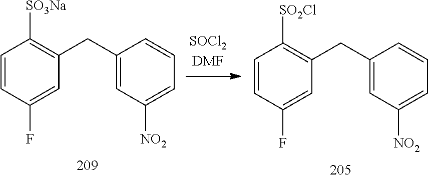

Compound 209 Formula (II-A-209) (10.0 g, 27.0 mmol) was dissolved in DMF (2.5 mL, 0.25 vol.) and SOCl₂ (70 mL, 7 vol.) was added. The mixture was stirred at 60° C. for 16 h. The reaction mixture was drained and transferred to a dropping funnel. Toluene (100 mL, 10 vol.) and water (100 mL, 100 vol.) were added to the reactor. The reaction mixture was quenched by dropwise addition to the biphasic mixture. The temperature was kept below 35° C. The organic layer was collected, dried over Na₂SO₄ and concentrated to give compound 205 as an off-white crystalline solid (8.25 g, 25.2 mmol, 87% corrected yield). NMR of compound 205 Formula (II-A-205) is as follows: ¹H-NMR (400 MHz, CDCl₃) δ [ppm]=8.25-8.11 (m, 2H), 8.08 (s, 1H), 7.56 (m, 2H), 7.18 (m, 1H), 6.91 (d, J=9.0, 1H), 4.65 (s, 2H). ¹⁹F NMR (400 MHz, CDCl₃) δ [ppm]=−98.9 (m). Alternative conditions for this reaction include, but are not limited to, other chlorinating agents (e.g., POCl₃ and PCl₅).

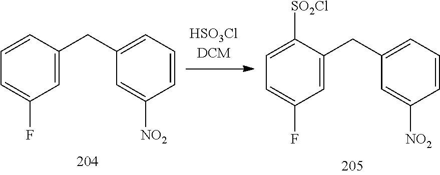 [SMILES: O=[N+]([O-])c1cccc(Cc2cccc(F)c2)c1 ; O=[N+]([O-])c1cccc(Cc2cc(F)ccc2S(=O)(=O)Cl)c1]

Alternatively, compound 205 Formula (II-A-205) may be synthesized starting with compound 204 Formula (II-A-204). The reaction vessel was charged with compound 204 (5.0 g, 21.6 mmol, 1.00 equiv.), purged with nitrogen and dissolved in anhydrous DCM (25 mL, 5 vol.) then cooled to 0° C. Next, chlorosulfonic acid (7.19 mL, 108.1 mmol, 5.00 equiv.) was added in one portion. The reaction mixture was warmed to 25° C. and after 2 h heated to 45° C. for 2 h. The reaction mixture was added to water (100 mL, 20 vol.) and extracted with TBME (200 mL, 40 vol.). The aqueous phase was extracted with TBME (50 mL, 10 vol.) and the combined organic phase was washed with aqueous 10 wt % NaCl solution (100 mL, 20 vol.) and water (100 mL, 20 vol.), dried over MgSO₄, and concentrated. Crude compound 205 was obtained as an off-white solid (6.0 g, 73% assay, corr. yield 62%). NMR of compound 205 is as follows: ¹H-NMR (400 MHz, CDCl₃) δ [ppm]=8.22 (dd, J=5.4, 9.0 Hz, 1H), 8.15-8.19 (m, 1H), 8.09 (s, 1H), 7.51-7.60 (m, 2H), 7.15-7.22 (m, 1H), 6.91 (dd, J=2.6, 9.2 Hz, 1H), 4.65 (s, 2H). ¹⁹F NMR (175 MHz, CDCl₃) δ [ppm]=−98.9 (m).

### Synthesis of Compound 214

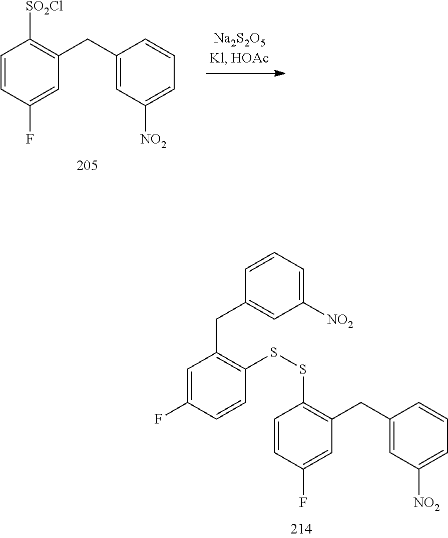 [SMILES: O=[N+]([O-])c1cccc(Cc2cc(F)ccc2S(=O)(=O)Cl)c1 ; O=[N+]([O-])c1cccc(Cc2cc(F)ccc2SSc2ccc(F)cc2Cc2cccc([N+](=O)[O-])c2)c1]

A vessel was charged with compound 205 Formula (II-A-205) (700.0 mg, 88% assay as a mixture of regioisomers, 1.85 mmol, 1.00 equiv.), Na₂S₂O₅ (2.11 g, 11.08 mmol, 6.00 equiv.) and KI (76.7 mg, 0.46 mmol, 0.25 equiv.), then HOAc (7 mL, 10 vol.) was added and heated to 105° C. for 2.5 h. The mixture was cooled to 20° C., then water (20 mL, 28.6 vol.) and TBME (20 mL, 28.6 vol.) were added and the phases separated. The aqueous phase was extracted with TBME (20 mL, 28.6 vol.) and the combined organic phase washed with aqueous 20 wt % K₂CO₃ solution (2×, 20 mL, 28.6 vol.) and water (20 mL, 28.6 vol.), then dried over Na₂SO₄ and concentrated. Crude 214 was obtained as a brown solid (510.0 mg, 83% assay, 0.81 mmol, 88% corr. yield). Crude 214 was recrystallized from toluene/n-heptane (1:1, 1 mL, 1.4 vol.) and obtained as a near colorless solid (326.0 mg, 92% assay, 0.57 mmol, 62% corr. yield). NMR of compound 214 Formula (II-A-214) is as follows: ¹H-NMR (400 MHz, CDCl₃) δ [ppm]=8.07 (dt, J=1.8, 7.6 Hz, 1H), 7.95 (s, 1H), 7.45-7.38 (m, 3H), 6.92 (td, J=2.8, 8.3 Hz, 1H), 6.86 (dd, J=2.8, 9.2 Hz, 1H), 4.17 (s, 2H). ¹⁹F NMR (175 MHz, CDCl₃) δ [ppm]=−110.2 (m). Alternative conditions for this reaction include, but are not limited to, the use of 6.00 equiv. of Na₂SO₃.

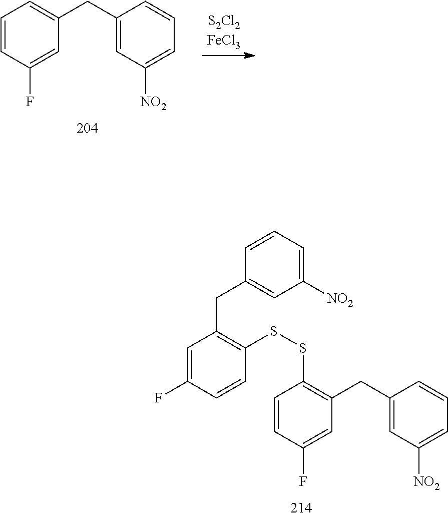 [SMILES: O=[N+]([O-])c1cccc(Cc2cccc(F)c2)c1 ; O=[N+]([O-])c1cccc(Cc2cc(F)ccc2SSc2ccc(F)cc2Cc2cccc([N+](=O)[O-])c2)c1]

Alternatively, compound 214 Formula (II-A-214) may be synthesized starting with compound 204 Formula (II-A-204). The reaction vessel was charged with anhydrous FeCl₃ (70.2 mg, 0.43 mmol, 1.00 equiv.) in a glovebox. The vessel was sealed and removed from the glovebox. Next, anhydrous DCM (1 mL, 10 vol.), compound 204 (100 mg, 0.43 mmol, 1.00 equiv.), and S₂Cl₂ (17.7 μL, 0.22 mmol, 0.50 equiv.) were added and the mixture was stirred at 25° C. for 18 h. Crude 214 was obtained as a mixture with different polysulfides and other sulfide species as an orange solid (98.0 mg, uncorr. yield 86%).

### Synthesis of Compound 210

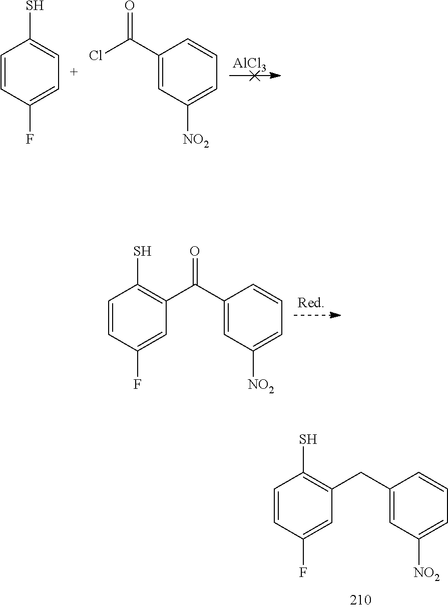

In attempts to make compound 210, electrophilic aromatic substitution was not successful between 4-fluorobenzenethiol and 3-nitrobenzoyl chloride.

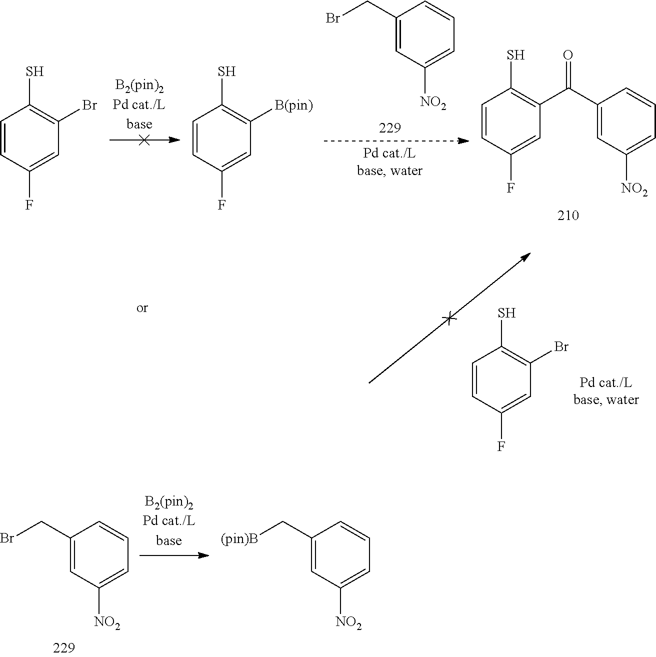

Further attempts to synthesize compound 210 were unsuccessful. Borylation of bromothiophenol could not be accomplished. Coupling of bromothiophenol with benzylic B(pin) was also unsuccessful. Reductive coupling approaches were also tested unsuccessfully.

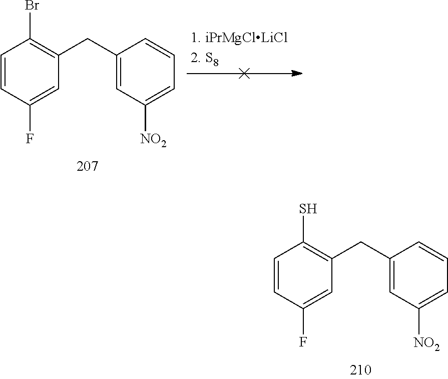 [SMILES: O=[N+]([O-])c1cccc(Cc2cc(F)ccc2Br)c1 ; O=[N+]([O-])c1cccc(Cc2cc(F)ccc2S)c1]

Additionally, in efforts to synthesize compound 210, Mg/Br exchange-thiolation was attempted but provided a complex mixture.

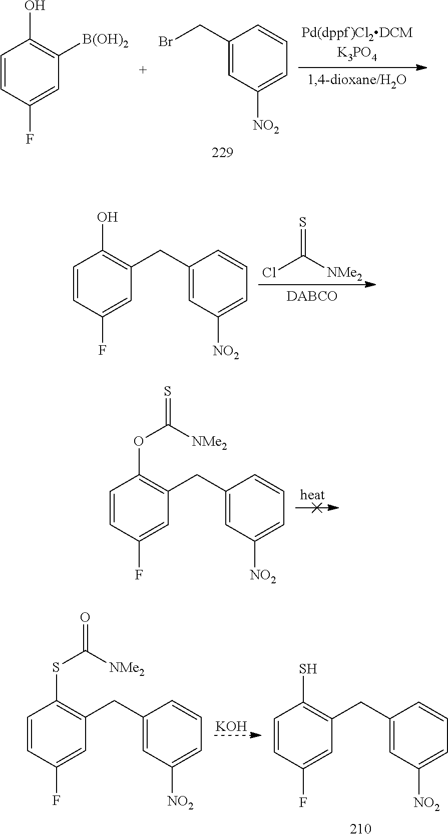

In further attempts to synthesize compound 210, a Newman-Kwart rearrangement was attempted, but reduction of the nitro group was observed instead of the desired rearrangement.

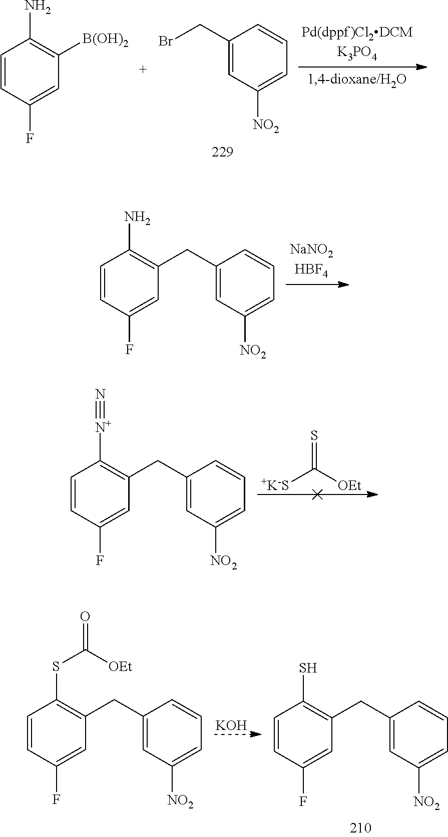

Moreover, in attempts to make compound 210, xanthate formation was attempted, but was found unsuccessful.

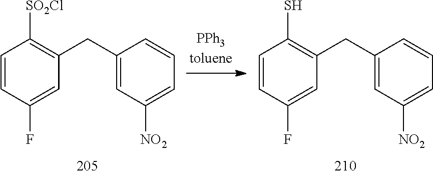 [SMILES: O=[N+]([O-])c1cccc(Cc2cc(F)ccc2S(=O)(=O)Cl)c1 ; O=[N+]([O-])c1cccc(Cc2cc(F)ccc2S)c1]

Unexpectedly, after many failed attempts to synthesize compound 210, a successful synthesis to compound 210 Formula (II-A-210) was found. A vessel was charged with compound 205 Formula (II-A-205) (100.0 mg, 0.30 mmol, 1.00 equiv.) and PPh₃ (238.6 mg, 0.91 mmol, 3.00 equiv.), then suspended in toluene (1.0 mL, 10 vol.) and heated to 60° C. for 45 min. Afterwards the mixture was cooled to 20° C., water (0.5 mL, 5 vol.) was added, the mixture was stirred for 30 min. and then transferred to a separatory funnel and TBME (10 mL, 100 vol.) and water (10 mL, 100 vol.) were added. The organic phase was extracted with aqueous 5 wt % NaOH solution (4×, 15 mL, 150 vol.), the combined aq. layers neutralized with aqueous 10 wt % HCl solution (40 mL, 800 vol.), and then extracted with TBME (2×, 20 mL, 200 vol.). The combined organic phase was dried over Na₂SO₄ and concentrated. Crude 210 was obtained as a yellow oil (72.0 mg, 88% assay, 0.24 mmol, corr. yield 79%). NMR of compound 210 is as follows: ¹H-NMR (400 MHz, CDCl₃) δ [ppm]=8.14-8.07 (m, 1H), 8.04 (s, 1H), 7.72-7.63 (m, 1H), 7.50-7.43 (m, 3H), 7.40-7.32 (m, 1H), 6.94-6.82 (m, 2H), 4.17 (s, 2H). ¹⁹F NMR (175 MHz, CDCl₃) δ [ppm]=−115.0 (m).

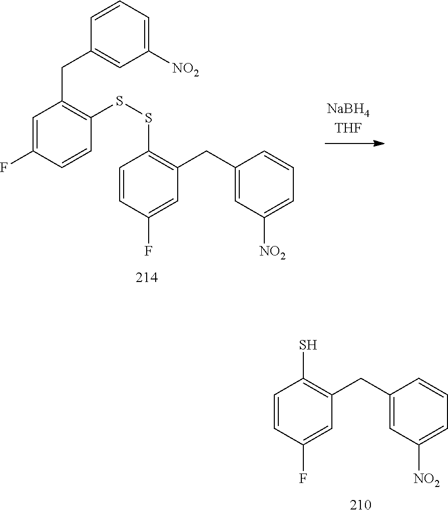 [SMILES: O=[N+]([O-])c1cccc(Cc2cc(F)ccc2SSc2ccc(F)cc2Cc2cccc([N+](=O)[O-])c2)c1 ; O=[N+]([O-])c1cccc(Cc2cc(F)ccc2S)c1]

Alternatively, compound 210 Formula (II-A-210) may be synthesized starting with compound 214 Formula (II-A-214). The reaction vessel was charged with crude 214 (98.0 mg, 0.19 mmol, 1.00 equiv.) from the previous step and purged with N₂. Then, anhydrous THF (1 mL, 10.2 vol.) was added. Subsequently NaBH₄ (28.3 mg, 0.75 mmol, 4.00 equiv.) was added in one portion and the mixture was stirred at 25° C. for 16 h. Next, 7 wt % HCl solution (20 mL, 204 vol.) was added over 5 min. and the mixture was extracted with iPrOAc (20 mL, 204 vol.). The organic phase was washed with water (20 mL, 204 vol.), dried over Na₂SO₄, and then concentrated in vacuo. Compound 210 was obtained as a yellow solid (109 mg, uncorr. yield 110%). NMR of compound 210 is as follows: ¹H-NMR (400 MHz, CDCl₃) δ [ppm]=8.14-8.07 (m, 1H), 8.04 (s, 1H), 7.72-7.63 (m, 1H), 7.50-7.43 (m, 3H), 7.40-7.32 (m, 1H), 6.94-6.82 (m, 2H), 4.17 (s, 2H). ¹⁹F NMR (175 MHz, CDCl₃) δ [ppm]=−115.0 (m).

### Synthesis of Compound 215

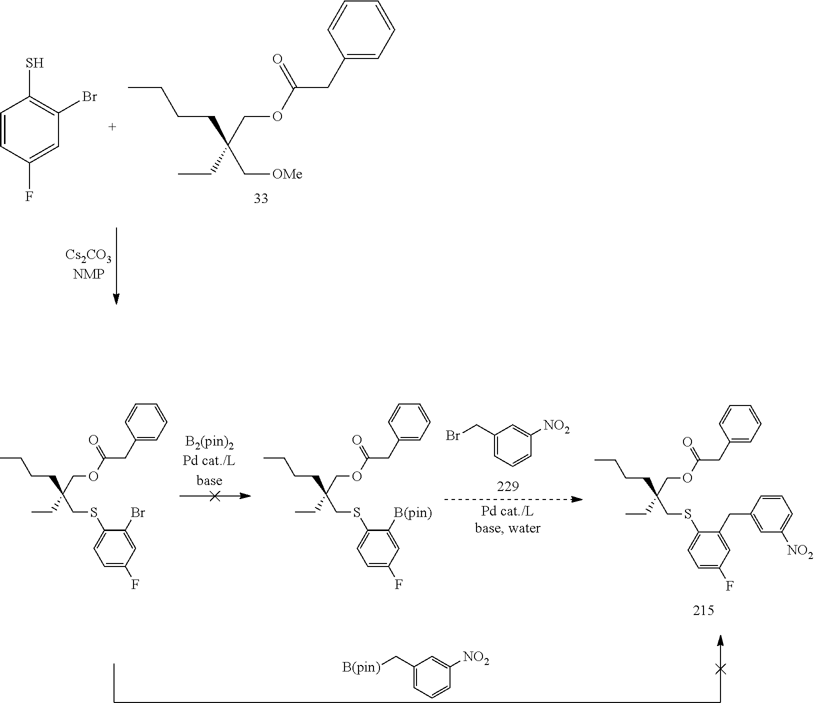

In attempts to make compound 215, borylation/coupling reactions were unsuccessful and reductive coupling approaches were also tested and found unsuccessful.

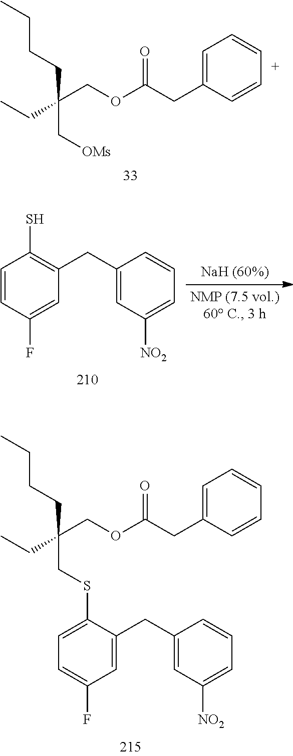 [SMILES: CCCC[C@@](CC)(COC(=O)Cc1ccccc1)COS(C)(=O)=O ; O=[N+]([O-])c1cccc(Cc2cc(F)ccc2S)c1 ; CCCC[C@@](CC)(COC(=O)Cc1ccccc1)CSc1ccc(F)cc1Cc1cccc([N+](=O)[O-])c1]

Unexpectedly, after failed attempts to synthesize compound 215, a successful synthesis to compound 215 was created. In a glovebox, compound 210 Formula (II-A-210) (222.0 mg, 0.84 mmol, 1.00 equiv.) was dissolved in NMP (0.5 mL, 2.3 vol.). A second vessel was charged with NaH (33.7 mg, 60 wt % in mineral oil, 0.84 mmol, 1.00 equiv.) and the solid was suspended in NMP (1.0 mL, 4.6 vol.). Then, the solution of 210 was added in portions over 1 min. Next, compound 33 (466.0 mg, 430 μL, 71 wt % in NMP, 0.93 mmol, 1.10 equiv.) was added and the mixture was heated to 60° C. for 3 h. The vessel was removed from the glovebox, the reaction mixture transferred to a separatory funnel and aqueous 1 wt % HCl solution (10 mL, 46 vol.) and ethyl acetate (25 mL, 115 vol.) were added. The phases were separated and the aqueous phase was extracted with ethyl acetate (10 mL, 46 vol.). The combined organic phase was washed with water (10 mL, 46 vol.), dried over Na₂SO₄ and concentrated. Crude 215 was then purified by flash chromatography and obtained as a colorless oil (306.0 mg, 0.58 mmol, 69% corr. yield). NMR of compound 215 is as follows: ¹H-NMR (400 MHz, CDCl₃) δ [ppm]=8.06 (dt, J=2.0, 7.5 Hz, 1H), 8.01 (s, 1H), 7.49-7.39 (m, 2H), 7.32 (dd, J=5.6, 8.7 Hz, 1H), 7.27-7.22 (m, 3H), 7.21-7.17 (m, 2H), 6.91 (td, J=2.9, 8.3 Hz, 1H), 6.81 (dd, J=2.8, 9.4 Hz, 1H), 4.20 (s, 2H), 3.93 (s, 2H), 3.54 (s, 2H), 2.71 (s, 2H), 1.30 (q, J=7.6 Hz, 2H), 1.24-1.14 (m, 4H), 1.12-1.10 (m, 2H), 0.83 (t, J=7.2 Hz, 3H), 0.73 (t, J=7.4 Hz, 3H). ¹⁹F NMR (175 MHz, CDCl₃) δ [ppm]=−114.7 (m). Alternative conditions for this reaction include, but are not limited to, other bases (e.g., LDA, NaHMDS, KHMDS) and other solvents (e.g., polar aprotic solvents).

### Synthesis of Compound 11

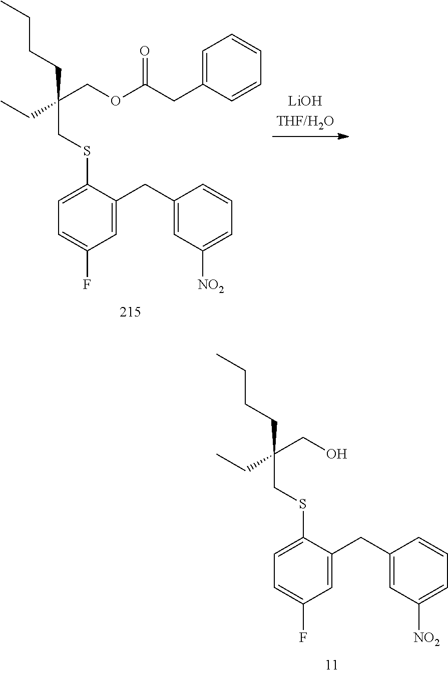 [SMILES: CCCC[C@@](CC)(COC(=O)Cc1ccccc1)CSc1ccc(F)cc1Cc1cccc([N+](=O)[O-])c1 ; CCCC[C@@](CC)(CO)CSc1ccc(F)cc1Cc1cccc([N+](=O)[O-])c1]

The reaction vessel was charged with compound 215 Formula (II-A-215) (300.0 mg, 0.57 mmol, 1.00 equiv.) and dissolved in THF (6.6 mL, 6.6 vol.). Next LiOH (68.6 mg, 2.86 mmol, 5.00 equiv.) in water (3.3 mL, 3.3 vol.) was added, and the reaction mixture heated to 80° C. for 17 h. The reaction mixture was subsequently cooled to 25° C. and aqueous 1 wt % NaOH solution (5 mL, 5 vol.) and TBME (10 mL, 10 vol.) were added. The phases were separated, the aqueous phase extracted with TBME (10 mL, 10 vol.) and the combined organic phase washed with water (10 mL, 10 vol.), dried over Na₂SO₄ and concentrated. Compound 11 (Formula (II-A-11)) was obtained as a colorless oil (230.0 mg, 0.56 mmol, 99% corr. yield). NMR of compound 11 is as follows: ¹H-NMR (400 MHz, CDCl₃) δ [ppm]=8.12-8.02 (m, 2H), 7.53-7.41 (m, 3H), 6.96 (td, J=2.5, 8.3 Hz, 1H), 6.84 (dd, J=2.5, 9.3 Hz, 1H), 4.26 (s, 2H), 3.47 (s, 2H), 2.84 (s, 2H), 1.43-1.10 (m, 10H), 0.89 (t, J=7.0 Hz, 3H), 0.81 (t, J=7.5 Hz, 3H). ¹⁹F NMR (400 MHz, CDCl₃) δ [ppm]=114.9 (m). Alternative conditions for this reaction include, but are not limited to, other bases (e.g., NaOH, KOH) and other solvents (e.g., ethers, polar aprotic solvents).

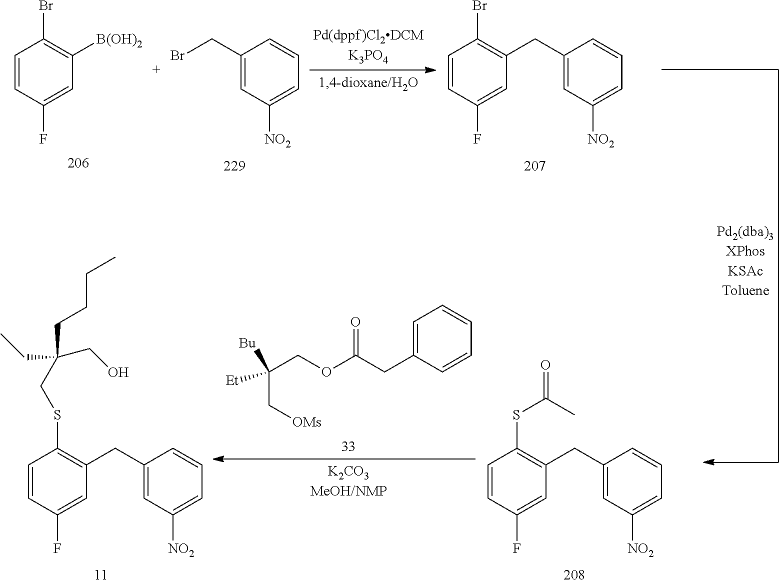

Further, an additional synthesis of compound 11 (Formula (II-A-11)) was discovered as described herein.

### Synthesis of Compound 207

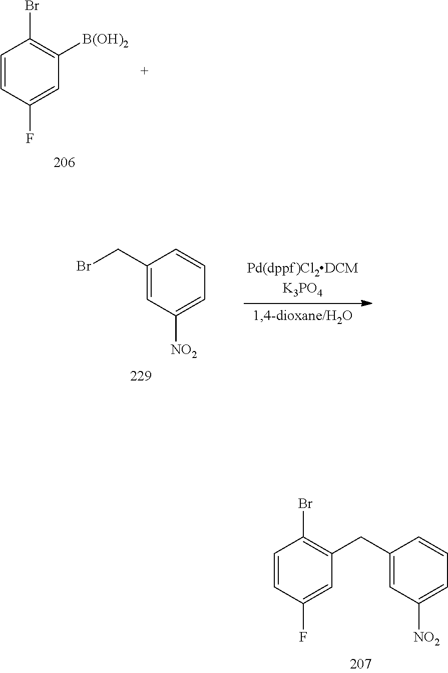 [SMILES: OB(O)c1cc(F)ccc1Br ; O=[N+]([O-])c1cccc(CBr)c1 ; O=[N+]([O-])c1cccc(Cc2cc(F)ccc2Br)c1]

Compound 229 (8.97 g, 1.0 eq.), 1,1′-bis(diphenylphosphino)ferrocene-palladium(II)dichloride dichloromethane complex (1.54 g, 0.05 eq.), compound 206 (10.0 g, 1.1 eq.) and K₃PO₄ (26.45 g, 3.0 eq.) were dissolved in 1,4-dioxane (80 mL)/water (9 mL). After stirring for 2 h at 90° C., EtOAc (90 mL) was added. The organic phase was separated and concentrated. The crude product was purified by column chromatography to obtain compound 207 as a solid (8.5 g, 59% yield). NMR of compound 207 is as follows: ¹H-NMR (400 MHz, CDCl₃) δ [ppm]=3.96 (s, 2H), 6.66 (m, 2H), 7.30 (m, 3H), 7.83 (m, 1H), 7.88 (dt, J=8.0, 2.0 Hz, 1H).

Alternative conditions for this reaction include, but are not limited to, performance with the following Pd precatalysts or the corresponding combination of ligand/Pd(0) or Pd(II) source: PPh₃, Brettphos, XantPhos, SPhos, RuPhos, XPhos, P(tBu)₃, and tBuBrettPhos; other bases: K₂CO₃, NaOH, NaHCO₃, K₃PO₄, Et₃N, and DIPEA; and other solvents: toluene, toluene/water mixtures, THF/water mixtures, 2-MeTHF/water mixtures, MIBK/water mixtures, IPA/water mixtures and n-Butanol/water mixtures.

### Synthesis of Compound 208

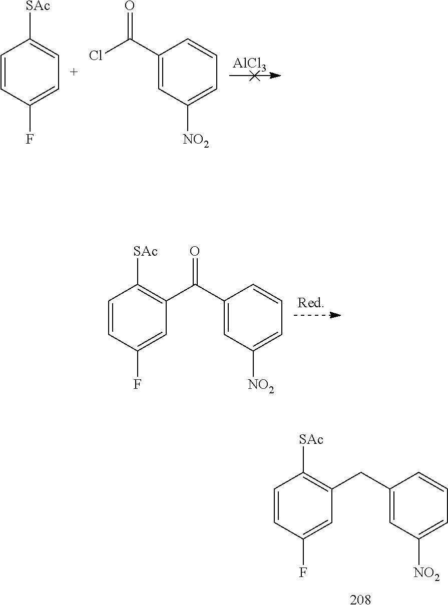

In attempts to make compound 208, electrophilic aromatic substitution was not successful between S-(4-fluorophenyl) ethanethioate and 3-nitrobenzoyl chloride.

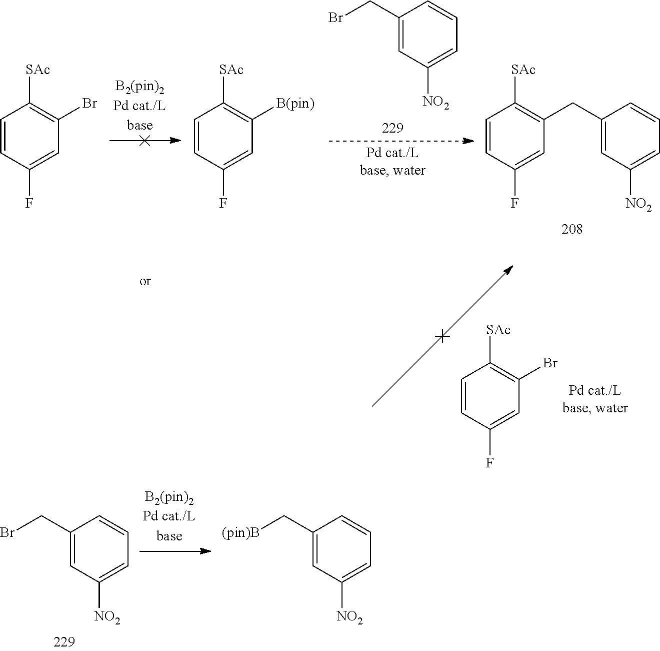

Further attempts to synthesize compound 208 were unsuccessful. Borylation of thioacetate could not be accomplished. Coupling of thioacetate with benzylic B(pin) was also unsuccessful. Reductive coupling approaches were also tested unsuccessfully.

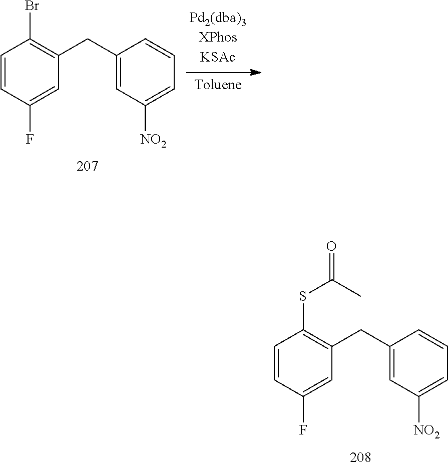 [SMILES: O=[N+]([O-])c1cccc(Cc2cc(F)ccc2Br)c1 ; CC(=O)Sc1ccc(F)cc1Cc1cccc([N+](=O)[O-])c1]

Unexpectedly, after failed attempts to synthesize compound 208 Formula (II-A-208), a successful synthesis to compound 208 was created. Compound 207 Formula (II-A-207) (5.0 g, 1.0 eq.), Pd₂(dba)₃ (0.73 g, 0.05 eq.) and XPhos (0.1 eq.) were dissolved in toluene (10 vol.), then KSAc (5.53 g, 2.0 eq.) was added into the reaction mixture. The resulting solution was stirred for 24 h at 110° C. Upon reaction completion, the mixture was diluted with EtOAc (50.0 mL) and filtered through a short pad of silica gel. The filtrate was concentrated under vacuum, and the crude residue was purified by column chromatography on silica gel eluting with hexane/ethyl acetate to obtain compound 208 (1.90 g, 39% yield). NMR of compound 208 is as follows: ¹H-NMR (400 MHz, CDCl₃) δ [ppm]=2.39 (s, 3H), 4.14 (s, 2H), 6.91 (dd, J=10.2 Hz, 1H), 7.03 (dd, J=10.2 Hz, 1H), 7.45 (m, 3H), 8.02 (m, 1H), 8.09 (m, 1H). HRMS: m/z calculated [M+Na]⁺: 328.0420, measured: 328.0316. Alternative conditions for this reaction include, but are not limited to, other ligands (e.g., CyPFtBu, XantPhos, DavePhos, SPhos, tBuXPhos, JohnPhos, RuPhos).

### Synthesis of Compound 11

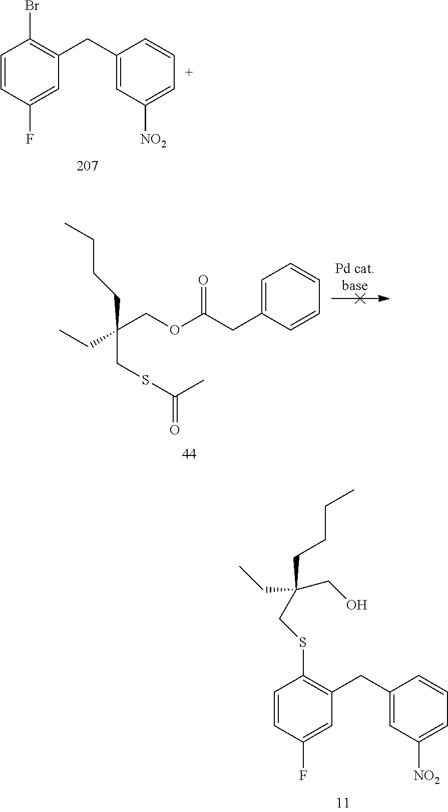 [SMILES: O=[N+]([O-])c1cccc(Cc2cc(F)ccc2Br)c1 ; CCCC[C@@](CC)(COC(=O)Cc1ccccc1)CSC(C)=O ; CCCC[C@@](CC)(CO)CSc1ccc(F)cc1Cc1cccc([N+](=O)[O-])c1]

In an attempt to synthesize compound 11, C—S coupling was tried with compound 207 and compound 44, but no product was observed.

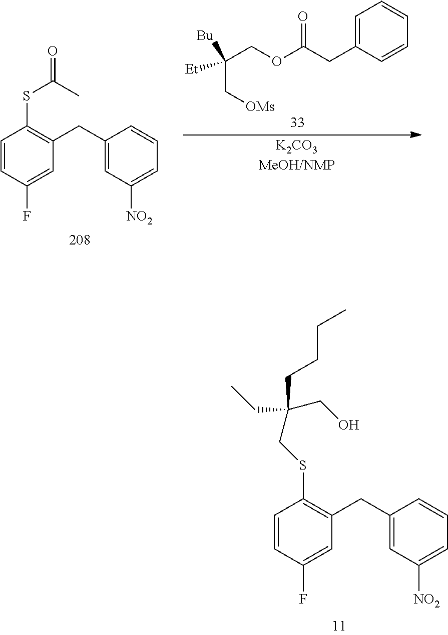

Unexpectedly, a successful synthesis to compound 11 (Formula (II-A-11)) was found. Compound 208 Formula (II-A-208) (0.1 g, 1.0 eq.) and compound 33 (0.14 g, 1.0 eq.) were dissolved in MeOH (0.5 mL, 5 vol.), then NMP (1 mL, 10 vol.) and K₂CO₃ (0.14 g, 3.0 eq.) were added. The reaction mixture was stirred at 85° C. for 2 h. Upon reaction completion, H₂O (2 mL) and EtOAc (10 mL) were added into the mixture. The organic phase was separated and concentrated to dryness. The crude product was purified by column chromatography to obtain compound 11 in 30% yield. Alternative conditions for this reaction include, but are not limited to, other bases (e.g., Cs₂CO₃, NaOMe, K₃PO₄, NH₄OH) and other solvents (e.g., mixtures of polar aprotic solvents and alcohols).

### Example 8. Synthesis of Compound 12 (Formula (II-A-12)) and Enantiomeric Mixture

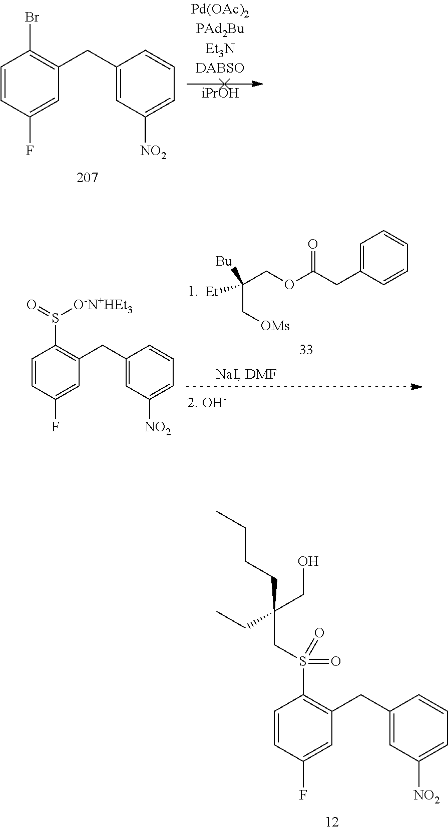

In an attempt to synthesize compound 12, synthesis of a sulfinate intermediate from compound 207 was attempted, but unsuccessful.

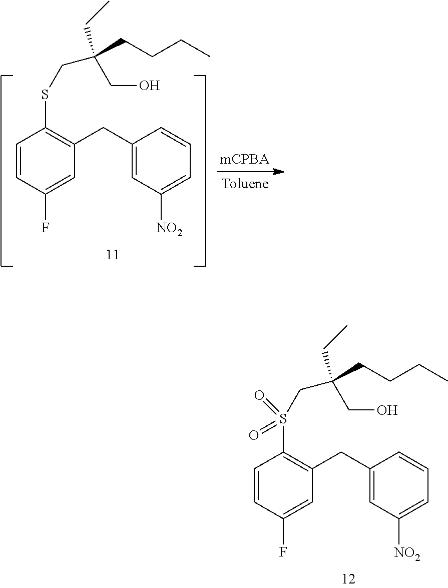

Unexpectedly, a successful synthesis to compound 12 was found. 11 (Formula (II-A-11)) solution from the last step (either after column chromatography purification or telescoped directly as described above) was concentrated and swapped to toluene solution (400 kg, 27.5 active, 68 mol). m-CPBA (active 36.9 kg, 215 mol) was added as solid portion-wise and stirred for 6 h at 30° C. The reaction mixture was quenched with Na₂SO₃ aq. solution (10%, 88 L) and filtered through celite to remove the by-product. The resulting toluene solution was washed with Na₂CO₃ aq. solution (5%, 184 L) twice and solvent was swapped to an IPA solution (55 L). The temperature was adjusted to 40° C. and n-heptane (165 L) was added dropwise over 3 h. The temperature was then adjusted to 15° C. over 3 h and aged for 10 h at 15° C. before filtration. The crude product was re-dissolved in IPA (28 L) and the temperature was adjusted to 40° C. Then n-heptane (165 L) was added dropwise at 40° C. over 3 h and the temperature was adjusted to 15° C. over 3 h and aged for 10 h at 15° C. before filtration. The wet cake was dried under vacuum at 40° C. for 24 h to afford the product (24.4 kg, 82%). Subsequent iterations of the synthesis of compound 12 yielded compound 12 in 91% yield. The reaction was also performed with Na₂WO₄·2H₂O (catalytic), PhPO₃H₂ (catalytic), TBAHS (catalytic), and H₂O₂ in toluene at 80° C., obtaining compound 12 in similar yield and purity. NMR of compound 12 (Formula (II-A-12)) is shown in FIG. 5C. ¹H-NMR (400 MHz, CDCl₃): δ0.76 (t, J=7.6 Hz, 3H), δ0.86 (t, J=7.2 Hz, 3H), δ1.11 (m, 2H), δ1.25 (m, 2H), δ1.37 (m, 2H), δ2.95 (m, 2H), δ3.71 (m, 2H), δ4.60 (m, 2H), δ6.91 (dd, J=9.2 Hz, 1H), δ7.14 (m, 1H), δ7.50 (m, 2H), δ8.05 (s, 1H), δ8.11 (m, 2H).

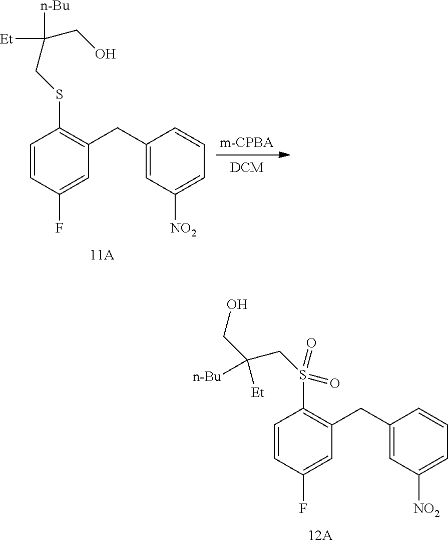

Also prepared was enantiomeric mixture 12A. m-CPBA (7.35 g, 36.2 mmol, 85% purity, 2.7 eq.) was added to compound 11A (Formula (II-B-11)) (5.44 g, 13.4 mmol, 1 eq.) in DCM (108 mL) at 20° C. and the reaction was stirred at 20° C. for 24 h. TLC indicated compound 11A (Formula (II-B-11)) was fully consumed and the reaction was transferred into NaHCO₃ (200 mL). The aqueous phase was extracted with DCM (100 mL×2). The combined organic phase was washed with brine (100 mL), dried with anhydrous Na₂SO₄, filtered, and concentrated under vacuum. The resulting residue was purified by column chromatography (petroleum ether:ethyl acetate 3:1) and compound 12A (Formula (II-B-12)) was obtained as a brown oil. NMR of compound 12A (Formula (II-B-12)) is as follows: ¹H-NMR (400 MHz, CDCl₃) δ ppm 8.22-8.10 (m, 2H), 8.09-7.97 (m, 1H), 7.67-7.49 (m, 2H), 7.20-7.10 (m, 1H), 6.93 (dd, J=2.6, 9.3 Hz, 1H), 4.62 (s, 2H), 3.74-3.66 (m, 2H), 3.00-2.89 (m, 2H), 1.53-1.09 (m, 8H), 0.91-0.87 (m, 3H), 0.82-0.76 (m, 3H).

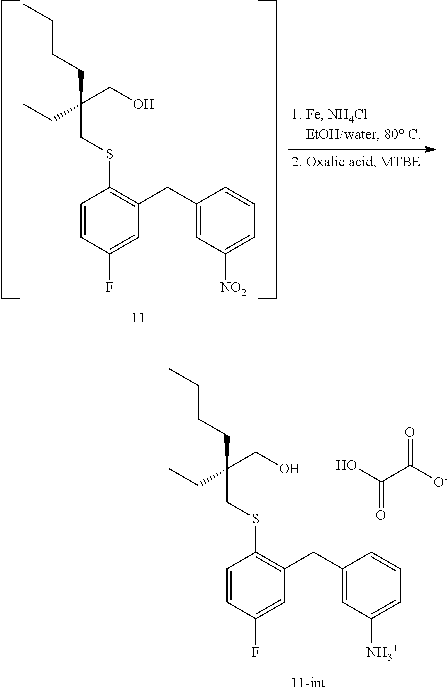

Alternatively, compound 12 can be synthesized from compound 11 via an oxalate salt intermediate, compound 11-int. To a toluene solution of 11 (217 g, active 50.0 g, 0.12 mol, 1.0 eq.), EtOH (200 mL) and saturated NH₄Cl solution (50 mL) were added. Then Fe (20.1 g, 0.36 mol, 10.0 eq.) was added. The resulting suspension was stirred at 80° C. for 5 h. Upon complete conversion, the mixture was cooled to 25° C. and filtered over a Celite pad, then EtOAc (400 mL) was added. The organic phase was separated and dried with Na₂SO₄. After concentration, the crude product was dissolved in MTBE (500 mL), oxalic acid (21.6 g, 0.24 mol, 2.0 eq.) was added and the mixture was stirred for 16 h at 25° C. 11-int oxalate salt was isolated by filtration and dried (60.05 g, assay 65%, yield 70%). Alternative reducing agents that may be used in this reaction include, but are not limited to, H₂ with Pd/C or Raney Ni as catalysts, SnCl₂/NH₄Cl, Zn/AcOH, and Na₂S₂O₄. This reaction was also performed with H₂ and Raney Ni in toluene/EtOH at 25° C., followed by reaction with oxalic acid in EtOAc, and resulted in 11-int in 76% yield.

NMR of compound 11-int is as follows: ¹H-NMR (400 MHz, CDCl₃) δ ppm 0.81 (t, J=7.6 Hz, 3H), 0.89 (t, J=7.0 Hz, 3H), 1.27 (m, 9H), 2.80 (s, 2H), 3.45 (s, 2H), 4.07 (s, 2H), 6.48 (m, 1H), 6.54 (d, J=8.0 Hz, 1H), 6.58 (d, J=8.0 Hz, 1H), 6.83 (dd, J=8.0 Hz, 1H), 6.94-6.86 (m, 1H), 7.08 (dd, J=8.0 Hz, 1H), 7.42 (dd, J=8.0 Hz, 1H). MS data of 11-int showed [M+H]⁺=376.2.

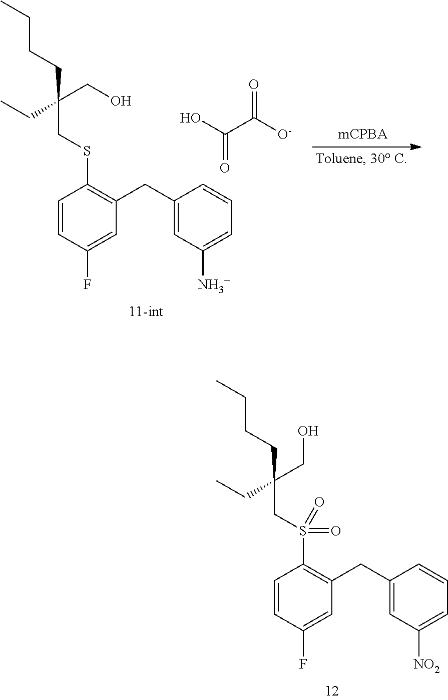 [SMILES: CCCC[C@@](CC)(CO)CSc1ccc(F)cc1Cc1cccc([NH3+])c1 ; O=C([O-])C(=O)O ; CCCC[C@@](CC)(CO)CS(=O)(=O)c1ccc(F)cc1Cc1cccc([N+](=O)[O-])c1]

To a solution of 11-int (active 20 g, 1.0 eq.) in toluene (400 mL) was added m-CPBA (45.96 g, 85%, 5.0 eq.) in portions. The resulting solution was stirred for 2 h at 30° C. Upon reaction completion, the mixture was washed with 5% sodium sulfite solution (100 mL) and 5% Na₂CO₃ solution (100 mL) in turn. The organic phase was concentrated to 60 mL and adjusted to 30° C., followed by the addition of n-heptane (20 mL). Seeds of compound 12 were then introduced. After stirring for 1 hour, n-heptane (80 mL) was added dropwise. Subsequently, the slurry was cooled to 15° C. and stirred for 16 hours. After filtration and drying, 12 was obtained as an off-white solid (12.8 g, 68% yield).

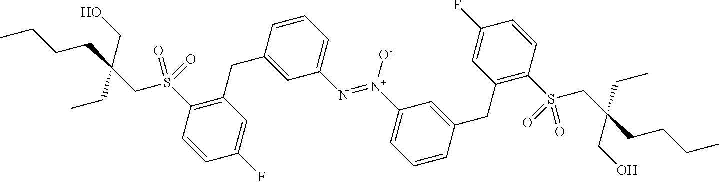 [SMILES: CCCC[C@@](CC)(CO)CS(=O)(=O)c1ccc(F)cc1Cc1cccc(/N=[N+](\[O-])c2cccc(Cc3cc(F)ccc3S(=O)(=O)C[C@](CC)(CO)CCCC)c2)c1]

An azoxy compound impurity was found to form during this oxidation with m-CPBA in up to 4% yield.

### Example 9. Synthesis of Compound 13 (Formula (II-A-13)) and Enantiomer

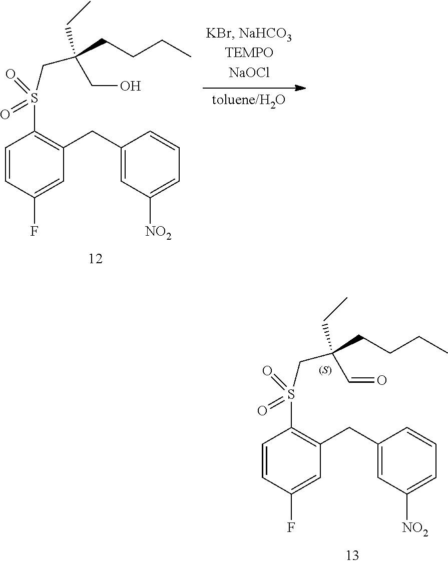

12 (Formula (II-A-12)) (2.92 mol), toluene (15 L) and water (2 L) were charged to a reactor and the contents were cooled to 0° C. NaHCO₃ (1.81), KBr (0.23 mol), TEMPO (0.11 mol) and NaClO (1.83 mol) were added, and the reaction was agitated at 0° C. until completion. Upon completion, the reaction was quenched with 10% Na₂S₂O₃ aqueous solution (3 L). The layers were separated, and the organic layer was washed twice with 5% Na₂SO₄ and 5% NaHCO₃ aqueous solution (6 L) in turn. The organic solution was passed through a CUNO filter and concentrated to 2.1V. The temperature was adjusted to 40° C. and n-heptane (1.8 L) was charged slowly. The content was cooled to −5° C., aged and filtered. The wet cake was washed with n-heptane (2 L) and dried under vacuum to afford the desired product in 89% yield. NMR of compound 13 (Formula (II-A-13)) is shown in FIG. 5D. ¹H-NMR (400 MHz, DMSO-d₆) δ ppm 0.70 (t, J=7.40 Hz, 3H), 0.82 (t, J=7.28 Hz, 3H), 0.94-1.10 (m, 2H), 1.11-1.28 (m, 2H), 1.47-1.78 (m, 4H), 3.53-3.64 (m, 2H), 4.54-4.66 (m, 2H), 7.31 (dd, J=10.04, 2.51 Hz, 1H), 7.44 (td, J=8.41, 2.51 Hz, 1H), 7.59-7.67 (m, 1H), 7.67-7.74 (m, 1H), 8.05 (dd, J=8.91, 5.90 Hz, 1H), 8.09-8.19 (m, 2H), 9.46-9.53 (m, 1H).

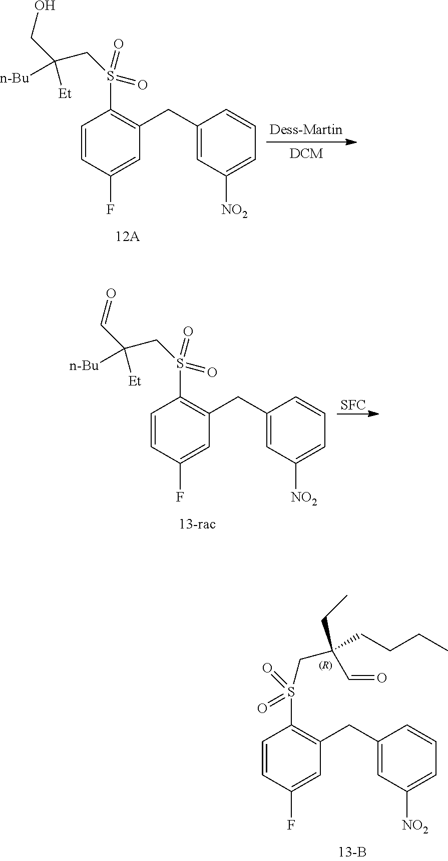

Also prepared was enantiomer 13B. To a mixture of compound 12A (Formula (II-B-12)) (17.4 g, 39.7 mmol, 1 eq.) in DCM (248 mL) was charged Dess-Martin (17.7 g, 41.7 mmol, 12.9 mL, 1.05 eq.) in portions at 0° C. The reaction was agitated at 0° C. for 1 hour then warmed to 20° C. and agitated for an addition 1 hour. LCMS indicated compound 12A (Formula (II-B-12)) was fully consumed and the reaction was transferred into NaHCO₃ (50 mL). The aqueous phase was extracted with DCM (100 mL×2) and dried with anhydrous Na₂SO₄, filtered, and concentrated under vacuum. The residue was purified by column chromatography (Petroleum ether:ethyl acetate 3:1) to obtain crude compound 13-rac (Formula (II-B-13)), a racemic mixture of 13:13B. Compound 13-rac (Formula (II-B-13)) (4.74 g, 9.90 mmol, 24.9% yield, 91% purity) was obtained as brown oil. NMR of compound 13-rac (Formula (II-B-13)) is as follows: ¹H-NMR (400 MHz, CDCl₃) δ ppm 8.19-8.02 (m, 3H), 7.62-7.45 (m, 2H), 7.17 (ddd, J=2.6, 7.5, 8.9 Hz, 1H), 6.93 (dd, J=2.5, 9.3 Hz, 1H), 4.60 (s, 2H), 4.13 (q, J=7.1 Hz, 1H), 3.27-3.13 (m, 2H), 2.01-1.91 (m, 1H), 1.90-1.89 (m, 1H), 1.84-1.61 (m, 2H), 1.33-1.02 (m, 5H), 0.88 (td, J=7.4, 11.4 Hz, 6H).

Further, compound 13-rac (Formula (II-B-13)) was separated by SFC (Supercritical fluid chromatography) (condition: EtOH, column: DAICEL CHIRALPAK AD (250 mm*50 mm, 10 μm); mobile phase: [0.1% NH₃—H₂O EtOH]; B %: 35%-35%, 7.5 min.) and the organic layers were concentrated under reduced pressure to afford compound 13B (Formula (II-C-13)) (6 g, 10.4 mmol, 77.4% yield) as an off-white solid. NMR of compound 13B (Formula (II-C-13)) is as follows: ¹H-NMR (400 MHz, CDCl₃) δ ppm 8.19-8.02 (m, 3H), 7.62-7.45 (m, 2H), 7.17 (ddd, J=2.6, 7.5, 8.9 Hz, 1H), 6.93 (dd, J=2.5, 9.3 Hz, 1H), 4.60 (s, 2H), 4.13 (q, J=7.1 Hz, 1H), 3.27-3.13 (m, 2H), 2.01-1.91 (m, 1H), 1.90-1.89 (m, 1H), 1.84-1.61 (m, 2H), 1.33-1.02 (m, 5H), 0.88 (td, J=7.4, 11.4 Hz, 6H).

### Example 10. Synthesis of Compound 15 (Formula (II-E-15)) and Diastereomers

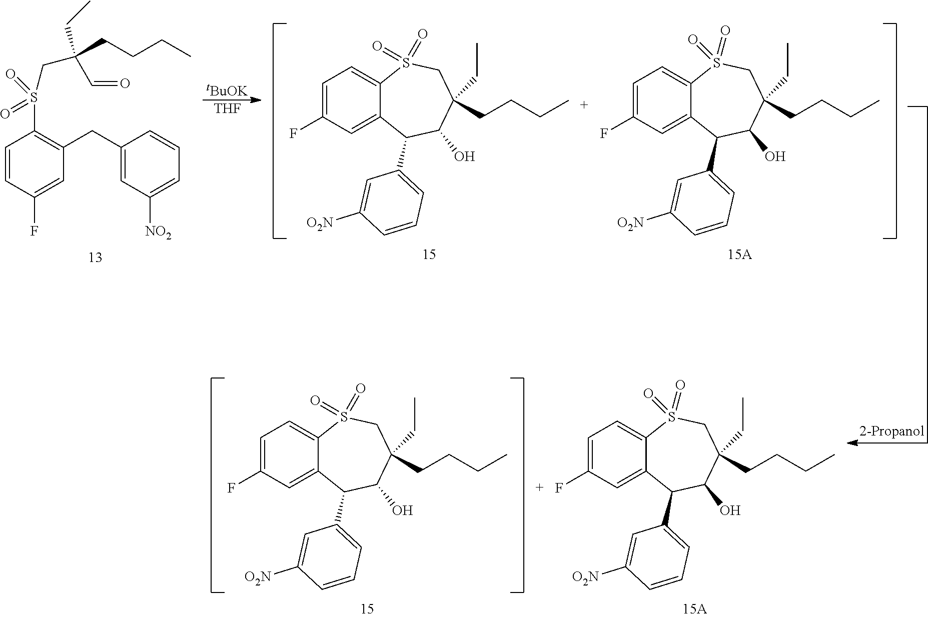

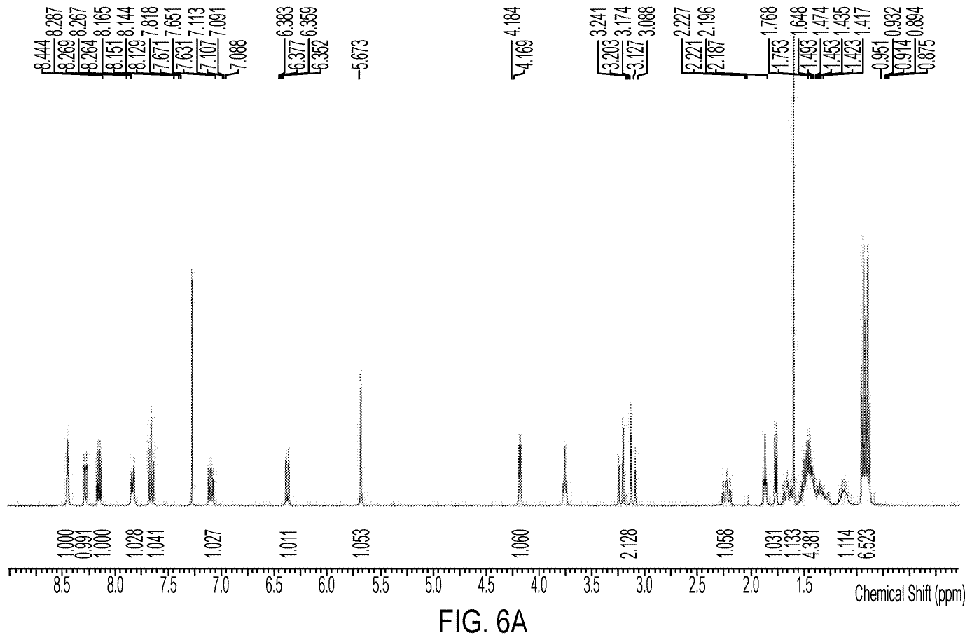

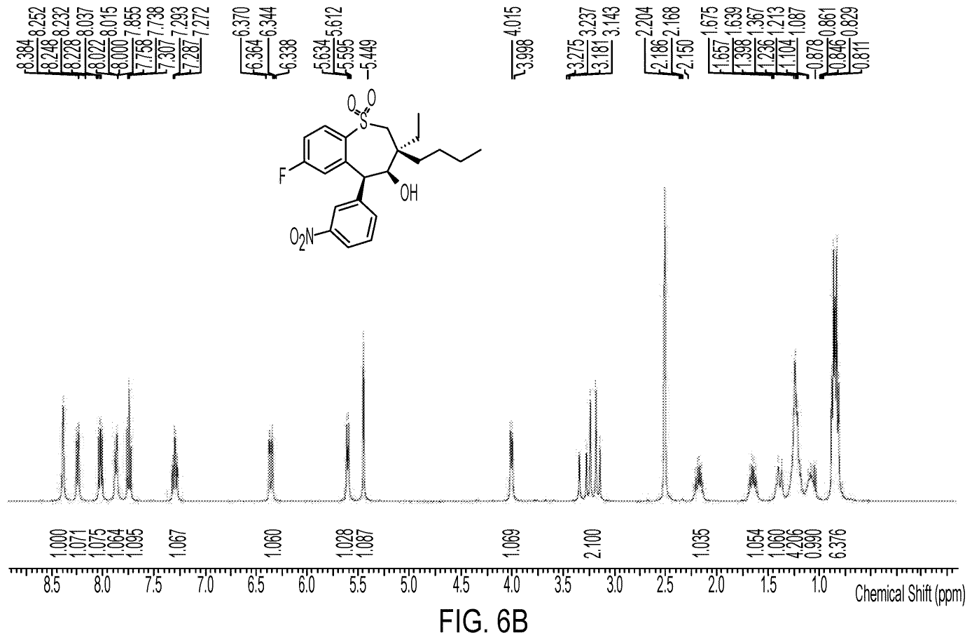

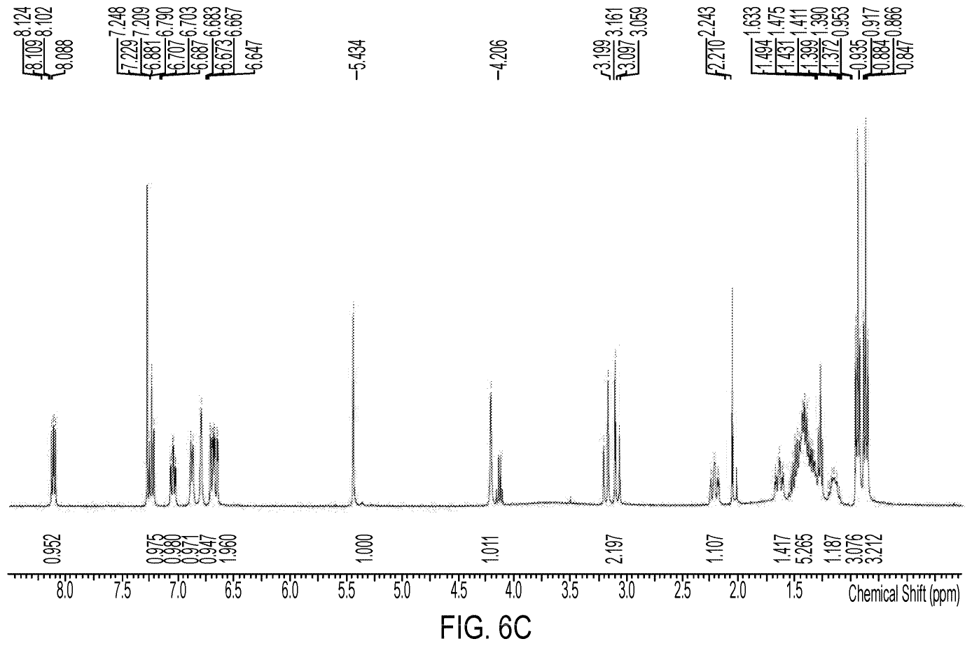

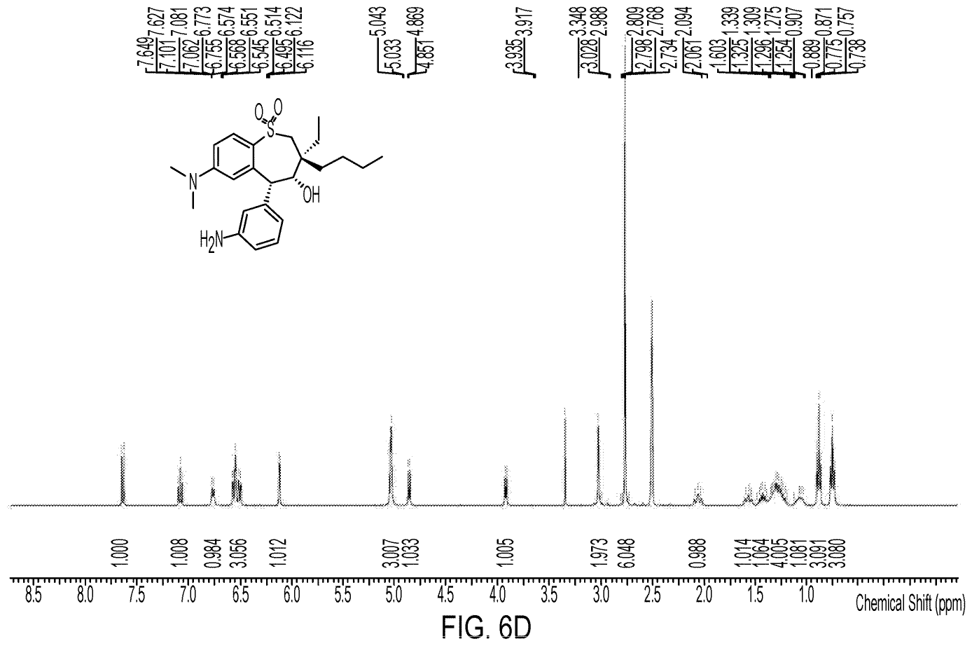

13 (Formula (II-A-13)) (2.3 mol) and THF (4 L) were charged to an inert reactor and agitated to dissolve. The solution was then adjusted to −15° C. and a 20 wt % t-BuOK solution in THF (0.12 L) was charged slowly. The reaction was agitated at −15° C. until completion then quenched with AcOH (10% in THF, 0.23 L). The solution was concentrated to 2V and 10 L IPA was charged. The solution was concentrated to 8V followed by two additional 3 L IPA co-distillations to 8V. The reaction temperature was adjusted to 60° C. then ramp cooled to 20° C. and aged. The resulting slurry was filtered, washed with IPA, and dried to afford compound 15 (Formula (II-E-15)) in the filtrate as a solution in IPA (52% yield) and compound 15A (Formula (II-F-15)) as a solid (42% yield). Compound 15 (Formula (II-E-15)) can be isolated as a solid in 47% yield by concentrating the IPA solution and charging n-heptane while cooling to 0° C. This process was repeated with compound 15A (Formula (II-F-15)) as the starting material to yield compounds 15 and 15A (Formula (II-F-15)) in similar yield and purity. NMR of compound 15 (Formula (II-E-15)) isolated as a solid is shown in FIG. 6A. ¹H-NMR (400 MHz, CDCl₃) δ ppm 0.84-1.00 (m, 6H), 1.04-1.18 (m, 1H), 1.26-1.36 (m, 1H), 1.38-1.55 (m, 4H), 1.60-1.70 (m, 1H), 1.76 (d, J=6.00 Hz, 1H), 1.73-1.79 (m, 1H), 1.83-1.90 (m, 1H), 2.16-2.29 (m, 1H), 3.06-3.27 (m, 2H), 3.45-3.99 (m, 1H), 3.69-3.81 (m, 1H), 4.18 (d, J=6.00 Hz, 1H), 5.67 (s, 1H), 6.37 (dd, J=9.94, 2.44 Hz, 1H), 7.05-7.14 (m, 1H), 7.65 (t, J=7.94 Hz, 1H), 7.83 (br d, J=7.63 Hz, 1H), 8.15 (dd, J=8.69, 5.69 Hz, 1H), 8.23-8.32 (m, 1H), 8.44 (s, 1H). NMR of 15A (Formula (II-F-15)) as a solid is shown in FIG. 6B. ¹H-NMR (400 MHz, DMSO-d₆) δ ppm 0.76-0.93 (m, 6H), 1.08 (m, 1H), 1.17-1.32 (m, 4H), 1.33-1.45 (m, 1H), 1.65 (dq, J=14.31, 7.11 Hz, 1H), 2.18 (dq, J=14.34, 7.18 Hz, 1H), 3.11-3.29 (m, 2H), 4.01 (br d, J=6.78 Hz, 1H), 5.45 (s, 1H), 5.60 (d, J=6.78 Hz, 1H), 6.35 (dd, J=10.42, 2.38 Hz, 1H), 7.29 (td, J=8.28, 2.51 Hz, 1H), 7.65-7.80 (m, 1H), 7.86 (br d, J=7.53 Hz, 1H), 8.02 (dd, J=8.66, 5.90 Hz, 1H), 8.24 (dd, J=8.03, 1.51 Hz, 1H), 8.38 (s, 1H).

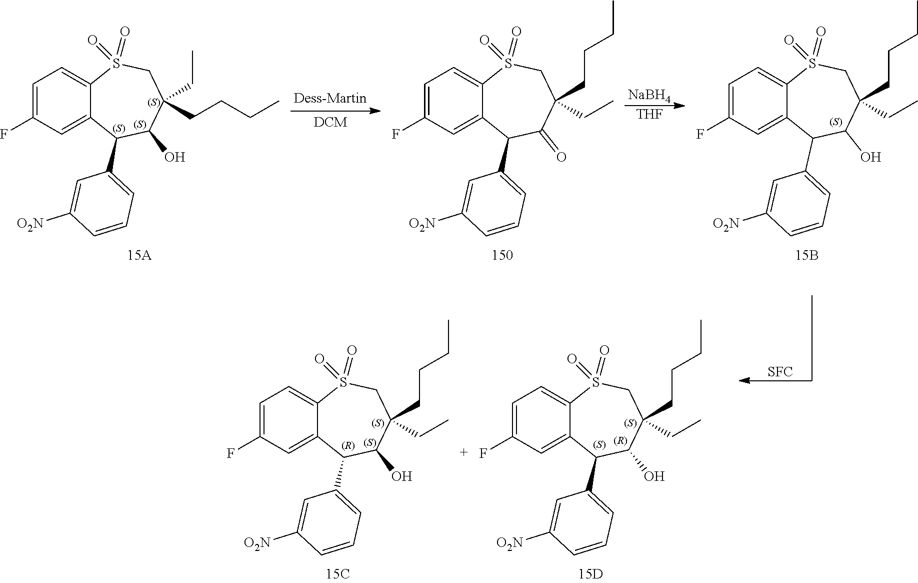

Further diastereomers were synthesized. To a solution of 15A (Formula (II-F-15)) (20.0 g, 45.9 mmol, 1.00 eq.) in DCM (200 mL) was added Dess-Martin (38.9 g, 91.8 mmol, 28.4 mL, 2.00 eq.). The mixture was stirred at 25° C. for 19 hours. TLC (Petroleum ether:Ethyl acetate=3:1) indicated compound 15A (Formula (II-F-15)) was consumed completely and one new spot formed. The reaction mixture was quenched by addition of NaHCO₃ (200 mL) at 25° C., and then diluted with DCM (30.0 mL) and extracted with DCM (90.0 mL, 30.0 mL×3). The extract was filtered and concentrated under reduced pressure to give a residue. The crude product compound 150 (Formula (II-A-150)) (25.1 g, 40.5 mmol, 88.2% yield, 70.0% purity) was obtained as a white solid and was used into the next step without further purification. NMR of compound 150 (Formula (II-A-150)) is as follows: ¹H-NMR (400 MHz, CDCl₃) δ ppm 0.92 (q, J=7.27 Hz, 6H), 1.08-1.20 (m, 2H), 1.22-1.32 (m, 6H), 1.36-1.49 (m, 3H), 1.59-1.68 (m, 1H), 2.13-2.29 (m, 2H), 2.83 (s, 6H), 3.00 (d, J=15.26 Hz, 1H), 3.12-3.21 (m, 1H), 4.12 (d, J=7.75 Hz, 1H) 4.79 (br s, 2H), 5.21 (s, 2H), 5.51 (s, 1H), 5.99 (d, J=2.50 Hz, 1H), 6.52 (dd, J=8.94, 2.62 Hz, 1H), 7.04 (d, J=8.70 Hz, 2H), 7.34-7.51 (m, 6H), 7.91 (d, J=8.82 Hz, 1H).

Further, to a solution of compound 150 (Formula (II-A-150)) (20.0 g, 46.1 mmol, 1.00 eq.) in THF (200 mL) was added NaBH₄ (3.63 g, 95.9 mmol, 2.08 eq.) in portions at 0° C. The mixture was stirred at 0° C. for 1 hour and then stirred at 25° C. for 16 hours and a yellow suspension was obtained. TLC (Petroleum ether:Ethyl acetate=3:1) indicated compound 150 (Formula (II-A-150)) was fully consumed. The reaction mixture was added into saturated NH₄Cl solution (50.0 mL) slowly at 0° C. and extracted with THF (100 mL×2). The organic layers were combined and dried over Na₂SO₄, filtered, and concentrated under reduced pressure to give a residue. Compound 15B (Formula (II-G-15)) was obtained as a yellow solid.

Then, compound 15B (Formula (II-G-15)) was purified by SFC 1 (column: DAICEL CHIRALPAK IG (250 mm*30 mm, 10 μm); mobile phase: [Neu-MeOH]; B %: 25%-25%, 8 min.) and further purified by SFC 2 (column: Phenomenex-Cellulose-2 (250 mm*30 mm, 10 μm); mobile phase: [Neu-ETOH]; B %: 35%-35%, 5 min.) to obtain compound 15C (Formula (II-H-15)) and compound 15D (Formula (II-I-15)). Compound 15C (Formula (II-H-15)) (1.51 g, 3.43 mmol, 99.0% purity) was obtained as a white solid. Compound 15D (Formula (II-I-15)) (1.0 g, 2.25 mmol, 98.0% purity) was obtained as a white solid. NMR of compound 15C (Formula (II-H-15)) is as follows: ¹H-NMR (400 MHz, CDCl₃) δ ppm 0.82-0.98 (m, 6H), 1.25-1.36 (m, 4H), 1.37 (br s, 2H), 1.57-1.66 (m, 2H), 1.75 (br s, 1H), 3.28-3.35 (m, 1H), 3.36-3.44 (m, 1H), 4.63 (br d, J=8.80 Hz, 1H), 5.06 (br d, J=8.93 Hz, 1H), 6.75 (dd, J=9.72, 2.38 Hz, 1H), 7.06-7.14 (m, 1H), 7.54-7.61 (m, 1H), 7.78 (d, J=7.82 Hz, 1H), 8.14 (dd, J=8.74, 5.69 Hz, 1H), 8.17-8.22 (m, 1H), 8.34 (t, J=1.90 Hz, 1H). NMR of compound 15D (Formula (II-I-15)) is as follows: ¹H-NMR (400 MHz, CDCl₃) δ ppm 0.82-0.90 (m, 3H), 0.95 (t, J=7.40 Hz, 3H), 1.18-1.35 (m, 6H), 1.64-1.78 (m, 3H), 3.29-3.37 (m, 1H), 3.39-3.47 (m, 1H), 4.63 (br d, J=8.93 Hz, 1H), 5.04 (br d, J=8.93 Hz, 1H), 6.75 (dd, J=9.78, 2.32 Hz, 1H), 7.07-7.15 (m, 1H), 7.54-7.61 (m, 1H), 7.77 (d, J=7.70 Hz, 1H), 8.14 (dd, J=8.80, 5.75 Hz, 1H), 8.18-8.22 (m, 1H), 8.34 (s, 1H).

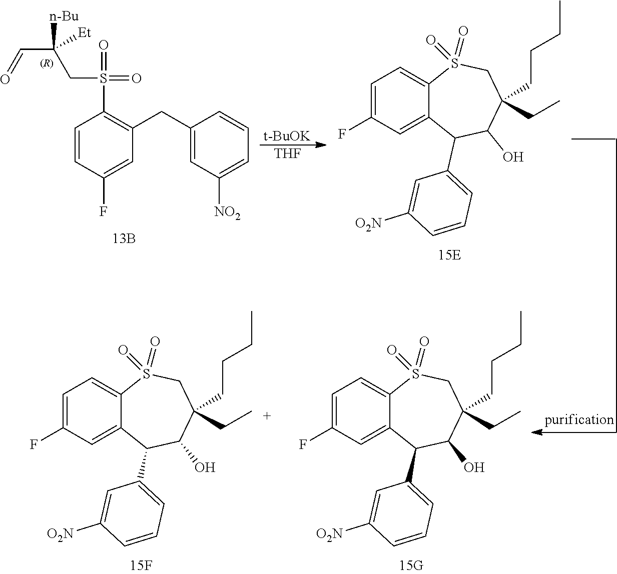

Further diastereomers were synthesized. To a mixture of compound 13B (Formula (II-C-13)) (4.74 g, 10.8 mmol, 1 eq.) in dry THF (47.4 mL) was charged t-BuOK (219 mg, 1.96 mmol, 0.18 eq.) drop-wise at 0° C. The reaction was agitated at 0° C. for 1 hour and TLC indicated compound 13B (Formula (II-C-13)) was fully consumed. The mixture was quenched with AcOH (496 mg, 8.27 mmol, 472 μL, 0.18 eq.) at 0° C., then was concentrated under reduced pressure to give a residue. The residue was purified by column chromatography (petroleum ether:ethyl acetate 3:1) to afford compound 15E (Formula (II-J-15)) (5.04 g) as a brown oil. Compound 15E (Formula (II-J-15)) (1 g, 5.75 mmol, 1 eq.) was further purified by column chromatography (petroleum ether:ethyl acetate 3:1) to afford compound 15F (Formula (II-K-15)) (237 mg, 544 μmol) as an off-white solid and compound 15G (Formula (II-L-15)) (237 mg, 544 μmol) as an off-white solid. NMR of compound 15F (Formula (II-K-15)) is as follows: ¹H-NMR (400 MHz, CDCl₃) δ ppm 8.43 (s, 1H), 8.27 (dd, J=1.3, 8.2 Hz, 1H), 8.15 (dd, J=5.7, 8.7 Hz, 1H), 7.82 (br d, J=7.6 Hz, 1H), 7.64 (t, J=8.0 Hz, 1H), 7.12-7.05 (m, 1H), 6.38 (dd, J=2.3, 10.0 Hz, 1H), 4.19 (d, J=5.7 Hz, 1H), 3.50 (s, 1H), 3.26-3.06 (m, 2H), 2.31 (qd, J=7.3, 14.6 Hz, 1H), 1.84-1.68 (m, 2H), 1.62-1.12 (m, 7H), 0.98-0.91 (m, 6H). NMR of compound 15G (Formula (II-L-15)) is as follows: ¹H-NMR (400 MHz, CDCl₃) δ ppm 8.44 (s, 1H), 8.28 (dd, J=1.4, 8.2 Hz, 1H), 8.15 (dd, J=5.7, 8.7 Hz, 1H), 7.83 (br d, J=7.5 Hz, 1H), 7.65 (t, J=8.0 Hz, 1H), 7.15-7.02 (m, 1H), 6.37 (dd, J=2.4, 10.0 Hz, 1H), 4.18 (d, J=6.0 Hz, 1H), 3.50 (d, J=4.5 Hz, 1H), 3.27-3.05 (m, 2H), 2.28-2.17 (m, 1H), 1.71-1.60 (m, 2H), 1.54-1.23 (m, 6H), 1.18-1.06 (m, 1H), 0.95-0.87 (m, 6H).

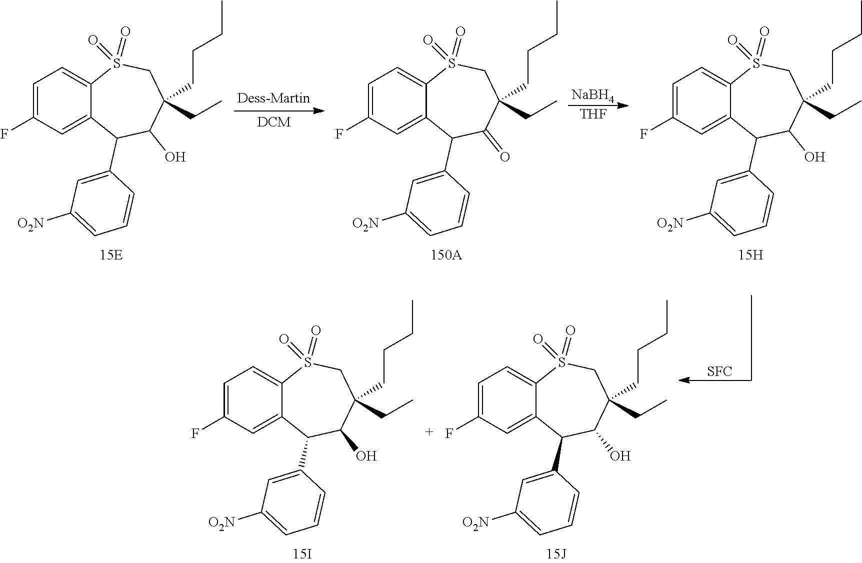

Further diastereomers were synthesized. To a mixture of compound 1E (Formula (II-J-15)) (4.04 g, 9.28 mmol, 1 eq.) in DCM (40.4 mL) was charged Dess-Martin (7.87 g, 18.5 mmol, 5.74 mL, 2 eq.) drop-wise at 0° C. The mixture was agitated at 0° C. for 1 hour and LCMS indicated compound 15E (Formula (II-J-15)) was fully consumed. The mixture was quenched with AcOH (496 mg, 8.27 mmol, 472 μL, 0.18 eq.) at 0° C., then was concentrated under reduced pressure to give a residue. The residue was purified by column chromatography (petroleum ether/ethyl acetate 3/1) to afford compound 150A (Formula (II-B-150)) (5.04 g) as a brown oil. NMR of compound 150A (Formula (II-B-150)) is as follows: ¹H-NMR (400 MHz, CDCl₃) δ ppm 8.63 (td, J=1.9, 5.9 Hz, 1H), 8.29-8.24 (m, 1H), 8.07-8.01 (m, 1H), 7.69-7.59 (m, 1H), 7.15-7.09 (m, 1H), 6.98 (s, 1H), 6.77-6.68 (m, 1H), 3.54 (dd, J=3.1, 15.0 Hz, 1H), 3.11 (dd, J=15.1, 16.6 Hz, 1H), 2.89-2.66 (m, 1H), 2.12-1.90 (m, 1H), 1.85-1.67 (m, 1H), 1.65-1.42 (m, 2H), 1.38-1.03 (m, 4H), 0.94-0.85 (m, 3H), 0.79-0.66 (m, 3H).

Further, NaBH₄ (352.59 mg, 9.32 mmol, 1 eq.) was charged in portions to a mixture of compound 150A (Formula (II-B-150)) (4.04 g, 9.32 mmol, 1 eq.) in THF (40.4 mL) at 0° C. The reaction mixture was agitated at 0° C. for 1 hour then warmed to 25° C. and agitated for 16 hours. TLC indicated compound 150A (Formula (II-B-150)) was fully consumed and the reaction mixture was then transferred into water (40 mL) and extracted with THF (40 mL×3). The combined organic layer was dried with Na₂SO₄, filtered, and concentrated under vacuum and a residue was obtained. The residue was purified by reverse-phase HPLC (column: Phenomenex Luna C18 200*40 mm*10 μm; mobile phase: [water (FA)-ACN]; B %: 30%-85%, 8 min.) followed by further separation by SFC to afford compound 15H (Formula (II-M-15)) as a brown oil.

Compound 15H (Formula (II-M-15)) was separated by SFC (column: DAICEL CHIRALPAK AD (250 mm*50 mm, 10 μm); mobile phase: [0.1% NH₃H₂O ETOH]; B %: 35%-35%, 7.5 min.). The organic layers were concentrated under reduced pressure and compound 151 (Formula (II-N-15)) (540 mg, 1.24 mmol, 13.3% yield) was obtained as a brown oil and compound 15J (Formula (II-O-15)) (850 mg, 1.24 mmol, 13.3% yield) was also obtained as a brown oil. NMR of compound 151 (Formula (II-N-15)) is as follows: ¹H-NMR (400 MHz, CDCl₃) δ ppm 8.34 (s, 1H), 8.23-8.10 (m, 2H), 7.78 (br d, J=7.9 Hz, 1H), 7.58 (t, J=7.9 Hz, 1H), 7.14-7.05 (m, 1H), 6.75 (dd, J=2.1, 9.7 Hz, 1H), 5.04 (br d, J=8.6 Hz, 1H), 4.69-4.56 (m, 1H), 3.52-3.30 (m, 2H), 1.79-1.52 (m, 3H), 1.35-1.20 (m, 5H), 0.99-0.80 (m, 6H). NMR of compound 15J (Formula (II-O-15)) is as follows: ¹H-NMR (400 MHz, CDCl₃) δ ppm 8.35 (t, J=1.8 Hz, 1H), 8.24-8.10 (m, 2H), 7.78 (d, J=7.7 Hz, 1H), 7.58 (t, J=7.9 Hz, 1H), 7.15-7.04 (m, 1H), 6.75 (dd, J=2.3, 9.7 Hz, 1H), 5.06 (br d, J=8.6 Hz, 1H), 4.63 (br dd, J=6.0, 8.3 Hz, 1H), 3.45-3.26 (m, 2H), 1.69-1.51 (m, 3H), 1.49-1.23 (m, 6H), 0.90 (t, J=7.2 Hz, 6H).

### Example 11. Synthesis of Compound 16 (Formula (II-E-16)) and Diastereomers

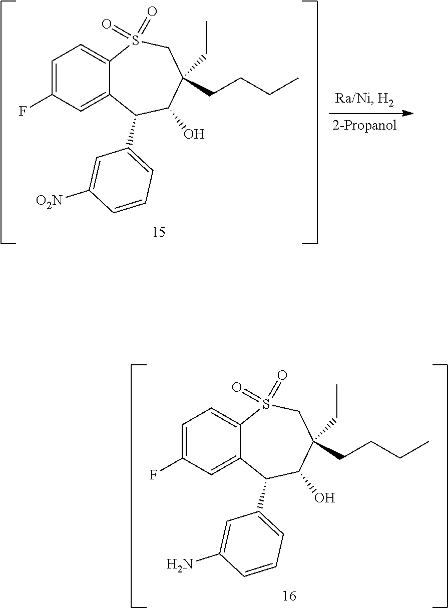

A solution of 15 (Formula (II-E-15)) (2.5 mol) in IPA (14 L) was combined with Raney Ni (0.74 mol) and agitated at 40° C. under 0.6 MPa H₂ until reaction completion. Upon completion, the contents were filtered to afford the desired compound as a solution in IPA in >99% yield. Compound 16 can be isolated as a solid in 85% yield by concentrating the IPA solution and charging n-heptane while cooling to 0° C. The reaction was also successfully performed using Pd(OH)₂/C as the catalyst. NMR of compound 16 (Formula (II-E-16)) is shown in FIG. 6C. ¹H-NMR (400 MHz, CDCl₃) δ ppm 0.87 (t, J=7.53 Hz, 3H), 0.93 (t, J=7.15 Hz, 3H), 1.08-1.20 (m, 1H), 1.30-1.56 (m, 7H), 1.57-1.69 (m, 1H), 2.16-2.28 (m, 1H), 3.02-3.23 (m, 2H), 4.21 (s, 1H), 5.43 (s, 1H), 6.60-6.73 (m, 2H), 6.79 (s, 1H), 6.87 (br d, J=7.53 Hz, 1H), 7.04 (td, J=8.16, 2.51 Hz, 1H), 7.23 (t, J=7.78 Hz, 1H), 8.11 (dd, J=8.66, 5.65 Hz, 1H).

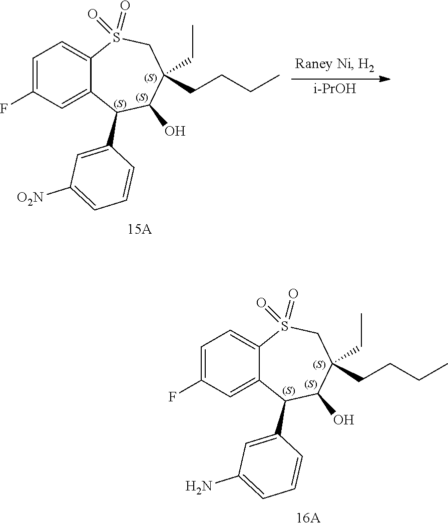

Further diastereomers were synthesized. To a solution of 15A (Formula (II-F-15)) (18.0 g, 41.3 mmol, 1 eq) in i-PrOH (180 mL) was added Raney nickel (8.99 g, 104.98 mmol, 2.54 eq). The mixture was stirred at 20° C. for 12 hours under H₂ (15 Psi). LCMS showed that 15A (Formula (II-F-15)) was consumed and the desired product, compound 16A (Formula (II-F-16)), was detected. The mixture was concentrated under reduced pressure to give a residue without further purification. Compound 16A (Formula (II-F-16)) (16.0 g, 39.2 mmol, 94.9% yield, 99.4% purity) was obtained as a white solid. NMR of compound 16A (Formula (II-F-16)) is as follows: ¹H-NMR (400 MHz, CDCl₃) δ ppm 8.11 (dd, J=5.7, 8.7 Hz, 1H), 7.25-7.19 (m, 1H), 7.08-7.00 (m, 1H), 6.92-6.83 (m, 1H), 6.82-6.76 (m, 1H), 6.74-6.59 (m, 2H), 5.50-5.36 (m, 1H), 4.27-4.18 (m, 1H), 4.01-3.41 (m, 2H), 3.28-3.01 (m, 2H), 2.36-2.20 (m, 1H), 1.83-1.70 (m, 1H), 1.50-1.38 (m, 2H), 1.27 (s, 4H), 1.24-1.17 (m, 1H), 0.93 (q, J=7.4 Hz, 6H).

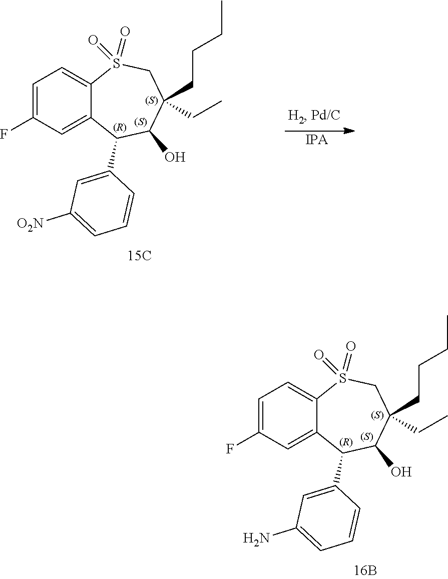

Also synthesized was compound 16B. A mixture of compound 15C (Formula (II-H-15)) (1.20 g, 2.76 mmol, 1.00 eq.) and Pd/C (0.50 g, 10.0% purity) in IPA (10 mL) was degassed and purged with H₂ (5.57 mg, 2.76 mmol, 1.00 eq.) at 20° C. The mixture was stirred at 35° C., 15 Psi for 12 hours and a yellow solution was obtained. TLC (Petroleum ether:Ethyl acetate=2:1) indicated compound 15C (Formula (II-H-15)) was consumed completely and LCMS indicated the target compound was obtained. The reaction mixture was filtered to remove Pd/C and the filtrate was concentrated under reduced pressure. Without further purification, compound 16B (Formula (II-G-16)) (1.20 g, crude) was obtained as a yellow oil.

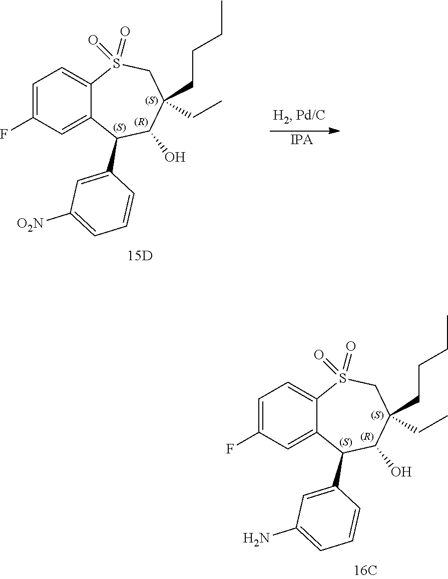

Also synthesized was compound 16C. A mixture of compound 15D (Formula (II-I-15)) (0.70 g, 1.61 mmol, 1.00 eq.) and Pd/C (0.50 g, 10.0% purity) in IPA (10 mL) was degassed and purged with H₂ (3.25 mg, 1.61 mmol, 1.00 eq.) at 20° C. The mixture was stirred at 35° C. and 15 Psi for 12 hours and a yellow solution was obtained. TLC (Petroleum ether:Ethyl acetate=2:1) indicated compound 15D (Formula (II-I-15)) was fully consumed and LCMS indicated 16C (Formula (II-H-16)) was obtained. The reaction mixture was filtered to remove Pd/C and the liquid was concentrated under reduced pressure and 16C (Formula (II-H-16)) (0.70 g, crude) was obtained as a yellow oil and used for the next step without purification.

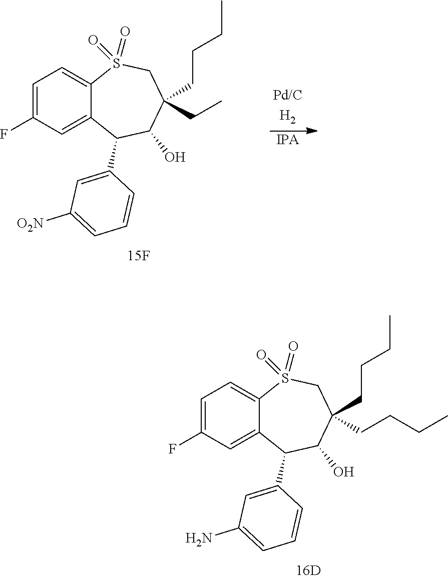

Also synthesized was compound 16D. Compound 15F (Formula (II-K-15)) (237 mg, 544 μmol, 1 eq.) was combined with Pd/C (0.3 g, 544 μmol, 10% purity, 1 eq.) in IPA (10 mL) at 20° C. and agitated at 45° C. for 18 hours. LCMS indicated compound 15F (Formula (II-K-15)) was fully consumed and the reaction mixture was filtered. The organic layer of the filtrate was concentrated under vacuum and a residue was obtained. The residue was purified by column chromatography (petroleum ether/ethyl acetate=3/1) and compound 16D (Formula (II-I-16)) was obtained as a brown oil. NMR of compound 16D (Formula (II-I-16)) is as follows: ¹H-NMR (400 MHz, CDCl₃) δ ppm 8.11 (dd, J=5.7, 8.6 Hz, 1H), 7.22 (t, J=7.7 Hz, 1H), 7.09-6.96 (m, 1H), 6.86 (br d, J=7.5 Hz, 1H), 6.78 (s, 1H), 6.73-6.62 (m, 2H), 4.22 (d, J=5.7 Hz, 1H), 3.76 (br s, 2H), 3.23-3.01 (m, 2H), 2.36-2.24 (m, 1H), 1.49-1.13 (m, 8H), 0.93 (q, J=7.5 Hz, 6H).

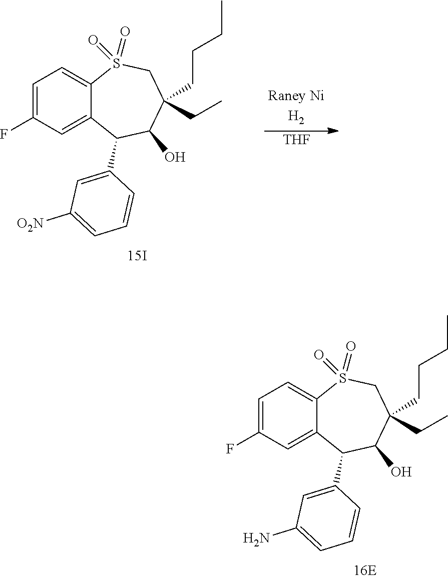 [SMILES: CCCC[C@@]1(CC)CS(=O)(=O)c2ccc(F)cc2[C@@H](c2cccc([N+](=O)[O-])c2)[C@@H]1O ; CCCC[C@@]1(CC)CS(=O)(=O)c2ccc(F)cc2[C@@H](c2cccc(N)c2)[C@@H]1O ; "THF"]

Also synthesized was compound 16E. Compound 151 (Formula (II-N-15)) (300 mg, 688 μmol, 1 eq.) was combined with Raney nickel (0.3 g, 5.11 mmol, 7.42 eq.) in THF (10 mL) at 20° C. The reaction mixture was degassed and purged with H₂ (462.86 μg, 229.62 μmol, 1 eq.) 3 times then agitated under 15 Psi at 45° C. for 12 hours. LCMS indicated compound 151 (Formula (II-N-15)) was fully consumed and the reaction mixture was filtered and concentrated under vacuum to afford compound 16E (Formula (II-J-16)) (222.5 mg) as a brown oil.

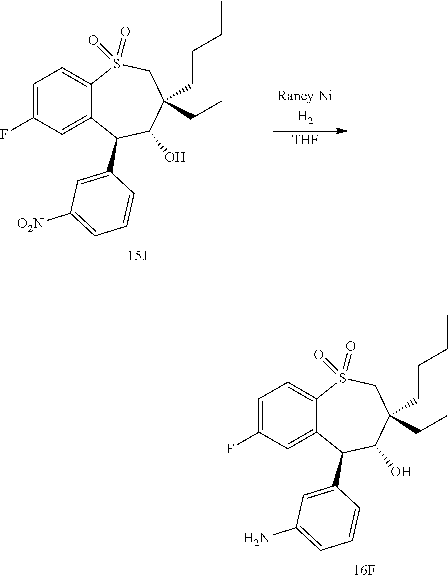 [SMILES: CCCC[C@@]1(CC)CS(=O)(=O)c2ccc(F)cc2[C@H](c2cccc([N+](=O)[O-])c2)[C@H]1O ; CCCC[C@@]1(CC)CS(=O)(=O)c2ccc(F)cc2[C@H](c2cccc(N)c2)[C@H]1O ; "THF"]

Further, was synthesized compound 16F. Compound 15J (Formula (II-O-15)) (100 mg, 229 μmol, 1 eq.) was combined with Raney nickel (100 mg, 1.70 mmol, 7.42 eq.) in THF (10 mL) at 20° C. The reaction mixture was degassed and purged with H₂ (462.86 μg, 229.62 μmol, 1 eq.) 3 times at 20° C. then agitated under 15 Psi for 12 hours. LCMS indicated compound 15J (Formula (II-O-15)) was fully consumed, the reaction mixture was filtered, and the filtrate was concentrated under vacuum to afford compound 16F (Formula (II-K-16)) (92.85 mg, crude) as a brown oil.

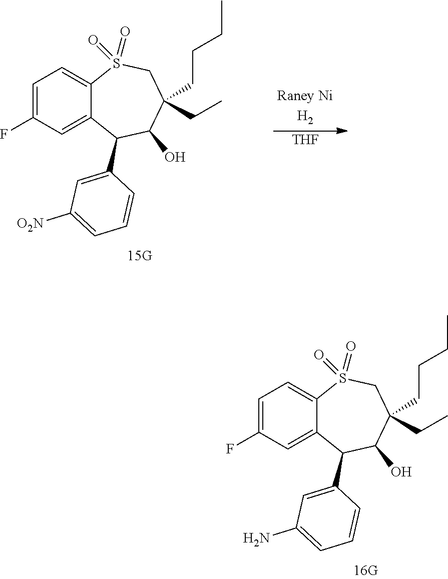 [SMILES: CCCC[C@@]1(CC)CS(=O)(=O)c2ccc(F)cc2[C@H](c2cccc([N+](=O)[O-])c2)[C@@H]1O ; CCCC[C@@]1(CC)CS(=O)(=O)c2ccc(F)cc2[C@H](c2cccc(N)c2)[C@@H]1O ; "THF"]

Further, was synthesized compound 16G. Compound 15G (Formula (II-L-15)) (84 mg, 192 μmol, 1 eq.) was combined with Raney nickel (200 mg, 3.41 mmol, 17.6 eq.) in THF (10 mL) at 20° C. The reaction mixture was degassed and purged with H₂ (1.10 mg, 544 μmol, 1 eq.) 3 times then agitated at 20° C. for 12 hours. LCMS indicated compound 15G (Formula (II-L-15)) was fully consumed, the reaction mixture was filtered, and the filtrate was concentrated under vacuum to afford compound 16G (Formula (II-L-16)) (200 mg) as an off-white solid. NMR of compound 16G (Formula (II-L-16)) is as follows: ¹H-NMR (400 MHz, CDCl₃) δ ppm 8.11 (dd, J=5.8, 8.6 Hz, 1H), 7.25-7.20 (m, 1H), 7.11-6.97 (m, 1H), 6.88 (br d, J=8.0 Hz, 1H), 6.81 (br s, 1H), 6.74-6.62 (m, 2H), 4.21 (br d, J=1.6 Hz, 1H), 4.03-3.90 (m, 2H), 3.84-3.67 (m, 1H), 3.23-2.99 (m, 2H), 1.94-1.77 (m, 3H), 1.54-1.33 (m, 6H), 0.97-0.81 (m, 6H).

### Example 12. Synthesis of Compound 17 (Formula (II-A-17)) and Diastereomers

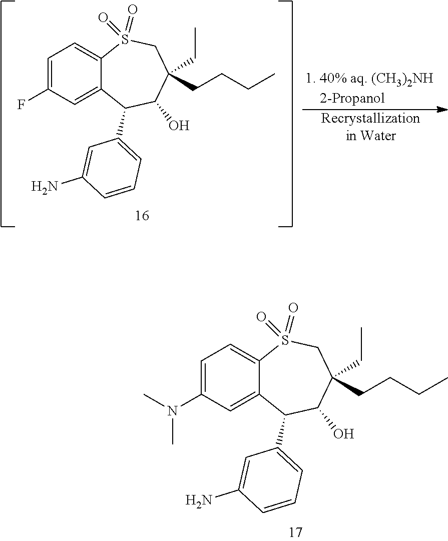

A solution of 16 (Formula (II-E-16)) (2.47 mol) in IPA and a 40 wt % HNMe₂ aqueous solution (4.8 L) were charged to a hydrogenation reactor and the reaction was agitated at 115° C. until completion. IPA (5.3 L) was charged, the temperature was adjusted to 45° C., and solution was aged. The reaction was then transferred to a crystallization reactor and concentrated to 18V. H₂O (2.3 L) was charged and the reaction was concentrated to 18V followed by an additional H₂O (3.5 L) co-distillation to 18V. The reaction content was adjusted to 30° C. and H₂O (3.5 L) was charged slowly. The resulting slurry was aged at 30° C., cooled to 18° C. and aged further. The slurry was filtered, washed with a 1:1 (v/v) IPA/H₂O (4.2 L) solution and dried to afford the desired product in 78% yield. NMR of compound 17 (Formula (II-A-17)) is shown in FIG. 6D. ¹H-NMR (400 MHz, DMSO-d₆) δ ppm 0.76 (t, J=7.28 Hz, 3H), 0.89 (t, J=7.15 Hz, 3H), 1.02-1.14 (m, 1H), 1.20-1.38 (m, 4H), 1.39-1.50 (m, 1H), 1.51-1.65 (m, 1H), 2.01-2.13 (m, 1H), 2.73-2.82 (m, 6H), 3.03 (s, 2H), 3.35 (s, 1H), 3.93 (d, J=7.03 Hz, 1H), 4.86 (d, J=7.03 Hz, 1H), 5.04 (d, J=4.02 Hz, 3H), 6.12 (d, J=2.51 Hz, 1H), 6.46-6.61 (m, 3H), 6.76 (br d, J=7.28 Hz, 1H), 7.08 (t, J=7.78 Hz, 1H), 7.64 (d, J=8.78 Hz, 1H).

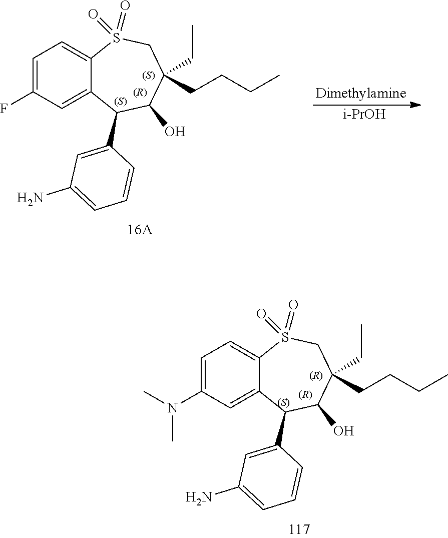

Further diastereomers were synthesized. Dimethylamine (66.7 g, 591.8 mmol, 74.9 mL, 40% purity, 15 eq) was added to a solution of 16A (Formula (II-F-16)) (16.0 g, 39.5 mmol, 1 eq) in i-PrOH (128 mL) at 20° C. The mixture was agitated at 115° C. for 12 hours. LCMS showed that 16A (Formula (II-F-16)) was consumed and the desired product, compound 117 (Formula (II-B-117)), was detected. The mixture was concentrated under reduced pressure to give a residue. The residue was purified by column chromatography (SiO2, Petroleum ether/Ethyl acetate=100/1 to 1/1) and compound 117 (Formula (II-B-117)) (15.0 g, 34.1 mmol, 86.5% yield, 98% purity) was obtained as a yellow solid. NMR of compound 117 (Formula (II-B-1-17)) is as follows: ¹H-NMR (400 MHz, CDCl₃) δ ppm 7.89 (d, J=8.9 Hz, 1H), 7.19 (t, J=7.8 Hz, 1H), 6.97-6.88 (m, 1H), 6.87-6.77 (m, 1H), 6.68-6.61 (m, 1H), 6.51 (dd, J=2.6, 8.9 Hz, 1H), 6.11 (d, J=2.6 Hz, 1H), 5.42 (s, 1H), 4.15 (s, 1H), 3.99-3.39 (m, 2H), 3.18-2.96 (m, 2H), 2.88-2.79 (m, 6H), 2.29 (qd, J=7.3, 14.5 Hz, 1H), 1.80-1.67 (m, 1H), 1.53-1.41 (m, 1H), 1.34-1.24 (m, 5H), 1.20-1.12 (m, 1H), 0.96-0.83 (m, 6H).

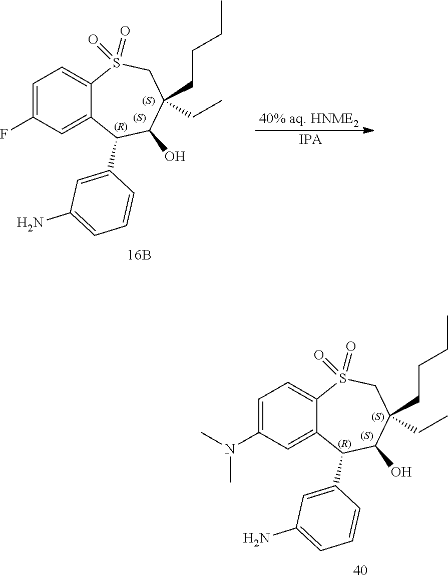

Also compound 40 was synthesized. To a solution of compound 16B (Formula (II-G-16)) (1.20 g, 2.96 mmol, 1.00 eq.) in IPA (12 mL) was added Me₂NH (5.00 g, 44.3 mmol, 5.62 mL, 40.0% purity, 15.0 eq.) at 20° C. The mixture was stirred at 145° C. for 13 hours and a yellow solution was obtained. TLC (Petroleum ether:Ethyl acetate=2:1) indicated compound 16B (Formula (II-G-16)) was fully consumed and LCMS indicated the target compound was obtained. The reaction mixture was concentrated under reduced pressure and the residue was purified by column chromatography (SiO2, Petroleum ether:Ethyl acetate=10:1 to 1:1). Compound 40 (Formula (II-C-40)) (0.49 g, 1.10 mmol, 37.2% yield, 96.9% purity) was obtained as a white solid. NMR of compound 40 (Formula (II-C-40)) is as follows: ¹H-NMR (400 MHz, CDCl₃) δ ppm 7.78 (d, J=8.94 Hz, 1H), 7.12 (t, J=7.75 Hz, 1H), 6.79 (d, J=7.63 Hz, 1H), 6.70 (s, 1H), 6.56 (dd, J=7.87, 1.79 Hz, 1H), 6.44 (dd, J=8.88, 2.56 Hz, 1H), 6.29 (d, J=2.50 Hz, 1H), 4.81 (br d, J=8.94 Hz, 1H), 4.24 (d, J=8.94 Hz, 1H), 3.28-3.37 (m, 1H), 3.13-3.22 (m, 1H), 1.50-1.69 (m, 2H), 1.17-1.25 (m, 6H), 0.79-0.86 (m, 3H), 0.75 (t, J=7.39 Hz, 3H).

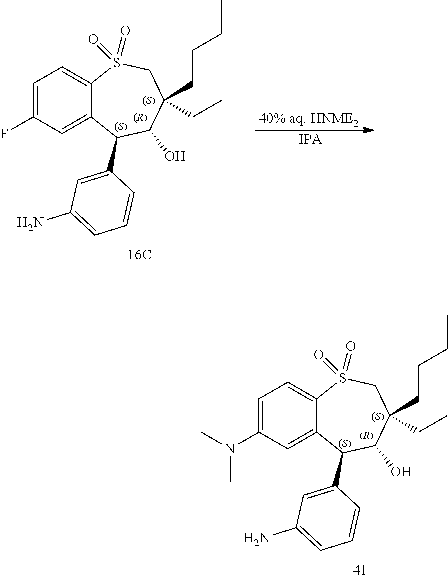

Also compound 41 was synthesized. To a solution of compound 16C (Formula (II-H-16)) (700 mg, 1.73 mmol, 1.00 eq.) in IPA (12 mL) was added Me₂NH (2.92 g, 25.8 mmol, 3.28 mL, 40.0% purity, 15.0 eq.) at 20° C. The mixture was stirred at 145° C. for 13 hours and a yellow solution was obtained. TLC (Petroleum ether:Ethyl acetate=2:1) indicated compound 16C (Formula (II-H-16)) was consumed completely and LCMS indicated compound 41 (Formula (II-D-41)) was obtained. The reaction mixture was concentrated under reduced pressure and the residue was purified by column chromatography (SiO2, Petroleum ether:Ethyl acetate=10:1 to 1:1) to obtain compound 41 (Formula (II-D-41)) (0.23 g, 503.69 μmol, 29.18% yield, 94.3% purity) as a yellow solid. NMR of compound 41 (Formula (II-D-41)) is as follows: ¹H-NMR (400 MHz, CDCl₃) δ ppm 7.78 (d, J=8.94 Hz, 1H), 7.12 (t, J=7.81 Hz, 1H), 6.79 (d, J=7.63 Hz, 1H), 6.71 (s, 1H), 6.56 (dd, J=7.81, 1.97 Hz, 1H), 6.45 (dd, J=8.94, 2.62 Hz, 1H), 6.29 (d, J=2.50 Hz, 1H), 4.78 (br d, J=9.06 Hz, 1H), 4.28 (d, J=8.94 Hz, 1H), 3.28-3.37 (m, 1H), 3.13-3.22 (m, 1H), 2.81 (s, 6H), 1.65 (q, J=7.11 Hz, 2H), 1.22-1.29 (m, 1H), 1.02-1.16 (m, 5H), 0.84 (t, J=7.39 Hz, 3H), 0.75 (t, J=6.97 Hz, 3H).

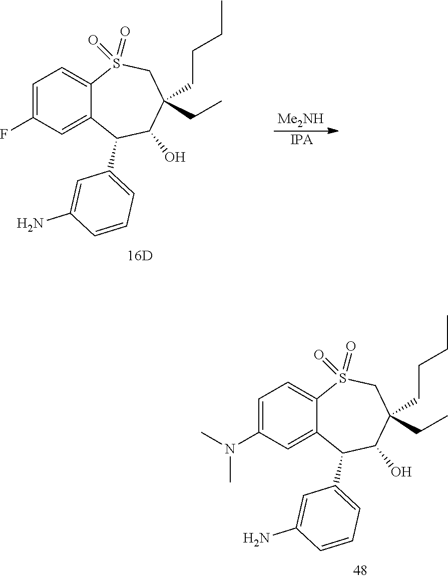 [SMILES: "Me2NH" ; CCCC[C@@]1(CC)CS(=O)(=O)c2ccc(F)cc2[C@@H](c2cccc(N)c2)[C@H]1O ; "IPA" ; CCCC[C@@]1(CC)CS(=O)(=O)c2ccc(N(C)C)cc2[C@@H](c2cccc(N)c2)[C@H]1O]

Further, compound 48 was synthesized. Dimethylamine (8.90 g, 197 mmol, 10 mL) was added to a mixture of compound 16D (Formula (II-I-16)) (70.0 mg, 172 μmol, 1 eq.) and IPA (10 mL) at 20° C. The reaction mixture was agitated at 145° C. for 12 h. TLC indicated compound 16D (Formula (II-I-16)) was fully consumed and the reaction mixture was concentrated under vacuum. The residue was purified by prep-TLC (Petroleum ether:ethyl acetate 1:1). Compound 48 (Formula (II-F-48)) (60 mg, 132 μmol, 76.9% yield, 95.2% purity) was obtained as a brown oil. NMR of compound 48 (Formula (II-F-48)) is as follows: ¹H-NMR (400 MHz, CDCl₃) δ ppm 7.91 (d, J=8.8 Hz, 1H), 7.20 (t, J=7.8 Hz, 1H), 6.94 (br d, J=7.5 Hz, 1H), 6.83 (br s, 1H), 6.67 (br d, J=7.9 Hz, 1H), 6.53 (dd, J=2.4, 8.9 Hz, 1H), 6.11 (d, J=2.3 Hz, 1H), 5.43 (s, 1H), 4.19-4.07 (m, 1H), 3.19-3.10 (m, 1H), 3.07-2.97 (m, 1H), 2.84 (s, 6H), 2.30 (qd, J=7.3, 14.5 Hz, 1H), 1.74 (qd, J=7.4, 14.4 Hz, 1H), 1.56-1.40 (m, 1H), 1.38-1.10 (m, 7H), 0.92 (q, J=7.0 Hz, 6H).

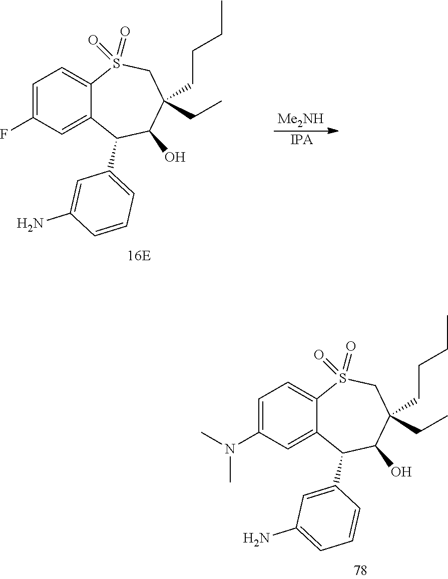 [SMILES: "Me2NH" ; CCCC[C@@]1(CC)CS(=O)(=O)c2ccc(F)cc2[C@@H](c2cccc(N)c2)[C@@H]1O ; "IPA" ; CCCC[C@@]1(CC)CS(=O)(=O)c2ccc(N(C)C)cc2[C@@H](c2cccc(N)c2)[C@@H]1O]

Further, compound 78 was synthesized. Dimethylamine (8.90 g, 197 mmol, 10 mL, 89.9 eq.) was charged to a mixture of compound 16E (Formula (II-J-16)) (0.89 g, 2.19 mmol, 1 eq.) and IPA (10 mL). The reaction was agitated at 145° C. for 12 h and TLC indicated compound 16E (Formula (II-J-16)) was fully consumed. The reaction was concentrated under vacuum and the residue was purified by prep-TLC (Petroleum ether:ethyl acetate 1:1). Compound 78 (Formula (II-H-78)) (180 mg, 381 μmol, 17.3% yield) was obtained as a brown oil. NMR of compound 78 (Formula (II-H-78)) is as follows: ¹H-NMR (400 MHz, CDCl₃) δ ppm 7.86 (d, J=8.9 Hz, 1H), 7.19 (t, J=7.8 Hz, 1H), 6.89-6.82 (m, 1H), 6.79-6.74 (m, 1H), 6.62 (dd, J=2.0, 7.9 Hz, 1H), 6.53 (dd, J=2.6, 8.9 Hz, 1H), 6.38 (d, J=2.4 Hz, 1H), 4.86 (br d, J=8.9 Hz, 1H), 4.36 (br d, J=8.9 Hz, 1H), 3.49 (s, 1H), 3.44-3.22 (m, 2H), 2.89 (s, 6H), 1.74 (q, J=7.0 Hz, 2H), 1.38-1.09 (m, 7H), 0.92 (t, J=7.3 Hz, 3H), 0.83 (br t, J=7.0 Hz, 3H).

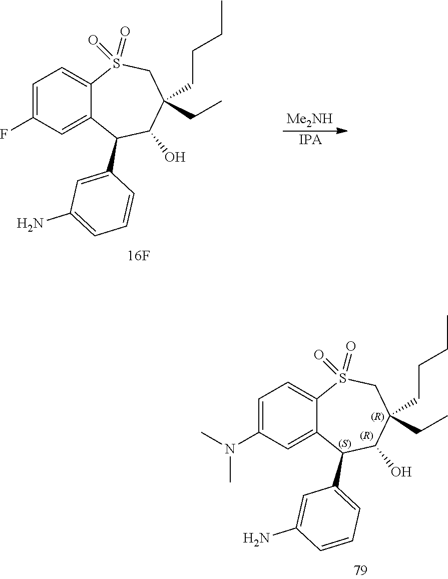

Further, compound 79 was synthesized. Dimethylamine (8.90 g, 197.41 mmol, 10 mL, 123.16 eq.) was charged to a mixture of compound 16F (Formula (II-K-16)) (650 mg, 1.60 mmol, 1 eq.) and IPA (10 mL). The reaction was agitated at 145° C. for 12 h and LCMS indicated compound 16F (Formula (II-K-16)) was fully consumed. The reaction mixture was concentrated under vacuum and the residue was purified by prep-TLC (petroleum ether:ethyl acetate 1:1). Compound 79 (Formula (II-G-79)) (100 mg, 232 μmol, 14.5% yield) was obtained as a brown oil. NMR of compound 79 (Formula (II-G-79)) is as follows: ¹H-NMR (400 MHz, CDCl₃) δ ppm 7.87 (d, J=8.9 Hz, 1H), 7.21 (t, J=7.8 Hz, 1H), 6.93-6.70 (m, 2H), 6.64 (br d, J=7.9 Hz, 1H), 6.53 (dd, J=2.4, 8.9 Hz, 1H), 6.37 (d, J=2.2 Hz, 1H), 4.90 (br d, J=8.9 Hz, 1H), 4.32 (br d, J=8.9 Hz, 1H), 3.46-3.18 (m, 2H), 2.89 (s, 6H), 1.76-1.45 (m, 2H), 1.40-1.17 (m, 6H), 0.93-0.77 (m, 6H).

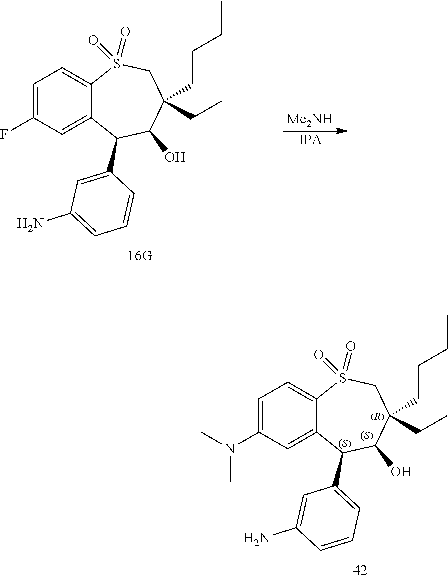

Further, compound 42 was synthesized. Dimethylamine 200 mg, 4.44 mmol, 224 μL, 9.00 eq.) was added to a mixture of compound 16G (Formula (II-L-16)) (200 mg, 493 μmol, 1 eq.) and IPA (10 mL) at 20° C. The reaction was agitated at 145° C. for 12 hours and LCMS indicated compound 16G (Formula (II-L-16)) was fully consumed. The reaction was concentrated under vacuum and the crude product was purified by reverse phase HPLC (column: Phenomenex C18 80*40 mm*3 μm; mobile phase: [water(NH₄HCO₃)-ACN]; B %: 30%-50%, 8 min.). Compound 42 (Formula (II-E-42)) (27.2 mg, 62 μmol, 12.6% yield, 98.4% purity) was obtained as an off-white solid. NMR of compound 42 (Formula (II-E-42)) is as follows: ¹H-NMR (400 MHz, CDCl₃) δ ppm 7.90 (d, J=8.9 Hz, 1H), 7.25-7.15 (m, 1H), 6.94 (br d, J=7.2 Hz, 1H), 6.89-6.76 (m, 1H), 6.66 (br d, J=7.8 Hz, 1H), 6.52 (dd, J=2.4, 8.9 Hz, 1H), 6.10 (d, J=2.2 Hz, 1H), 5.50-5.29 (m, 1H), 4.24-4.08 (m, 1H), 3.77-3.63 (m, 2H), 3.25-2.94 (m, 2H), 2.84 (s, 5H), 2.31-2.13 (m, 1H), 1.66-1.09 (m, 9H), 0.97-0.79 (m, 6H).

Further, similarly to compound 17,

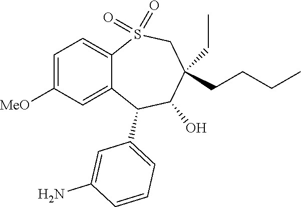 [SMILES: CCCC[C@]1(CC)CS(=O)(=O)c2ccc(OC)cc2[C@@H](c2cccc(N)c2)[C@H]1O]

analog 167 (Formula (II-A-167)) was synthesized.

### Example 13. Synthesis of Compound 65 (Formula (II-A-65)) in Flow Mode

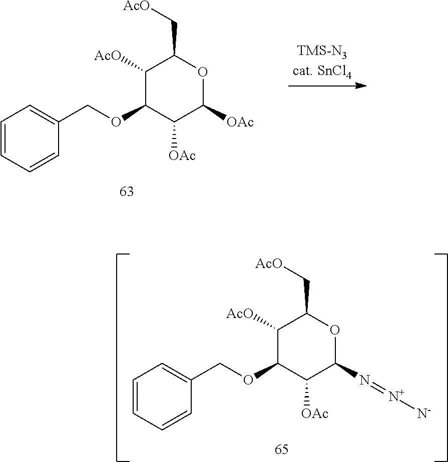

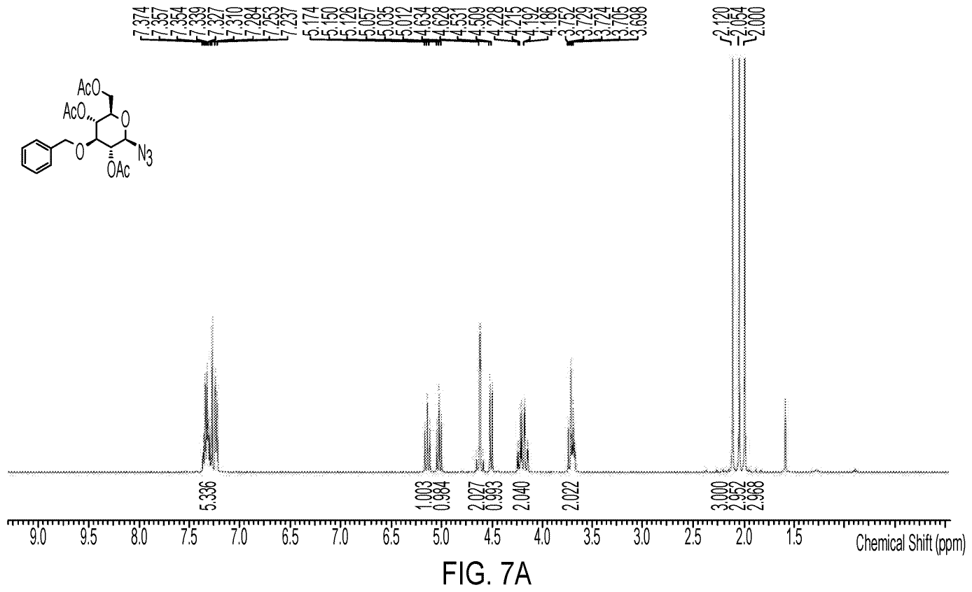

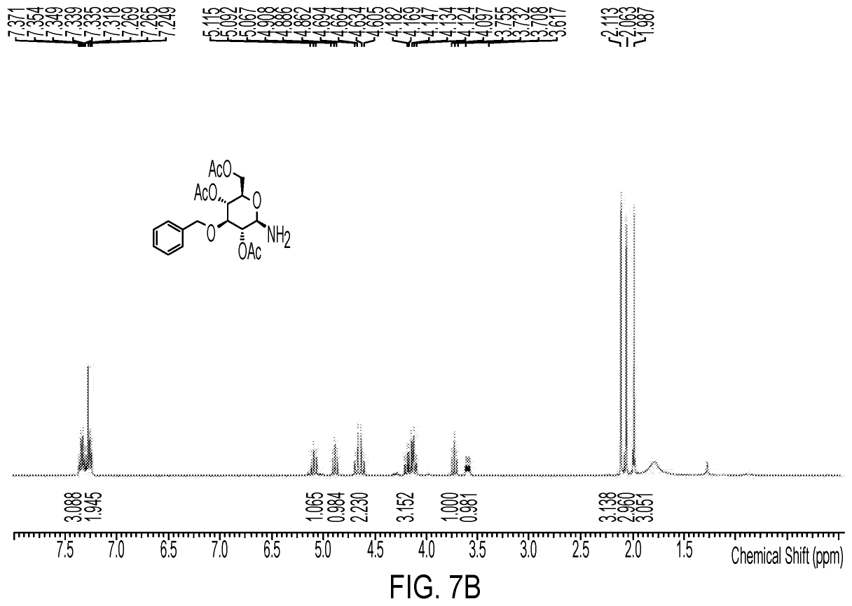

The feed solutions of 63 (Formula (II-A-63)) (43 kg, 98.1 mol, 1.0 equiv.) in toluene (5 V, 215 L, feed 1), SnCl₄ (3.83 kg, 14.7 mol, 0.15 equiv.) in toluene (2.5 V, 107.5 L, feed 2), TMSN₃ (18.1 kg, 156.9 mol, 1.6 equiv.) in toluene (2.5 V, 107.5 L, feed 3) and Rochelle salt (16.6 kg, 58.8 mol. 0.6 eq.) and Na₂CO₃ (20.8 kg, 196.2 mol. 2.0 eq.) in process water (20 V, 860 L, feed 4) were prepared and charged into separate feed tanks. Feed 1 was mixed in a T-piece with feed 2, followed by the addition of feed 3 in a second T-piece, and the mixture was reacted in a 150 m PFA coil (ID: 4 mm) at 20 to 30° C. for 15 min. Then, feed 4 was charged through a third T-piece, and the mixture was quenched and basified in a second 10 m PFA coil (ID: 4 mm) at 20 to 30° C. to avoid the formation of HN₃ in the headspace of FLR1. The outlet stream overflowed into a continuous stir tank reactor (FLR1) for further stirring. The pH of this solution was monitored by in-line pH meter and maintained to be more than 10. To monitor the conversion, a small sample of the reaction stream was taken from the sampling point after FLR1 and the organic solution was analyzed using HPLC, showing 98% conversion to product 65 (Formula (II-A-65)). The organic phase was separated in mixer-settler 1 and washed with process water (5 V) by twice in mixer-settler 2 and mixer-settler 3. The aqueous solutions from 3 stage mixer-settlers were collected in waste drum. The organic phase from mixer-settler 3 was collected in product drum without further workup and used for next step directly. NMR of compound 65 (Formula (II-A-65)) is shown in FIG. 7A. ¹H-NMR (400 MHz, CDCl₃): δ2.00 (s, 3H), δ2.05 (s, 3H), δ2.12 (s, 3H), δ3.70 (m, 2H), δ4.19 (m, 2H), δ4.51 (d, J=8.8 Hz, 1H), δ4.63 (m, 2H), δ5.01 (t, J=9.2 Hz, 1H), δ5.13 (t, J=9.6 Hz, 1H), δ7.24 (m, 5H).

### Example 14. Synthesis of Compound 66 (Formula (II-A-66))

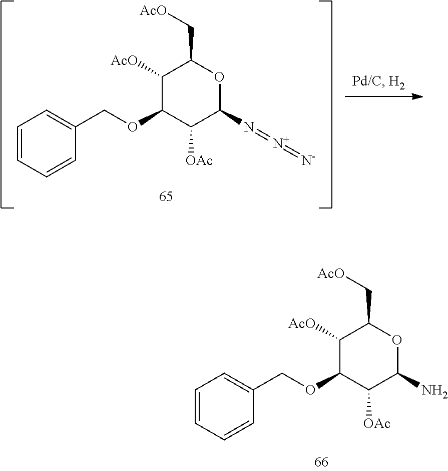

A 65 (Formula (II-A-65)) solution (2.4 mol 65 (Formula (II-A-65))) in toluene, Pd/C (1 wt %), TEA (3.0 mol), and THF (3.0 L) were charged to a hydrogenation reactor. The contents were agitated at 30° C. under 50 psi H₂ pressure until reaction completion. Upon completion, the filtrate was concentrated to 4.5V then IPA (8.0 L) was charged and the temperature adjusted to 45° C. After a cooling ramp to 0° C., the slurry was filtered, washed with IPA (2.0 L) and dried to afford the desired product. NMR of compound 66 (Formula (II-A-66)) is shown in FIG. 7B. ¹H-NMR (400 MHz, CDCl₃) δ1.99 (s, 3H), 2.06 (s, 3H), 2.11 (s, 3H), 3.56-3.64 (m, 1H), 3.73 (t, J=9.41 Hz, 1H), 4.08-4.22 (m, 3H), 4.65 (q, J=11.80 Hz, 2H), 4.89 (t, J=9.16 Hz, 1H), 5.05-5.13 (m, 1H), 5.05-5.13 (m, 1H), 7.23-7.28 (m, 2H), 7.28 (s, 1H), 7.30-7.39 (m, 3H).

Similarly to compound 66 (Formula (II-A-66)), additional diastereomers were synthesized, including:

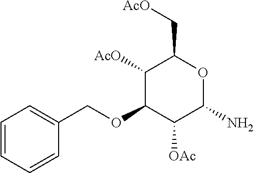 [SMILES: CC(=O)OC[C@H]1O[C@H](N)[C@H](OC(C)=O)[C@@H](OCc2ccccc2)[C@@H]1OC(C)=O]

compound 166 and

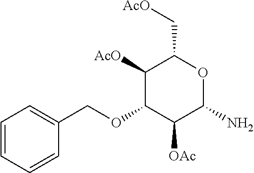 [SMILES: CC(=O)OC[C@@H]1O[C@H](N)[C@@H](OC(C)=O)[C@H](OCc2ccccc2)[C@H]1OC(C)=O]

compound 77.

### Example 15. Synthesis of Compound 67 (Formula (II-67))

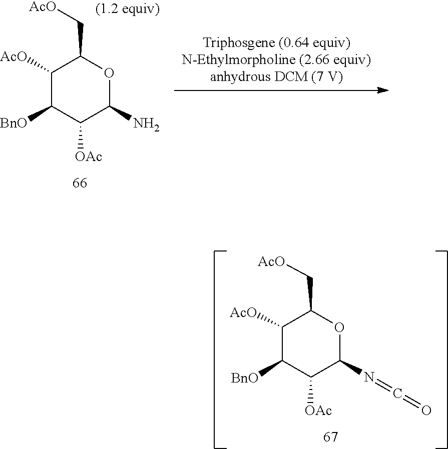

Triphosgene (1.4 mol) was added into an inert reactor 1, followed by DCM (7.0 L), then the solution was stirred at 0° C. for NLT 0.5 h. 66 (Formula (II-A-66)) (2.8 mol) was added into an inert reactor 2, followed by DCM (7.0 L) and n-ethylmorpholine (6.2 mol). The 66 (Formula (II-A-66)) solution was transferred to reactor 1 over 2 h while maintaining reactor content at 0° C. Following the transfer, the reaction mixture was stirred at 0° C. for 1 h, warmed to 20° C. and agitated for another 1 h. Upon completion of the reaction, the solution was concentrated at NMT 25° C. to remove residual phosgene. The product, 67 (Formula (II-67)), was stored as a solution in DCM for use in the subsequent step.

### Example 16. Synthesis of Compound 20 (Formula (II-A-20))

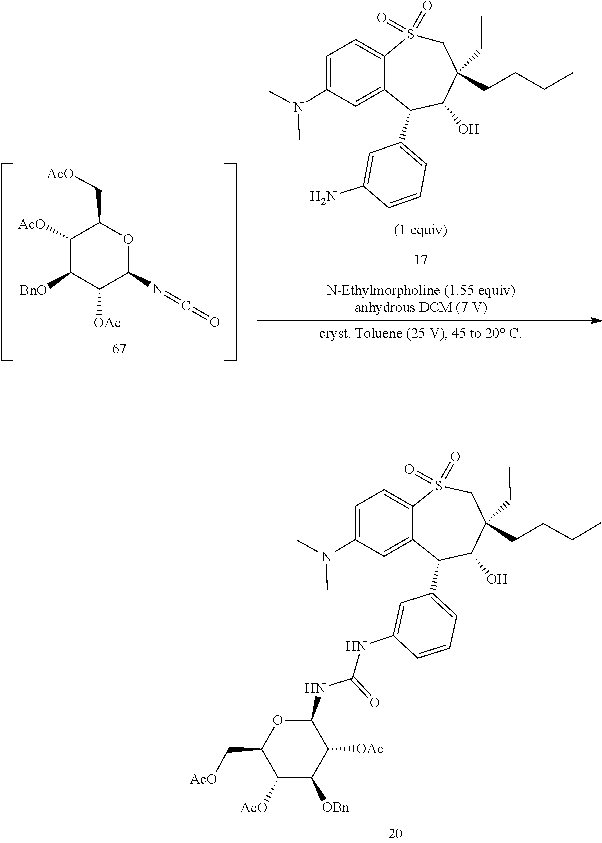

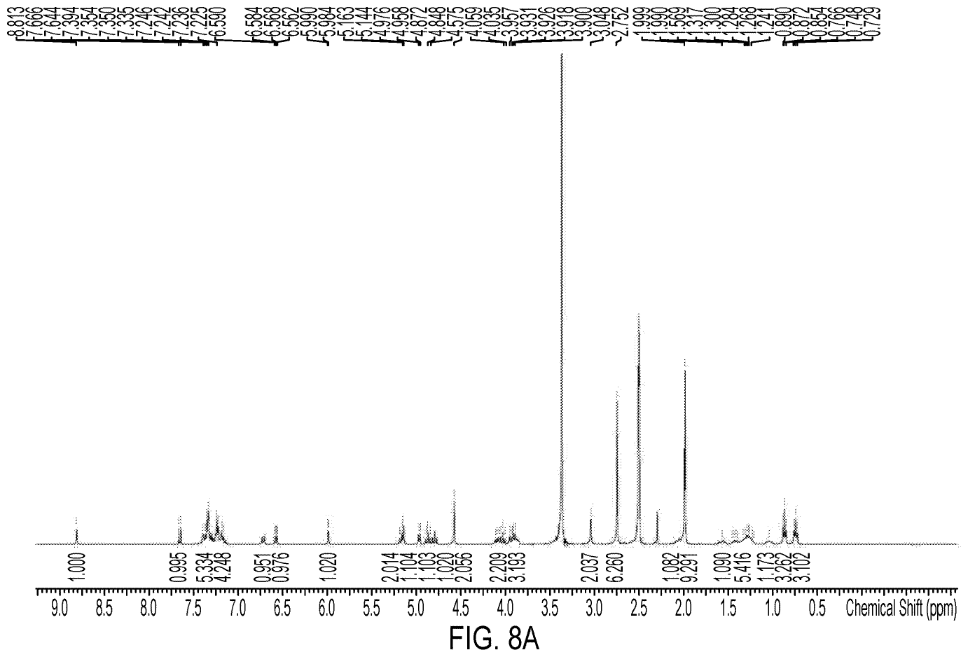

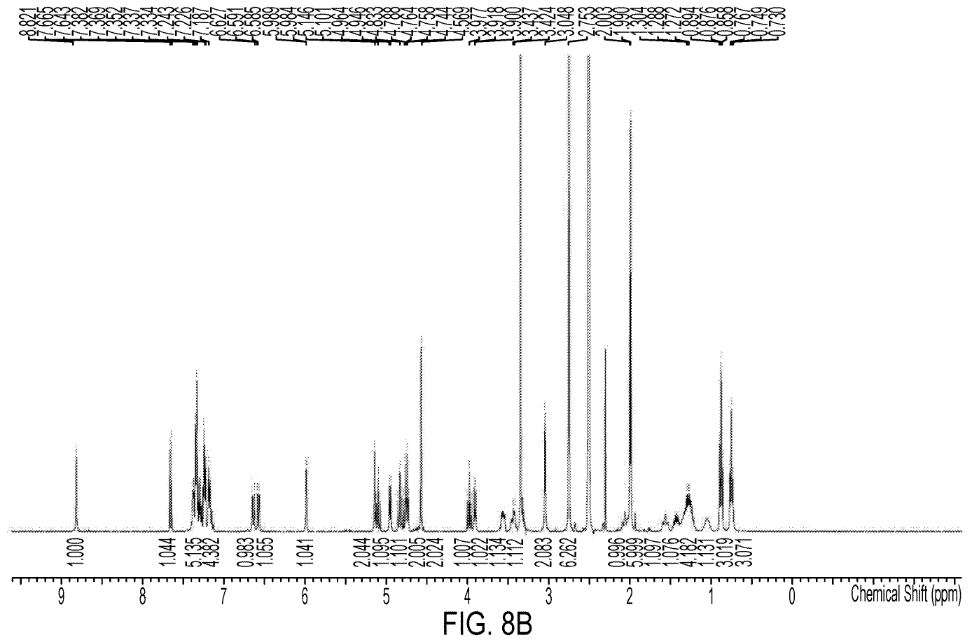

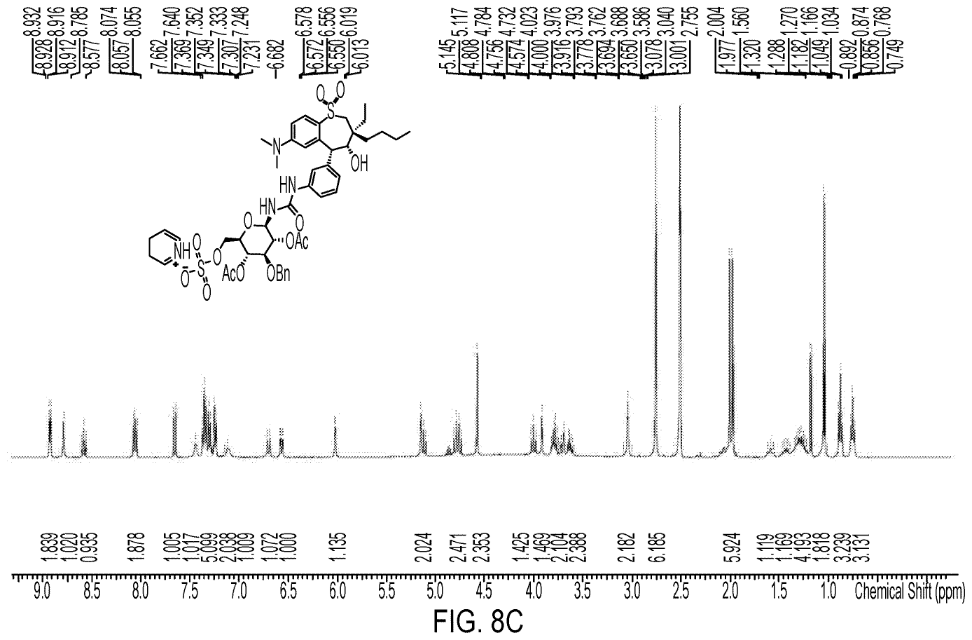

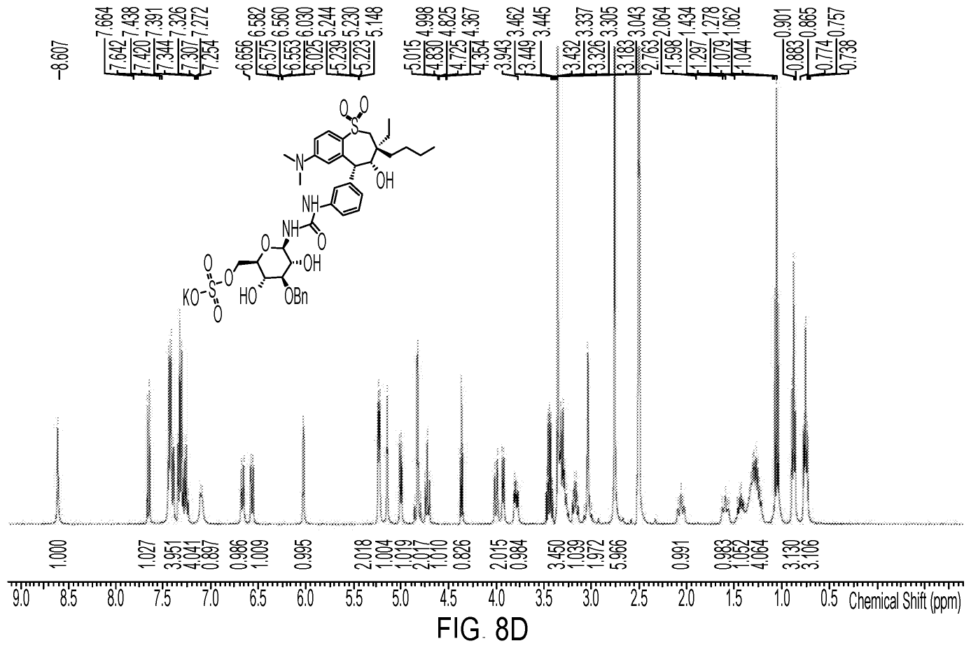

17 (Formula (II-A-17)) (2.3 mol), DCM (7.0 L), and N-ethylmorpholine (3.7 mol) were added to reactor 2 and agitated at 20° C. The 17 (Formula (II-A-17)) solution was transferred over 3 h at 20° C. to the 67 (Formula (II-67)) solution from the previous step and agitated until the reaction was complete. The reaction mixture was then washed with an aqueous NaHCO₃ solution (5% wt/wt), the layers were separated, and the organic layer was washed twice with purified water. The resulting DCM solution was concentrated to 5V while maintaining an internal temperature at NMT 25° C. 10 L toluene was charged slowly resulting in precipitation of solid. The mixture was concentrated to 10V while maintaining internal temperature at NMT 35° C. followed by an additional 5 L toluene co-distillation. After concentrating the mixture back to 10V, an additional 10 L toluene was charged and the reaction content was adjusted to 45° C. and agitated for 3 h. The contents were then cooled to 20° C. over 4 h and aged. An additional 5 L toluene was charged and the mixture was aged and filtered. The resulting product was washed with toluene and dried at under vacuum. The yield over 2 steps (compound 66 (Formula (II-A-66)) to compound 67 (Formula (II-67)) and compound 67 (Formula (II-67)) to compound 20 (Formula (II-A-20))) was 90%. NMR of compound 20 (Formula (II-A-20)) is shown in FIG. 8A. ¹H-NMR (400 MHz, DMSO-d₆) ¹H NMR (400 MHz, DMSO-d₆) δ ppm 0.75 (br t, J=7.38 Hz, 3H), 0.84-0.95 (m, 3H), 1.05 (br s, 1H), 1.21-1.48 (m, 5H), 1.57 (br s, 1H), 1.96-2.04 (m, 8H), 2.03-2.04 (m, 1H), 2.05-2.12 (m, 1H), 2.75 (s, 6H), 3.05 (s, 2H), 3.85-3.99 (m, 3H), 4.01-4.15 (m, 2H), 4.58 (s, 2H), 4.76-4.83 (m, 1H), 4.87 (t, J=9.69 Hz, 1H), 4.97 (d, J=7.00 Hz, 1H), 5.10-5.24 (m, 2H), 5.99 (d, J=2.25 Hz, 1H), 6.58 (dd, J=8.94, 2.44 Hz, 1H), 6.68-6.78 (m, 1H), 7.13-7.25 (m, 4H), 7.31-7.42 (m, 5H), 7.66 (d, J=8.76 Hz, 1H), 8.81 (s, 1H).

Similarly to compound 20 (Formula (II-A-20)), additional diastereomers were synthesized, including:

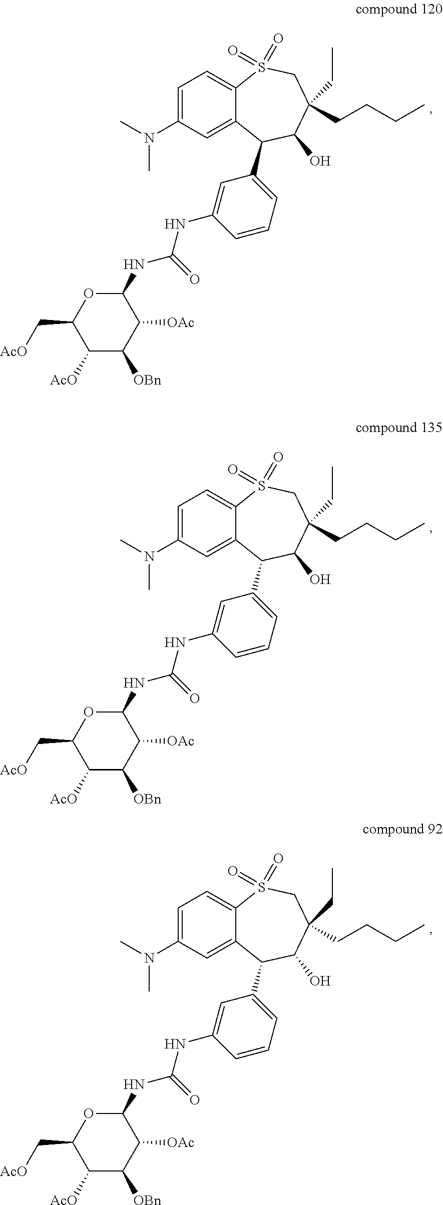 [SMILES: CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@@H]1O ; CCCC[C@@]1(CC)CS(=O)(=O)c2ccc(N(C)C)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

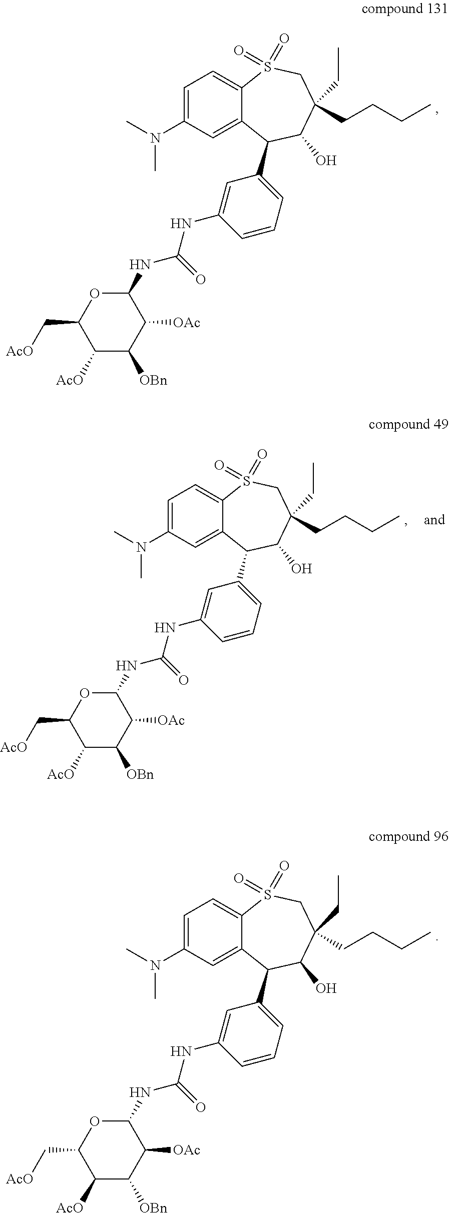 [SMILES: CCCC[C@]1(CC)CS(=O)(=O)c2ccc(N(C)C)cc2[C@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](COC(C)=O)[C@@H](OC(C)=O)[C@H](OCc4ccccc4)[C@H]3OC(C)=O)c2)[C@H]1O ; CCCC[C@@]1(CC)CS(=O)(=O)c2ccc(N(C)C)cc2[C@H](c2cccc(NC(=O)N[C@H]3O[C@@H](COC(C)=O)[C@H](OC(C)=O)[C@@H](OCc4ccccc4)[C@@H]3OC(C)=O)c2)[C@@H]1O]

### Further,

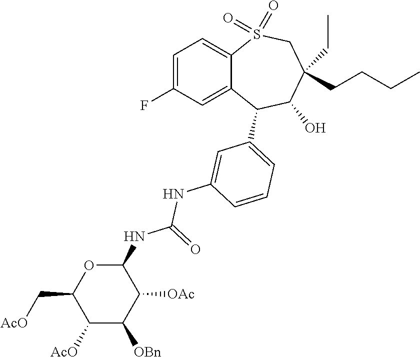 [SMILES: CCCC[C@]1(CC)CS(=O)(=O)c2ccc(F)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

analog 51 was synthesized using compound 16 (Formula (II-E-16)) in place of compound 17 (Formula (II-A-17)) and

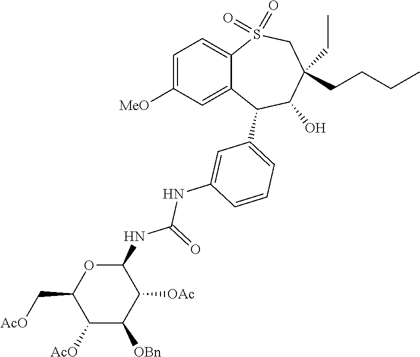 [SMILES: CCCC[C@]1(CC)CS(=O)(=O)c2ccc(OC)cc2[C@@H](c2cccc(NC(=O)N[C@@H]3O[C@H](COC(C)=O)[C@@H](OC(C)=O)[C@H](OCc4ccccc4)[C@H]3OC(C)=O)c2)[C@H]1O]

analog 176 was synthesized using compound 167 (Formula (II-A-167)) in place of compound 17 (Formula (II-A-17)).

### Example 17. Synthesis of Compound 25 (Formula (II-A-25))

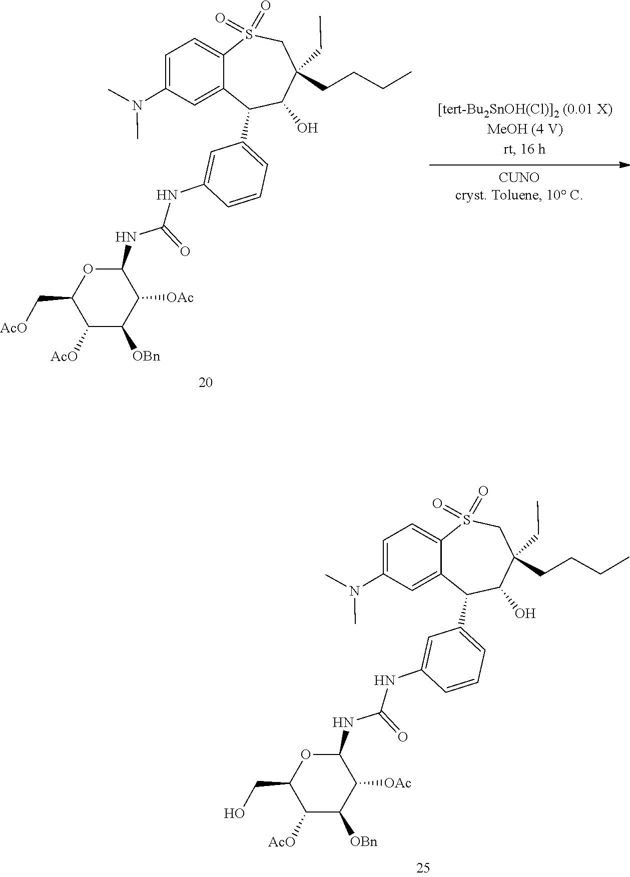

To an inert reactor, 20 (Formula (II-A-20)) (1.2 mol) was combined with MeOH (4.0 L) and [tBu₂SnOH(Cl)]₂ (0.00002 mol). The mixture was agitated at 20° C. followed by cooling to 10° C. and an IPC sample was taken. Upon completion of the reaction, AcOH (0.00033 mol) and MeOH (4.0 L) were charged, and the mixture was agitated at 20° C. The reaction content was recirculated through a CUNO filter for 3 h and the resulting solution was concentrated under vacuum to 4V while maintaining the internal temperature at NMT 35° C. The contents were co-distilled with toluene to reduce the residual MeOH content. Toluene (4.0 L) was charged and the contents were adjusted to 10° C. over 2 h and aged. The resulting slurry was filtered and washed with toluene then dried under vacuum to afford the desired compound. The reaction yield was 90%. Experimentation with various reaction solvents revealed that the use of MeOH only yielded the greatest conversion and purity of the product compared to THF/MeOH, toluene/MeOH, and IPA/THF. Further experimentation revealed that quenching the reaction mixture with acetic acid and filtering the reaction mixture through CUNO reduced over reaction during workup and removed particles from the solution before crystallization. NMR of compound 25 (Formula (II-A-25)) is shown in FIG. 8B. ¹H-NMR (400 MHz, DMSO-d₆) δ ppm 0.75 (t, J=7.40 Hz, 3H), 0.88 (t, J=7.15 Hz, 3H), 0.99-1.13 (m, 1H), 1.20-1.37 (m, 4H), 1.39-1.49 (m, 1H), 1.51-1.65 (m, 1H), 2.00 (d, J=5.27 Hz, 6H), 2.03-2.12 (m, 1H), 2.75 (s, 6H), 3.05 (s, 2H), 3.43 (br d, J=5.27 Hz, 1H), 3.52-3.63 (m, 1H), 3.91 (d, J=7.28 Hz, 1H), 3.98 (t, J=9.41 Hz, 1H), 4.57 (s, 2H), 4.71-4.79 (m, 2H), 4.83 (t, J=9.66 Hz, 1H), 4.96 (d, J=7.03 Hz, 1H), 5.07-5.19 (m, 2H), 5.99 (d, J=2.26 Hz, 1H), 6.58 (dd, J=9.03, 2.51 Hz, 1H), 6.64 (d, J=10.04 Hz, 1H), 7.13-7.25 (m, 4H), 7.32-7.42 (m, 5H), 7.65 (d, J=8.78 Hz, 1H), 8.82 (s, 1H).

Similarly to compound 25 (Formula (II-A-25)), additional diastereomers were synthesized, including:

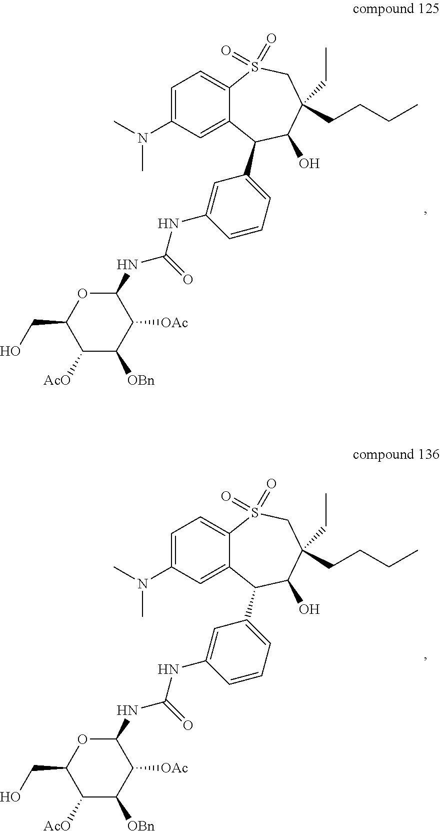 [SMILES: CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@@H]1O ; CCCC[C@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@@H]1O]

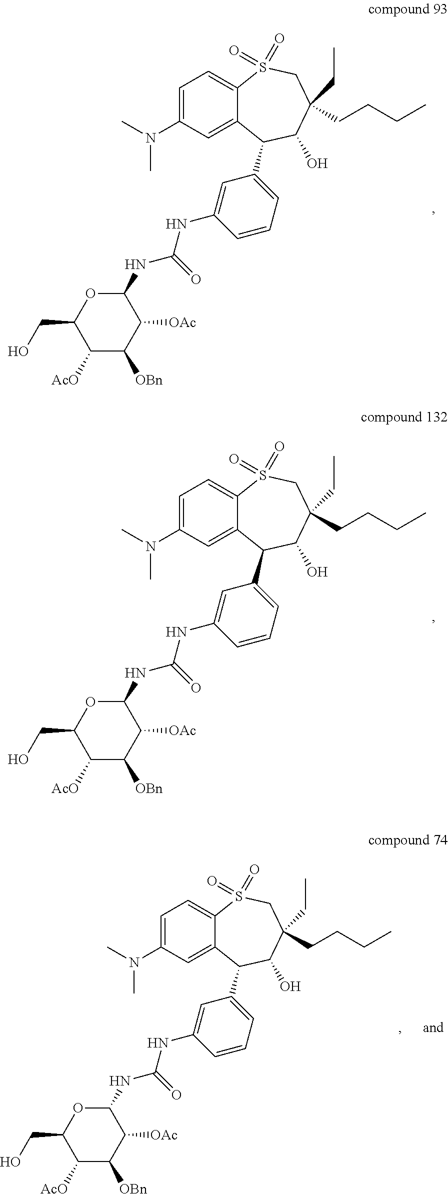 [SMILES: CCCC[C@@]1(CC)CS(=O)(=O)c2ccc(N(C)C)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@H](c2cccc(NC(=O)N[C@@H]3O[C@H](CO)[C@@H](OC(C)=O)[C@H](OCc4ccccc4)[C@H]3OC(C)=O)c2)[C@H]1O ; CCCC[C@]1(CC)CS(=O)(=O)c2ccc(N(C)C)cc2[C@@H](c2cccc(NC(=O)N[C@H]3O[C@H](CO)[C@@H](OC(C)=O)[C@H](OCc4ccccc4)[C@H]3OC(C)=O)c2)[C@H]1O]

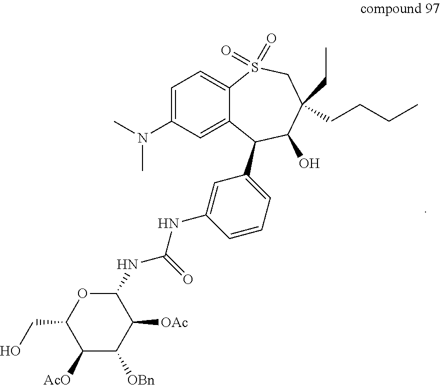 [SMILES: CCCC[C@@]1(CC)CS(=O)(=O)c2ccc(N(C)C)cc2[C@H](c2cccc(NC(=O)N[C@H]3O[C@@H](CO)[C@H](OC(C)=O)[C@@H](OCc4ccccc4)[C@@H]3OC(C)=O)c2)[C@@H]1O]

### Further,

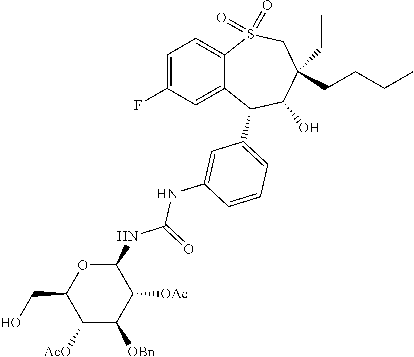 [SMILES: CCCC[C@]1(CC)CS(=O)(=O)c2ccc(F)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

analog 53 and

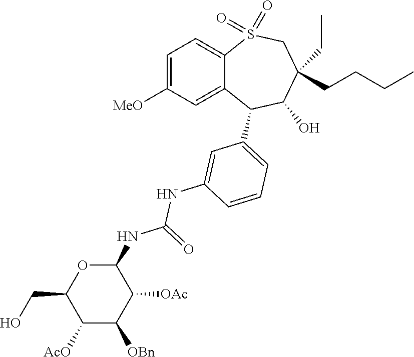 [SMILES: CCCC[C@]1(CC)CS(=O)(=O)c2ccc(OC)cc2[C@@H](c2cccc(NC(=O)N[C@@H]3O[C@H](CO)[C@@H](OC(C)=O)[C@H](OCc4ccccc4)[C@H]3OC(C)=O)c2)[C@H]1O]

analog 177 were synthesized.

### Example 18. Synthesis of Compound 26 (Formula (II-A-26))

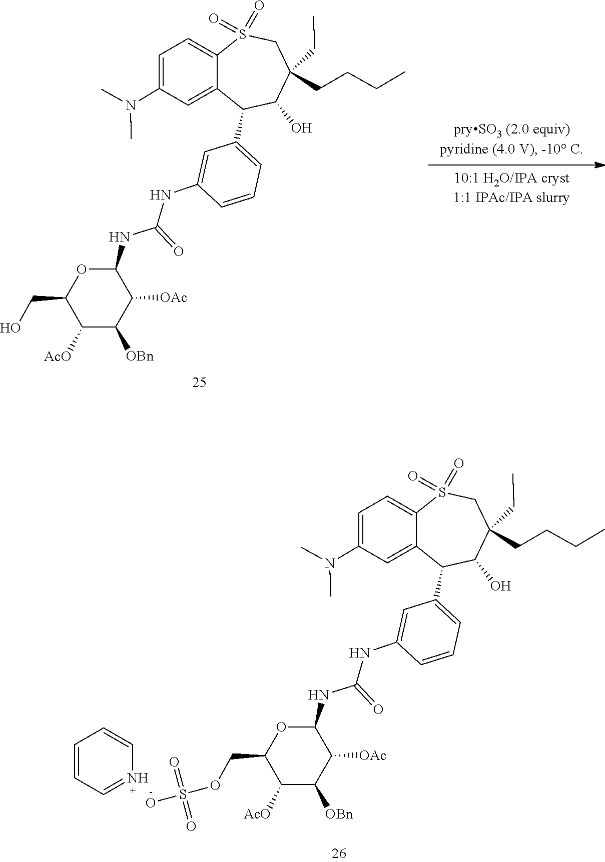

To an inert reactor 25 (Formula (II-A-25)) (1.2 mol) was dissolved in pyridine (4.0 L). The reaction temperature was adjusted to −10° C. and pyridine sulfur trioxide (2.5 mol) was added in one portion. The reaction mixture was agitated at −10° C. until reaction completion. Upon completion, the reaction mixture was quenched with water (1.0 L) over 3 h then agitated at −10° C. for 3 h. The temperature was adjusted to 0° C. and a 10:1 (v/v) H₂O/IPA (16.2 L) solution was charged over 4 h. The slurry was filtered and washed twice with 1:1 (v/v) H₂O/IPA (3.0 L). The wet cake was transferred back to the reactor and IPA (4.7 L) and IPAc (5.3 L) were charged. The slurry was agitated at 20° C. then filtered. The cake was washed twice with 1:1 (v/v) IPA/IPAc (2.5 L) and dried under vacuum to obtain the desired 26 (Formula (II-A-26)) product. Experimentation revealed that only two equivalents of the sulfur trioxide-pyridine complex were necessary for proper conversion to product vs. the use of 2.5 equivalents of the sulfur trioxide-pyridine complex. Further experimentation revealed that a lower reaction temperature of −10° C. minimized impurity formation vs. a reaction temperature of 0° C., a lower reaction quenching temperature of −10° C. minimized impurity formation vs. a reaction quenching temperature of 0° C., and a lower crystallization temperature of 0° C. minimized impurity formation vs. a crystallization temperature of 60° C. It was further discovered that creating a slurry of the wet cake product after recrystallization in isopropanol and isopropyl acetate improved product purity, improved solid properties, and reduced residual solvent levels. The yield of this reaction was 90%. NMR of compound 26 (Formula (II-A-26)) is shown in FIG. 8C. ¹H-NMR (400 MHz, DMSO-d₆) δ ppm 0.75 (br t, J=7.32 Hz, 3H), 0.82-0.92 (m, 3H), 1.04 (d, J=6.13 Hz, 5H), 1.17 (d, J=6.25 Hz, 2H), 1.21-1.38 (m, 4H), 1.43 (dt, J=14.32, 7.35 Hz, 1H), 1.51-1.65 (m, 1H), 1.99 (d, J=10.76 Hz, 6H), 2.75 (s, 6H), 2.98-3.09 (m, 2H), 3.57-3.74 (m, 1H), 3.60-3.67 (m, 1H), 3.60-3.66 (m, 1H), 3.60-3.66 (m, 1H), 3.60-3.66 (m, 1H), 3.60-3.66 (m, 1H), 3.67-3.73 (m, 1H), 3.68-3.73 (m, 1H), 3.68-3.73 (m, 1H), 3.68-3.73 (m, 1H), 3.74-3.85 (m, 1H), 3.76-3.84 (m, 1H), 3.92 (s, 1H), 4.00 (t, J=9.38 Hz, 1H), 3.97-4.03 (m, 1H), 4.54-4.60 (m, 1H), 4.57 (s, 1H), 4.72-4.83 (m, 1H), 4.72-4.82 (m, 1H), 5.06-5.21 (m, 1H), 5.07-5.19 (m, 1H), 6.02 (d, J=2.38 Hz, 1H), 6.56 (dd, J=8.94, 2.44 Hz, 1H), 6.69 (br d, J=10.01 Hz, 1H), 7.04-7.15 (m, 1H), 7.21-7.27 (m, 2H), 7.28-7.40 (m, 5H), 7.44 (br s, 1H), 7.65 (d, J=8.88 Hz, 1H), 8.06 (dd, J=7.75, 6.63 Hz, 2H), 8.58 (tt, J=7.83, 1.55 Hz, 1H), 8.79 (s, 1H), 8.92 (dd, J=6.50, 1.38 Hz, 2H).

Similarly to compound 26 (Formula (II-A-26)), additional diastereomers were synthesized, including:

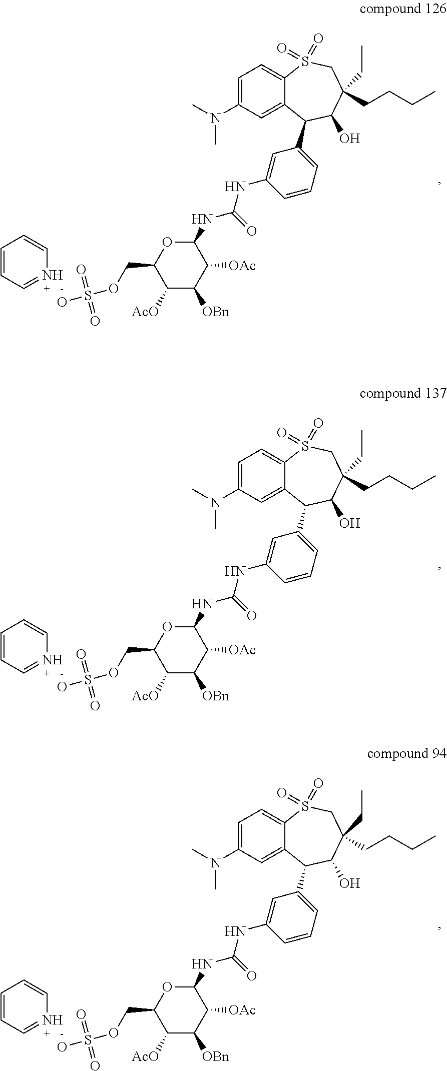

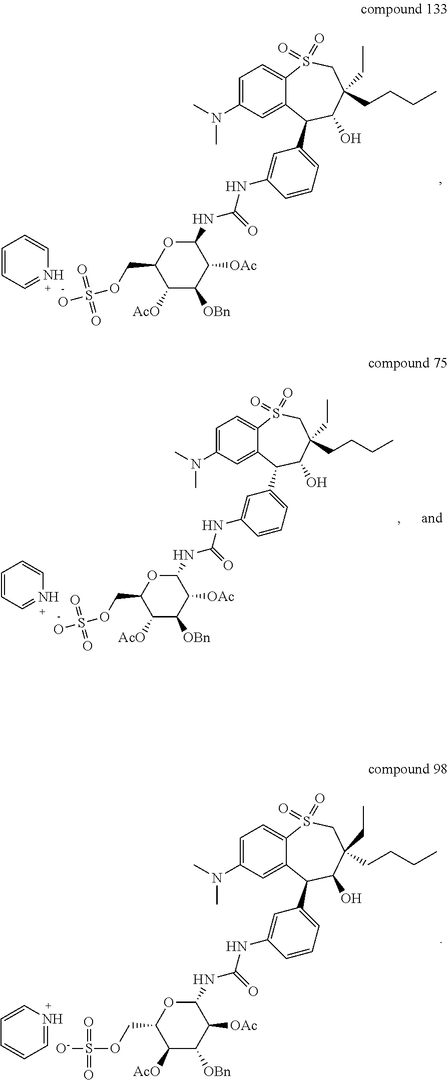

### Further,

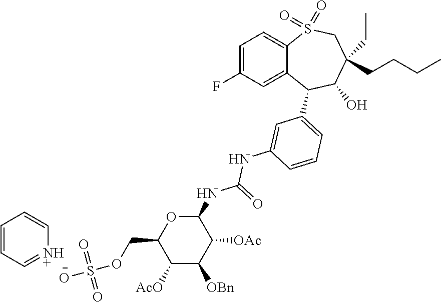

analog 174 and

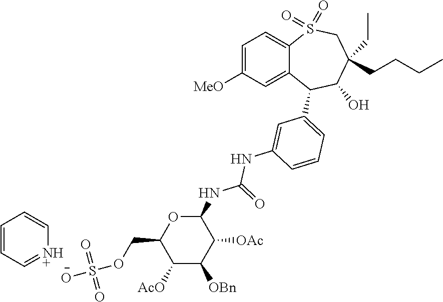

analog 178 were synthesized.

### Example 19. Synthesis of Compound 27 (Formula (II-A-27))

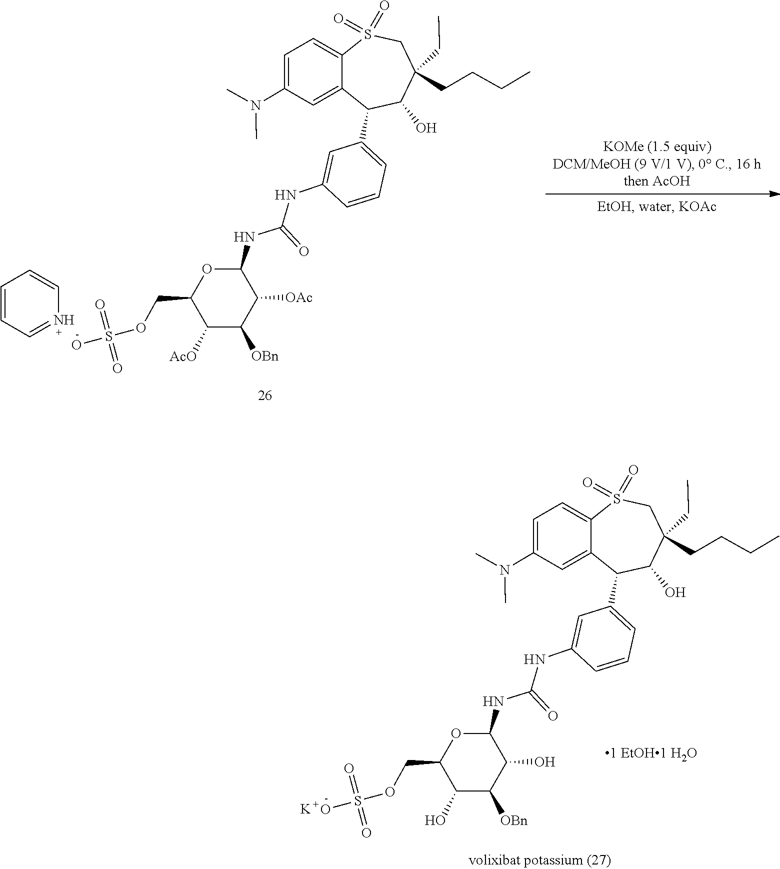

To an inert reactor 26 (Formula (II-A-26)) (1.0 mol) was dissolved in DCM (9.9 L) and MeOH (0.8 L) at 20° C. The resulting solution was cooled to 0° C. and KOMe in MeOH (0.4 L) was charged over 4 h while maintaining the internal temperature at NMT 0° C. The reaction was agitated at 0° C. and upon completion of the reaction, AcOH was charged to adjust the pH to 4-7. The mixture was warmed to 20° C. and MeOH (10 L) was charged followed by concentration to 10V by vacuum distillation. The resulting solution was polish filtered to the crystallization reactor and rinsed forward with MeOH and EtOH. A KOAc/H₂O/EtOH solution was charged, and the reaction mixture was concentrated to 12V. The KOAc/H₂O/EtOH co-distillations were repeated six times followed by six H₂O/EtOH co-distillations to 12V. The reaction contents were adjusted to 55° C. and 27 (Formula (II-A-27)) seed (0.005×) was charged. Six EtOH co-distillations to 12V were performed to reduce the KF (Karl Fischer) to <1.7%. The reaction was then ramp cooled from 55° C. to 5° C., aged and filtered. The wet cake was washed twice with 1 wt % H₂O/EtOH (1.8 L) and dried under vacuum followed by humidified drying to remove residual solvents and convert the product to the desired form. The yield of this reaction was 90%. Experimentation with various reaction solvents revealed that the use of DCM/MeOH yielded the greatest conversion and purity of the product compared to MeOH alone, ACN, THF, 2-MeTHF, DCM alone, ACN/MeOH, and THF/MeOH. Further experimentation revealed that a lower reaction temperature of about 0-10° C. minimized the formation of impurities vs. a reaction temperature of about 20° C. This synthesis fulfills GMP standards and has been performed on batch sizes ranging from 15 kg to 35 kg. NMR of compound 27 (Formula (II-A-27)) is shown in FIG. 8D. 1H NMR (400 MHz, DMSO-d₆) δ ppm 0.76 (br t, J=7.28 Hz, 3H), 0.88 (t, J=7.15 Hz, 3H), 0.96-1.13 (m, 1H), 1.06 (t, J=7.03 Hz, 3H), 1.19-1.39 (m, 4H), 1.43 (dt, J=14.24, 7.31 Hz, 1H), 1.41-1.42 (m, 1H), 1.54-1.67 (m, 1H), 2.01-2.15 (m, 1H), 2.76 (s, 6H), 2.99-3.09 (m, 2H), 3.12-3.21 (m, 1H), 3.24-3.35 (m, 3H), 3.40-3.51 (m, 1H), 3.45 (qd, J=6.94, 5.02 Hz, 1H), 3.80 (dd, J=11.04, 5.52 Hz, 1H), 3.87-4.06 (m, 2H), 4.37 (t, J=5.02 Hz, 1H), 4.72 (t, J=9.16 Hz, 1H), 4.78-4.90 (m, 2H), 5.01 (d, J=7.03 Hz, 1H), 5.15 (s, 1H), 5.23 (dd, J=5.90, 2.38 Hz, 2H), 6.03 (d, J=2.26 Hz, 1H), 6.57 (dd, J=8.78, 2.51 Hz, 1H), 6.67 (br d, J=9.29 Hz, 1H), 7.10 (br d, J=6.02 Hz, 1H), 7.22-7.36 (m, 4H), 7.37-7.49 (m, 4H), 7.65 (d, J=8.78 Hz, 1H), 8.61 (s, 1H).

Similarly to compound 27 (Formula (II-A-27)), additional diastereomers were synthesized, including:

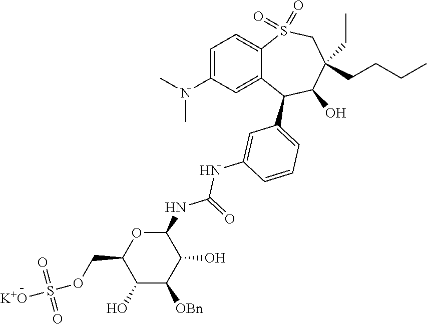

compound 123 (Formula (II-A-123)),

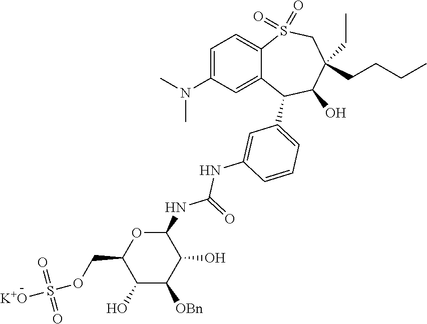

compound 138 (Formula (II-C-138)),

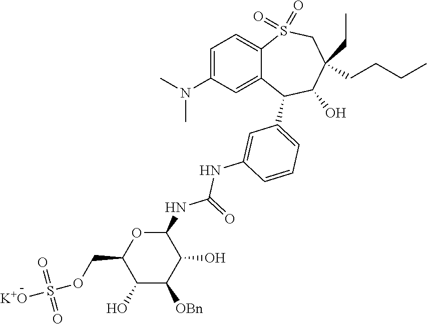

compound 95 (Formula (II-D-95)),

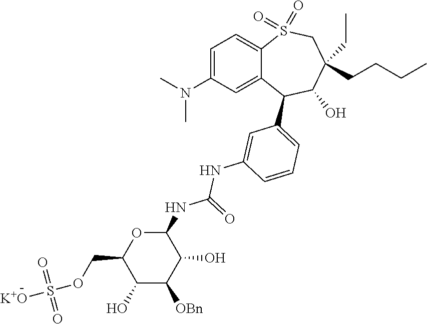

compound 134 (Formula (II-F-134)),

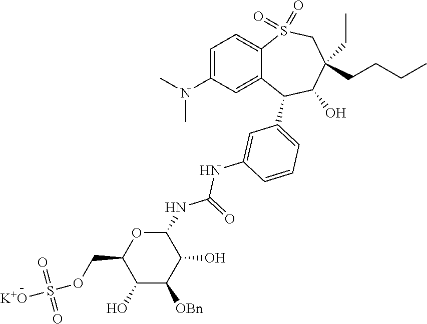

compound 76 (Formula (II-G-76)), and

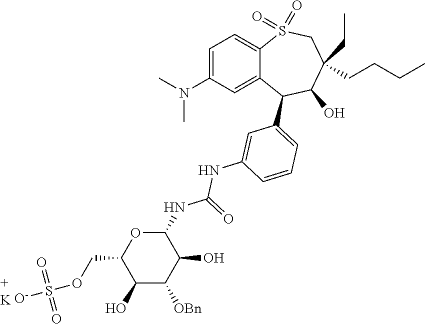 [SMILES: [K+] ; CCCC[C@@]1(CC)CS(=O)(=O)c2ccc(N(C)C)cc2[C@H](c2cccc(NC(=O)N[C@H]3O[C@@H](COS(=O)(=O)[O-])[C@H](O)[C@@H](OCc4ccccc4)[C@@H]3O)c2)[C@@H]1O]

compound 99 (Formula (II-H-99)). Further,

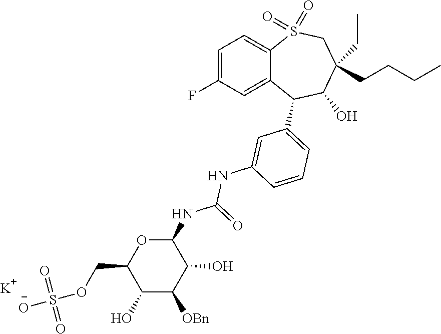 [SMILES: [K+] ; CCCC[C@]1(CC)CS(=O)(=O)c2ccc(F)cc2[C@@H](c2cccc(NC(=O)N[C@@H]3O[C@H](COS(=O)(=O)[O-])[C@@H](O)[C@H](OCc4ccccc4)[C@H]3O)c2)[C@H]1O]

analog 175 (Formula (II-B-175)) and

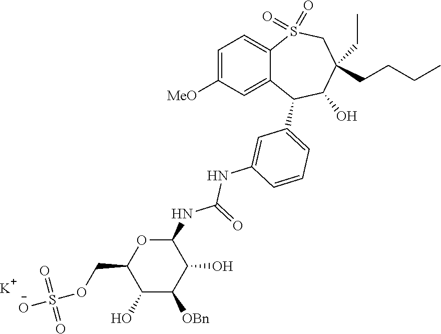 [SMILES: [K+] ; CCCC[C@]1(CC)CS(=O)(=O)c2ccc(OC)cc2[C@@H](c2cccc(NC(=O)N[C@@H]3O[C@H](COS(=O)(=O)[O-])[C@@H](O)[C@H](OCc4ccccc4)[C@H]3O)c2)[C@H]1O]

analog 179 (Formula (II-E-179)) were synthesized.

### Example 20. Solid State Polymorph Study of Volixibat Potassium

This polymorph study identified polymorphs and pseudo-polymorphs of the volixibat potassium salt (C₃₈H₅₀KN₃O₁₂S₂, molecular weight=844.04) in ethanol/water solvent mixtures, studied interconversion relationship among different polymorphs, confirmed suitable storage and manufacture condition of Form I, and provided guidance on how to avoid certain crystalline forms.

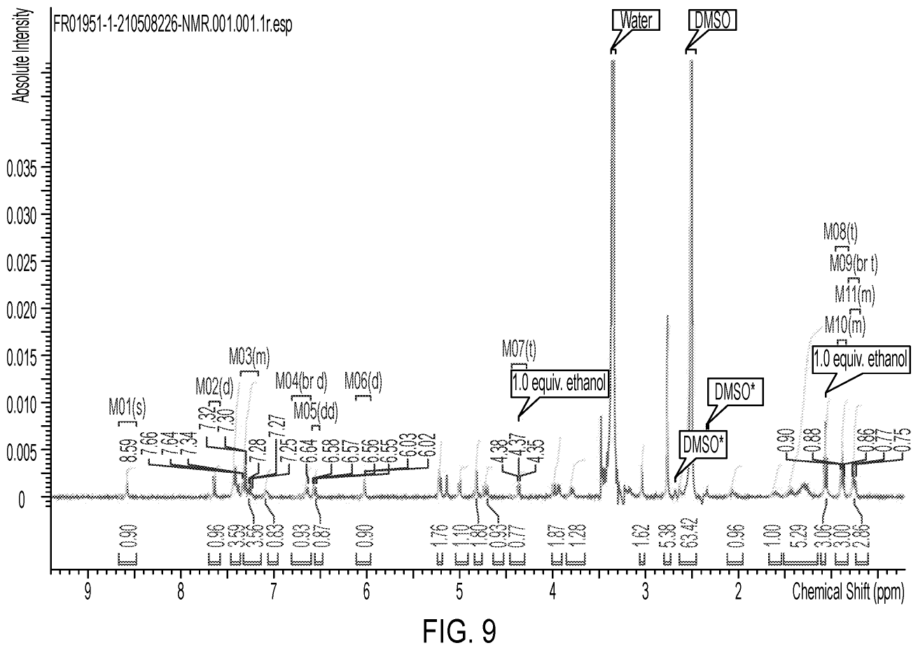

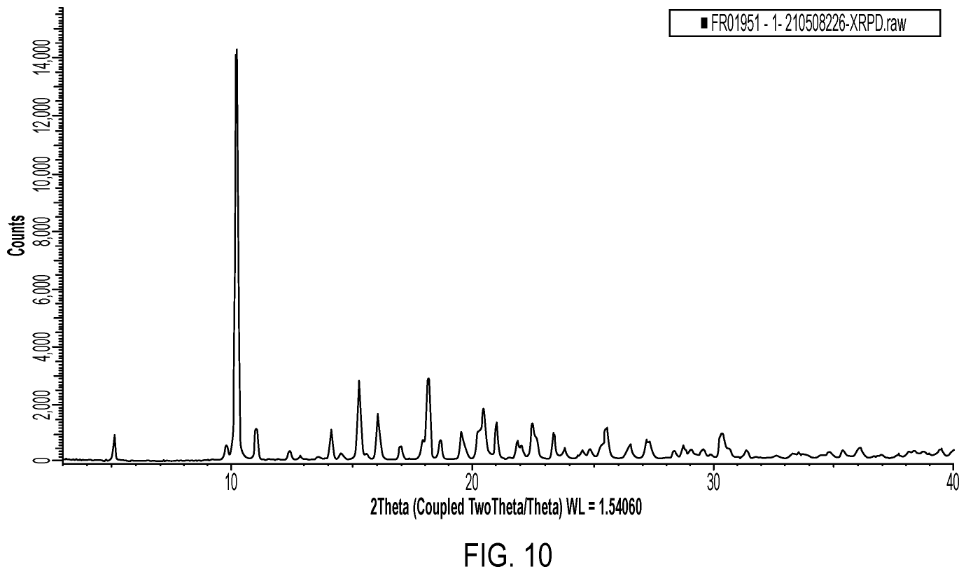

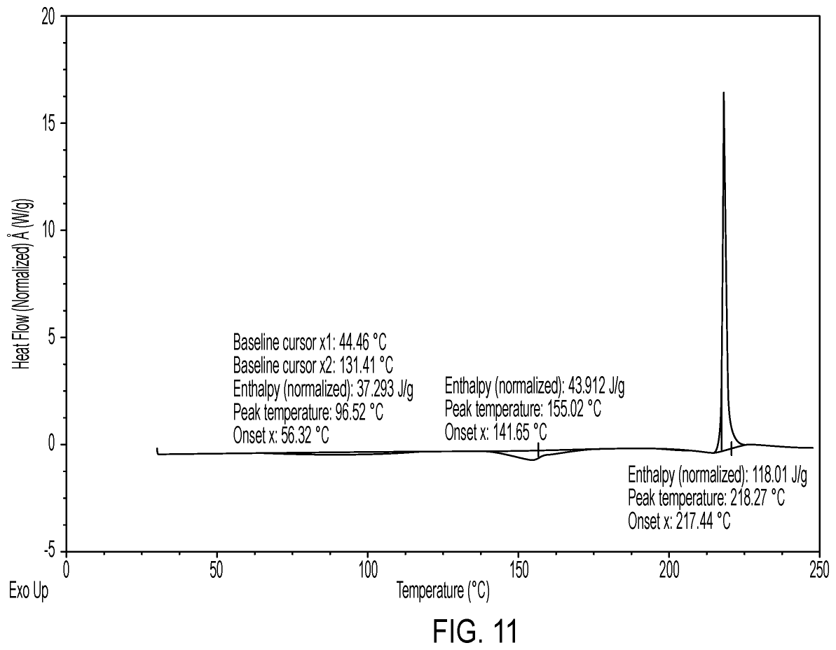

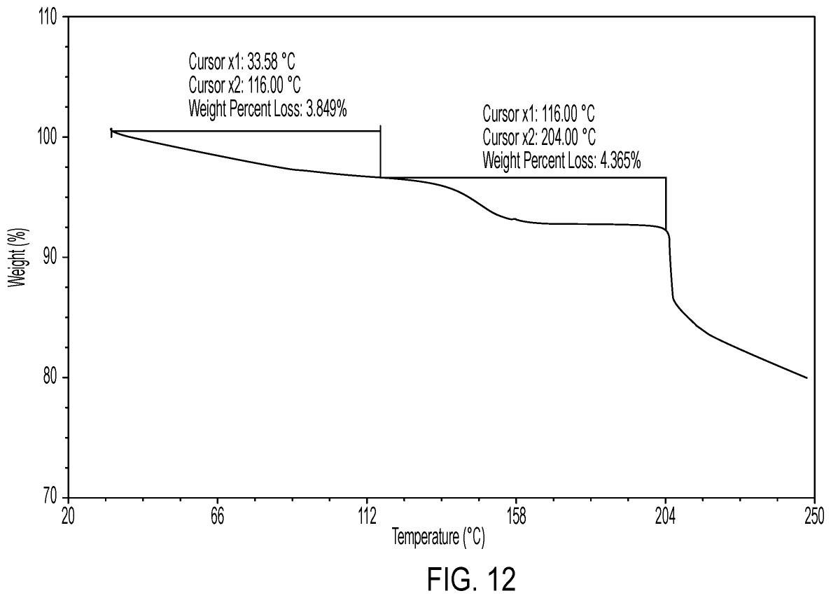

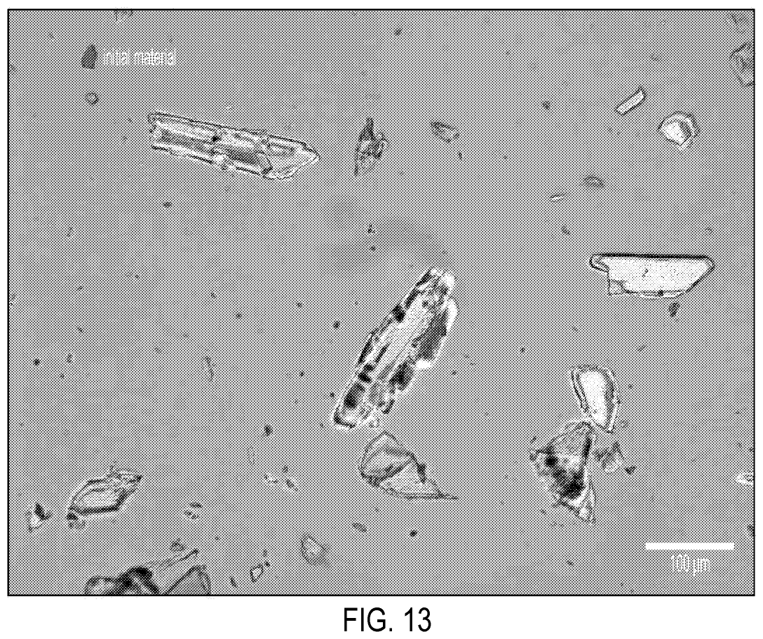

Volixibat potassium salt Form I was used as starting material in the polymorph screening. Starting material was shown to be 99.1% pure via HPLC. ¹H-NMR showed 1.0 equiv. ethanol (FIG. 9). Stoichiometry was shown in the free form of K⁺ to be 1:1 via ion chromatography (IC). Water content was shown to be 3.8% by weight according to KF (Karl Fisher) result. Starting material was of high crystallinity as shown via XRPD (FIG. 10). DSC (10° C./min) showed two broad endothermic peaks at Tₒₙₛₑₜ of 56.3° C. (enthalpy of 37 J/g) and at Tₒₙₛₑₜ of 141.7° C. (enthalpy of 44 J/g), and decomposition I at Tₒₙₛₑₜ of 217.4° C. (FIG. 11). Thermogravimetric analysis (TGA, (10° C./min) shows a two-stage weight loss of 3.8% weight loss at about 116° C. and of 4.4% weight loss between 116.0° C. and 204.0° C. (FIG. 12). Starting material has a morphology of prism-like crystals as shown via PLM (FIG. 13). Particle size distribution (PSD) was shown via dry dispersion to be Dx (10) (μm)=36.5, Dx (50) (μm)=99.9, Dx (90) (μm)=173.

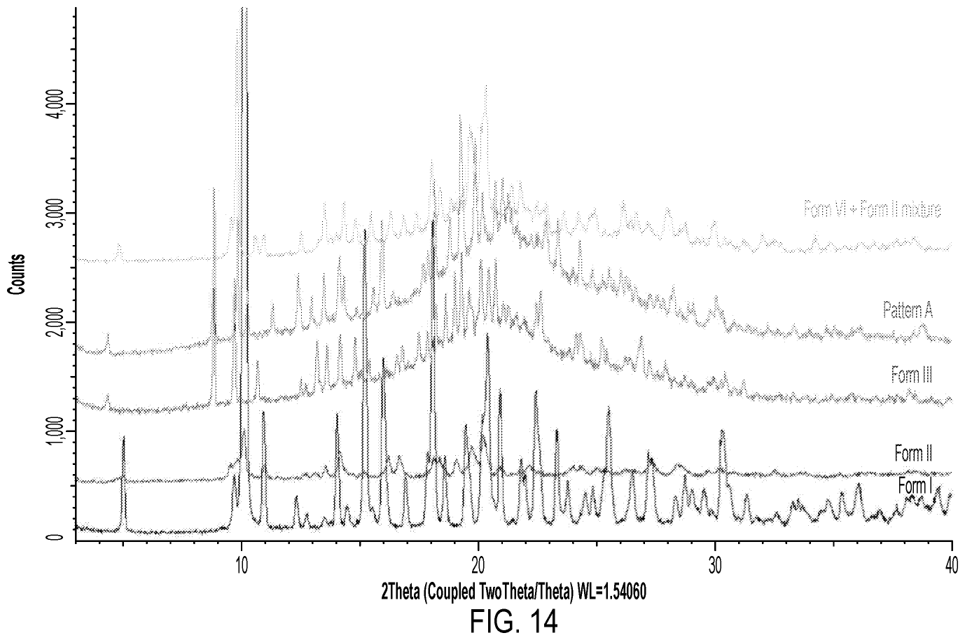

Polymorphic behaviors of volixibat potassium salt Form I were investigated by equilibration in ethanol/water system at different temperatures. In total, 5 crystalline forms were identified to be polymorphs or pseudo-polymorphs of the volixibat potassium salt, including Pattern A, Form I, Form II, Form III and Form VI. Amorphous form of volixibat potassium salt was also obtained by heating Form I at 150° C. for about 1 hour. XRPD overlay of polymorphs is shown in FIG. 14.

### Example 21. Volixibat Potassium Salt Pattern A

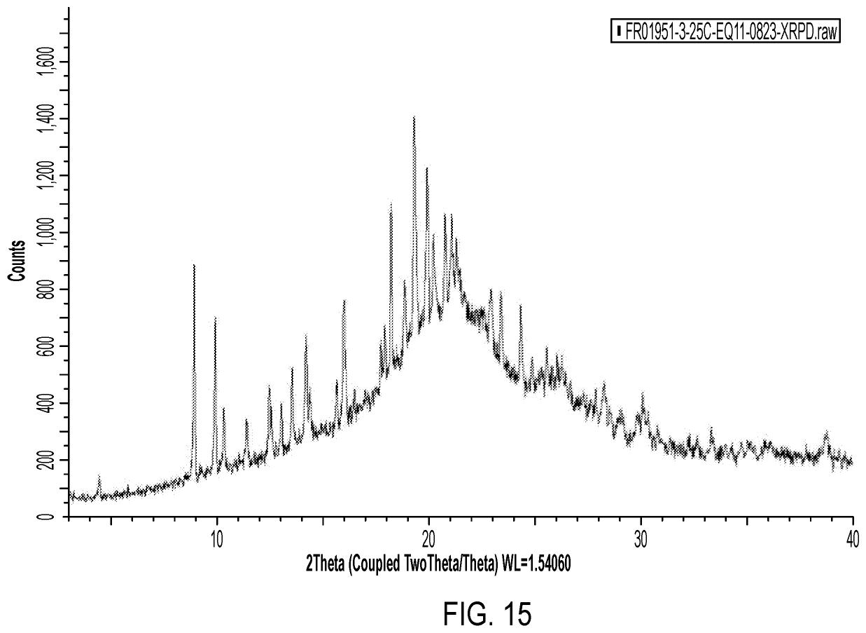

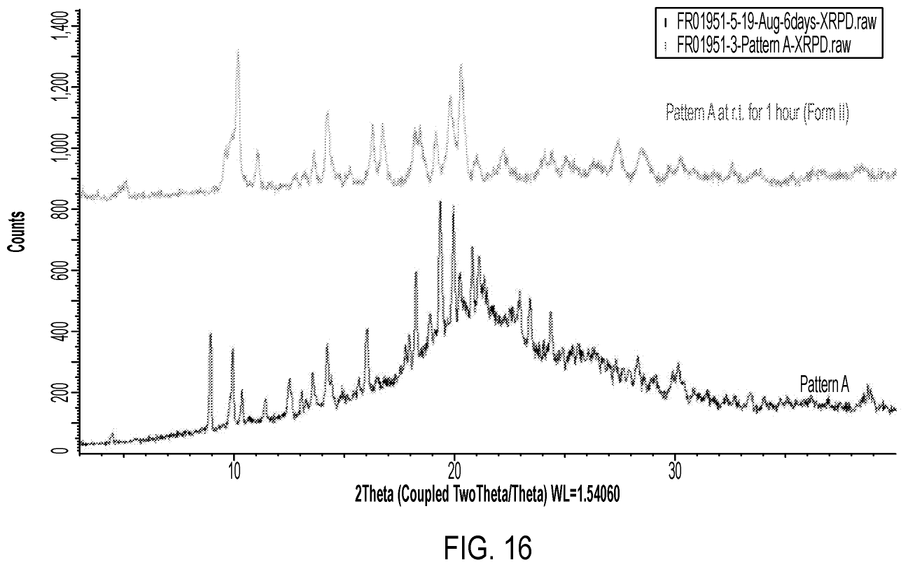

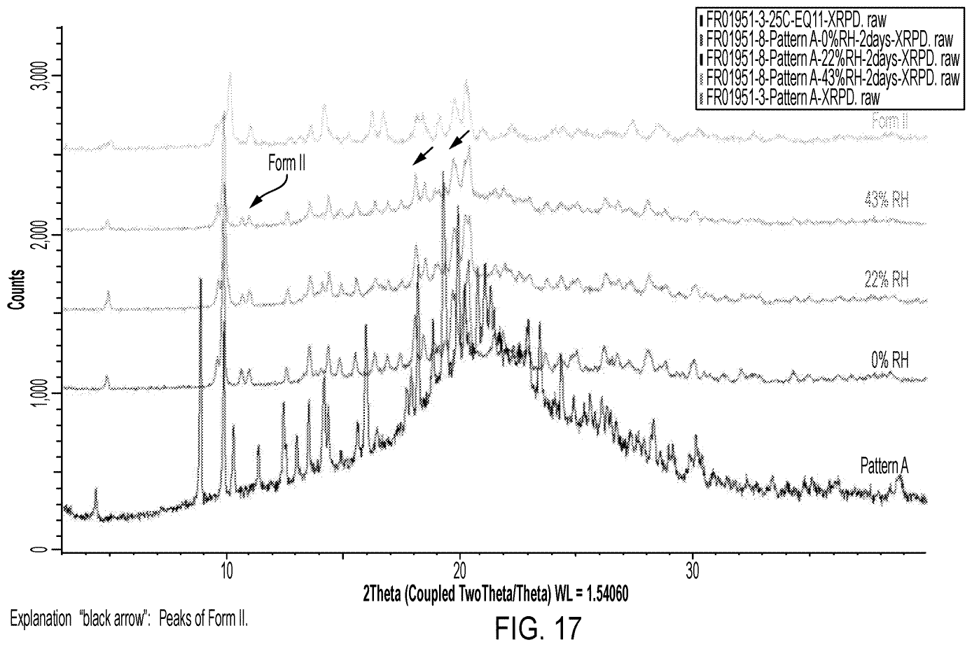

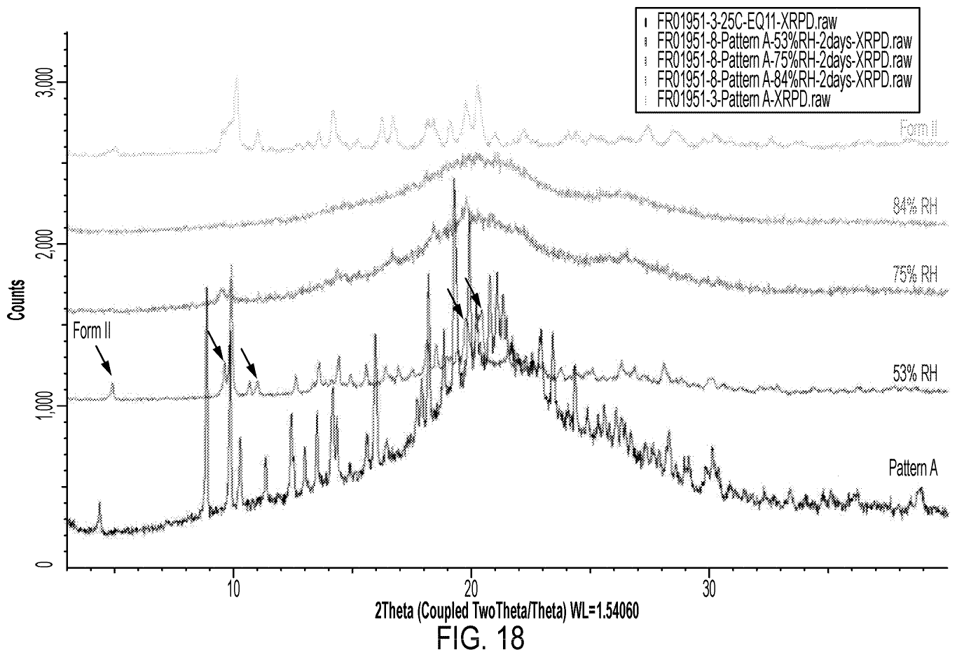

Pattern A is likely an ethanol solvate. It was obtained from ethanol/water systems (v:v=98.7:1.3) by equilibration at 5° C. for 4 days and from pure ethanol by equilibration at 25° C. Upon adding 300 mg amorphous form into pure ethanol saturated by Form I, and stirring the mixture at 5° C. for 1 day, centrifuging the suspension at 14,000 rpm, covering the solid part (wet cake) by Kapton film, and investigating by XRPD, it was determined that Pattern A was formed. Pattern A is of medium crystallinity as shown via XRPD (FIG. 15). Pattern A is the stable form in ethanol/water system at low temperature and in very low water activity (in a.w.<0.34 at 5° C.; in a.w.<0.05 at 25° C.). Pattern A is unstable and converted to Form II under ambient condition (20-30° C.; 40-75% RH) in about 1 hour (FIG. 16). 30 mg of Pattern A were placed in 0%, 22.5%, 43%, 53%, 75%, and 84% RH at 25° C. for 2 days and samples were characterized by XRPD without Kapton film. This showed that Pattern A can convert to a physical mixture of Form II and Form VI in 65% RH or below at 25° C. and can also convert to amorphous form in 86% RH or above at 25° C. (FIG. 17, FIG. 18, Table 1).

TABLE 1
Evaluation of Pattern A in different humidity at 25° C.

| | | Actual |
|---|---|---|
| Theoretical humidity | XRPD | humidity |
| 0% RH (phosphoric pentoxide) | Form VI (major) | 16% RH |
| | and Form II | |
| 22.5% RH (potassium acetate) | Form VI (major) | 37% RH |
| | and Form II | |
| 43% RH (potassium carbonate) | Form VI (major) | 60% RH |
| | and Form II | |
| 53% RH (magnesium nitrate) | Form VI (major) | 65% RH |
| | and Form II | |
| 75% RH (sodium chloride) | Almost amorphous form | 86% RH |
| 84% RH (potassium chloride) | Amorphous form | 94% RH |

### Example 22. Volixibat Potassium Salt Form I

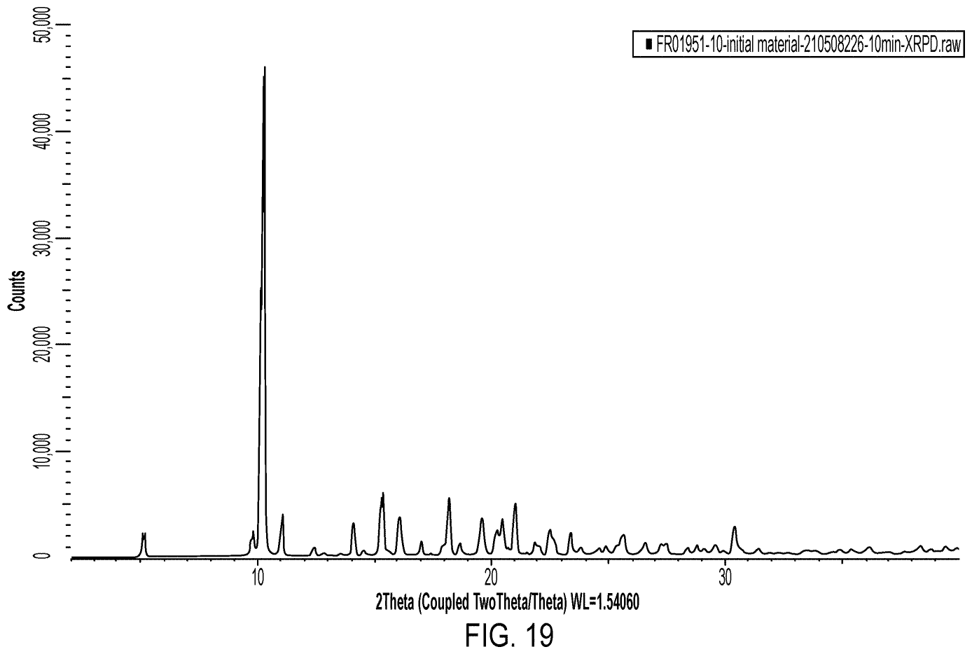

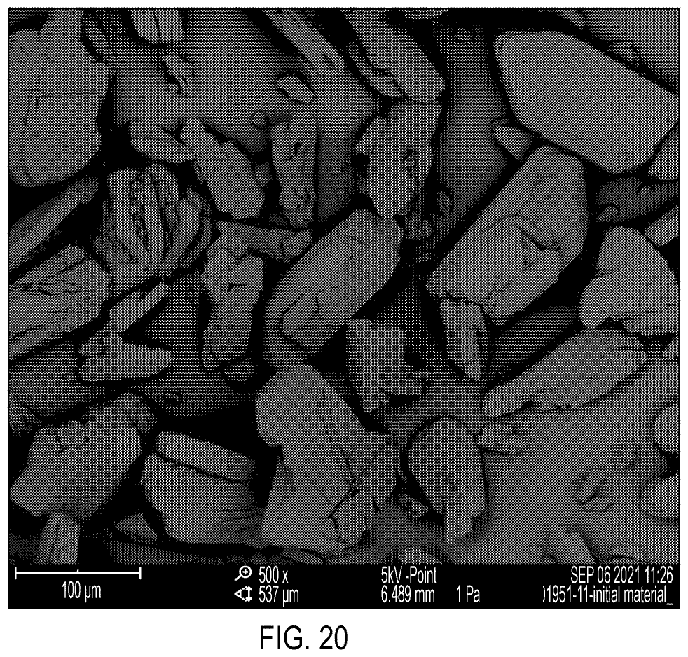

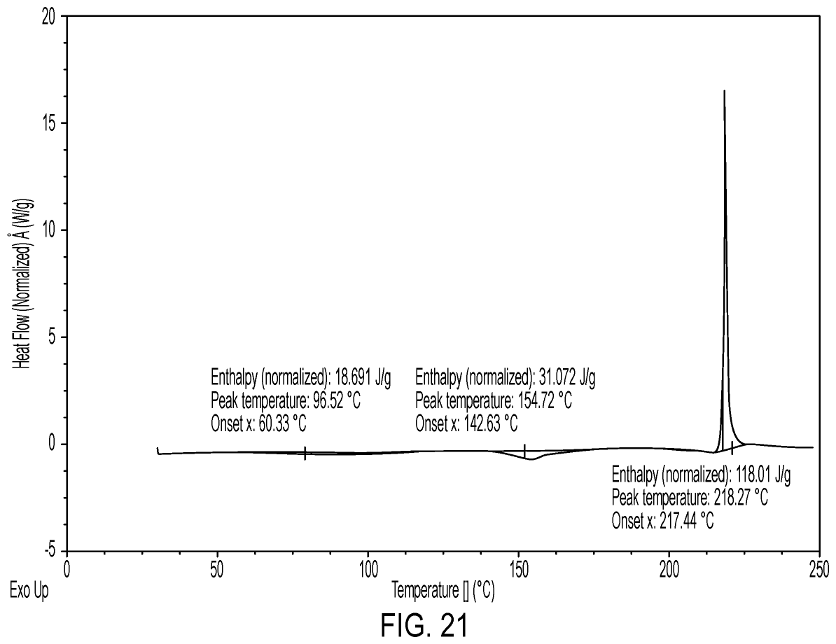

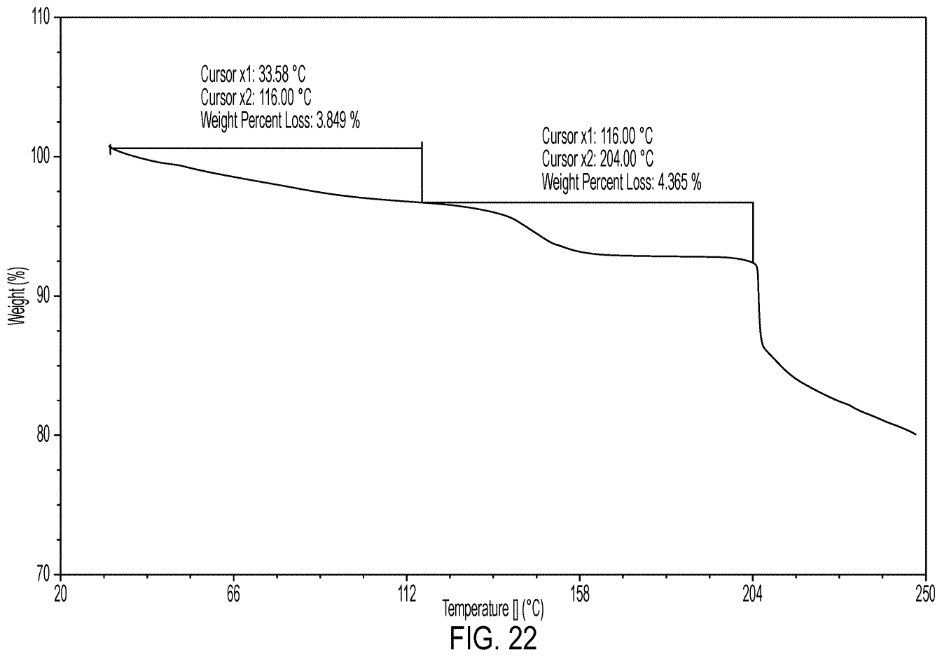

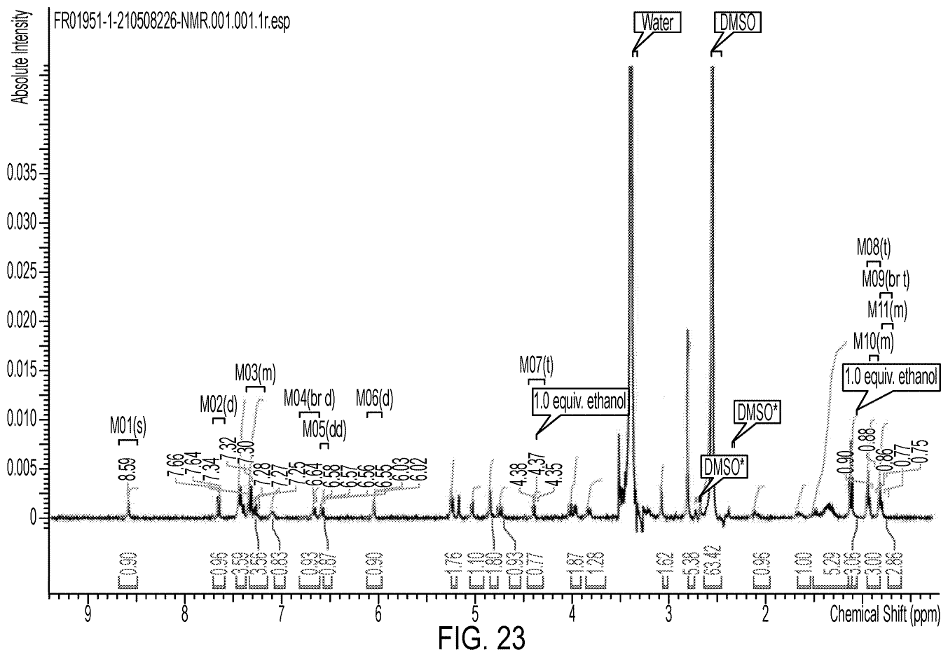

Form I is an ethanol/water solvate. It was obtained from exposure of Form III to ambient conditions (20-30° C.; 40-75% RH) for about 1 hour. Form I is of high crystallinity as shown via XRPD (FIG. 19) and has a morphology of prism-like crystals as shown via PLM (FIG. 13) and SEM (FIG. 20). It contains about 2.0 equivalent (3.8% by weight) of water according to KF result. DSC shows two broad endothermic peaks at Tₒₙₛₑₜ of 56.3° C. and at Tₒₙₛₑₜ of 141.7° C., which correspond to dehydration and desolvation, respectively, and decomposition occurs at Tₒₙₛₑₜ of 217.4° C. (FIG. 21). TGA shows a two-stage weight loss before decomposition. One is 3.8% weight loss at about 116° C. and the other one is 4.4% weight loss between 116.0° C. and 204.0° C. (FIG. 22). ¹H-NMR shows 1.0 equiv. ethanol (FIG. 23). Form I is stable under ambient conditions at 20-30° C. and 40-75% RH. Form I is unstable in a high humidity condition and is sensitive to manually grinding. Amorphous form was obtained after exposure to above 80% RH at 25° C. by DVS or after manually grinding.

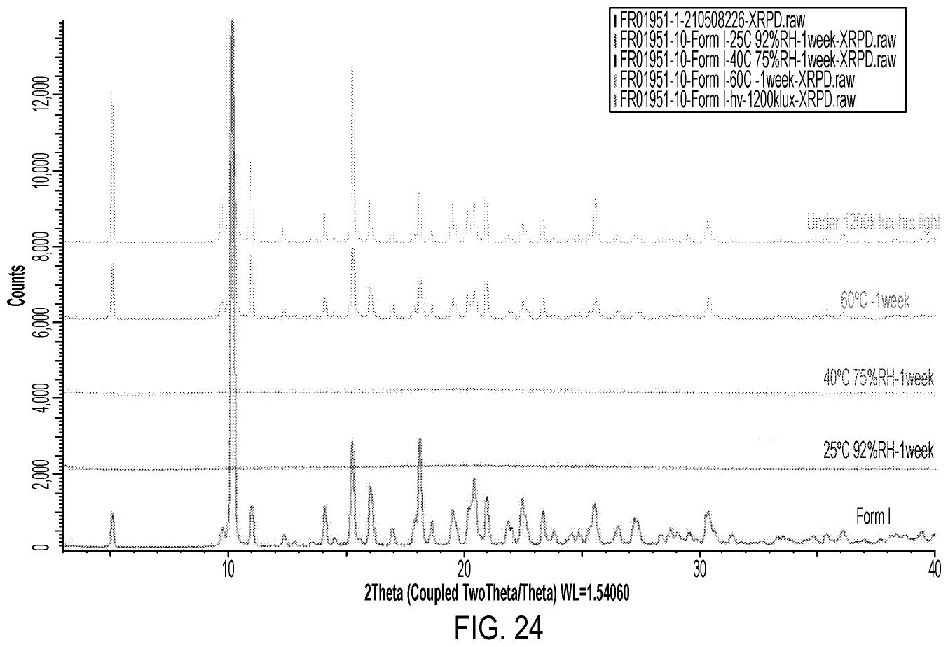

Bulk stability of volixibat potassium salt Form I was evaluated at 25° C. and 92% RH in an open container, at 40° C. and 75% RH in an open container and at 60° C. in a tight container over 1 week, and under visible light for 1.2 million lux-hrs at 25° C. Samples were characterized by XRPD without Kapton film and HPLC and inspected for color change. No color change was observed under any of these conditions. Volixibat potassium salt Form I is physically and chemically stable at 60° C. over 1 week (FIG. 24). It is chemically stable but physically unstable after stressed at 25° C./92% RH and 40° C./75% RH over 1 week, as amorphous form was obtained (FIG. 24). Form I (FIG. 24) is chemically unstable under visible light (HPLC purity reduced to 97.6%) and this photostability issue can be overcome under protection from light (HPLC purity of 99.2% when vial covered by aluminum foil).

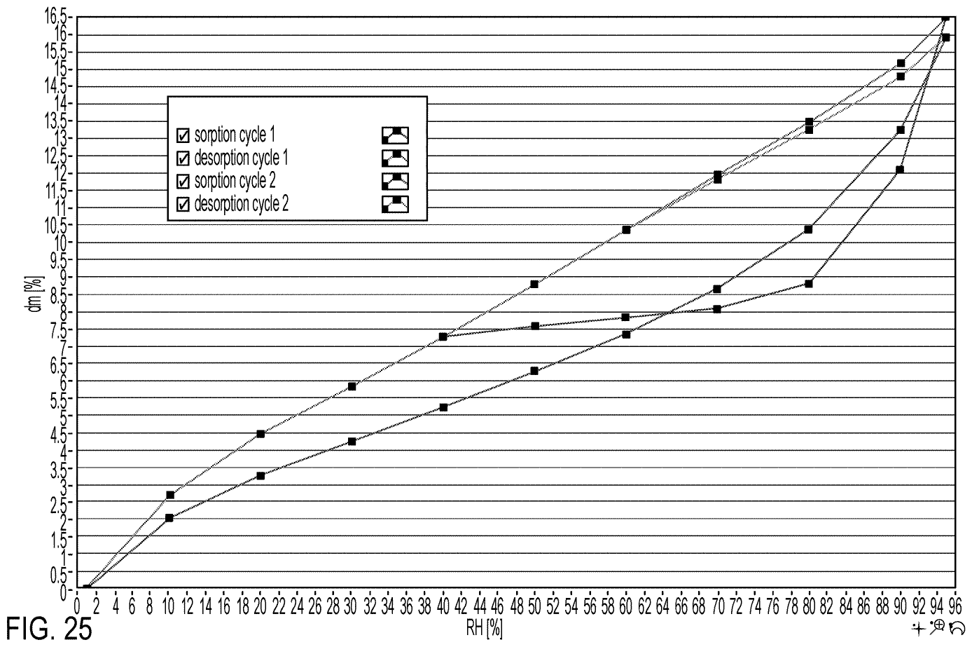

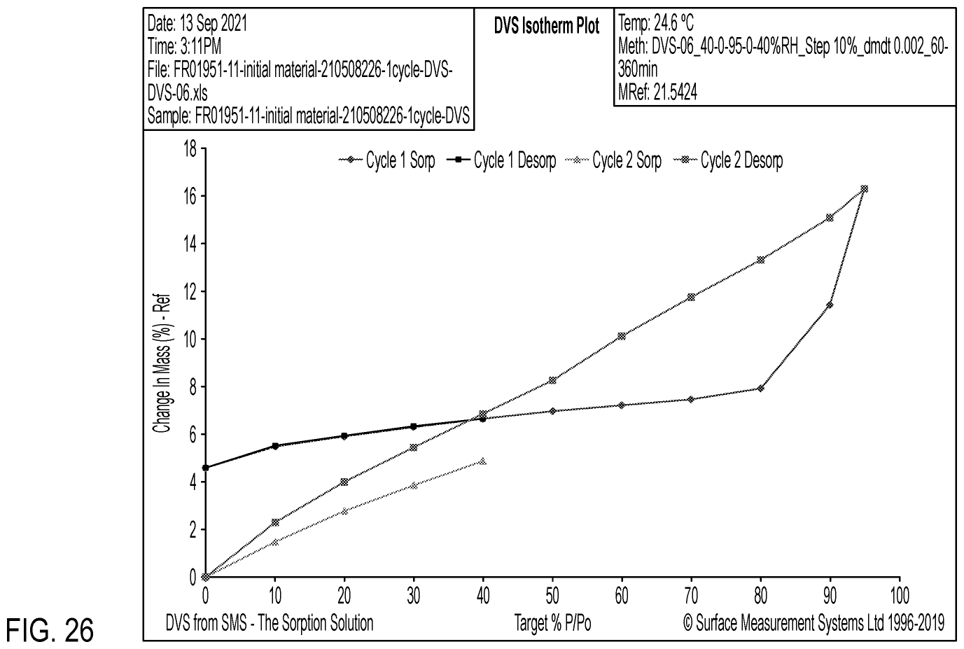

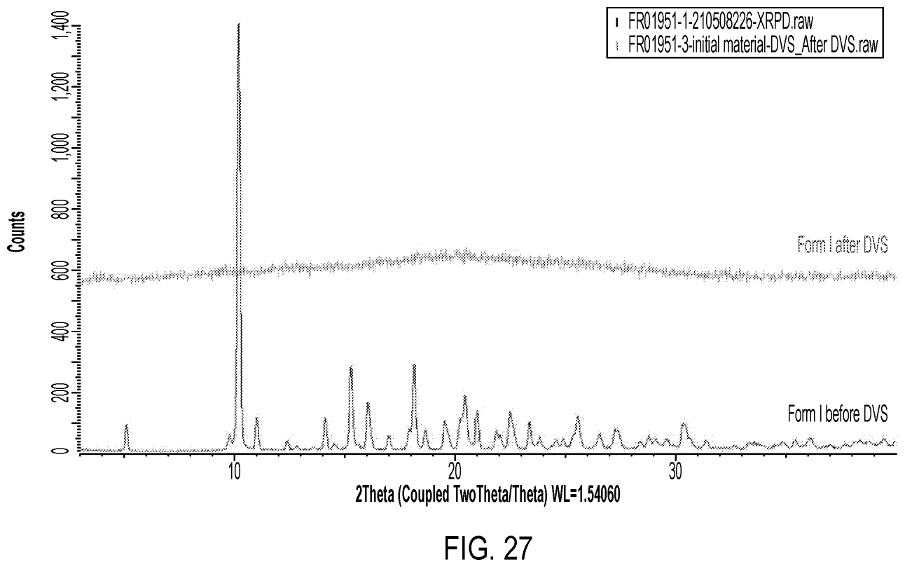

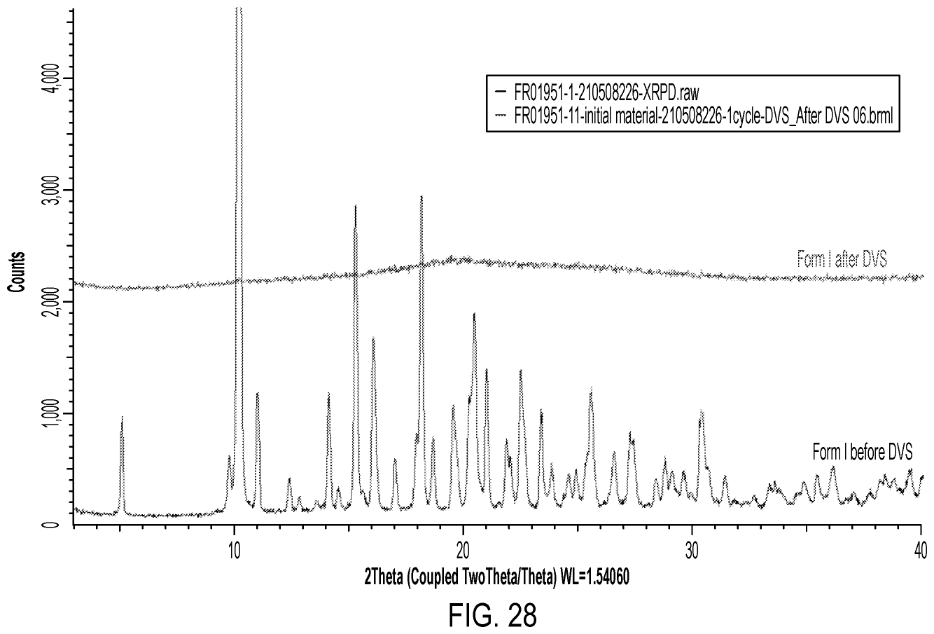

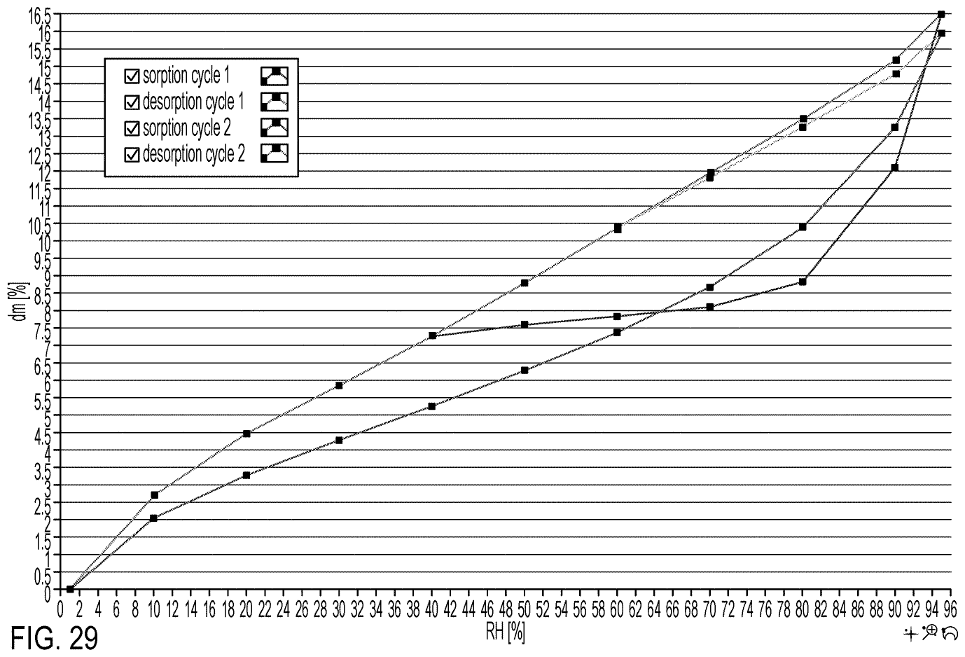

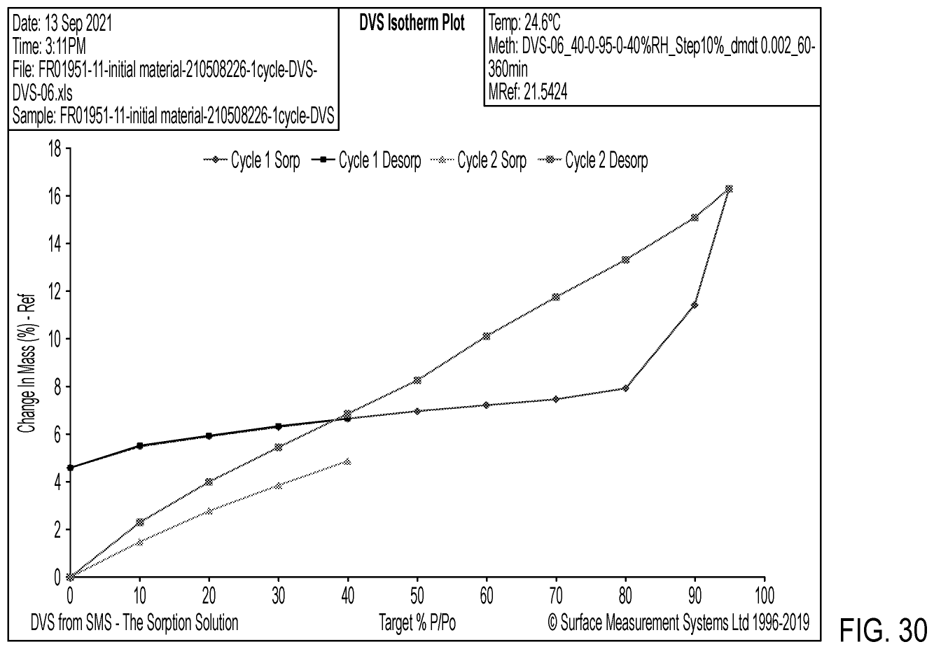

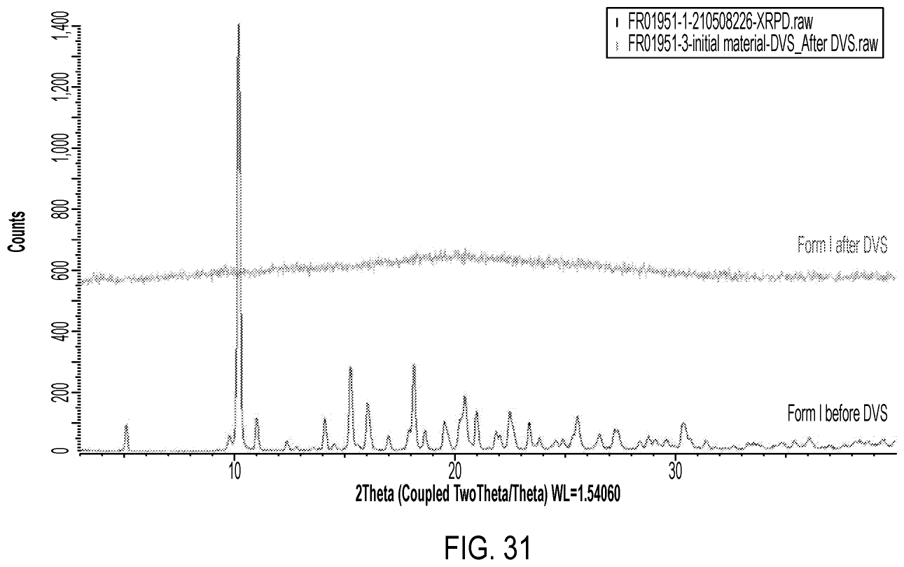

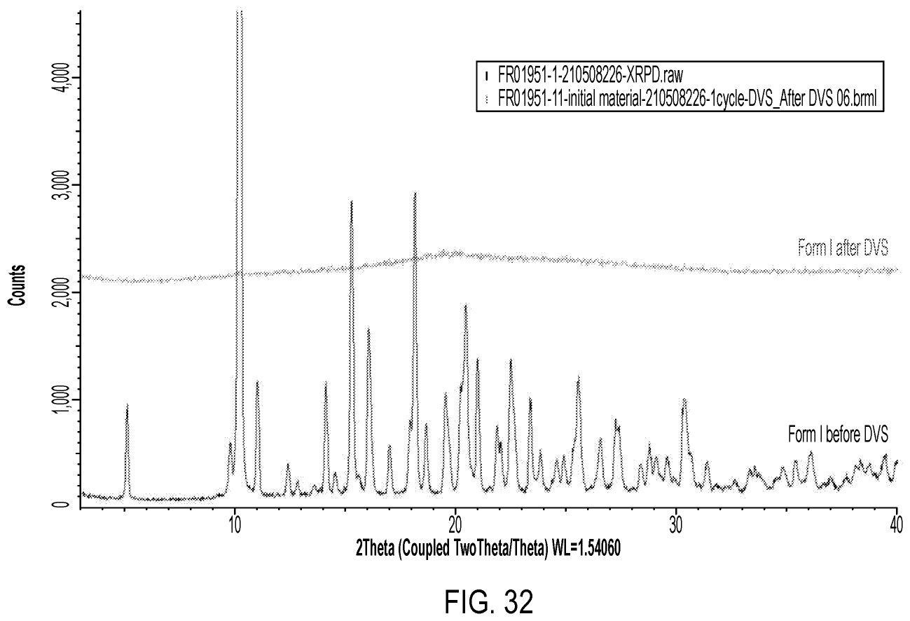

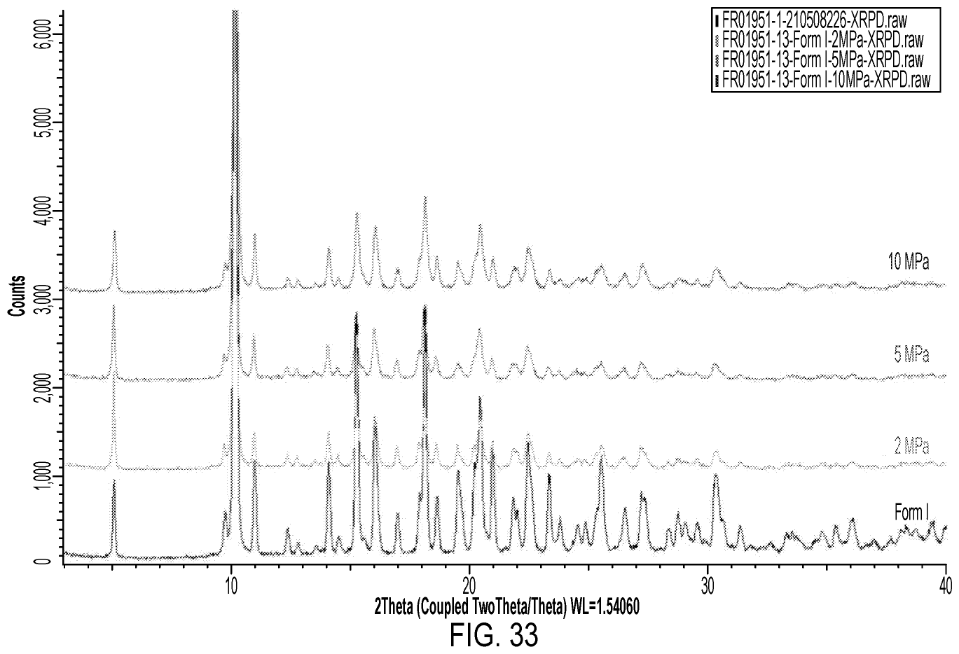

Hygroscopicity of volixibat potassium salt Form I was evaluated by dynamic vapor sorption (DVS) test at 25° C. with a cycle of 40-95-0-95-40% RH, dm/dt 0.002, equilibration time 240 min. (FIG. 25, Table 2) or a cycle of 40-0-95-0-40% RH, dm/dt 0.002, equilibration time 60 min. and max. equilibration time 360 min. (FIG. 26, Table 3). XRPD without Kapton film was measured after the DVS tests to determine form change (FIG. 27, FIG. 29). Volixibat potassium salt Form I is slightly hygroscopic in 80% RH or below. However, it becomes hygroscopic and starts desolvation in above 80% RH. It absorbs about 9.2% water from 40% RH to 95% RH at 25° C. (1.5% from 40% RH to 80% RH and 7.7% from 80% RH to 95% RH, FIG. 29) and absorbs about 1170 water from 0% RH to 95% RH at 25° C. (3.3% from 0% RH to 80% RH and 8.4% from 80% RH to 95% RH, FIG. 30). After the DVS test, obtained sample was amorphous form (FIG. 31, FIG. 32). Storage of Form I is suggested at low humidity (≤80% RH) at 25° C.

TABLE 2
Water sorption and desorption experiment of Form I.

| Relative | 1^{st} sorption | 1^{st} desorption | 2^{nd} sorption | 2^{nd} desorption |
|---|---|---|---|---|
| humidity | weight % | weight % | weight % | weight % |
| at 25° C. | change | change | change | change |
| 0% | N/A | 0 | 0 | N/A |
| 10% | N/A | 2.7 | 2.1 | N/A |
| 20% | N/A | 4.5 | 3.3 | N/A |
| 30% | N/A | 5.8 | 4.3 | N/A |
| 40% | 7.3 | 7.3 | 5.3 | 7.3 |
| 50% | 7.6 | 8.8 | 6.3 | 8.8 |
| 60% | 7.8 | 10.4 | 7.4 | 10.3 |
| 70% | 8.1 | 12.0 | 8.7 | 11.8 |
| 80% | 8.8 | 13.5 | 10.4 | 13.3 |
| 90% | 12.1 | 15.2 | 13.2 | 14.8 |
| 95% | 16.5 | 16.5 | 16.0 | 16.0 |

TABLE 3
Additional water sorption and desorption experiment of Form I.

| Relative | 1^{st} desorption | 1^{st} sorption | 2^{nd} desorption | 2^{nd} sorption |
|---|---|---|---|---|
| humidity | weight % | weight % | weight % | weight % |
| at 25° C. | change | change | change | change |
| 0% | 4.6 | 4.6 | 0.0 | 0.0 |
| 10% | 5.5 | 5.5 | 2.3 | 1.5 |
| 20% | 5.9 | 5.9 | 4.0 | 2.8 |
| 30% | 6.3 | 6.3 | 5.5 | 3.9 |
| 40% | 6.7 | 6.7 | 6.9 | 4.9 |
| 50% | N/A | 7.0 | 8.3 | N/A |
| 60% | N/A | 7.2 | 10.1 | N/A |
| 70% | N/A | 7.5 | 11.8 | N/A |
| 80% | N/A | 7.9 | 13.3 | N/A |
| 90% | N/A | 11.4 | 15.1 | N/A |
| 95% | N/A | 16.3 | 16.3 | N/A |

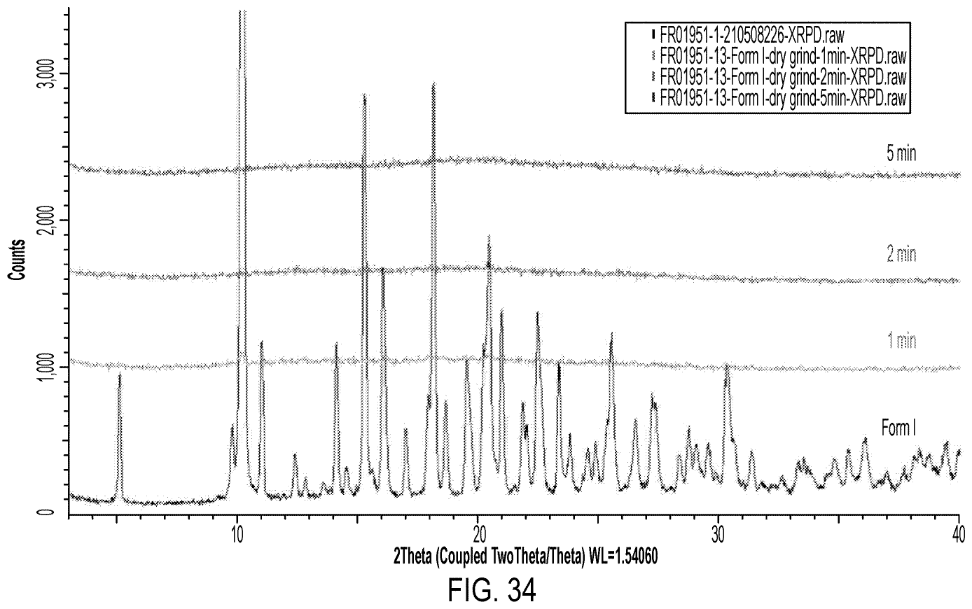

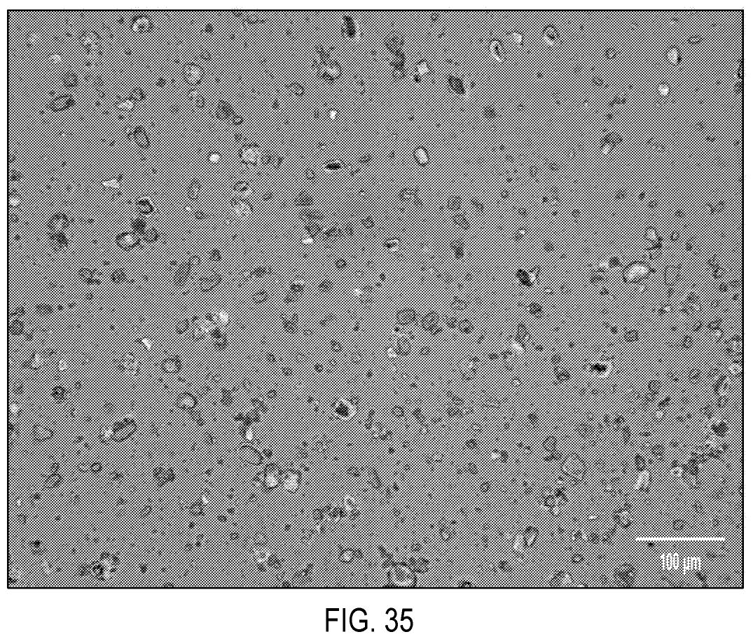

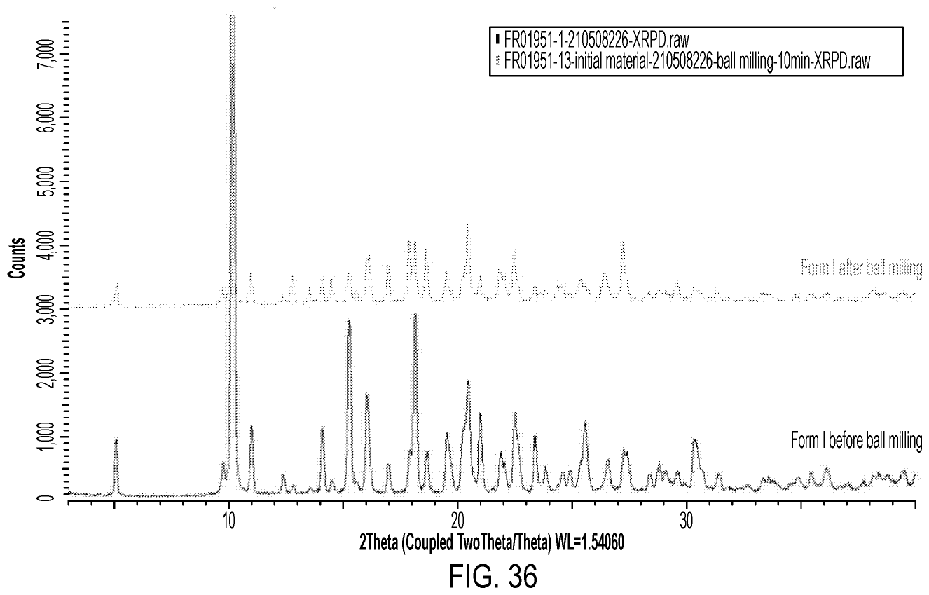

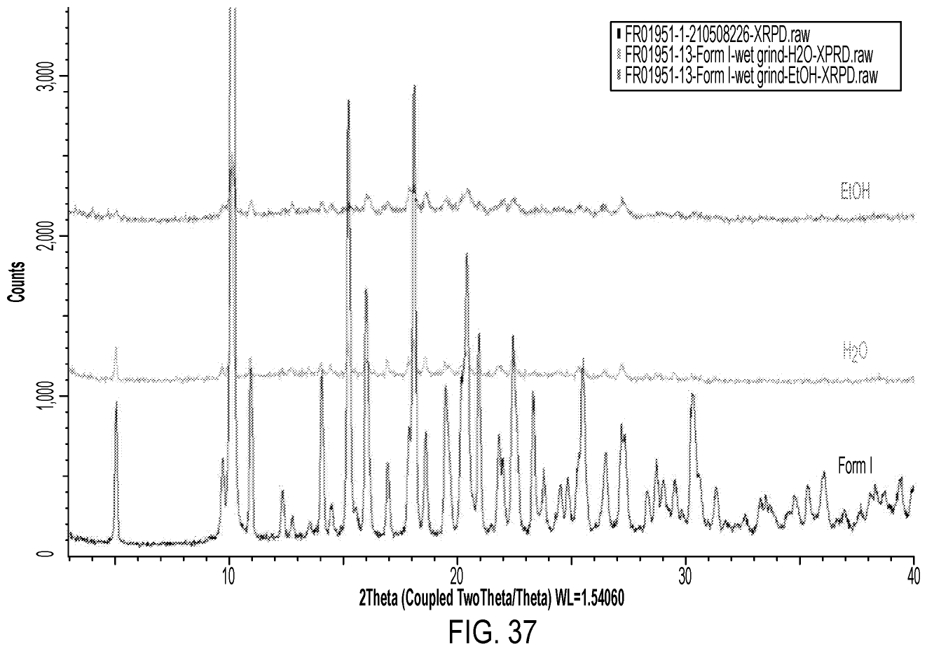

Feasibility of formulation process for volixibat potassium salt Form I were evaluated by compression, manual grinding, manual granulation simulation, and ball milling experiments. Volixibat potassium salt Form I showed good tolerance to compression with no obvious crystallinity decrease (FIG. 33) when 20 mg of Form I were compressed for 5 minutes under 2 MPa, 5 MPa, and 10 MPa with a hydraulic press and samples were evaluated by XRPD without Kapton film. However, Form I converted to amorphous form after dry grinding (FIG. 34) when 20 mg of Form I were ground manually with a mortar and a pestle for 1, 2, and 5 minutes and samples were evaluated by XRPD without Kapton film. To reduce particle size, ball milling was found to better achieve that purpose (PLM before ball milling, FIG. 13 to PLM after ball milling, FIG. 35) and had no significant impact on crystallinity (FIG. 36) at the same time. Ball milling experiments were run on about 130 mg of Form I with added grinding media, followed by grinding by ball-milling (via instrument Retsch MM400) at 25 Hz for 10 minutes. Sample was evaluated by XRPD without Kapton film and PLM. Granulation experiments were performed by adding water or ethanol dropwise to about 20 mg Form I until the sample was wetted sufficiently. The wet samples were ground gently with a mortar and a pestle. Samples were then dried under ambient conditions for 10 minutes and samples were evaluated by XRPD without Kapton film. After undergoing granulation with water, Form I showed no form change, but a slight crystallinity decrease (FIG. 37). After undergoing granulation with ethanol, Form I showed no form change, but a significant crystallinity decrease (FIG. 37). It is suggested to monitor crystallinity of the volixibat potassium salt Form I and establish a quantitative method to determine amorphous content during the formulation process.

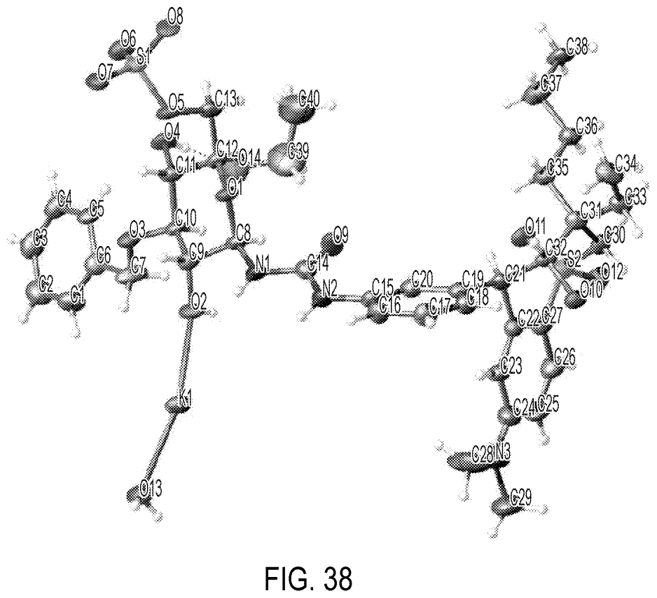

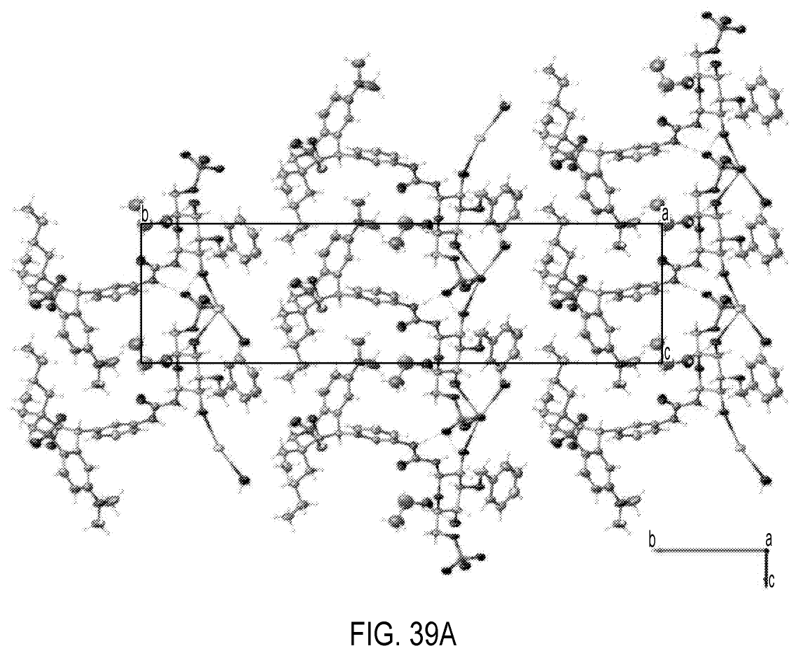

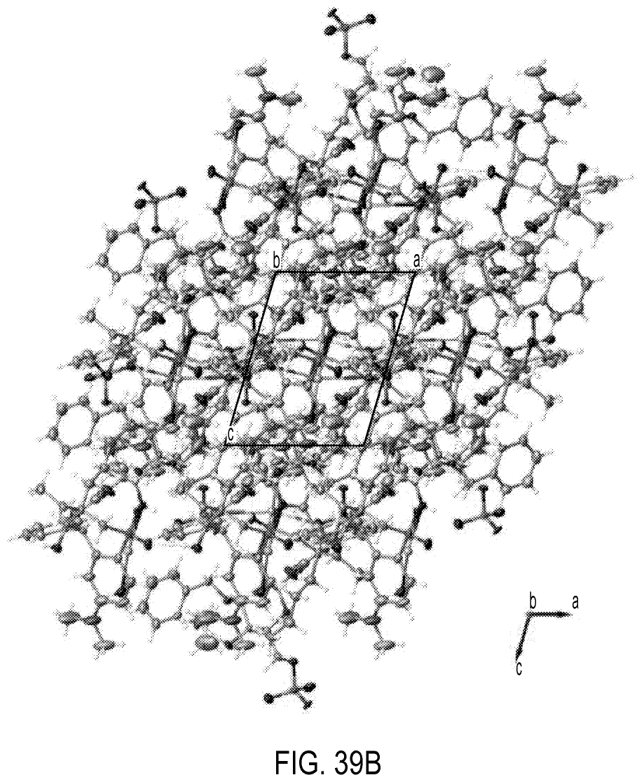

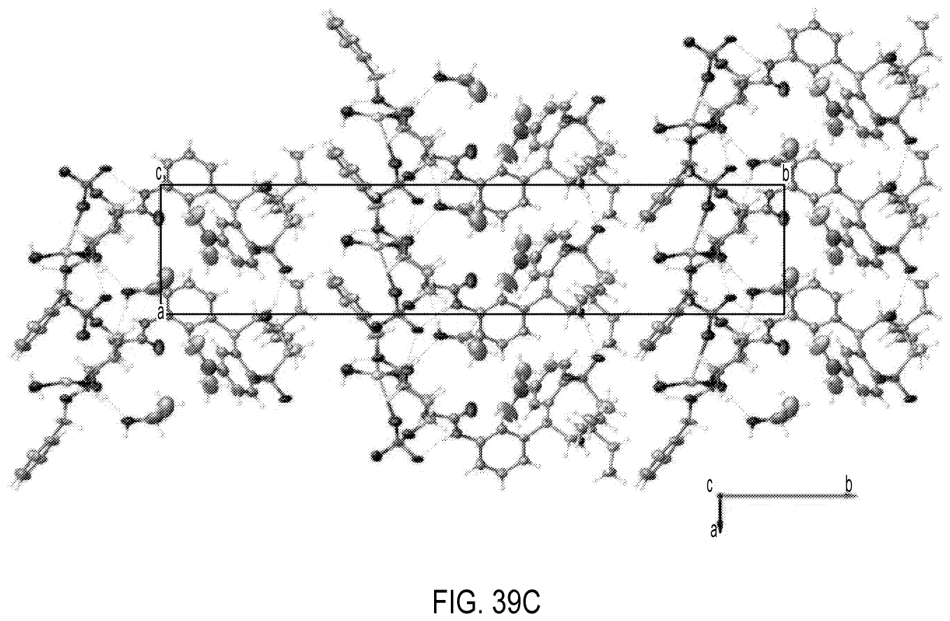

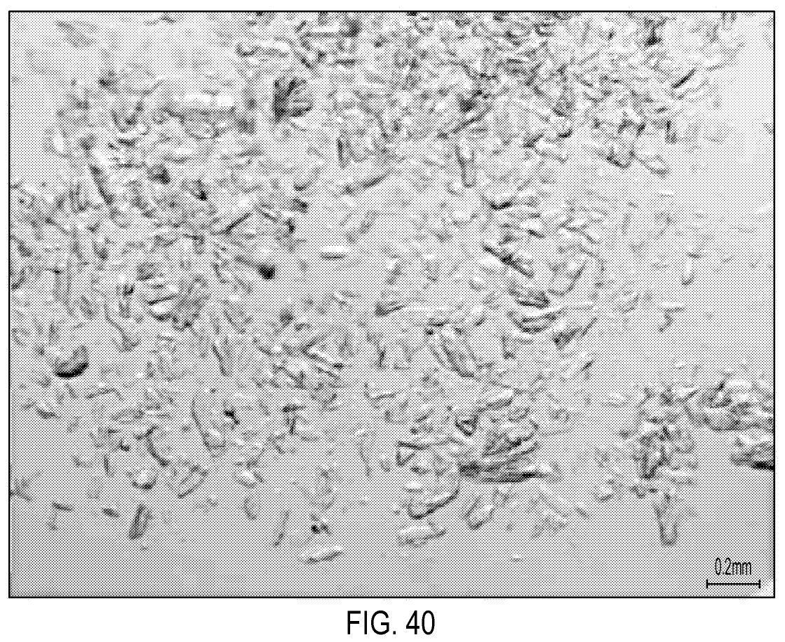

X-ray crystallographic analysis showed the crystal of Form I was a colourless block with the following dimensions: 0.20×0.10×0.04 mm³. The symmetry of the crystal structure was assigned the monoclinic space group P2₁ with the following parameters: a=7.3465(3) Å, b=34.1888(14) Å, c=9.4817(5) Å, α=90°, β=106.059(5)°, γ=90°, cell volume V=2288.57(19) Å³, cell formula units Z=2, crystal density Dc=1.318 g/cm³, F(000)=964.0, absorption coefficient μ(CuKα)=2.425 mm⁻¹, and cell measurement temperature T=149.99(10) K. ORTEP structure is shown in FIG. 38, packing diagrams are shown in FIGS. 39A-39C, and crystals are shown in FIG. 40. Atomic coordinates and equivalent isotropic displacement parameters are displayed in Table 4, bond lengths are shown in Table 5, bond angles are shown in Table 6, hydrogen bonds are shown in Table 7, and torsion angles are shown in Table 8.

TABLE 4
Atomic coordinates (×10\hat{ }4) and equivalent isotropic displacement
parameters (A\hat{ }2 × 10\hat{ }3).

| Atom | x | y | z | U(eq) |
|---|---|---|---|---|
| S(1) | 10007(3) | 3838.9(6) | 14089(2) | 28.4(4) |
| S(2) | 5005(3) | 6766.2(6) | 4843(2) | 31.9(5) |
| O(1) | 7864(7) | 4293.0(17) | 9617(5) | 27.8(12) |
| O(11) | 4886(7) | 6529.4(18) | 6073(6) | 31.1(13) |
| O(3) | 3420(7) | 3510.3(19) | 8937(6) | 32.2(12) |
| O(4) | 4368(8) | 3977.7(18) | 11510(6) | 35.8(14) |
| O(2) | 4641(7) | 3816.3(19) | 6443(6) | 32.6(13) |
| O(12) | 3417(8) | 7028.0(19) | 4239(7) | 39.6(15) |
| O(5) | 9331(8) | 3995.8(18) | 12456(6) | 33.1(13) |
| O(7) | 11011(8) | 3485.3(18) | 13933(6) | 35.5(13) |
| O(8) | 11248(7) | 4143.6(17) | 14941(6) | 31.8(13) |
| O(10) | 9838(8) | 6716.4(18) | 4025(6) | 32.0(13) |
| O(9) | 7416(11) | 4997.8(19) | 7378(8) | 49.2(18) |
| O(6) | 8328(8) | 3785(2) | 14584(6) | 40.2(15) |
| N(1) | 7777(9) | 4345(2) | 7151(7) | 27.6(15) |
| O(13) | 4336(9) | 3016(2) | 1549(7) | 42.6(15) |
| N(2) | 9265(10) | 4775(2) | 5966(7) | 30.0(15) |
| C(14) | 8090(13) | 4729(3) | 6869(10) | 34.9(19) |
| C(8) | 6633(12) | 4275(3) | 8131(8) | 30.9(18) |
| C(9) | 5673(10) | 3882(3) | 7937(8) | 27.0(17) |
| C(30) | 7017(12) | 7075(2) | 5334(9) | 31.8(18) |
| C(11) | 5524(10) | 3946(3) | 10550(8) | 27.8(18) |
| C(36) | 8175(13) | 7057(3) | 8707(9) | 34.2(19) |
| C(19) | 9627(11) | 5836(2) | 5159(8) | 26.8(17) |
| C(35) | 8893(12) | 6759(3) | 7769(9) | 33.2(18) |
| C(10) | 4350(11) | 3874(3) | 8946(8) | 28.5(17) |
| C(16) | 11630(13) | 5138(3) | 5196(10) | 37(2) |
| C(17) | 12310(13) | 5485(3) | 4800(10) | 38(2) |
| C(20) | 8947(13) | 5484(2) | 5553(9) | 31.7(18) |
| C(13) | 8168(12) | 4343(3) | 12182(8) | 31.6(19) |
| C(18) | 11325(12) | 5833(3) | 4768(9) | 31.6(18) |
| C(12) | 6792(11) | 4307(3) | 10650(8) | 28.9(17) |
| C(5) | 93(12) | 3304(3) | 9821(9) | 32.4(19) |
| C(21) | 8428(12) | 6203(2) | 5053(9) | 28.5(17) |
| C(31) | 8936(11) | 6910(2) | 6254(9) | 28.9(18) |
| C(32) | 9667(11) | 6578(2) | 5400(8) | 25.9(17) |
| C(15) | 9921(13) | 5139(3) | 5594(9) | 32.4(18) |
| C(27) | 5295(11) | 6474(3) | 3433(9) | 32.1(18) |
| C(6) | 214(11) | 3252(3) | 8384(10) | 34.8(19) |
| C(26) | 3925(13) | 6485(3) | 2087(10) | 43(2) |
| C(1) | −991(14) | 2997(3) | 7461(11) | 46(2) |
| C(23) | 7006(13) | 6007(3) | 2419(9) | 35.8(19) |
| C(3) | −2495(14) | 2849(3) | 9322(12) | 48(3) |
| N(3) | 5865(16) | 5795(3) | −79(10) | 67(3) |
| C(22) | 6909(12) | 6228(2) | 3616(9) | 30.0(18) |
| C(4) | −1258(12) | 3097(3) | 10266(11) | 40(2) |
| C(37) | 8217(15) | 6900(3) | 10197(11) | 49(3) |
| C(24) | 5643(16) | 6018(3) | 1061(11) | 48(2) |
| C(2) | −2377(15) | 2794(3) | 7916(13) | 51(3) |
| C(7) | 1654(11) | 3478(4) | 7848(9) | 45(2) |
| C(38) | 7238(15) | 7175(3) | 11034(10) | 47(2) |
| C(33) | 10312(11) | 7265(3) | 6425(10) | 33.1(19) |
| C(25) | 4062(16) | 6262(3) | 921(10) | 49(3) |
| C(34) | 12266(12) | 7210(3) | 7460(11) | 44(2) |
| C(28) | 7590(30) | 5573(5) | 81(15) | 110(7) |
| C(29) | 4500(20) | 5823(4) | −1535(13) | 84(5) |
| O(14) | 1699(9) | 4510(2) | 10151(9) | 55.3(19) |
| C(40) | 2770(30) | 5098(6) | 11381(19) | 115(7) |
| C(39) | 1910(20) | 4923(5) | 9987(18) | 95(5) |
| K(1) | 4743(3) | 3503.7(6) | 3864.1(19) | 37.5(4) |

TABLE 5
Bond lengths [A].

| Atom | Atom | Length/Å | Atom | Atom | Length/Å |
|---|---|---|---|---|---|
| S(1) | O(5) | 1.583(5) | C(19) | C(20) | 1.393(12) |
| S(1) | O(7) | 1.445(6) | C(19) | C(18) | 1.396(11) |
| S(1) | O(8) | 1.470(6) | C(19) | C(21) | 1.519(11) |
| S(1) | O(6) | 1.448(6) | C(35) | C(31) | 1.535(11) |
| S(1) | K(1)¹ | 3.725(3) | C(16) | C(17) | 1.379(13) |
| S(2) | O(11) | 1.442(6) | C(16) | C(15) | 1.408(13) |
| S(2) | O(12) | 1.456(6) | C(17) | C(18) | 1.388(12) |
| S(2) | C(30) | 1.770(9) | C(20) | C(15) | 1.374(12) |
| S(2) | C(27) | 1.729(9) | C(13) | C(12) | 1.528(11) |
| O(1) | C(8) | 1.450(9) | C(5) | C(6) | 1.401(12) |
| O(1) | C(12) | 1.417(9) | C(5) | C(4) | 1.377(12) |
| O(3) | C(10) | 1.419(10) | C(21) | C(32) | 1.554(11) |
| O(3) | C(7) | 1.421(9) | C(21) | C(22) | 1.506(11) |
| O(4) | C(11) | 1.410(9) | C(31) | C(32) | 1.572(11) |
| O(4) | K(1)² | 2.710(6) | C(31) | C(33) | 1.560(11) |
| O(2) | C(9) | 1.428(9) | C(27) | C(26) | 1.390(12) |
| O(2) | K(1) | 2.688(6) | C(27) | C(22) | 1.425(12) |
| O(5) | C(13) | 1.444(10) | C(6) | C(1) | 1.372(13) |
| O(7) | K(1)¹ | 2.761(6) | C(6) | C(7) | 1.507(12) |
| O(10) | C(32) | 1.425(9) | C(26) | C(25) | 1.370(14) |
| O(9) | C(14) | 1.207(11) | C(1) | C(2) | 1.395(14) |
| O(6) | K(1)² | 2.708(6) | C(23) | C(22) | 1.381(12) |
| N(1) | C(14) | 1.370(11) | C(23) | C(24) | 1.396(13) |
| N(1) | C(8) | 1.436(10) | C(3) | C(4) | 1.377(15) |
| O(13) | K(1) | 2.706(7) | C(3) | C(2) | 1.372(16) |
| N(2) | C(14) | 1.383(11) | N(0) | C(24) | 1.369(14) |
| N(2) | C(15) | 1.416(11) | N(3) | C(28) | 1.447(18) |
| C(8) | C(9) | 1.506(12) | N(3) | C(29) | 1.469(15) |
| C(9) | C(10) | 1.541(10) | C(37) | C(38) | 1.533(13) |
| C(30) | C(31) | 1.546(11) | C(24) | C(25) | 1.407(16) |
| C(11) | C(10) | 1.546(10) | C(33) | C(34) | 1.510(12) |
| C(11) | C(12) | 1.535(11) | O(14) | C(39) | 1.434(19) |
| C(36) | C(35) | 1.538(11) | C(40) | C(39) | 1.43(2) |
| C(36) | C(37) | 1.503(12) | | | |

¹1 + X, +Y, 1 + Z;
²+X, +Y, 1 + Z

TABLE 6
Bond angles [°].

| Atom | Atom | Atom | Angle/° | Atom | Atom | Atom | Angle/° |
|---|---|---|---|---|---|---|---|
| O(5) | S(1) | K(1)1 | 95.4(2) | C(13) | C(12) | C(11) | 110.7(7) |
| O(7) | S(1) | O(5) | 102.4(3) | C(4) | C(5) | C(6) | 118.5(8) |
| O(7) | S(1) | O(8) | 113.1(3) | C(19) | C(21) | C(32) | 111.8(7) |
| O(7) | S(1) | O(6) | 115.6(4) | C(22) | C(21) | C(19) | 112.2(7) |
| O(7) | S(1) | K(1)¹ | 39.2(3) | C(22) | C(21) | C(32) | 112.9(7) |
| O(8) | S(1) | O(5) | 106.1(3) | C(30) | C(31) | C(32) | 111.2(7) |
| O(8) | S(1) | K(1)¹ | 78.6(2) | C(30) | C(31) | C(33) | 103.7(7) |
| O(6) | S(1) | O(5) | 107.1(3) | C(35) | C(31) | C(30) | 113.5(6) |
| O(6) | S(1) | O(8) | 111.5(4) | C(35) | C(31) | C(32) | 109.8(7) |
| O(6) | S(1) | K(1)¹ | 150.8(3) | C(35) | C(31) | C(33) | 110.2(7) |
| O(11) | S(2) | O(12) | 116.2(3) | C(33) | C(31) | C(32) | 108.2(6) |
| O(11) | S(2) | C(30) | 110.8(4) | O(10) | C(32) | C(21) | 106.0(6) |
| O(11) | S(2) | C(27) | 110.4(4) | O(10) | C(32) | C(31) | 111.1(7) |
| O(12) | S(2) | C(30) | 104.9(4) | C(21) | C(32) | C(31) | 116.2(6) |
| O(12) | S(2) | C(27) | 108.0(4) | C(16) | C(15) | N(2) | 117.0(8) |
| C(27) | S(2) | C(30) | 106.0(4) | C(20) | C(15) | N(2) | 123.5(8) |
| C(12) | O(1) | C(8) | 110.9(6) | C(20) | C(15) | C(16) | 119.4(8) |
| C(10) | O(3) | C(7) | 114.5(7) | C(26) | C(27) | S(2) | 118.7(7) |
| C(11) | O(4) | K(1)² | 122.9(5) | C(26) | C(27) | C(22) | 119.7(8) |
| C(9) | O(2) | K(1) | 145.5(4) | C(22) | C(27) | S(2) | 121.6(6) |
| C(13) | O(5) | S(1) | 117.7(5) | C(5) | C(6) | C(7) | 120.2(8) |
| S(1) | O(7) | K(1)¹ | 121.5(4) | C(1) | C(6) | C(5) | 119.9(8) |
| S(1) | O(6) | K(1)² | 145.1(4) | C(1) | C(6) | C(7) | 120.0(8) |
| C(14) | N(1) | C(8) | 116.5(7) | C(25) | C(26) | C(27) | 121.9(9) |
| C(14) | N(2) | C(15) | 124.6(7) | C(6) | C(1) | C(2) | 121.0(9) |
| O(9) | C(14) | N(1) | 122.8(8) | C(22) | C(23) | C(24) | 123.9(9) |
| O(9) | C(14) | N(2) | 123.8(8) | C(2) | C(25) | C(26) | 120.6(9) |
| N(1) | C(14) | N(2) | 113.4(7) | C(24) | N(3) | C(28) | 120.0(10) |
| O(1) | C(8) | C(9) | 107.5(6) | C(24) | N(3) | C(29) | 120.3(11) |
| N(1) | C(8) | O(1) | 107.6(6) | C(28) | N(3) | C(27) | 119.0(11) |
| N(1) | C(8) | C(9) | 114.1(7) | C(27) | C(22) | C(21) | 121.8(7) |
| O(2) | C(9) | C(8) | 111.3(6) | C(23) | C(22) | C(21) | 121.3(8) |
| O(2) | C(9) | C(10) | 111.1(6) | C(23) | C(22) | C(27) | 116.9(8) |
| C(8) | C(9) | C(10) | 107.1(7) | C(3) | C(4) | C(5) | 121.3(9) |
| C(31) | C(30) | S(2) | 119.7(6) | C(36) | C(37) | C(38) | 112.1(8) |
| O(4) | C(11) | C(10) | 112.1(6) | C(23) | C(24) | C(25) | 117.7(9) |
| O(4) | C(11) | C(12) | 111.1(7) | N(3) | C(24) | C(23) | 120.1(10) |
| C(12) | C(11) | C(10) | 110.6(6) | N(3) | C(24) | C(25) | 122.1(10) |
| C(37) | C(36) | C(35) | 112.7(8) | C(3) | C(2) | C(1) | 118.7(10) |
| C(20) | C(19) | C(18) | 118.6(8) | O(3) | C(7) | C(6) | 112.3(7) |
| C(20) | C(19) | C(21) | 119.1(7) | C(34) | C(33) | C(31) | 116.3(8) |
| C(18) | C(19) | C(21) | 122.2(8) | C(26) | C(25) | C(24) | 119.8(9) |
| C(31) | C(35) | C(36) | 114.7(7) | O(14) | C(39) | C(40) | 110.3(14) |
| O(3) | C(10) | C(9) | 113.4(7) | O(4)³ | K(1) | O(7)⁴ | 99.06(18) |
| O(3) | C(10) | C(11) | 106.3(6) | O(2) | K(1) | O(4)³ | 119.3(2) |
| C(9) | C(10) | C(11) | 109.4(6) | O(2) | K(1) | O(7)⁴ | 73.13(17) |
| C(17) | C(16) | C(15) | 119.2(8) | O(2) | K(1) | O(6)³ | 84.23(18) |
| C(16) | C(17) | C(18) | 121.2(8) | O(2) | K(1) | O(13) | 163.3(2) |
| C(15) | C(20) | C(19) | 121.8(8) | O(6)³ | K(1) | O(4)³ | 82.26(18) |
| O(5) | C(13) | C(12) | 107.9(7) | O(6)³ | K(1) | O(7)⁴ | 154.8(2) |
| C(17) | C(18) | C(19) | 119.9(8) | O(13) | K(1) | O(4)³ | 74.74(19) |
| O(1) | C(12) | C(11) | 111.9(7) | O(13) | K(1) | O(7)⁴ | 96.73(19) |
| O(1) | C(12) | C(13) | 108.2(6) | O(13) | K(1) | O(6)³ | 107.8(2) |

¹1 + X, +Y, 1 + Z;
²+X, +Y, 1 + Z;
³+X, +Y, −1 + Z;
⁴−1 + X, +Y, −1 + Z

TABLE 7
Hydrogen bonds.

| D | H | A | d(D − H)/Å | d(H − A)/Å | d(D − A)/Å | D − H − A/° |
|---|---|---|---|---|---|---|
| O(4) | H(4) | O(14) | 0.804(6) | 1.921(7) | 2.723(9) | 175.8(4) |
| O(2) | H(2) | O(8)¹ | 0.854(14) | 1.99(3) | 2.744(7) | 147(6) |
| O(10) | H(10) | O(12)² | 0.82 | 1.99 | 2.792(8) | 164.9 |
| O(13) | H(13B) | O(3)³ | 0.86 | 2.14 | 2.918(8) | 150.9 |
| N(2) | H(2A) | O(8)³ | 0.86 | 2.08 | 2.916(9) | 163.4 |
| O(14) | H(14) | O(1)⁴ | 0.82 | 2.01 | 2.821(8) | 169.5 |

¹−1 + X, +Y, −1 + Z;
²1 + X, +Y, +Z;
³+X, +Y, −1 + Z;
⁴−1 + X, +Y, +Z

TABLE 8
Torsion angles [°].

| A | B | C | D | Angle/° | A | B | C | D | Angle/° |
|---|---|---|---|---|---|---|---|---|---|
| S(1) | O(5) | C(13) | C(12) | −146.9(5) | C(10) | O(3) | C(7) | C(6) | −142.9(8) |
| S(2) | C(30) | C(31) | C(35) | −61.2(9) | C(10) | C(11) | C(12) | O(1) | −50.6(9) |
| S(2) | C(30) | C(31) | C(32) | 63.1(8) | C(10) | C(11) | C(12) | C(13) | −171.4(7) |
| S(2) | C(30) | C(31) | C(33) | 179.2(6) | C(16) | C(17) | C(18) | C(19) | 0.8(14) |
| S(2) | C(27) | C(26) | C(25) | 179.9(8) | C(17) | C(16) | C(15) | N(2) | 178.9(8) |
| S(2) | C(27) | C(22) | C(21) | 1.3(11) | C(17) | C(16) | C(15) | C(20) | 1.2(14) |
| S(2) | C(27) | C(22) | C(23) | −179.7(7) | C(20) | C(19) | C(18) | C(17) | −0.9(12) |
| O(1) | C(8) | C(9) | O(2) | −171.6(6) | C(20) | C(19) | C(21) | C(32) | 150.7(7) |
| O(1) | C(8) | C(9) | C(10) | 66.7(8) | C(20) | C(19) | C(21) | C(22) | −81.3(9) |
| O(11) | S(2) | C(30) | C(31) | 43.7(7) | C(18) | C(19) | C(20) | C(15) | 1.3(12) |
| O(11) | S(2) | C(27) | C(26) | 119.2(7) | C(18) | C(19) | C(21) | C(32) | −34.1(10) |
| O(11) | S(2) | C(27) | C(22) | −61.8(8) | C(18) | C(19) | C(21) | C(22) | 93.8(9) |
| O(4) | C(11) | C(10) | O(3) | −62.7(9) | C(12) | O(1) | C(8) | N(1) | 167.9(6) |
| O(4) | C(11) | C(10) | C(9) | 174.6(7) | C(12) | O(1) | C(8) | C(9) | −68.7(8) |
| O(4) | C(11) | C(12) | O(1) | −175.7(6) | C(12) | C(11) | C(10) | O(3) | 172.7(6) |
| O(4) | C(11) | C(12) | C(13) | 63.5(8) | C(12) | C(11) | C(10) | C(9) | 50.0(9) |
| O(2) | C(9) | C(10) | O(3) | 61.5(9) | C(5) | C(6) | C(1) | C(2) | 1.8(15) |
| O(2) | C(9) | C(10) | C(11) | 179.9(7) | C(5) | C(6) | C(7) | O(3) | 39.5(13) |
| O(12) | S(2) | C(30) | C(31) | 169.8(6) | C(21) | C(19) | C(20) | C(15) | 176.6(8) |
| O(12) | S(2) | C(27) | C(26) | −8.8(8) | C(21) | C(19) | C(18) | C(17) | −176.1(8) |
| O(12) | S(2) | C(27) | C(22) | 170.2(7) | C(32) | C(21) | C(22) | C(27) | −71.6(10) |
| O(5) | S(1) | O(7) | K(1)¹ | 83.3(4) | C(32) | C(21) | C(22) | C(23) | 109.4(9) |
| O(5) | S(1) | O(6) | K(1)² | 44.9(8) | C(32) | C(31) | C(33) | C(34) | −69.9(9) |
| O(5) | C(13) | C(12) | O(1) | −62.7(8) | C(15) | N(2) | C(14) | O(9) | 4.0(14) |
| O(5) | C(13) | C(12) | C(11) | 60.3(8) | C(15) | N(2) | C(14) | N(1) | −175.2(7) |
| O(7) | S(1) | O(5) | C(13) | 176.7(6) | C(15) | C(16) | C(17) | C(18) | −0.9(15) |
| O(7) | S(1) | O(6) | K(1)² | −68.5(7) | C(27) | S(2) | C(30) | C(31) | −76.1(7) |
| O(8) | S(1) | O(5) | C(13) | −64.5(6) | C(27) | C(26) | C(25) | C(24) | −1.3(15) |
| O(8) | S(1) | O(7) | K(1)¹ | −30.4(5) | C(6) | C(5) | C(4) | C(3) | −1.2(14) |
| O(8) | S(1) | O(6) | K(1)² | 160.5(6) | C(6) | C(1) | C(2) | C(3) | −1.3(16) |
| O(6) | S(1) | O(5) | C(13) | 54.6(7) | C(26) | C(27) | C(22) | C(21) | −179.7(8) |
| O(6) | S(1) | O(7) | K(1)¹ | −160.6(3) | C(26) | C(27) | C(22) | C(23) | −0.6(12) |
| N(1) | C(8) | C(9) | O(2) | −52.3(9) | C(1) | C(6) | C(7) | O(3) | −140.8(9) |
| N(1) | C(8) | C(9) | C(10) | −174.0(6) | C(23) | C(24) | C(25) | C(26) | 1.5(15) |
| C(14) | N(1) | C(8) | O(1) | −84.7(9) | N(3) | C(24) | C(25) | C(26) | −178.8(10) |
| C(14) | N(1) | C(8) | C(9) | 156.1(7) | C(22) | C(21) | C(32) | O(10) | −35.3(9) |
| C(14) | N(2) | C(15) | C(16) | 154.9(8) | C(22) | C(21) | C(32) | C(31) | 88.7(8) |
| C(14) | N(2) | C(15) | C(20) | −27.5(13) | C(22) | C(27) | C(26) | C(25) | 0.9(14) |
| C(8) | O(1) | C(12) | C(11) | 60.1(8) | C(22) | C(23) | C(24) | N(3) | 178.9(10) |
| C(8) | O(1) | C(12) | C(13) | −177.6(7) | C(22) | C(23) | C(24) | C(25) | −1.3(15) |
| C(8) | N(1) | C(14) | O(9) | −1.8(12) | C(4) | C(5) | C(6) | C(1) | −0.5(13) |
| C(8) | N(1) | C(14) | N(2) | 177.5(7) | C(4) | C(5) | C(6) | C(7) | 179.1(9) |
| C(8) | C(9) | C(10) | O(3) | −176.8(6) | C(4) | C(3) | C(2) | C(1) | −0.5(16) |
| C(8) | C(9) | C(10) | C(11) | −58.4(8) | C(37) | C(36) | C(35) | C(31) | −178.2(8) |
| C(30) | S(2) | C(27) | C(26) | −120.8(7) | C(24) | C(23) | C(22) | C(21) | 180.0(9) |
| C(30) | S(2) | C(27) | C(22) | 58.3(8) | C(24) | C(23) | C(22) | C(27) | 0.9(13) |
| C(30) | C(31) | C(32) | O(10) | 57.3(8) | C(2) | C(3) | C(4) | C(5) | 1.7(15) |
| C(30) | C(31) | C(32) | C(21) | −64.0(9) | C(7) | O(3) | C(10) | C(9) | −89.4(8) |
| C(30) | C(31) | C(33) | C(34) | 171.9(7) | C(7) | O(3) | C(10) | C(11) | 150.4(7) |
| C(36) | C(35) | C(31) | C(30) | −54.1(10) | C(7) | C(6) | C(1) | C(2) | −177.9(10) |
| C(36) | C(35) | C(31) | C(32) | −179.2(7) | C(33) | C(31) | C(32) | O(10) | −56.1(8) |
| C(36) | C(35) | C(31) | C(33) | 61.7(9) | C(33) | C(31) | C(32) | C(21) | −177.3(7) |
| C(19) | C(20) | C(15) | N(2) | −179.0(8) | C(28) | N(3) | C(24) | C(23) | −5.4(19) |
| C(19) | C(20) | C(15) | C(16) | −1.4(13) | C(28) | N(3) | C(24) | C(25) | 174.9(13) |
| C(19) | C(21) | C(32) | O(10) | 92.3(7) | C(29) | N(3) | C(24) | C(23) | −176.2(11) |
| C(19) | C(21) | C(32) | C(31) | −143.8(7) | C(29) | N(3) | C(24) | C(25) | 4.1(18) |
| C(19) | C(21) | C(22) | C(27) | 161.0(7) | K(1)¹ | S(1) | O(5) | C(13) | −144.2(5) |
| C(19) | C(21) | C(22) | C(23) | −18.0(11) | K(1)¹ | S(1) | O(6) | K(1)² | −94.0(7) |
| C(35) | C(36) | C(37) | C(38) | −171.8(8) | K(1)² | O(4) | C(11) | C(10) | 122.1(6) |
| C(35) | C(31) | C(32) | O(10) | −176.3(6) | K(1)² | O(4) | C(11) | C(12) | −113.6(6) |
| C(35) | C(31) | C(32) | C(21) | 62.4(9) | K(1) | O(2) | C(9) | C(8) | 94.0(10) |
| C(35) | C(31) | C(33) | C(34) | 50.1(9) | K(1) | O(2) | C(9) | C(10) | −146.8(7) |

¹1 + X, +Y, 1 + Z;
²+X, +Y, 1 + Z

### Example 23. Volixibat Potassium Salt Form II

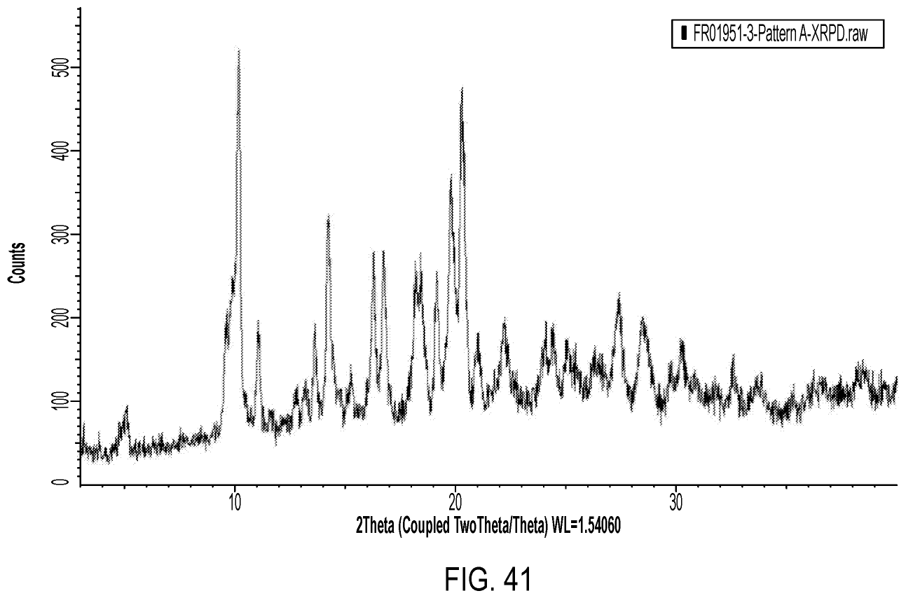

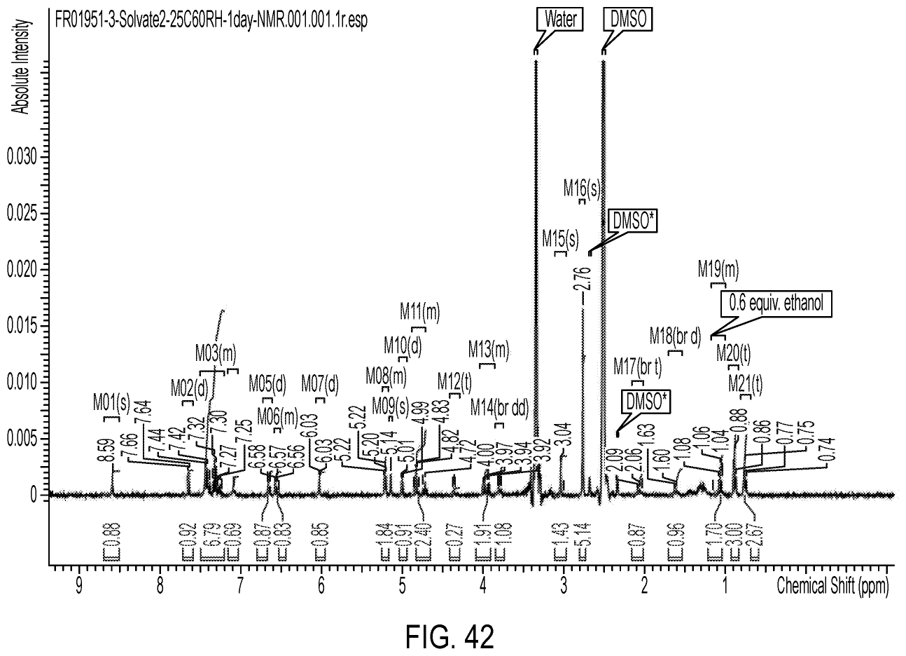

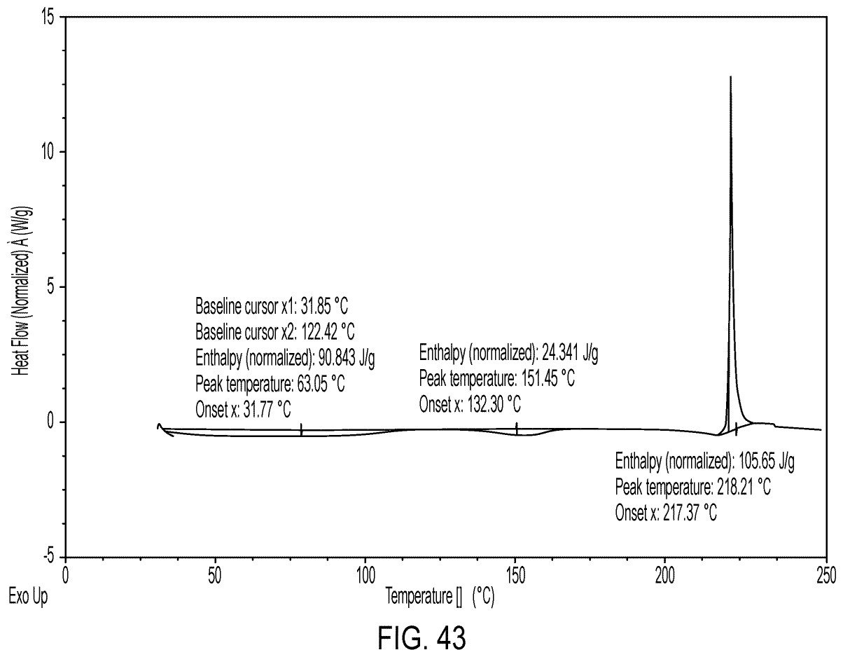

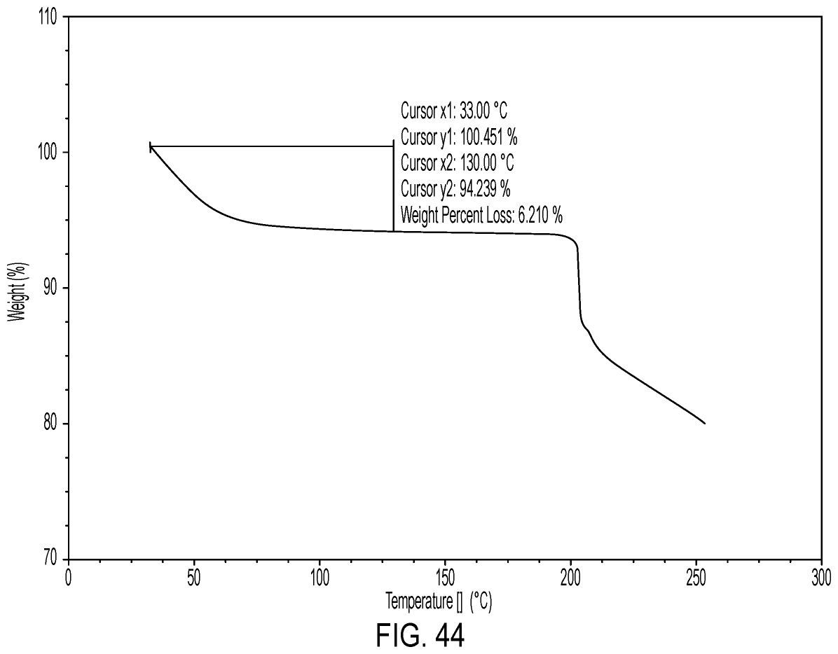

Form II is an ethanol/water solvate. It was obtained from exposure of Pattern A to ambient conditions (20-30° C.; 40-75% RH) for about 1 hour. Form II is of low to medium crystallinity as shown via XRPD (FIG. 41). It contains about 1.5 equivalent (33% by weight) of water according to KF result and 0.6 equivalent ethanol by ¹H-NMR (FIG. 42). DSC shows two broad endothermic peaks at Tₒₙₛₑₜ of 33.8° C. and at Tₒₙₛₑₜ of 132.3° C. (FIG. 43). Decomposition occurs at Tₒₙₛₑₜ of 217.4° C. (FIG. 43). TGA shows about 6.20% weight loss at 130° C. (FIG. 44).

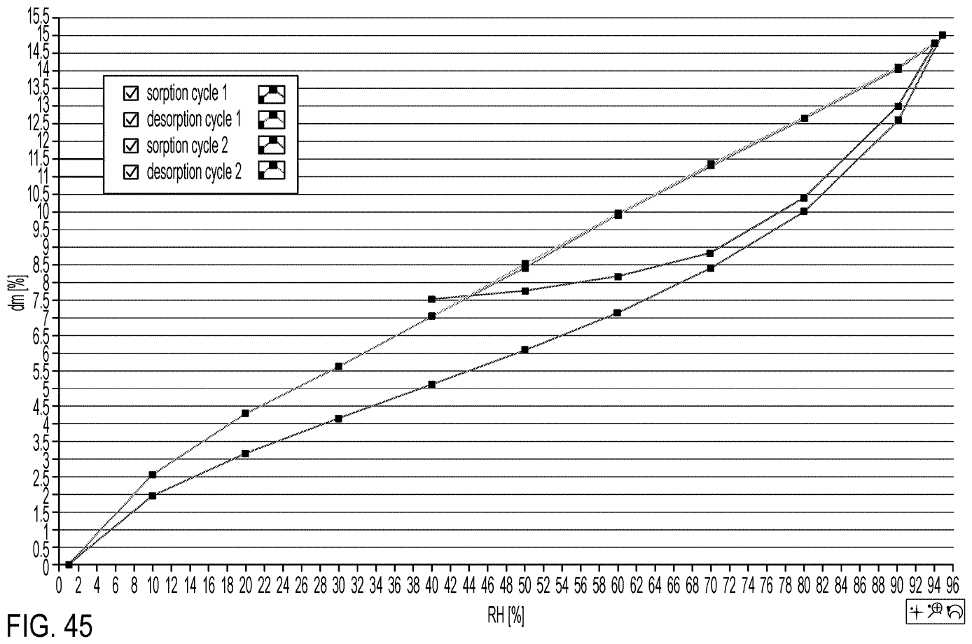

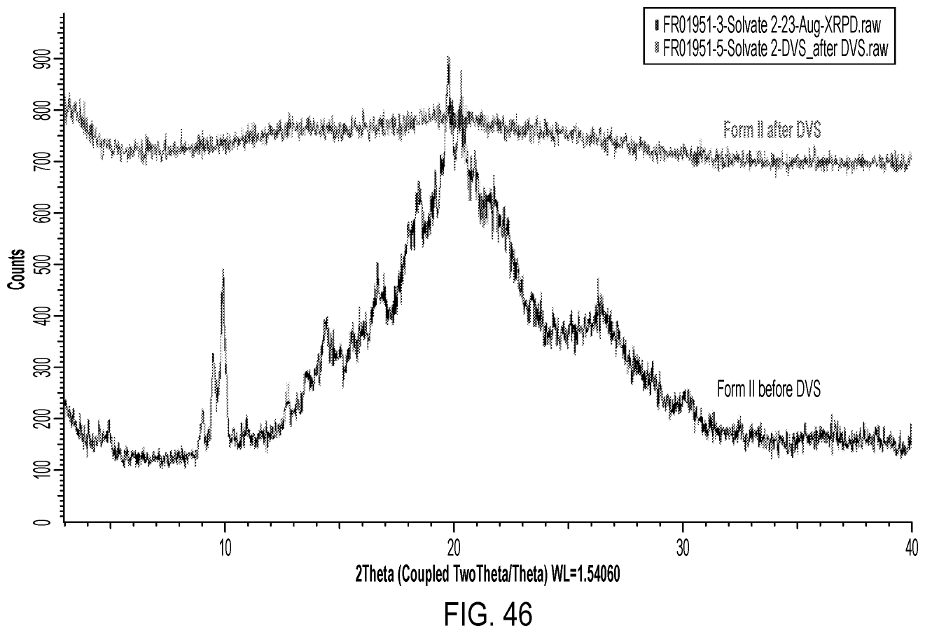

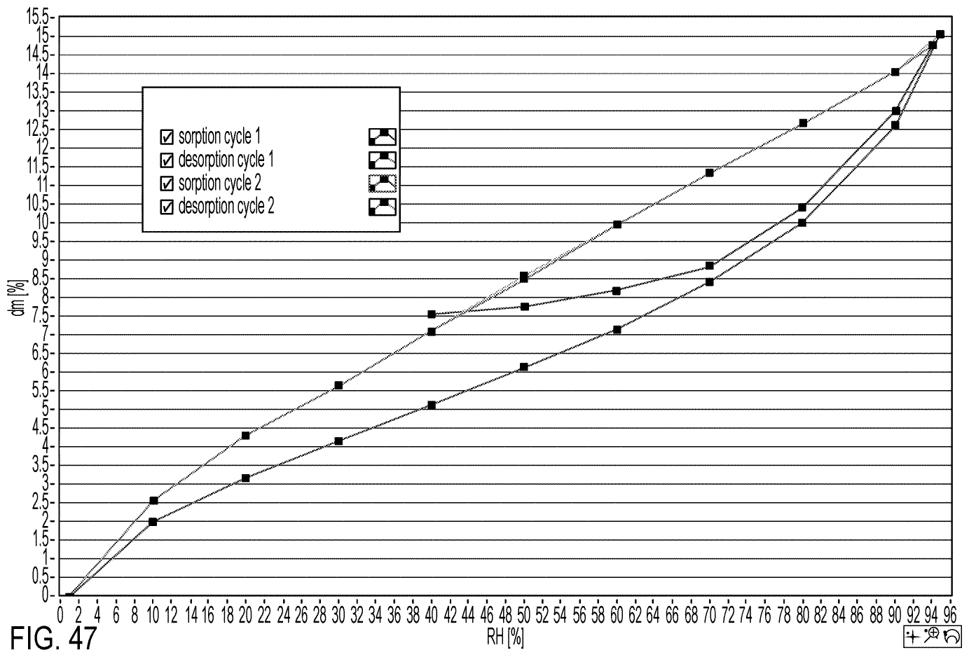

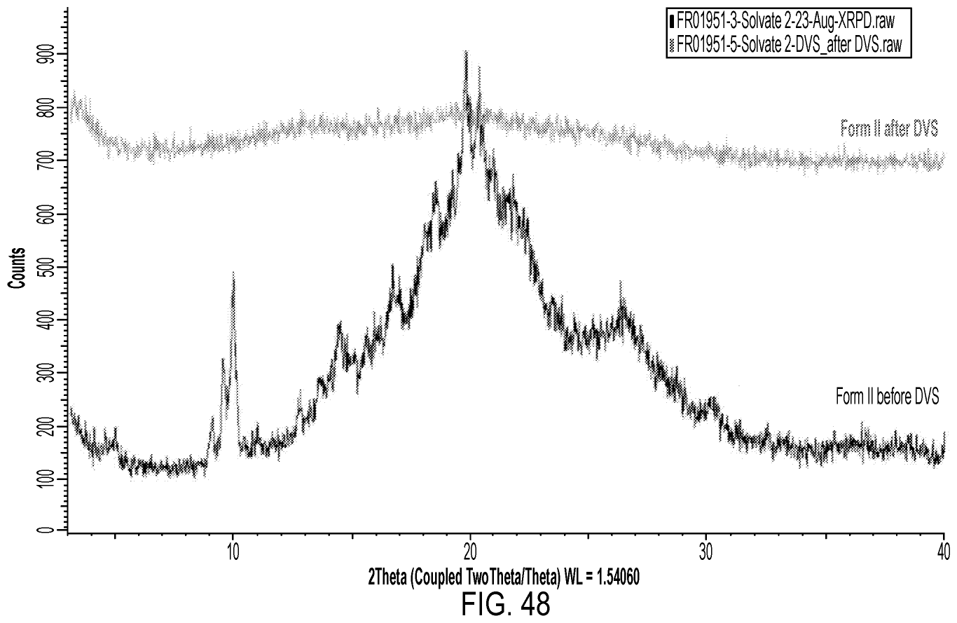

Hygroscopicity of volixibat potassium salt Form II was evaluated by dynamic vapor sorption (DVS) test at 25° C. with a cycle of 40-95-0-95-40% RH, dm/dt 0.002, equilibration time 240 min. (FIG. 45, Table 9). XRPD without Kapton film was measured after the DVS test to determine form change (FIG. 46). Form II absorbs about 7.3% water from 40% RH to 95% RH at 25° C. (1.4% from 40% RH to 70% RH and 5.9% from 70% RH to 95% RH, FIG. 47). After the DVS test, obtained sample was amorphous form (FIG. 48).

TABLE 9
Water sorption and desorption experiment of Form II.

| Relative | 1^{st} sorption | 1^{st} desorption | 2^{nd} sorption | 2^{nd} desorption |
|---|---|---|---|---|
| humidity | weight % | weight % | weight % | weight % |
| at 25° C. | change | change | change | change |
| 0% | N/A | 0 | 0 | N/A |
| 10% | N/A | 2.6 | 2.0 | N/A |
| 20% | N/A | 4.3 | 3.2 | N/A |
| 30% | N/A | 5.6 | 4.1 | N/A |
| 40% | 7.5 | 7.1 | 5.1 | 7.1 |
| 50% | 7.8 | 8.4 | 6.1 | 8.5 |
| 60% | 8.2 | 9.9 | 7.1 | 10.0 |
| 70% | 8.9 | 11.3 | 8.4 | 11.4 |
| 80% | 10.4 | 12.7 | 10.0 | 12.7 |
| 90% | 13.0 | 14.0 | 12.6 | 14.1 |
| 95% | 14.8 | 14.8 | 15.1 | 15.1 |

### Example 24. Volixibat Potassium Salt Form III

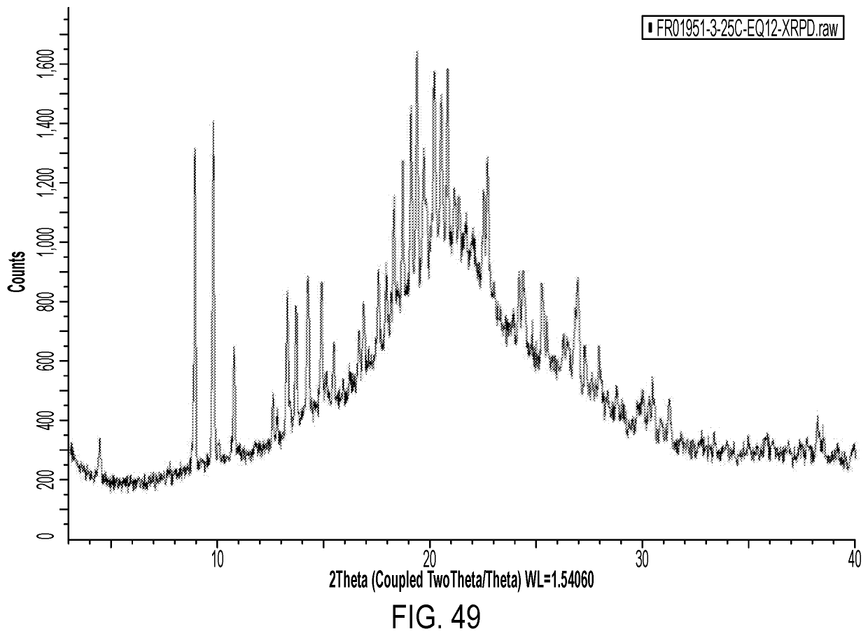

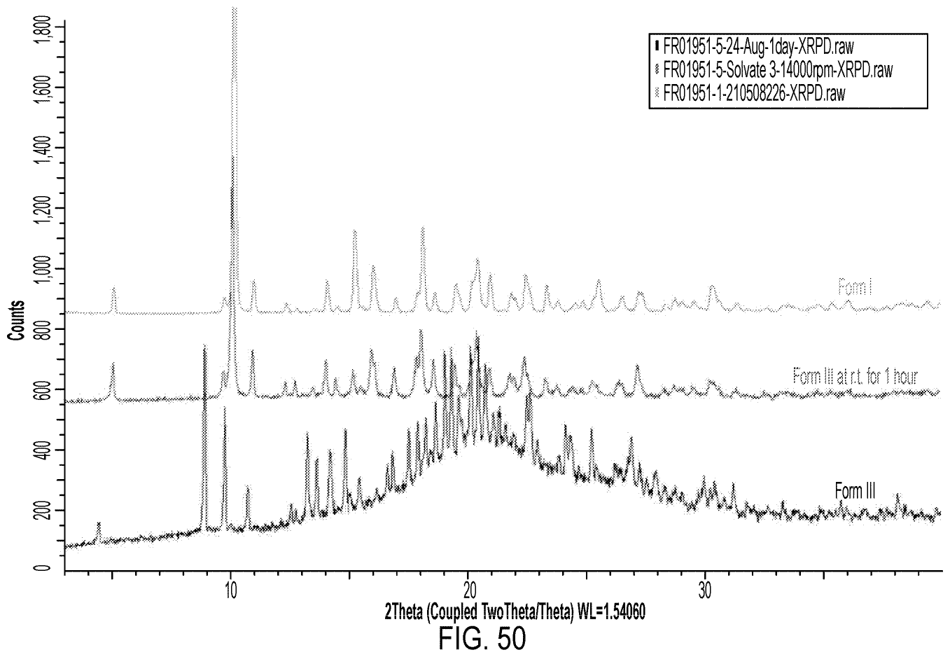

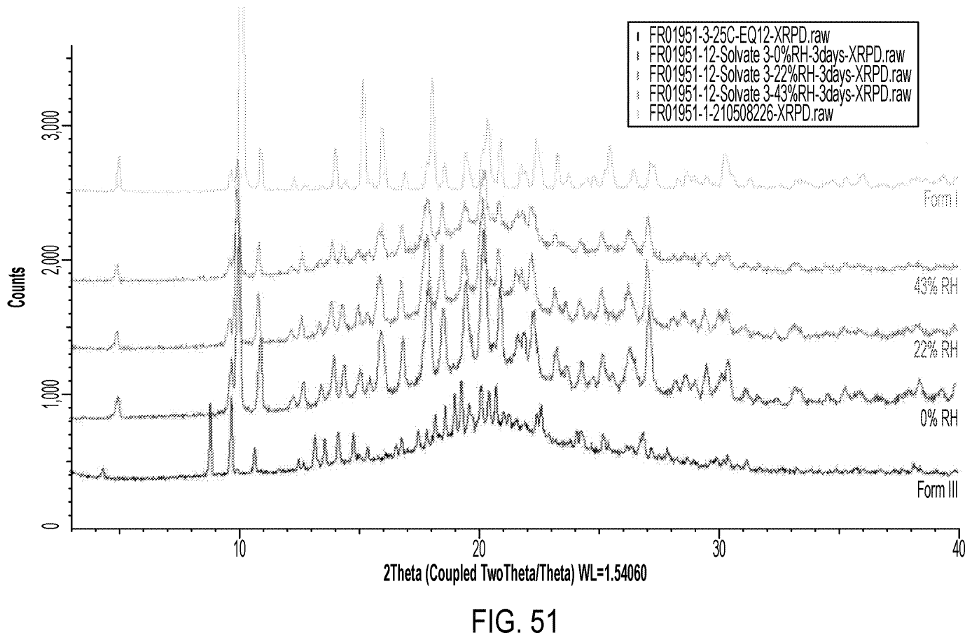

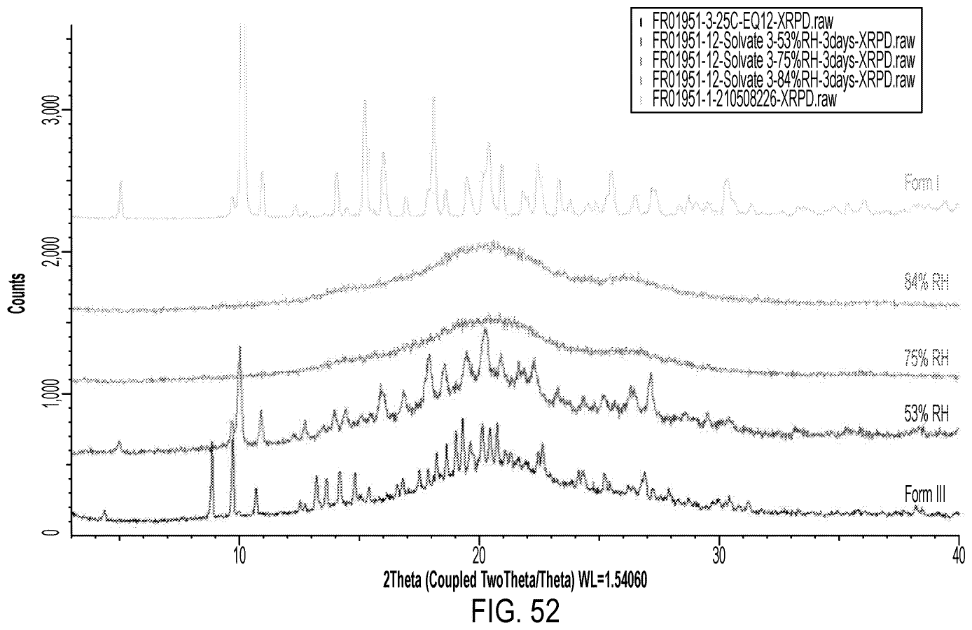

Form III is an ethanol trisolvate. It was obtained from an ethanol/water system (v:v=92.7:7.3) by equilibration experiments at 25° C. and at 50° C. for 1 day. Form III is of medium crystallinity as shown via XRPD (FIG. 49) and contains about 3 equivalent of ethanol. Form III is the stable form in ethanol/water system at high temperature and in relatively high water activity (in a.w.>0.42 at 5° C.; in a.w.>0.12 at 25° C.; in a.w.>0.08 at 50° C.). Form III is unstable and converted to Form I under ambient condition (20-30° C.; 40-75% RH) in about 1 hour (FIG. 50). 30 mg of Form III were placed in 0%, 22.5%, 43%, 53%, 75%, and 84% RH at 25° C. for 2 days and samples were characterized by XRPD without Kapton film. This showed that Form III can convert to Form I in 65% RH or below at 20-30° C. or to amorphous form in 86% RH or above at 20-30° C. (FIG. 51, FIG. 52, Table 10).

TABLE 10
Evaluation of Form III in different humidity at 25° C.

| Theoretical humidity | XRPD | Actual humidity |
|---|---|---|
| 0% RH (phosphoric pentoxide) | Form I | 16% RH |
| 22.5% RH (potassium acetate) | Form I | 37% RH |
| 43% RH (potassium carbonate) | Form I | 60% RH |
| 53% RH (magnesium nitrate) | Form I | 65% RH |
| 75% RH (sodium chloride) | Amorphous form | 86% RH |
| 84% RH (potassium chloride) | Amorphous form | 94% RH |

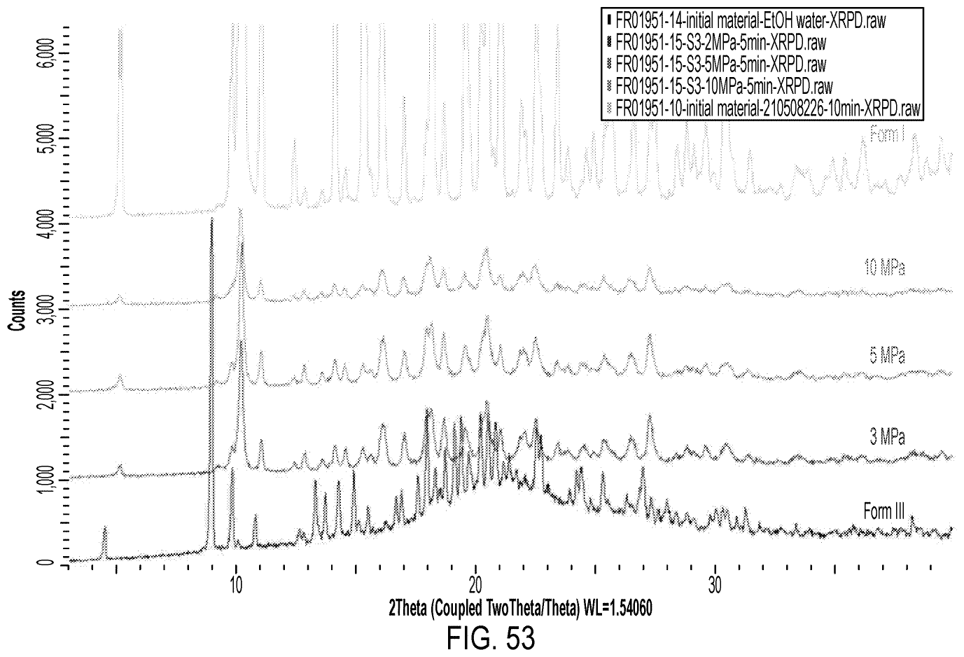

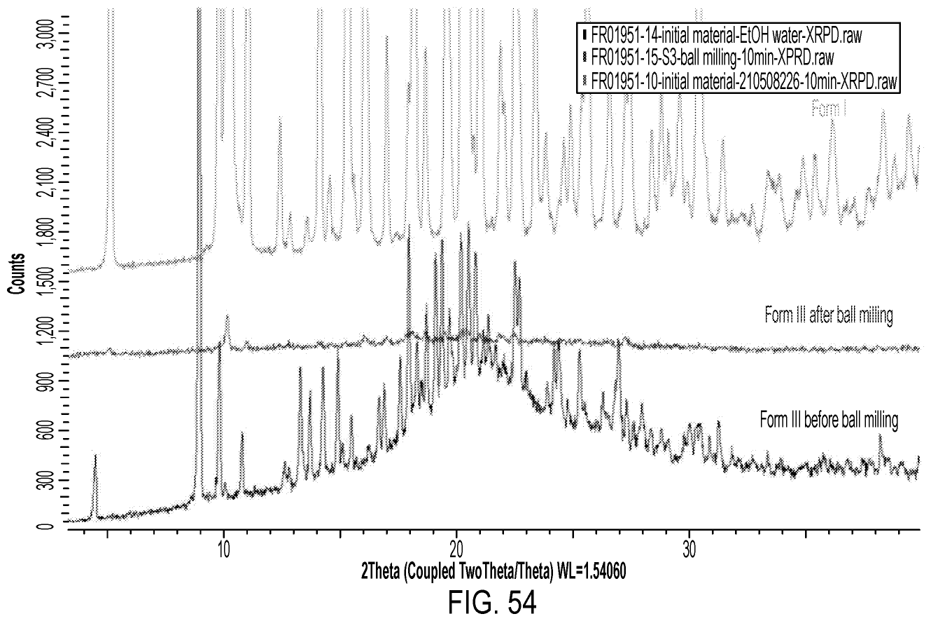

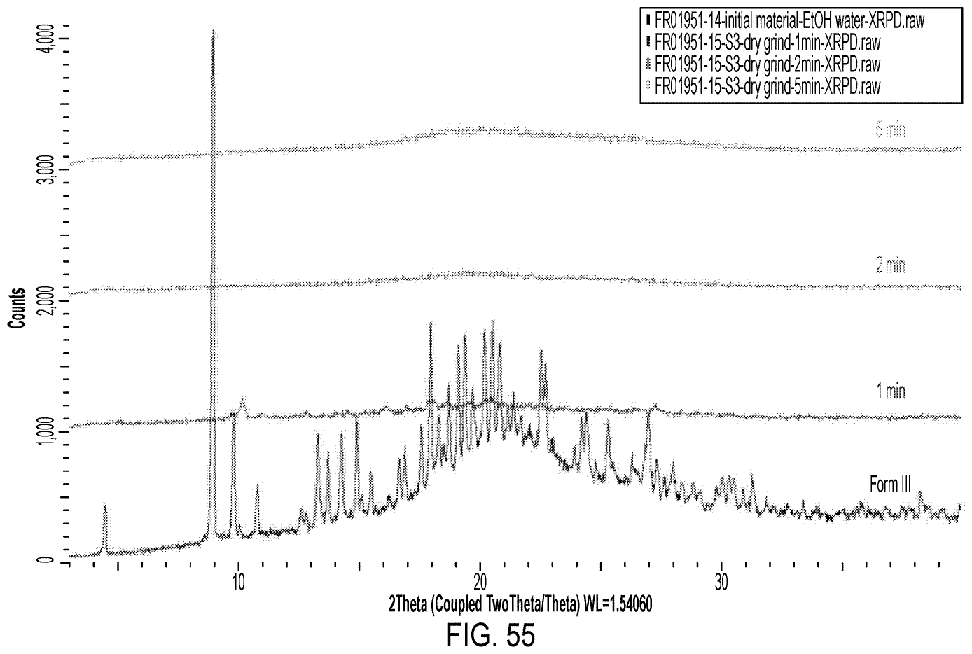

Feasibility of formulation process for volixibat potassium salt Form III were evaluated by compression, manual grinding, manual granulation simulation, and ball milling experiments. Volixibat potassium salt Form III was sensitive to compression, dry grinding, and ball milling. Form III converted to Form I after compression simulation or ball milling. 20 mg of Form III were compressed for 5 minutes under 2 MPa, 5 MPa, and 10 MPa with a hydraulic press and samples were evaluated by XRPD without Kapton film, showing a conversion to Form I and a slight decrease in crystallinity (FIG. 53). Ball milling experiments were run on about 130 mg of Form III with added grinding media, followed by grinding by ball-milling (via instrument Retsch MM400) at 25 Hz for 10 minutes. Sample was evaluated by XRPD without Kapton film, showing a conversion to Form I and a significant decrease in crystallinity (FIG. 54). Form III converted to amorphous form after dry grinding (FIG. 55) when 20 mg of Form III were ground manually with a mortar and a pestle for 1, 2, and 5 minutes and samples were evaluated by XRPD without Kapton film.

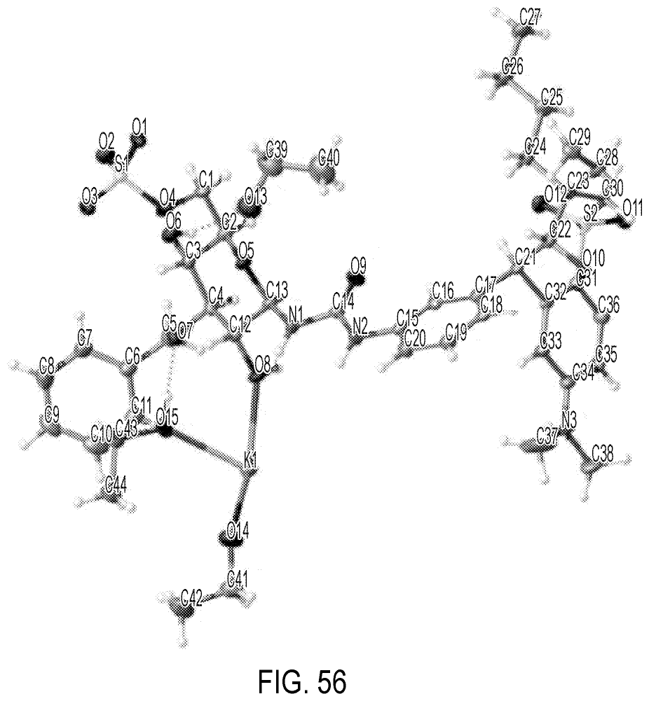

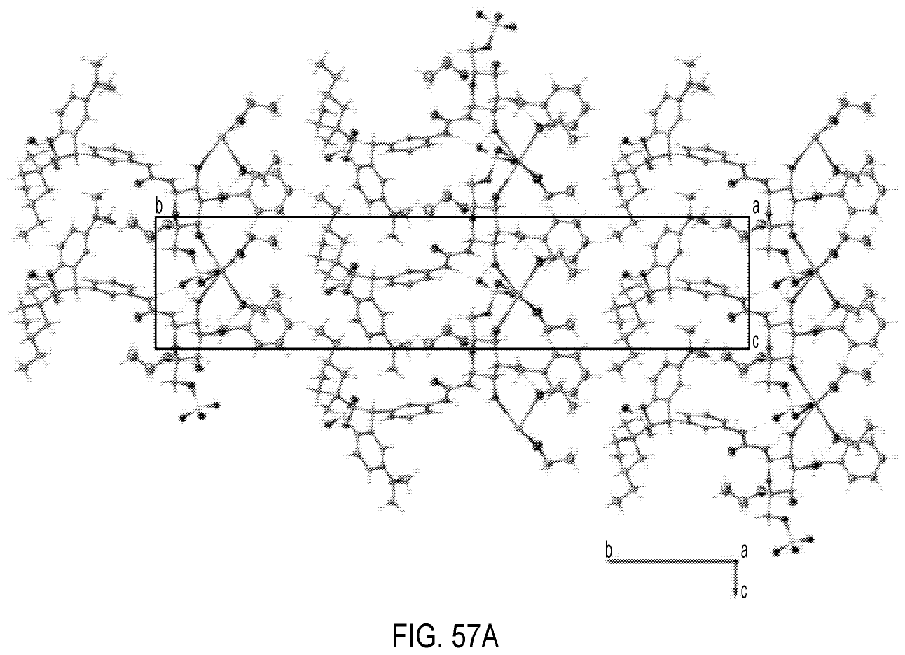

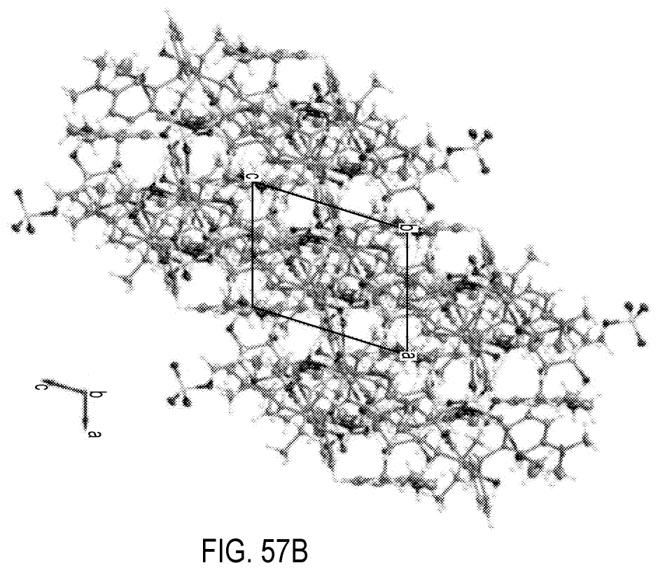

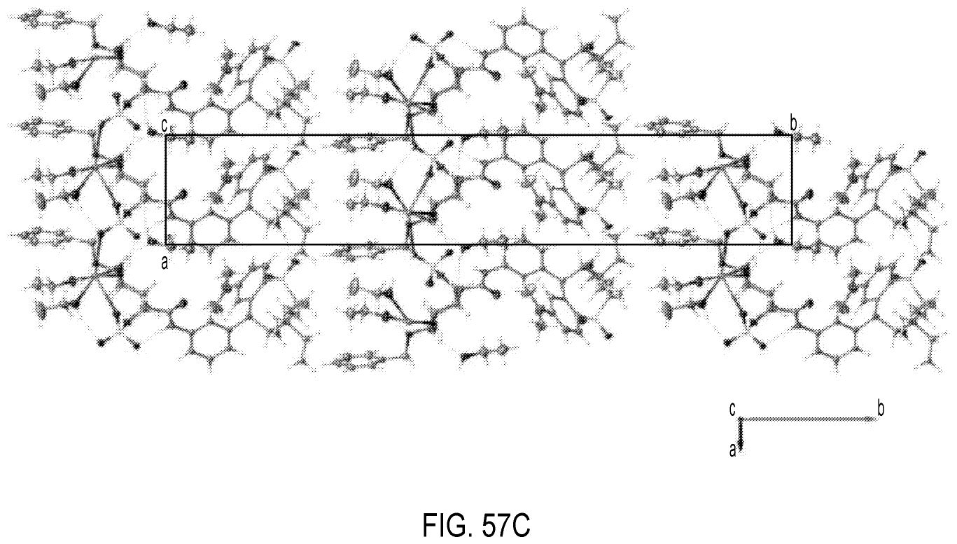

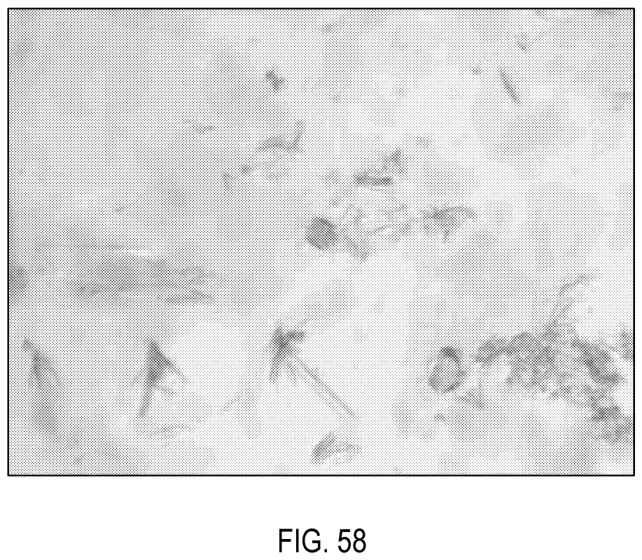

X-ray crystallographic analysis showed the crystal of Form III was a colourless block with the following dimensions: 0.20×0.20×0.10 mm³. The symmetry of the crystal structure was assigned the monoclinic space group P2₁ with the following parameters: a=7.20920(10) Å, b=39.7125(5) Å, c=9.30450(10) Å, α=90ø, β=106.548(2)°, γ=90°, cell volume V=2553.50(6) Å³, cell formula units Z=2, crystal density Dc=1.277 g/cm³, F(OOO)=1048.0, absorption coefficient μ(CuKα)=2.226 mm¹, and cell measurement temperature T=99.97(10) K. ORTEP structure is shown in FIG. 56, packing diagrams are shown in FIGS. 57A-57C, and crystals are shown in FIG. 58. Atomic coordinates and equivalent isotropic displacement parameters are displayed in Table 11, bond lengths are shown in Table 12, bond angles are shown in Table 13, hydrogen bonds are shown in Table 14, and torsion angles are shown in Table 15.

TABLE 11
Atomic coordinates (×10\hat{ }4) and equivalent isotropic displacement
parameters (A\hat{ }2 × 10\hat{ }3).

| Atom | x | y | z | U(eq) |
|---|---|---|---|---|
| K(1) | 7090(2) | 3879.1(15) | 5927.8(17) | 21.2(3) |
| S(1) | 2007(2) | 4271.6(15) | −4360.4(19) | 16.7(4) |
| S(2) | 6836(2) | 6845.4(15) | 4963(2) | 17.2(4) |
| O(5) | 4258(7) | 4660.7(19) | 164(5) | 17.3(10) |
| O(8) | 7183(7) | 4271(2) | 3635(5) | 19.2(10) |
| O(12) | 7044(7) | 6644.6(19) | 3727(6) | 20.9(11) |
| O(3) | 990(8) | 3969.0(19) | −4172(6) | 23.1(12) |
| O(1) | 727(7) | 4537.6(19) | −5142(6) | 21.7(11) |
| O(10) | 1932(7) | 6776.4(19) | 5698(5) | 17.9(11) |
| O(11) | 8406(7) | 7080(2) | 5640(6) | 22.5(11) |
| O(4) | 2873(8) | 4394(2) | −2674(6) | 21.1(11) |
| O(9) | 4584(8) | 5287(2) | 2409(6) | 27.4(13) |
| O(6) | 7824.89 | 4254.09 | −1433.8 | 22.5(11) |
| O(7) | 8209(7) | 3889.6(19) | 1368(6) | 23.0(11) |
| O(2) | 3624(7) | 4222(2) | −4979(6) | 22.3(11) |
| O(13) | 10228(8) | 4789(2) | −582(7) | 34.2(14) |
| N(1) | 4093(9) | 4727(2) | 2602(7) | 17.9(13) |
| O(15) | 6608(10) | 3521(2) | 3401(7) | 35.6(15) |
| N(2) | 2568(9) | 5105(2) | 3762(7) | 18.7(13) |
| O(14) | 4548(9) | 3560(2) | 7172(8) | 37.1(15) |
| C(13) | 5362(10) | 4652(2) | 1739(7) | 14.6(14) |
| C(14) | 3792(10) | 5060(2) | 2899(8) | 17.3(15) |
| C(12) | 6242(10) | 4303(2) | 2080(8) | 18.1(15) |
| N(3) | 6030(12) | 5976(2) | 9901(8) | 35.8(19) |
| C(22) | 2110(11) | 6660(2) | 4313(8) | 18.2(15) |
| C(21) | 3370(10) | 6338(2) | 4641(8) | 17.6(9) |
| C(6) | 10514(8) | 3451.9(17) | 1661(5) | 25.2(17) |
| C(11) | 11045(8) | 3302.8(19) | 3071(5) | 29.2(18) |
| C(10) | 11205(9) | 2954.6(19) | 3201(5) | 38(2) |
| C(9) | 10834(10) | 2755.5(17) | 1922(7) | 41(2) |
| C(8) | 10303(9) | 2904.5(18) | 512(6) | 39(2) |
| C(7) | 10143(9) | 3252.7(19) | 381(4) | 30.0(19) |
| C(1) | 4227(10) | 4671(2) | −2394(8) | 18.6(15) |
| C(23) | 2810(10) | 6943(2) | 3428(8) | 18.0(15) |
| C(17) | 2183(6) | 6018.3(17) | 4581(5) | 17.6(9) |
| C(18) | 510(6) | 6013.7(17) | 5041(5) | 17.6(9) |
| C(19) | −457(6) | 5711.9(18) | 5067(6) | 21.9(16) |
| C(20) | 250(6) | 5414.7(17) | 4633(6) | 21.8(16) |
| C(15) | 1923(6) | 5419.3(17) | 4174(5) | 17.5(15) |
| C(16) | 2889(5) | 5721.1(18) | 4148(5) | 18.2(15) |
| C(4) | 7594(10) | 4232(2) | 1116(7) | 18.9(15) |
| C(30) | 4728(10) | 7098(2) | 4399(8) | 15.2(14) |
| C(25) | 3529(11) | 7086(2) | 916(8) | 21.4(16) |
| C(24) | 2943(11) | 6816(2) | 1896(8) | 19.0(15) |
| C(5) | 10217(11) | 3829(3) | 1509(10) | 28.5(18) |
| C(3) | 6532(10) | 4286(2) | −551(7) | 19.4(15) |
| C(26) | 3949(13) | 6947(3) | −460(9) | 27.1(18) |
| C(2) | 5508(11) | 4629(2) | −783(8) | 18.3(15) |
| C(28) | 1360(11) | 7240(2) | 3170(8) | 18.0(15) |
| C(31) | 6553(7) | 6580.5(18) | 6382(4) | 19.0(15) |
| C(32) | 4989(6) | 6363.4(18) | 6187(4) | 14.0(14) |
| C(33) | 4843(6) | 6159.0(18) | 7362(5) | 18.2(15) |
| C(34) | 6261(7) | 6171.7(19) | 8733(4) | 22.9(16) |
| C(35) | 7826(6) | 6389(2) | 8928(4) | 25.1(17) |
| C(36) | 7972(6) | 6593.2(19) | 7753(5) | 24.2(17) |
| C(27) | 4546(14) | 7219(3) | −1368(10) | 33(2) |
| C(29) | −646(11) | 7176(2) | 2051(9) | 22.8(16) |
| C(43) | 6109(13) | 3189(2) | 2872(9) | 28.8(18) |
| C(38) | 7393(16) | 5992(3) | 11416(10) | 41(2) |
| C(41) | 4475(13) | 3382(2) | 8468(10) | 35(2) |
| C(40) | 9638(17) | 5382(3) | −441(15) | 58(3) |
| C(39) | 9821(16) | 5088(3) | −1447(13) | 50(3) |
| C(44) | 6345(18) | 2959(3) | 4231(10) | 45(3) |
| C(42) | 3920(30) | 3015(3) | 8101(15) | 77(5) |
| C(37) | 4220(20) | 5795(4) | 9703(13) | 75(5) |

TABLE 12
Bond lengths [A].

| Atom | Atom | Length/Å | Atom | Atom | Length/Å |
|---|---|---|---|---|---|
| K(1) | O(8) | 2.657(5) | C(22) | C(23) | 1.562(10) |
| K(1) | O(3)¹ | 2.862(6) | C(21) | C(17) | 1.522(8) |
| K(1) | O(6)² | 2.790(3) | C(21) | C(32) | 1.576(7) |
| K(1) | O(2)² | 2.757(5) | C(6) | C(11) | 1.3900 |
| K(1) | O(15) | 2.684(6) | C(6) | C(7) | 1.3900 |
| K(1) | O(14) | 2.740(6) | C(6) | C(5) | 1.512(9) |
| S(1) | O(3) | 1.444(5) | C(11) | C(10) | 1.3900 |
| S(1) | O(1) | 1.454(5) | C(10) | C(9) | 1.3900 |
| S(1) | O(4) | 1.591(5) | C(9) | C(8) | 1.3900 |
| S(1) | O(2) | 1.454(5) | C(8) | C(7) | 1.3900 |
| S(2) | O(12) | 1.442(5) | C(1) | C(2) | 1.529(10) |
| S(2) | O(11) | 1.463(5) | C(23) | C(30) | 1.548(10) |
| S(2) | C(30) | 1.772(7) | C(23) | C(24) | 1.541(10) |
| S(2) | C(31) | 1.745(4) | C(23) | C(28) | 1.549(10) |
| O(5) | C(13) | 1.455(8) | C(17) | C(18) | 1.3900 |
| O(5) | C(2) | 1.435(9) | C(17) | C(16) | 1.3900 |
| O(8) | C(12) | 1.418(8) | C(18) | C(19) | 1.3900 |
| O(10) | C(22) | 1.409(9) | C(19) | C(20) | 1.3900 |
| O(4) | 7C(1) | 1.444(9) | C(20) | C(15) | 1.3900 |
| O(9) | C(14) | 1.221(9) | C(0) | C(13) | 1.3900 |
| O(6) | C(3) | 1.413(7) | C(0) | C(3) | 1.536(6) |
| O(7) | C(4) | 1.428(9) | C(25) | C(24) | 1.542(6) |
| O(7) | C(5) | 1.436(9) | C(25) | C(26) | 1.502(10) |
| O(13) | C(39) | 1.419(12) | C(3) | C(2) | 1.534(10) |
| N(1) | C(13) | 1.410(9) | C(26) | C(27) | 1.509(12) |
| N(1) | C(14) | 1.382(10) | C(28) | C(29) | 1.543(10) |
| O(15) | C(43) | 1.420(10) | C(31) | C(32) | 1.3900 |
| N(2) | C(14) | 1.363(10) | C(31) | C(36) | 1.3900 |
| N(2) | C(15) | 1.422(7) | C(32) | C(33) | 1.3900 |
| O(14) | C(41) | 1.412(11) | C(33) | C(34) | 1.3900 |
| C(13) | C(12) | 1.522(10) | C(34) | C(35) | 1.3900 |
| C(12) | C(4) | 1.527(10) | C(35) | C(36) | 1.3900 |
| N(3) | C(34) | 1.384(8) | C(43) | C(44) | 1.528(6) |
| N(3) | C(38) | 1.473(11) | C(41) | C(42) | 1.528(7) |
| N(3) | C(37) | 1.455(14) | C(40) | C(39) | 1.524(7) |
| C(22) | C(21) | 1.547(10) | | | |

¹1 + X, +Y, 1 + Z;
²+X, +Y, 1 + Z

TABLE 13
Bond angles [°].

| Atom | Atom | Atom | Angle/° | Atom | Atom | Atom | Angle/° |
|---|---|---|---|---|---|---|---|
| O(8) | K(1) | O(3)¹ | 69.60(15) | C(11) | C(6) | C(7) | 120.0 |
| O(8) | K(1) | O(6)² | 110.59(16) | C(11) | C(6) | C(5) | 120.2(5) |
| O(8) | K(1) | O(2)² | 72.13(16) | C(7) | C(6) | C(5) | 119.7(4) |
| O(8) | K(1) | O(15) | 68.50(17) | C(10) | C(11) | C(6) | 120.0 |
| O(8) | K(1) | O(14) | 141.50(18) | C(11) | C(10) | C(9) | 120.0 |
| O(6)² | K(1) | O(3)¹ | 91.13(12) | C(8) | C(9) | C(10) | 120.0 |
| O(2)² | K(1) | O(3)¹ | 137.87(16) | C(7) | C(8) | C(9) | 120.0 |
| O(2)² | K(1) | O(6)² | 86.25(13) | C(8) | C(7) | C(6) | 120.0 |
| O(15) | K(1) | O(3)¹ | 85.46(18) | O(4) | C(1) | C(2) | 106.3(6) |
| O(15) | K(1) | O(6)² | 176.58(16) | C(30) | C(23) | C(22) | 110.4(6) |
| O(15) | K(1) | O(2)² | 96.49(19) | C(30) | C(23) | C(28) | 103.8(6) |
| O(15) | K(1) | O(14) | 101.0(2) | C(24) | C(23) | C(22) | 111.5(6) |
| O(14) | K(1) | O(3)¹ | 148.50(18) | C(24) | C(23) | C(30) | 113.0(6) |
| O(14) | K(1) | O(6)² | 81.78(17) | C(24) | C(23) | C(28) | 108.9(6) |
| O(14) | K(1) | O(2)² | 72.56(17) | C(28) | C(23) | C(22) | 109.0(6) |
| O(3) | S(1) | O(1) | 113.2(3) | C(18) | C(17) | C(21) | 121.8(4) |
| O(3) | S(1) | O(4) | 101.8(3) | C(18) | C(17) | C(16) | 120.0 |
| O(3) | S(1) | O(2) | 115.2(3) | C(16) | C(17) | C(21) | 118.0(4) |
| O(1) | S(1) | O(4) | 106.6(3) | C(17) | C(3) | C(4) | 120.0 |
| O(2) | S(1) | O(1) | 111.6(3) | C(20) | C(19) | C(18) | 120.0 |
| O(2) | S(1) | O(4) | 107.4(3) | C(19) | C(20) | C(15) | 120.0 |
| O(12) | S(2) | O(11) | 116.9(3) | C(20) | C(15) | N(2) | 116.8(4) |
| O(12) | S(2) | C(30) | 110.7(3) | C(20) | C(15) | C(16) | 120.0 |
| O(12) | S(2) | C(31) | 109.3(3) | C(16) | C(15) | N(2) | 123.2(4) |
| O(11) | S(2) | C(30) | 105.0(3) | C(15) | C(16) | C(17) | 120.0 |
| O(11) | S(2) | C(31) | 107.5(3) | O(7) | C(4) | C(12) | 107.4(6) |
| C(31) | S(2) | C(30) | 106.9(3) | O(7) | C(4) | C(3) | 110.0(6) |
| C(2) | O(5) | C(13) | 110.9(5) | C(12) | C(4) | C(3) | 110.4(5) |
| C(12) | O(8) | K(1) | 139.2(4) | C(23) | C(30) | S(2) | 119.7(5) |
| S(1) | O(3) | K(1)3 | 130.2(3) | C(26) | C(25) | C(24) | 113.9(6) |
| C(1) | O(4) | S(1) | 117.8(4) | C(23) | C(24) | C(25) | 114.7(6) |
| C(3) | O(6) | K(1)4 | 124.6(3) | O(7) | C(5) | C(6) | 106.7(6) |
| C(4) | O(7) | C(5) | 115.7(6) | O(6) | C(3) | C(4) | 110.6(5) |
| S(1) | O(2) | K(1)4 | 134.9(3) | O(6) | C(3) | C(2) | 111.6(6) |
| C(14) | N(1) | C(13) | 118.6(6) | C(2) | C(3) | C(4) | 110.4(6) |
| C(43) | O(15) | K(1) | 138.8(5) | C(25) | C(26) | C(27) | 111.9(7) |
| C(14) | N(2) | C(15) | 126.1(6) | O(5) | C(2) | C(1) | 106.4(6) |
| C(41) | O(14) | K(1) | 141.7(5) | O(5) | C(2) | C(3) | 110.7(6) |
| O(5) | C(13) | C(12) | 107.9(5) | C(1) | C(2) | C(3) | 111.6(6) |
| N(1) | C(13) | O(5) | 108.1(6) | C(29) | C(28) | C(23) | 116.1(6) |
| N(1) | C(13) | C(12) | 111.9(6) | C(32) | C(31) | S(2) | 122.7(3) |
| O(9) | C(14) | N(1) | 120.9(7) | C(32) | C(31) | C(36) | 120.0 |
| O(9) | C(14) | N(2) | 125.0(7) | C(36) | C(31) | S(2) | 117.3(3) |
| N(2) | C(14) | N(1) | 114.0(6) | C(31) | C(32) | C(21) | 121.5(4) |
| O(8) | C(12) | C(13) | 110.0(6) | C(31) | C(32) | C(33) | 120.0 |
| O(8) | C(12) | C(4) | 112.6(5) | C(33) | C(32) | C(21) | 118.5(4) |
| C(13) | C(12) | C(4) | 110.1(6) | C(34) | C(33) | C(32) | 120.0 |
| C(34) | N(3) | C(38) | 122.0(7) | N(3) | C(34) | C(33) | 118.5(4) |
| C(34) | N(3) | C(37) | 118.8(7) | N(3) | C(34) | C(35) | 121.4(4) |
| C(37) | N(3) | C(38) | 118.2(8) | C(33) | C(34) | C(35) | 120.0 |
| O(10) | C(22) | C(21) | 107.0(6) | C(36) | C(35) | C(34) | 120.0 |
| O(10) | C(22) | C(23) | 111.9(6) | C(35) | C(36) | C(31) | 120.0 |
| C(21) | C(22) | C(23) | 116.1(6) | O(15) | C(43) | C(44) | 108.1(7) |
| C(22) | C(21) | C(32) | 111.5(6) | O(14) | C(41) | C(42) | 111.0(8) |
| C(17) | C(21) | C(22) | 112.9(6) | O(13) | C(39) | C(40) | 109.4(9) |
| C(17) | C(21) | C(32) | 110.5(5) | | | | |

¹1 + X, +Y, 1 + Z;
²+X, +Y, 1 + Z;
3−1 + X, +Y, −1 + Z;
4+X, +Y, −1 + Z

TABLE 14
Hydrogen bonds.

| D | H | A | d(D − H)/Å | d(H − A)/Å | d(D − A)/Å | D − H − A/° |
|---|---|---|---|---|---|---|
| O(8) | H(8) | O(1)¹ | 0.870(14) | 1.88(3) | 2.699(7) | 156(5) |
| O(10) | H(10) | O(11)² | 0.84 | 1.98 | 2.800(7) | 165.4 |
| O(6) | H(6) | O(13) | 0.8399(0) | 1.956(8) | 2.713(8) | 149.52(19) |
| O(13) | H(13A) | O(5)³ | 0.84 | 2.12 | 2.834(8) | 142.1 |
| N(1) | H(1) | O(2)⁴ | 0.88 | 2.34 | 3.105(8) | 144.7 |
| O(15) | H(15) | O(7) | 0.873(14) | 2.11(7) | 2.879(8) | 147(11) |
| N(2) | H(2) | O(1)⁴ | 0.88 | 2.07 | 2.940(8) | 170.8 |
| O(14) | H(14) | O(3)⁴ | 0.872(14) | 2.15(4) | 2.991(8) | 161(9) |

¹1 + X, +Y, 1 + Z;
²−1 + X, +Y, +Z;
³1 + X, +Y, +Z;
⁴+X, +Y, 1 + Z

TABLE 15
Torsion angles [°].

| A | B | C | D | Angle/° | A | B | C | D | Angle/° |
|---|---|---|---|---|---|---|---|---|---|
| K(1) | O(8) | C(12) | C(13) | 123.4(6) | C(22) | C(23) | C(28) | C(29) | −70.1(8) |
| K(1) | O(8) | C(12) | C(4) | −113.4(6) | C(21) | C(22) | C(23) | C(30) | −68.0(8) |
| K(1)¹ | O(6) | C(3) | C(4) | 133.3(5) | C(21) | C(22) | C(23) | C(24) | 58.4(8) |
| K(1)¹ | O(6) | C(3) | C(2) | −103.4(6) | C(21) | C(22) | C(23) | C(28) | 178.6(6) |
| K(1) | O(15) | C(43) | C(44) | −16.7(12) | C(21) | C(17) | C(18) | C(19) | −175.5(5) |
| K(1) | O(14) | C(41) | C(42) | −113.4(11) | C(21) | C(17) | C(16) | C(15) | 175.6(5) |
| S(1) | O(4) | C(1) | C(2) | −153.4(5) | C(21) | C(32) | C(33) | C(34) | 179.0(5) |
| S(2) | C(31) | C(32) | C(21) | 1.2(5) | C(6) | C(11) | C(10) | C(9) | 0.0 |
| S(2) | C(31) | C(32) | C(33) | −179.8(4) | C(11) | C(6) | C(7) | C(8) | 0.0 |
| S(2) | C(31) | C(36) | C(35) | 179.9(4) | C(11) | C(6) | C(5) | O(7) | −90.9(6) |
| O(5) | C(13) | C(12) | O(8) | −174.5(5) | C(11) | C(10) | C(9) | C(8) | 0.0 |
| O(5) | C(13) | C(12) | C(4) | 60.9(7) | C(10) | C(9) | C(8) | C(7) | 0.0 |
| O(8) | C(12) | C(4) | O(7) | 62.9(7) | C(9) | C(8) | C(7) | C(6) | 0.0 |
| O(8) | C(12) | C(4) | C(3) | −177.2(6) | C(7) | C(6) | C(11) | C(10) | 0.0 |
| O(12) | S(2) | C(30) | C(23) | 44.3(6) | C(7) | C(6) | C(5) | O(7) | 85.3(6) |
| O(12) | S(2) | C(31) | C(32) | −62.9(4) | C(23) | C(22) | C(21) | C(17) | −144.5(6) |
| O(12) | S(2) | C(31) | C(36) | 117.2(3) | C(23) | C(22) | C(21) | C(32) | 90.4(7) |
| O(3) | S(1) | O(4) | C(1) | 171.6(5) | C(17) | C(21) | C(32) | C(31) | 163.4(4) |
| O(3) | S(1) | O(2) | K(1)¹ | −62.0(5) | C(17) | C(21) | C(32) | C(33) | −15.6(6) |
| O(1) | S(1) | O(3) | K(1)² | −25.0(5) | C(17) | C(18) | C(19) | C(20) | 0.0 |
| O(1) | S(1) | O(4) | C(1) | −69.5(6) | C(18) | C(17) | C(16) | C(15) | 0.0 |
| O(1) | S(1) | O(2) | K(1)¹ | 167.2(4) | C(18) | C(19) | C(20) | C(15) | 0.0 |
| O(10) | C(22) | C(21) | C(17) | 89.8(7) | C(19) | C(20) | C(15) | N(2) | 179.4(5) |
| O(10) | C(22) | C(21) | C(32) | −35.3(8) | C(19) | C(20) | C(15) | C(16) | 0.0 |
| O(10) | C(22) | C(23) | C(30) | 55.3(8) | C(20) | C(15) | C(16) | C(17) | 0.0 |
| O(10) | C(22) | C(23) | C(24) | −178.3(6) | C(15) | N(2) | C(14) | O(9) | 3.6(12) |
| O(10) | C(22) | C(23) | C(28) | −58.1(8) | C(15) | N(2) | C(14) | N(1) | −176.5(6) |
| O(11) | S(2) | C(30) | C(23) | 171.3(5) | C(16) | C(17) | C(18) | C(19) | 0.0 |
| O(11) | S(2) | C(31) | C(32) | 169.2(3) | C(4) | O(7) | C(5) | C(6) | −176.2(5) |
| O(11) | S(2) | C(31) | C(36) | −10.6(4) | C(4) | C(3) | C(2) | O(5) | −54.4(8) |
| O(4) | S(1) | O(3) | K(1)² | 89.0(4) | C(4) | C(3) | C(2) | C(1) | −172.7(6) |
| O(4) | S(1) | O(2) | K(1)¹ | 50.7(5) | C(30) | S(2) | C(31) | C(32) | 56.9(4) |
| O(4) | C(1) | C(2) | O(5) | −63.4(7) | C(30) | S(2) | C(31) | C(36) | −122.9(3) |
| O(4) | C(1) | C(2) | C(3) | 57.4(8) | C(30) | C(23) | C(24) | C(25) | −57.9(8) |
| O(6) | C(3) | C(2) | O(5) | −177.9(5) | C(30) | C(23) | C(28) | C(29) | 172.2(6) |
| O(6) | C(3) | C(2) | C(1) | 63.8(7) | C(24) | C(23) | C(30) | S(2) | −61.7(8) |
| O(7) | C(4) | C(3) | O(6) | −67.5(8) | C(24) | C(23) | C(28) | C(29) | 51.6(8) |
| O(7) | C(4) | C(3) | C(2) | 168.4(6) | C(24) | C(25) | C(26) | C(27) | −179.1(7) |
| O(2) | S(1) | O(3) | K(1)² | −155.1(3) | C(5) | O(7) | C(4) | C(12) | −138.4(6) |
| O(2) | S(1) | O(4) | C(1) | 50.2(6) | C(5) | O(7) | C(4) | C(3) | 101.4(7) |
| N(1) | C(13) | C(12) | O(8) | −55.7(8) | C(5) | C(6) | C(11) | C(10) | 176.2(6) |
| N(1) | C(13) | C(12) | C(4) | 179.7(6) | C(24) | C(5) | C(6) | C(7) | −176.2(6) |
| N(2) | C(15) | C(16) | C(17) | −179.3(5) | C(26) | C(25) | C(24) | C(23) | 171.0(7) |
| C(13) | O(5) | C(2) | C(1) | −175.2(5) | C(2) | O(5) | C(13) | N(1) | 172.7(6) |
| C(13) | O(5) | C(2) | C(3) | 63.3(7) | C(2) | O(5) | C(13) | C(12) | −66.1(7) |
| C(13) | N(1) | C(14) | O(9) | 1.0(10) | C(28) | C(23) | C(30) | S(2) | −179.5(5) |
| C(13) | N(1) | C(14) | N(2) | −178.9(6) | C(28) | C(23) | C(24) | C(25) | 56.9(8) |
| C(13) | C(12) | C(4) | O(7) | −174.0(5) | C(31) | S(2) | C(30) | C(23) | −74.7(6) |
| C(13) | C(12) | C(4) | C(3) | −54.0(8) | C(31) | C(8) | C(9) | C(10) | 0.0 |
| C(14) | N(1) | C(13) | O(5) | −87.1(7) | C(32) | C(21) | C(17) | C(18) | 91.3(5) |
| C(14) | N(1) | C(13) | C(12) | 154.2(6) | C(32) | C(21) | C(17) | C(16) | −84.3(5) |
| C(14) | N(2) | C(15) | C(20) | 160.0(6) | C(32) | C(31) | C(36) | C(35) | 0.0 |
| C(14) | N(2) | C(15) | C(16) | −20.6(9) | C(32) | C(33) | C(34) | N(3) | 177.2(6) |
| C(12) | C(4) | C(3) | O(6) | 174.1(6) | C(32) | C(33) | C(34) | C(35) | 0.0 |
| C(12) | C(4) | C(3) | C(2) | 50.0(8) | C(33) | C(11) | C(12) | C(13) | 0.0 |
| N(3) | C(34) | C(35) | C(36) | −177.1(6) | C(34) | C(35) | C(36) | C(31) | 0.0 |
| C(22) | C(21) | C(17) | C(18) | −34.4(7) | C(36) | C(31) | C(32) | C(21) | −179.0(5) |
| C(22) | C(21) | C(17) | C(16) | 150.0(5) | C(36) | C(31) | C(32) | C(33) | 0.0 |
| C(22) | C(21) | C(32) | C(31) | −70.1(6) | C(38) | N(3) | C(34) | C(33) | −175.7(7) |
| C(22) | C(21) | C(32) | C(33) | 110.8(5) | C(38) | N(3) | C(34) | C(35) | 1.5(11) |
| C(22) | C(23) | C(30) | S(2) | 63.8(7) | C(37) | N(3) | C(34) | C(33) | −7.0(12) |
| C(22) | C(23) | C(24) | C(25) | 177.1(6) | C(37) | N(3) | C(34) | C(35) | 170.2(9) |

¹+X, +Y, −1 + Z;
²−1 + X, +Y, −1 + Z

### Example 25. Volixibat Potassium Salt Form VI

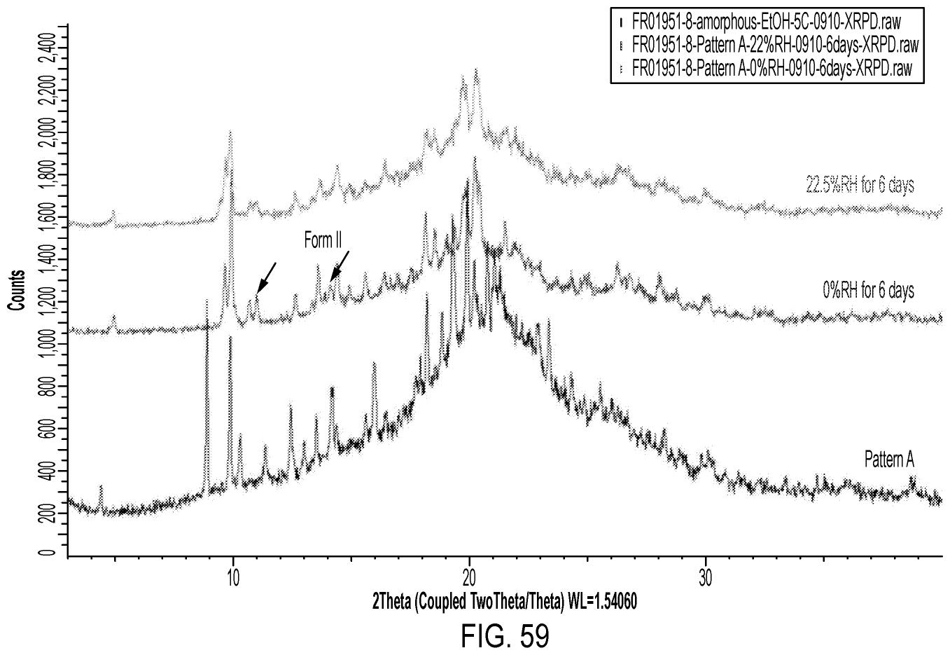

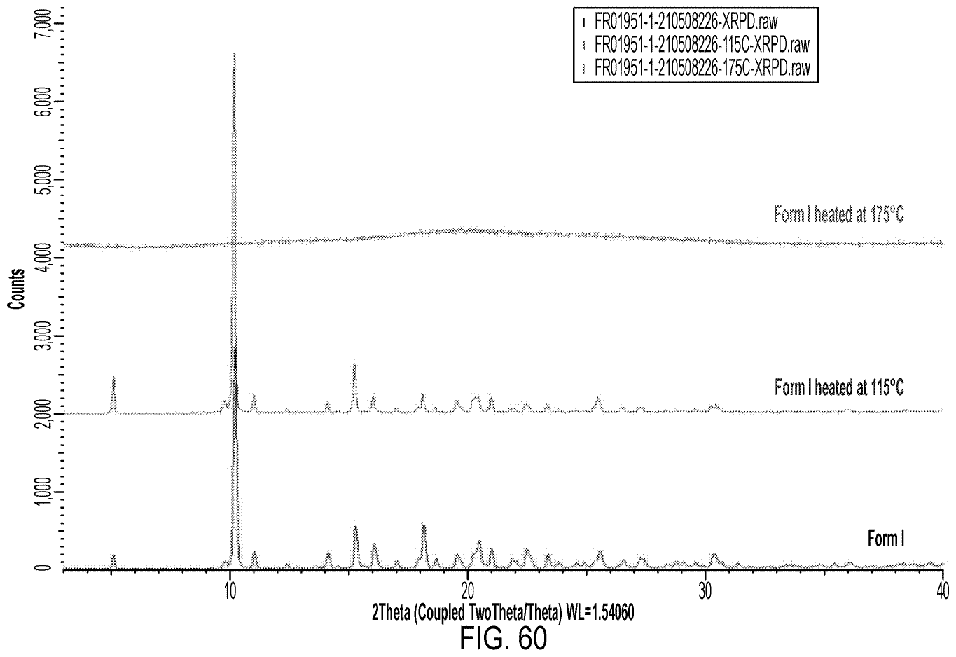

Form VI is an ethanol/water solvate. Form VI was formed by placing Pattern A in 0% RH (as shown by phosphoric pentoxide, actual humidity of 16% RH) and 22.5% RH (as shown by phosphoric pentoxide, actual humidity of 37% RH) chambers at room temperature (20-30° C.) after 6 days. Solid parts were covered by Kapton film and investigated by XRPD, showing a mixture of Form VI and Form II, and showing no pure Form VI (FIG. 59).

### Example 26. Volixibat Potassium Salt Amorphous Form

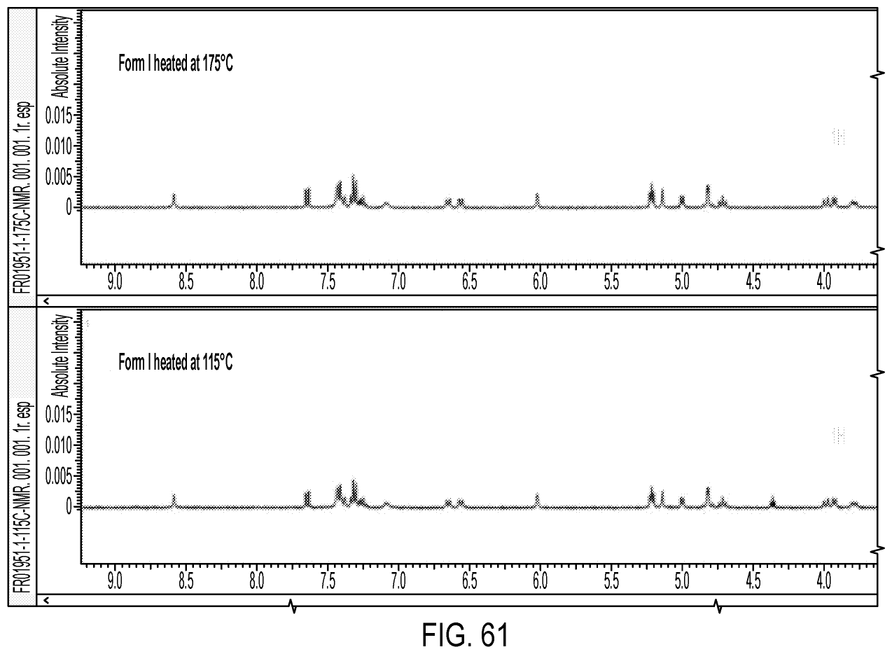

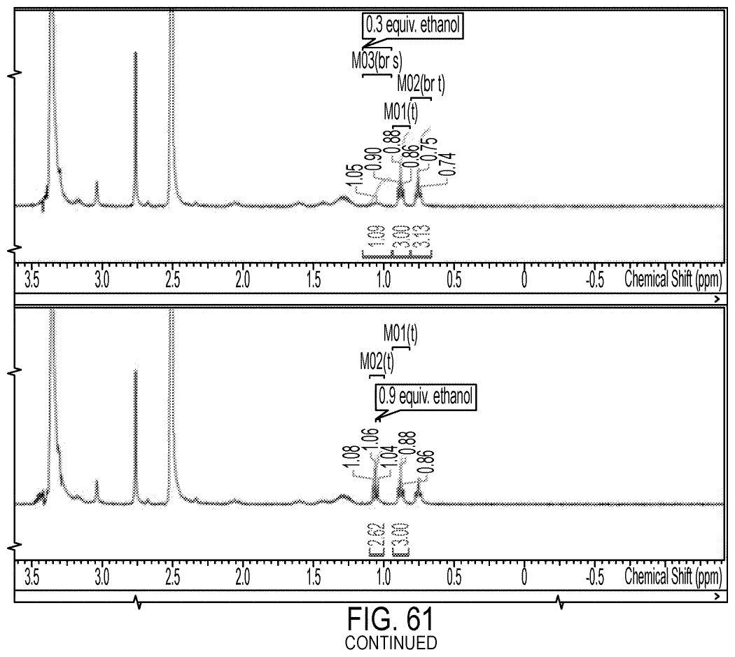

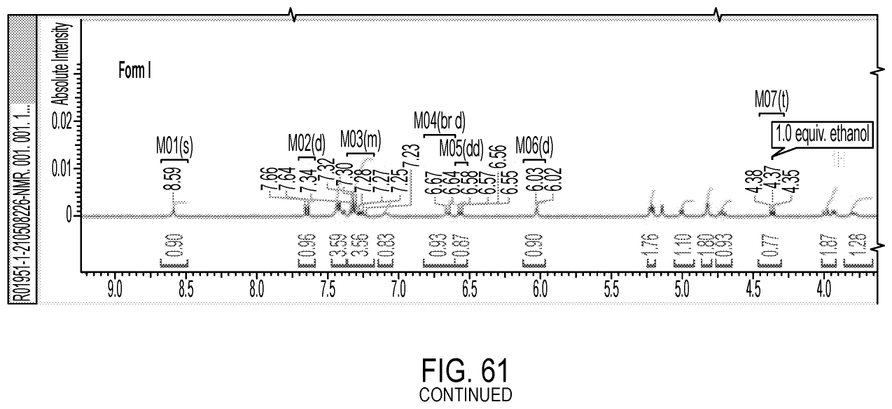

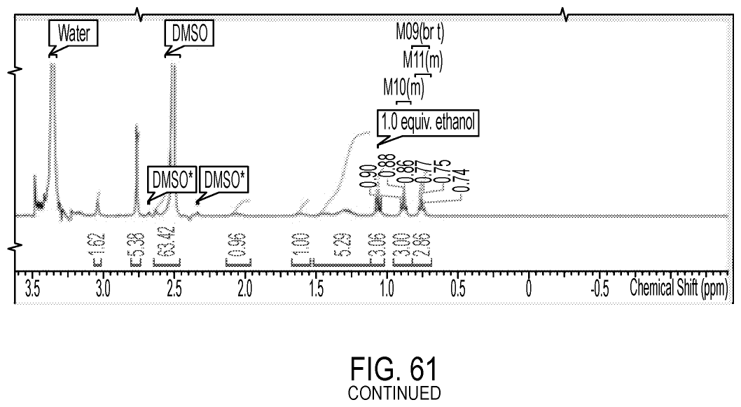

Based on the TGA data of Form I that show two stage weight loss, Form I was heated to 115° C. and 175° C., signifying the completions of the two stages of weight loss. Samples were cooled to room temperature (20-25° C.) after heating and XRPD (FIG. 60) and ¹H-NMR (FIG. 61) were conducted. These data showed no form change and no decrease of ethanol content when heated to 115° C., suggesting the first weight loss step belongs to dehydration and water molecules of Form I could be in a channel structure. Likely, after dehydrating, Form I re-hydrates when cooled to room temperature (20-25° C.) and exposed to ambient conditions (40-75% RH). These data showed that amorphous form was obtained when heated to 175° C. The ¹H-NMR data showed decreased ethanol content at 0.3 equiv. ethanol, suggesting amorphous form could be obtained when ethanol was removed from the crystal structure of Form I and therefore, ¹H-NMR could be applied to confirm conversion from Form I to amorphous form.

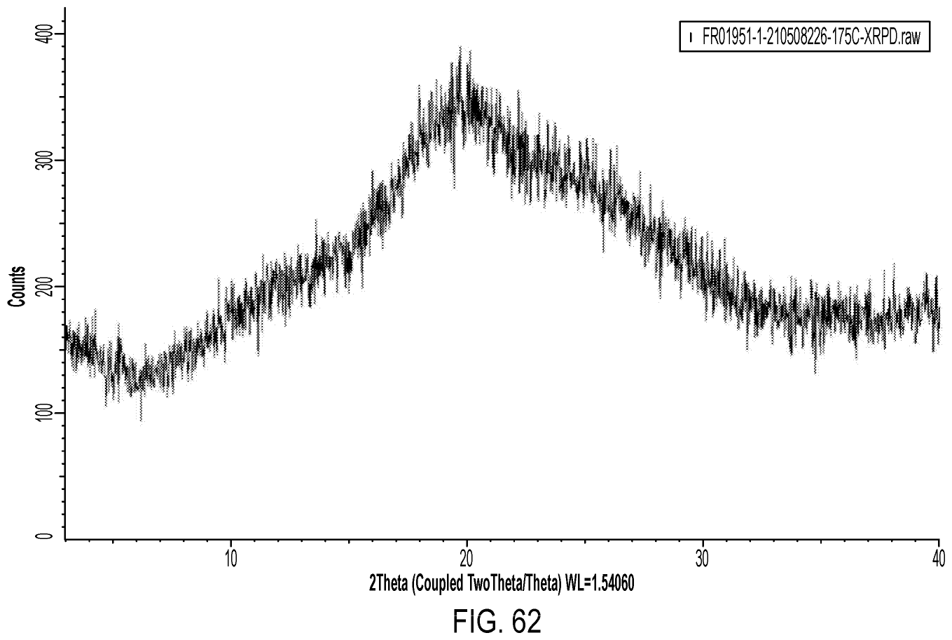

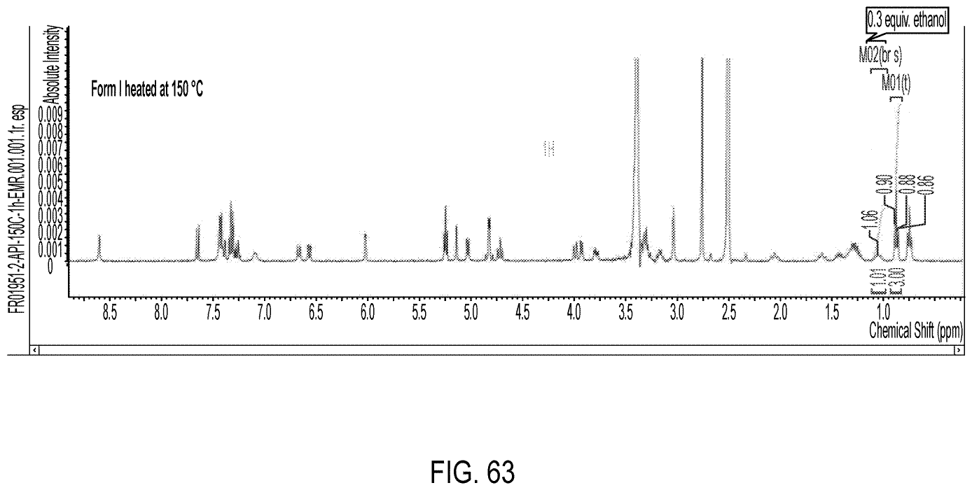

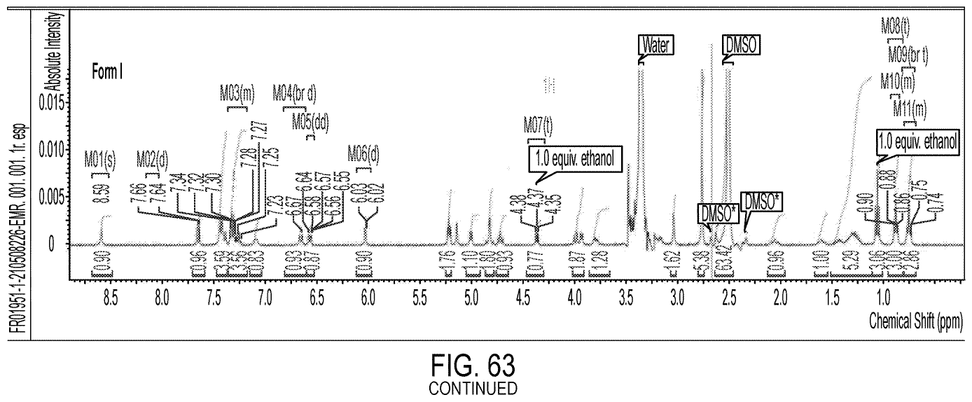

Amorphous form of volixibat potassium salt was obtained by heating Form I under N₂ at 150° C. for about 1 hour. Amorphous form was shown via XRPD (FIG. 62) and ¹H-NMR showed 0.3 equiv. ethanol, a decreased ethanol content that confirmed formation of amorphous form (FIG. 63).

### Example 27. Interrelationships of Volixibat Potassium Polymorphs

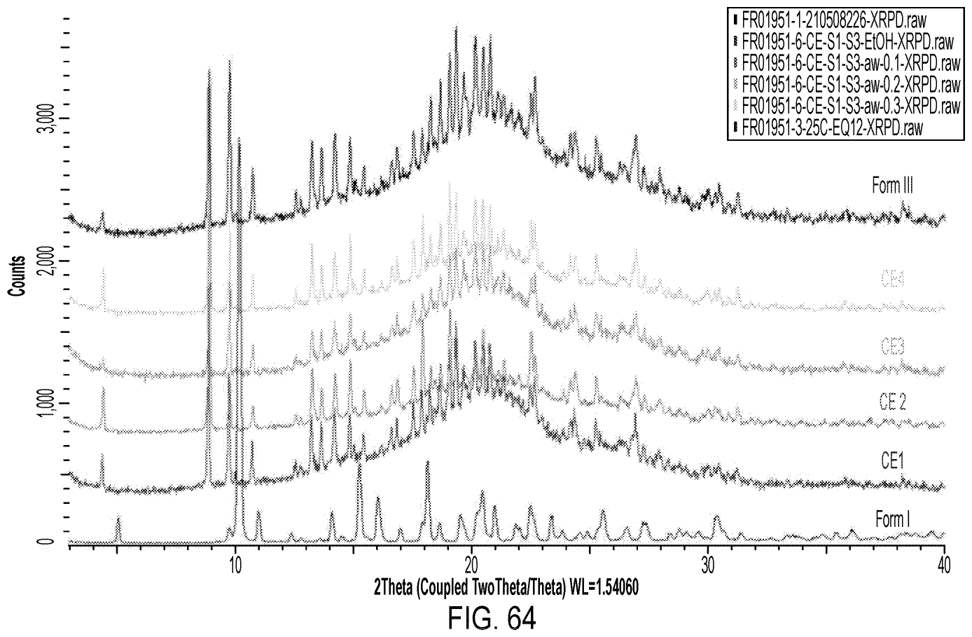

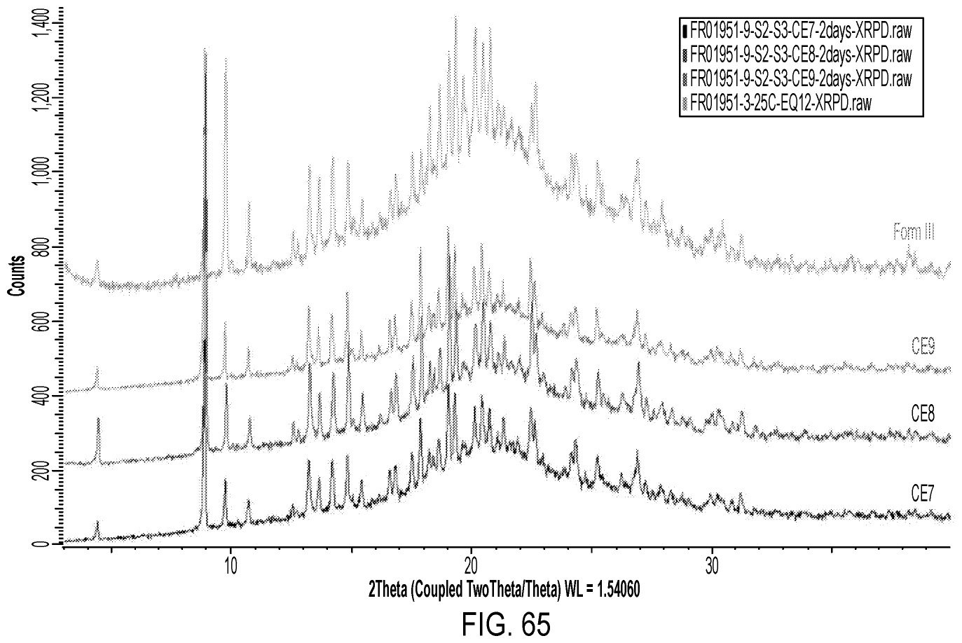

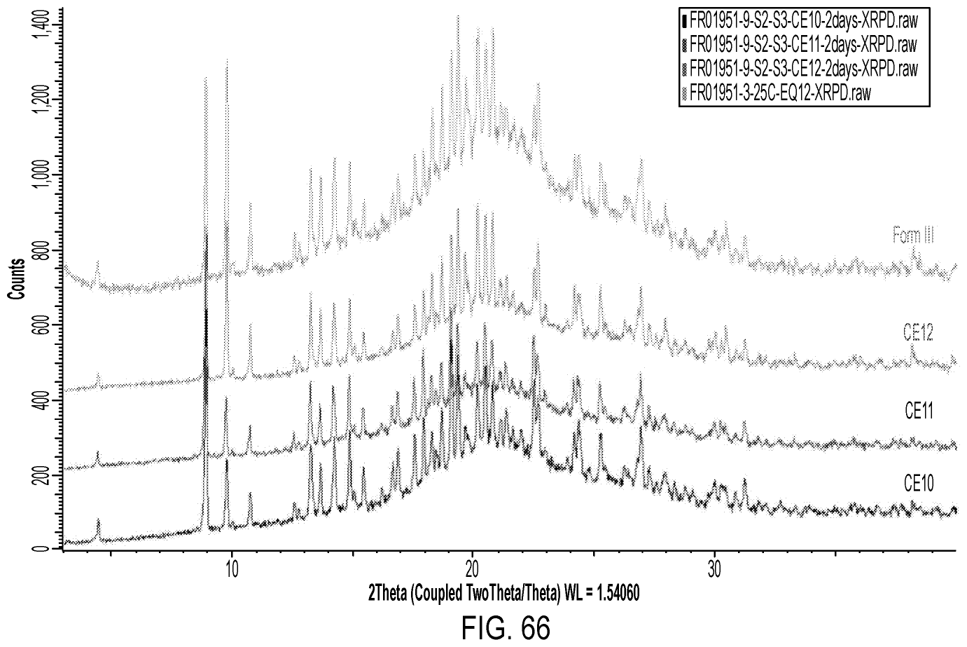

Relative stability of Form I, Form III, and Pattern A was investigated with competitive equilibration experiments in differing ethanol/water systems with different water activities (0.05≤a.w.≤0.32) (Table 16). 5 mg Form I were added into the suspensions of CE1-CE6, suspensions were stirred at 25° C. for 3 days and centrifuged at 4,000 rpm, and solid parts (wet cakes) were covered by Kapton film and investigated by XRPD. Relative stability of Form II and Form III was investigated with competitive equilibration experiments in differing ethanol/water systems with different water activities (0.32≤a.w.≤0.47) (Table 16). 5 mg Form II and 5 mg Form III were added to the selected solvent saturated by Form I (CE7-CE12), suspensions were stirred at 25° C. for 2 days and centrifuged at 4,000 rpm, and solid parts (wet cakes) were covered by Kapton film and investigated by XRPD. Relative stability of Pattern A/Form III and Form I showed only Form III was obtained (FIG. 64) and relative stability of Form II and Form III showed only Form III was obtained (FIG. 65, FIG. 66).

TABLE 16
Competitive equilibration experiments.

| Exp. ID | Solvents | Starting material | XRPD | a.w. |
|---|---|---|---|---|
| CE1 | Ethanol | Pattern A and Form I | Form III | 0.05* |
| CE2 | Ethanol/water | Form III and Form I | Form III | 0.12* |
| | (v:v = 98.7:1.3) | | | |
| CE3 | Ethanol/water | Form III and Form I | Form III | 0.22* |
| | (v:v = 97.1:2.9) | | | |
| CE4 | Ethanol/water | Form III and Form I | Form III | 0.32* |
| | (v:v = 95.1:4.9) | | | |
| CE5 | Ethanol/water | Few solids, not enough for XRPD | | 0.40* |
| | (v:v = 92.7:7.3) | characterization | | |
| CE6 | Ethanol/water | Few solids, not enough for XRPD | | 0.47* |
| | (v:v = 89.7:10.3) | characterization | | |
| CE7 | Ethanol | Form III and Form II | Form III | 0.05* |
| CE8 | Ethanol/water | Form III and Form II | Form III | 0.12* |
| | (v:v = 98.7:1.3) | | | |
| CE9 | Ethanol/water | Form III and Form II | Form III | 0.22* |
| | (v:v = 97.1:2.9) | | | |
| CE10 | Ethanol/water | Form III and Form II | Form III | 0.32* |
| | (v:v = 95.1:4.9) | | | |
| CE11 | Ethanol/water | Form III and Form II | Form III | 0.40* |
| | (v:v = 92.7:7.3) | | | Few solids, not enough for |
| | | | | XRPD characterization |
| CE12 | Ethanol/water | Form III and Form II | Form III | 0.47* |
| | (v:v = 89.7:10.3) | | | Few solids, not enough for |
| | | | | XRPD characterization |

*The water activity of a binary solvent system is calculated based on UNIFAC method (UNIQUAC Functional-group Activity Coefficients).

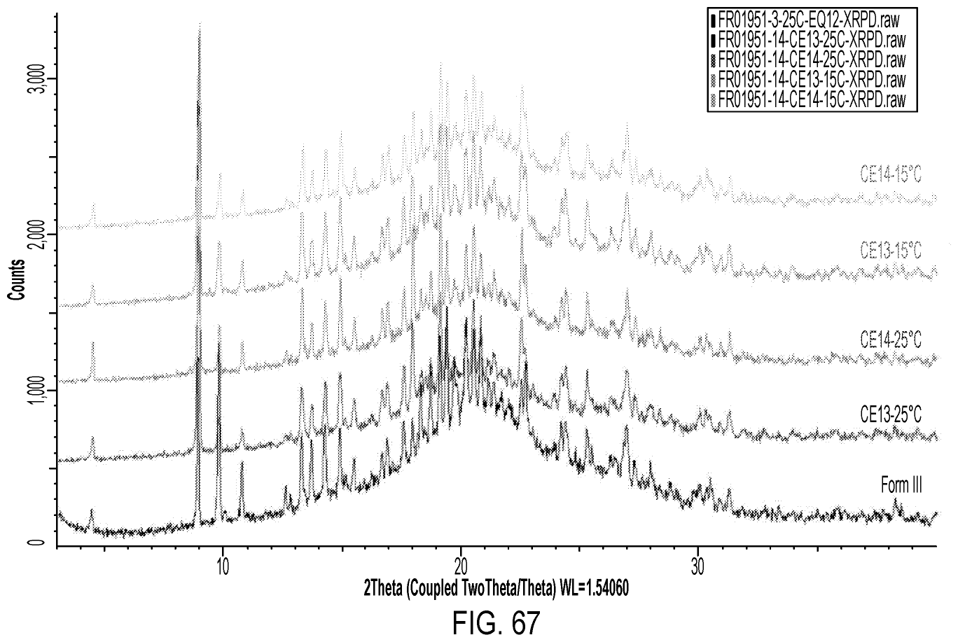

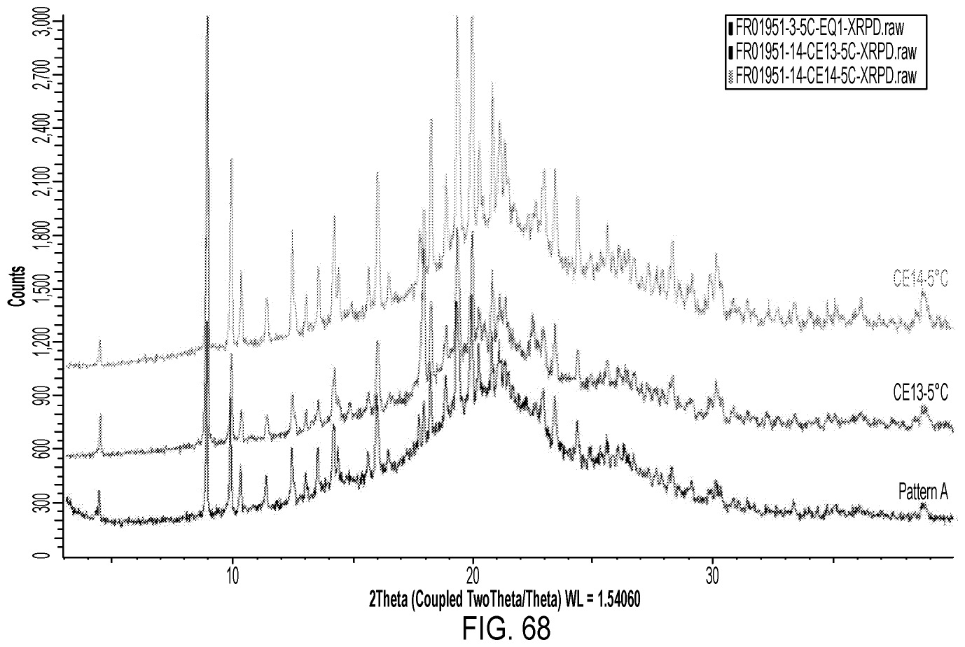

Relative stability of Form VI, Form II, and Form I was investigated with competitive equilibration experiments in ethanol/water system (a.w.=0.2 and 0.3) at 25° C., 15° C., and 5° C. (Table 17). 6 mg of solid part mixture of Form VI and Form II and 6 mg Form I were added to the selected solvent saturated by Form I (CE13-CE14), suspensions were stirred for 5 days and centrifuged at 14,000 rpm, and solid parts (wet cakes) were covered by Kapton film and investigated by XRPD. Form III was obtained at 25° C. and at 15° C. (FIG. 67) and Pattern A was obtained at 5° C. (FIG. 68).

TABLE 17
Competitive equilibration experiments among Form VI, Form II, and Form I.

| | | | | XRPD | | |
|---|---|---|---|---|---|---|
| Exp. ID | Solvents | Starting material | a.w. | 25° C. | 15° C. | 5° C. |
| CE13 | Ethanol/water | Form VI and Form | 0.2* | Form III | Form III | Pattern A |
| | (v:v = 97.1:2.9) | II and Form I | | | | |
| CE14 | Ethanol/water | Form VI and Form | 0.3* | Form III | Form III | Pattern A |
| | (v:v = 95.1:4.9) | II and Form I | | | | |

*The water activity of a binary solvent system is calculated based on UNIFAC method (UNIQUAC Functional-group Activity Coefficients).

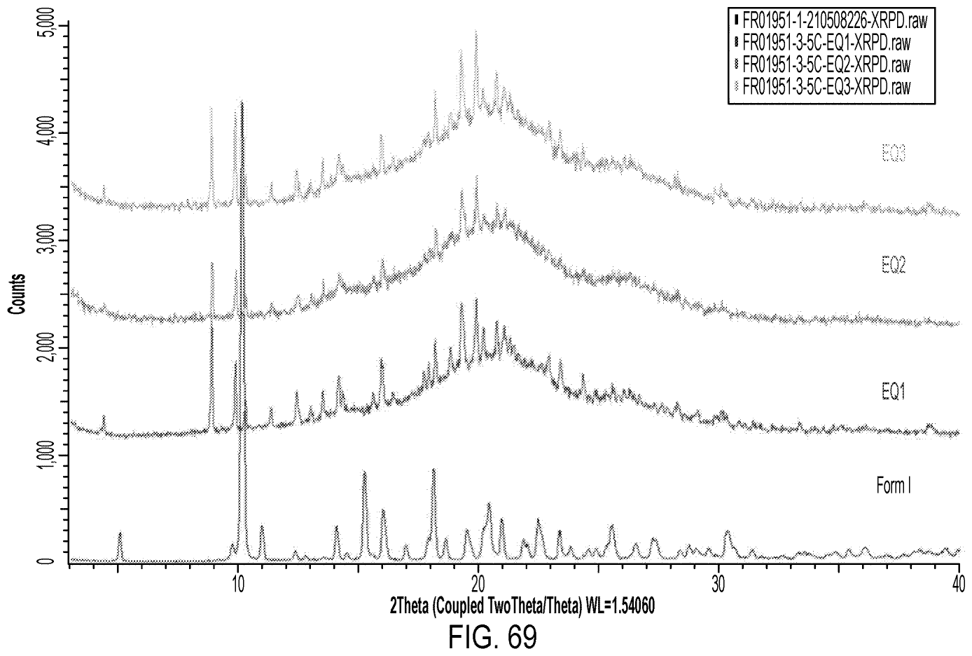

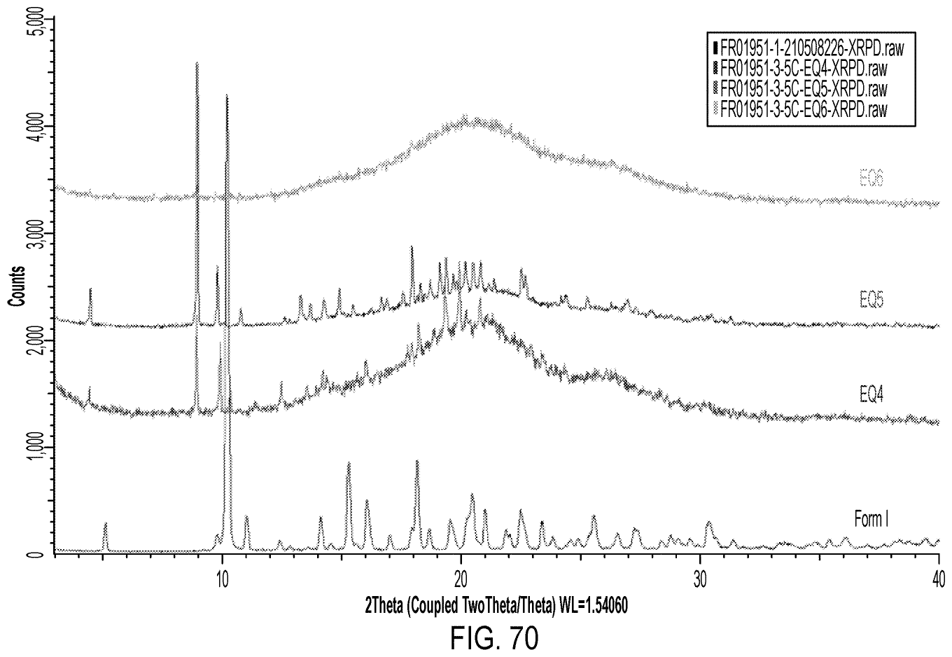

Equilibration experiments in ethanol/water system at 5° C., 25° C., and 50° C. were conducted by adding 30 mg of volixibat potassium salt amorphous form to 0.3-0.5 mL of saturated solutions, saturated by Form I, for 1 week stirring at 300 rpm. Obtained suspensions were centrifuged at 4,000 rpm and solid parts (wet cakes) covered by Kapton film were investigated by XRPD. At 5° C., Pattern A is the stable form in a.w.≤0.34, Form III is the stable form at a.w.=0.42, and amorphous form is the stable form at a.w.=0.47 (FIG. 69, FIG. 70, Table 18).

TABLE 18
Equilibration in ethanol/water system with
different water activities at 5° C.

| Exp. | | | Actual a.w. |
|---|---|---|---|
| ID | Solvents | XRPD | by KF |
| EQ1 | Ethanol | Pattern A | 0.07* |
| EQ2 | Ethanol/water | Pattern A | 0.16* |
| | (v:v = 98.6:1.4) | | |
| EQ3 | Ethanol/water | Pattern A | 0.24* |
| | (v:v = 97.0:3.0) | | |
| EQ4 | Ethanol/water | Pattern A | 0.34* |
| | (v:v = 94.9:5.1) | | |
| EQ5 | Ethanol/water | Form III | 0.42* |
| | (v:v = 92.5:7.5) | | |
| EQ6 | Ethanol/water | Almost amorphous | 0.47* |
| | (v:v = 89.5:10.5) | form, deliquesced in | |
| | | ambient condition | |
| | | (20-30° C., 40-75% RH) | |

| | | |
|---|---|---|
| EQ7 | Ethanol/water | Solubility: >200 mg/mL |
| | (v:v = 85.6:14.4) | |
| EQ8 | Ethanol/water | Solubility: >200 mg/mL |
| | (v:v = 80.2:19.8) | |
| EQ9 | Ethanol/water | Solubility: >200 mg/mL |
| | (v:v = 71.2:28.8) | |
| EQ10 | Ethanol/water | Solubility: >200 mg/mL |
| | (v:v = 47.0:53.0) | |

*The water activity of a binary solvent system is calculated based on UNIFAC method (UNIQUAC Functional-group Activity Coefficients).

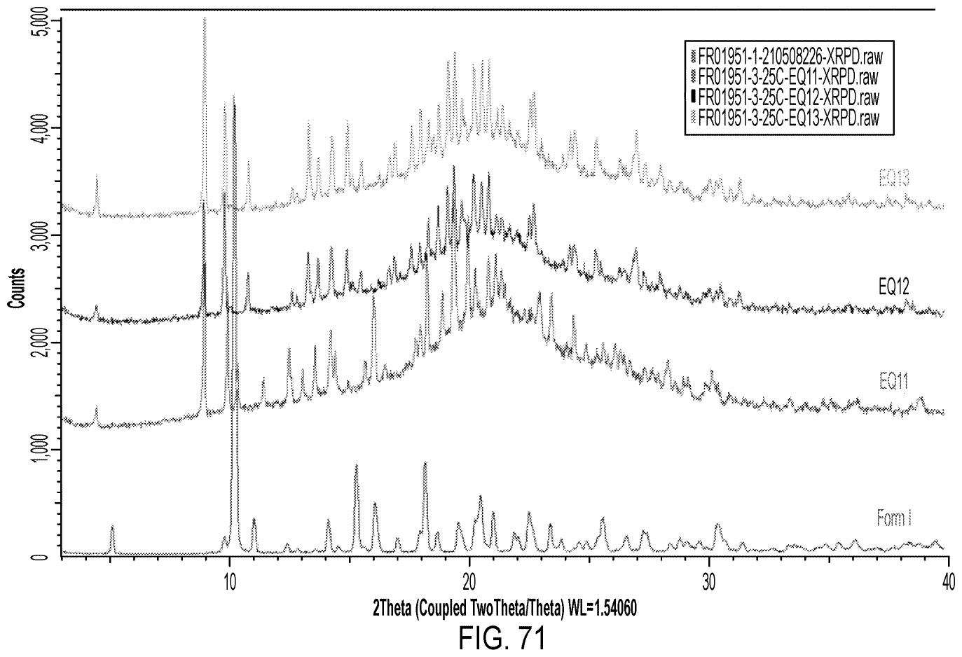

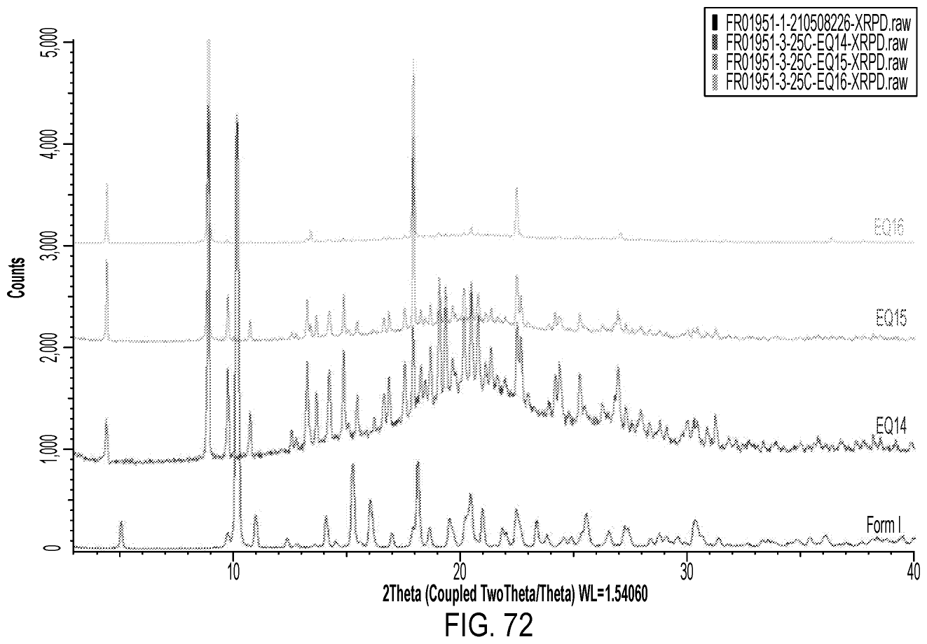

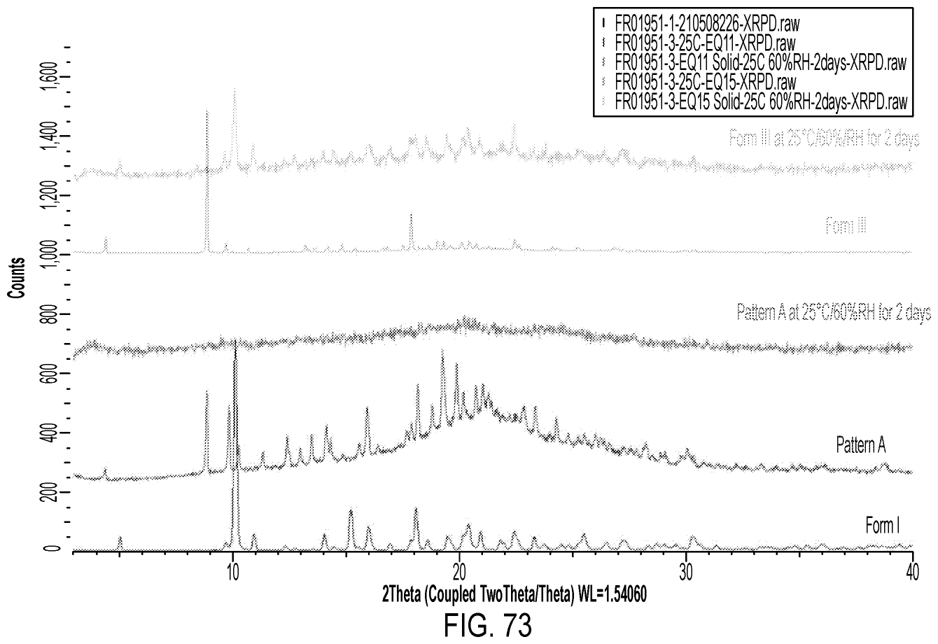

At 25° C., Pattern A is the stable form in a.w.=0.05 and Form III is the stable form at a.w.≥0.12 (FIG. 71, FIG. 72, Table 19). Pattern A powders obtained in EQ11 were placed at 25° C. and 60% RH for 2 days, converting to amorphous form and Form III powders obtained in EQ15 were placed at 25° C. and 60% RH for 2 days, converting to Form I (FIG. 73).

TABLE 19
Equilibration in ethanol/water system with
different water activities at 25° C.

| | | | Actual a.w. |
|---|---|---|---|
| Exp. ID | Solvents | XRPD | by KF |
| EQ11 | Ethanol | Pattern A | 0.05* |
| EQ12 | Ethanol/water (v:v = 98.7:1.3) | Form III | 0.12* |
| EQ13 | Ethanol/water (v:v = 97.1:2.9) | Form III | 0.22* |
| EQ14 | Ethanol/water (v:v = 95.1:4.9) | Form III | 0.32* |
| EQ15 | Ethanol/water (v:v = 92.7:7.3) | Form III | 0.40* |
| EQ16 | Ethanol/water (v:v = 89.7:10.3) | Form III | 0.47* |

| | | |
|---|---|---|
| EQ17 | Ethanol/water (v:v = 85.8:14.2) | Solubility: >200 mg/mL |
| EQ18 | Ethanol/water (v:v = 80.2:19.8) | Solubility: >200 mg/mL |
| EQ19 | Ethanol/water (v:v = 70.8:29.2) | Solubility: >200 mg/mL |
| EQ20 | Ethanol/water (v:v = 45.0:55.0) | Solubility: >200 mg/mL |

*The water activity of a binary solvent system is calculated based on UNIFAC method (UNIQUAC Functional-group Activity Coefficients).

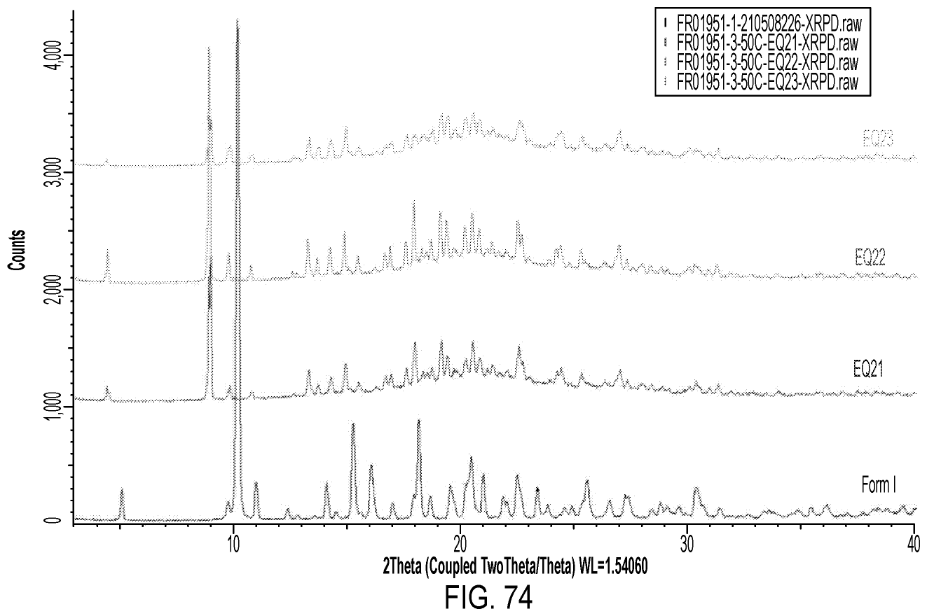

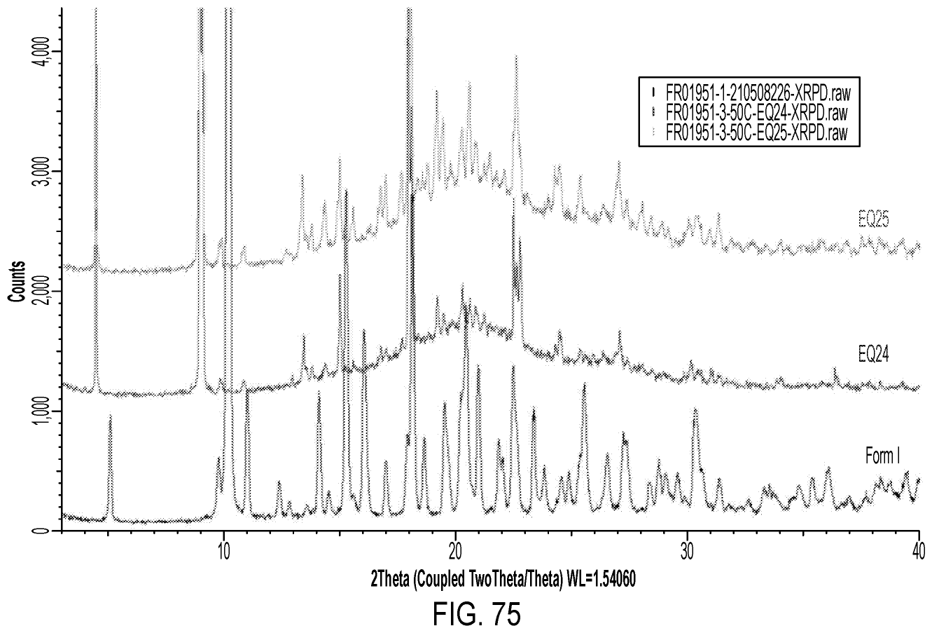

At 50° C., Form III is the stable form at a.w.≥0.08 (FIG. 74, FIG. 75, Table 20).

TABLE 20
Equilibration in ethanol/water system with
different water activities at 50° C.

| | | | Actual a.w. |
|---|---|---|---|
| Exp. ID | Solvents | XRPD | by KF |
| EQ21 | Ethanol | Form III | 0.08* |
| EQ22 | Ethanol/water (v:v = 98.7:1.3) | Form III | 0.15* |
| EQ23 | Ethanol/water (v:v = 97.2:2.8) | Form III | 0.25* |
| EQ24 | Ethanol/water (v:v = 95.3:4.7) | Form III | 0.32* |
| EQ25 | Ethanol/water (v:v = 92.9:7.1) | Form III | 0.34* |

| | | |
|---|---|---|
| EQ26 | Ethanol/water (v:v = 89.9:10.1) | Solubility: >200 mg/mL |
| EQ27 | Ethanol/water (v:v = 85.9:14.1) | Solubility: >200 mg/mL |
| EQ28 | Ethanol/water (v:v = 80.1:19.9) | Solubility: >200 mg/mL |
| EQ29 | Ethanol/water (v:v = 70.2:29.8) | Solubility: >200 mg/mL |
| EQ30 | Ethanol/water (v:v = 43.0:57.0) | Solubility: >200 mg/mL |

*The water activity of a binary solvent system is calculated based on UNIFAC method (UNIQUAC Functional-group Activity Coefficients).

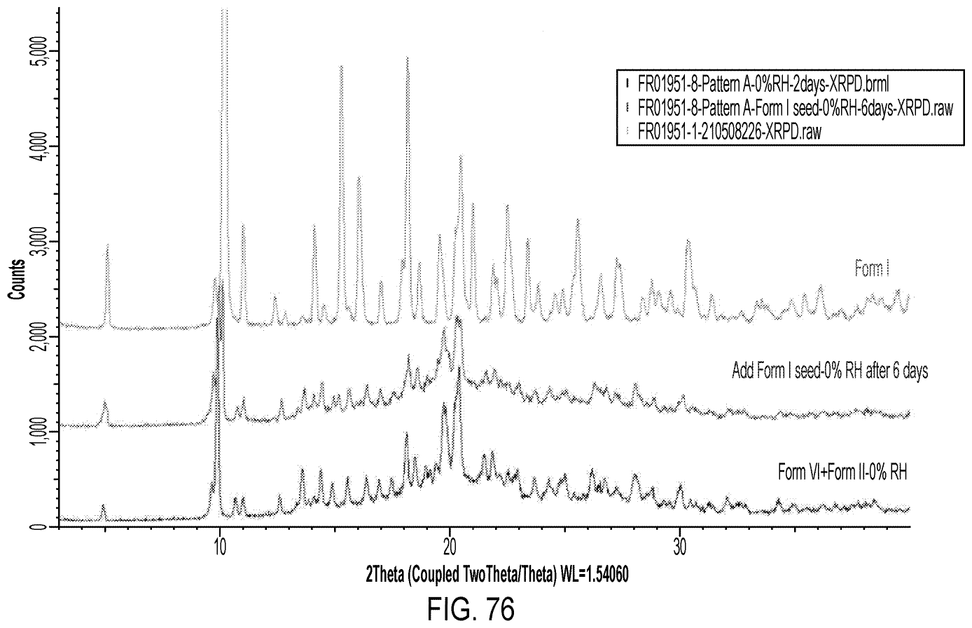

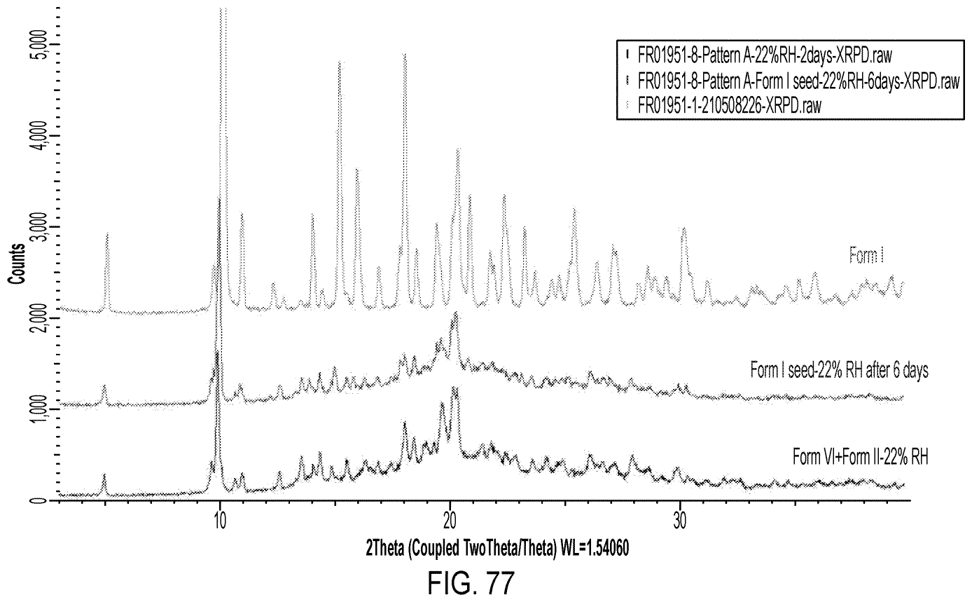

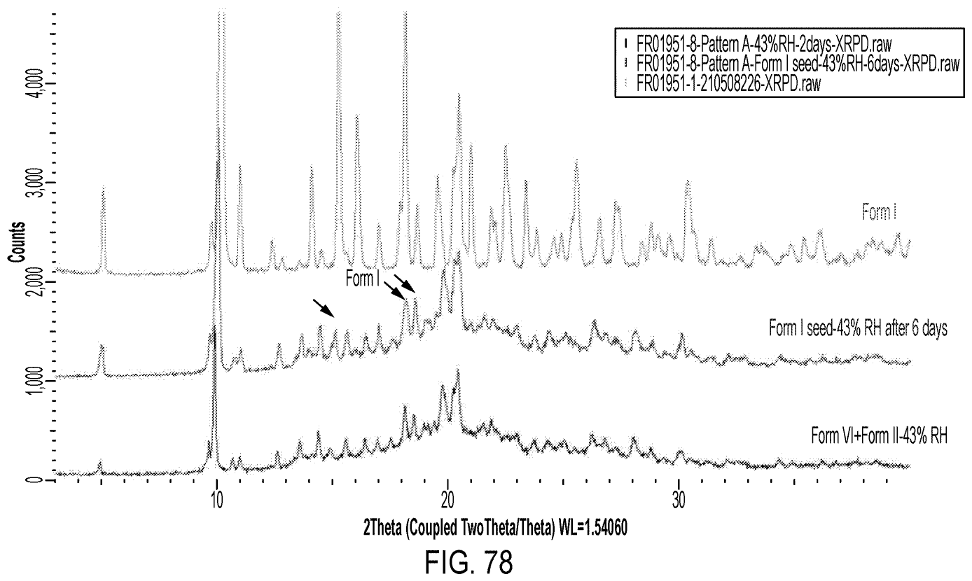

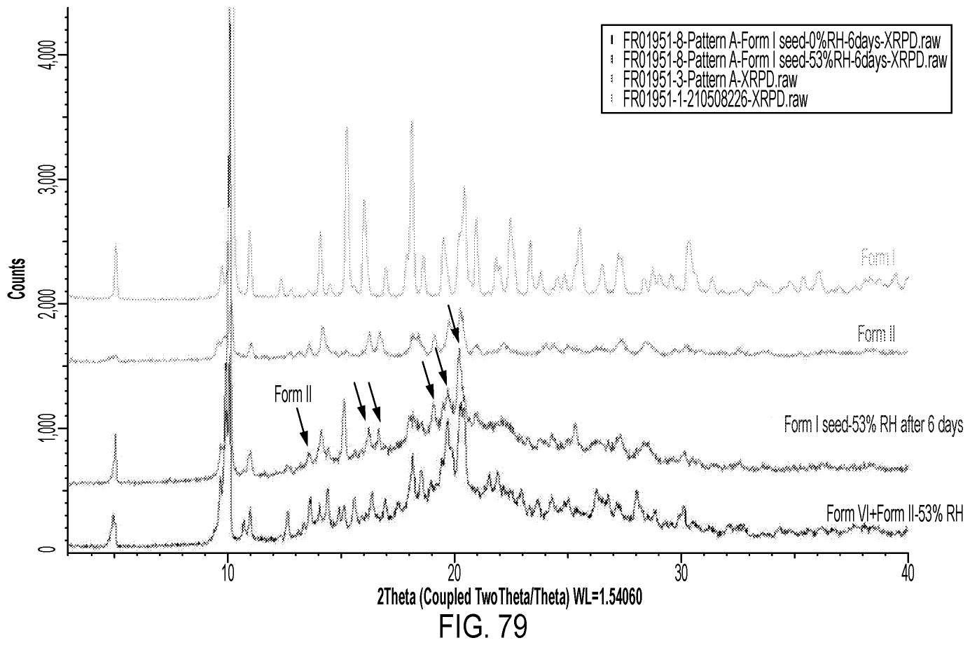

10 mg of Form I were added as seed to mixture samples of Form VI and Form II to evaluate the relative stability among Form VI, Form II, and Form I in different humidities in solid-state. Samples were placed in 0%, 22.5%, 43%, and 53% RH at 25° C. for 6 days and samples were characterized by XRPD without Kapton film. This resulted in mixtures of Form VI, Form II, and Form I at 60% RH and below, but a mixture of Form II and Form I at 65% RH (FIG. 76, FIG. 77, FIG. 78, FIG. 79, Table 21).

TABLE 21
Evaluation of Form VI, Form II, and Form
I in different humidity at 25° C.

| | | | Actual |
|---|---|---|---|
| Theoretical humidity | Starting material | XRPD | humidity |
| 0% RH (phosphoric | Form VI (major) | Form VI and | 16% RH |
| pentoxide) | and Form II and | Form II and | |
| | Form I (⅓) | Form I | |
| 22.5% RH (potassium | Form VI (major) | Form VI and | 37% RH |
| acetate) | and Form II and | Form II and | |
| | Form I (⅓) | Form I | |
| 43% RH (potassium | Form VI (major) | Form VI and | 60% RH |
| carbonate) | and Form II and | Form II and | |
| | Form I (⅓) | Form I | |
| 53% RH (magnesium | Form VI (major) | Form II and | 65% RH |
| nitrate) | and Form II and | Form I | |
| | Form I (⅓) | | |

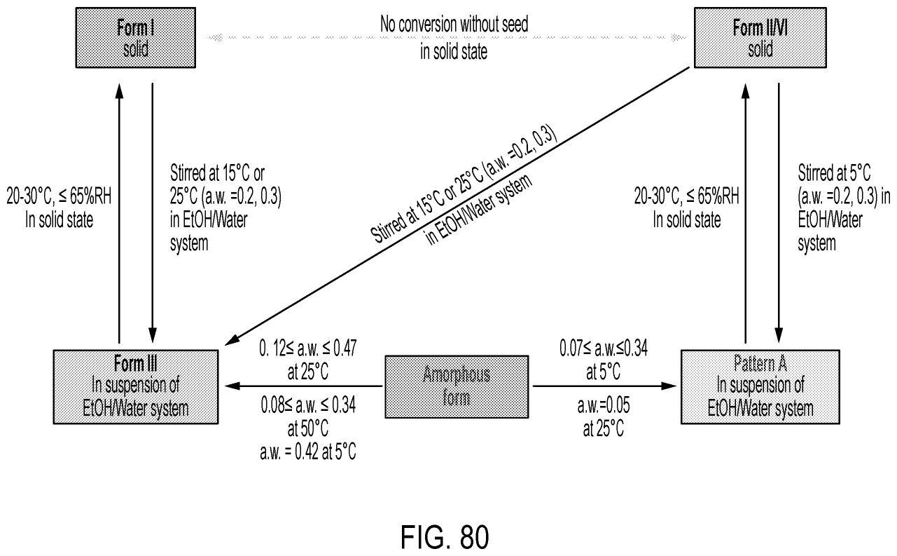

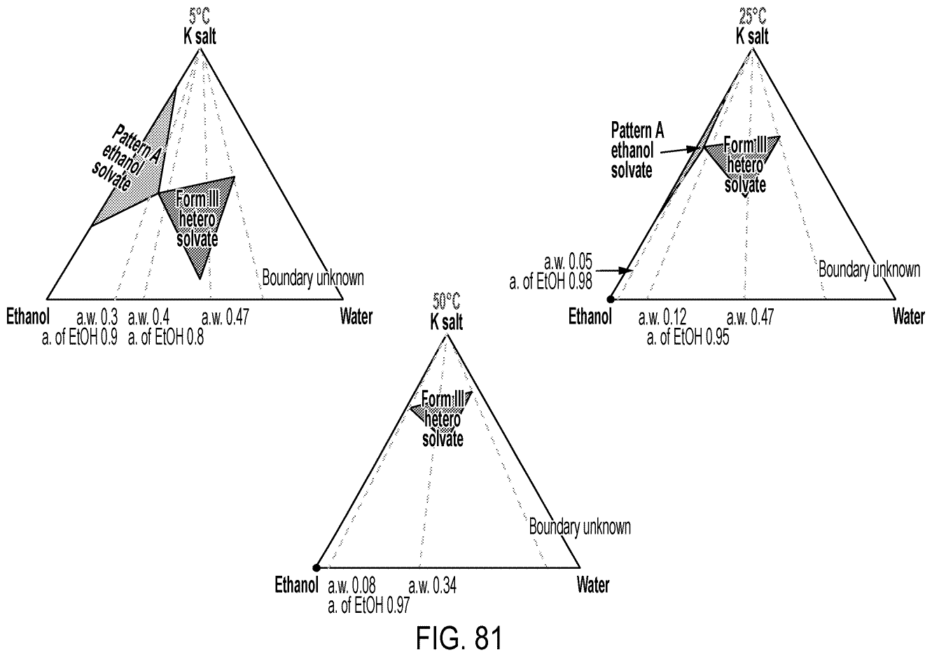

Based on above results, Pattern A is the stable form at low temperature and under low a.w. in ethanol/water system (in a.w.≤0.34 at 5° C.; in a.w.≤0.05 at 25° C.) while Form III is the stable form at higher temperature and under relatively high a.w. in ethanol/water system (in a.w.≥0.42 at 5° C.; in a.w.≥0.12 at 25° C.; in a.w.≥0.08 at 50° C.). Form III can convert to Form I at ambient condition (20-30° C.; 40-75% RH) within about 1 hour. While Pattern A, Form II, and Form VI cannot convert to Form I in solid state even with Form I seeds. Therefore, higher temperature and higher a.w. in ethanol/water system (in a.w.≥0.42 at 5° C.; in a.w.≥0.12 at 25° C.; in a.w.≥0.08 at 50° C.) is suggested during manufactory of Form I. A schematic diagram of the interconversion relationship of polymorphs is shown in FIG. 80 and a schematic phase diagram of volixibat potassium salt is shown in FIG. 81.

Below are the methods used in the Examples described above.

XRPD methods are shown in Table 22.

TABLE 22
XRPD methods.

| | |
|---|---|
| Instrument | Bruker D8 Advance |

Method 1

| | |
|---|---|
| X-ray geometry | Reflection |
| Detector | LYNXEYE_XE_T(1D mode) |
| Open angle | Max |
| Radiation | Cu/K-Alpha1 (λ = 1.5406 Å) |
| X-ray generator power | 40 kV, 40 mA |
| Primary beam path slits | Twin_Primary motorized slit 10.0 mm by |
| | sample length; SollerMount axial soller 2.5° |
| Secondary beam path slits | Detector OpticsMount soller slit 2.5°; |
| | Twin_Secondary motorized slit 5.2 mm |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Monocrystalline silicon, flat surface |

Method 2

| | |
|---|---|
| X-ray geometry | Reflection |
| Detector | LYNXEYE_XE_T(1D mode) |
| Open angle | Max |
| Radiation | Cu/K-Alpha1 (λ = 1.5406 Å) |
| X-ray generator power | 40 kV, 40 mA |
| Primary beam path slits | Twin_Primary motorized slit 10.0 mm by |
| | sample length; SollerMount axial soller 2.5° |
| Secondary beam path slits | Detector OpticsMount soller slit 2.5°; |
| | Twin_Secondary motorized slit 5.2 mm |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Monocrystalline silicon, flat surface |
| | (with or without Kapton film) |

DSC methods are shown in Table 23.

TABLE 23
DSC methods.

| Instrument | TA Discovery 2500 or Q2000 | |
|---|---|---|
| Sample pan | Tzero pan and Tzero hermetic lid with | |
| | a pin hole of 0.7 mm in diameter | |
| Temperature range | 30 to 250° C. or before decomposition | |
| Heating rate | 10° | C./min |
| Nitrogen flow | 50 | mL/min |

| | |
|---|---|
| Sample mass | About 0.5-1.5 mg |

TGA methods are shown in Table 24.

TABLE 24
TGA methods.

| | |
|---|---|
| Instrument | Discovery 5500 or Q5000 |
| Sample pan | Aluminum, open |
| Start temperature | Ambient condition (below 35° C.) |
| Final temperature | 300° C. or abort next segment if weight <80% |
| | (w/w) (The weight loss of the compound is no |
| | more than 20% (w/w)) |
| Heating rate | 10° C./min |
| Nitrogen flow | Balance 10 mL/min; sample chamber 25 mL/min |
| Sample mass | About 2-10 mg |

DVS methods are shown in Table 25.

TABLE 25
DVS methods.
Method 1

| Instrument | SPSadv-1μ | |
|---|---|---|
| Total gas flow | 4000 | ml/min |
| Oven temperature | 25° | C. |
| Solvent | Water | |
| Method | Cycle: 40-95-0-95-40% RH | |
| | Stage Step: 10% | |
| | dm/dt = 0.002%/min | |
| | Equilibrium: 240 min for each step | |
| Sample mass | About 10-100 mg | |
| Method 2 | | |
| Instrument | Intrinsic | |
| Total gas flow | 200 | sccm |
| Oven temperature | 25° | C. |

| | |
|---|---|
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |
| Sample mass | About 5-20 mg |

KF methods are shown in Table 26.

TABLE 26
KF methods.

| | |
|---|---|
| Instrument | Mettler Toledo Coulometric |
| | KF Titrator C30 |
| Method | Coulometric |
| Sample mass | About 2-6 mg |

SEM methods are shown in Table 27.

TABLE 27
SEM methods.

| | |
|---|---|
| Instrument | Phenom Prox SEM-EDS |
| Detector | BSD Full |
| Magnification | 200x-10000x |

PLM methods are shown in Table 28.

TABLE 28
PLM methods.

| | |
|---|---|
| Instrument | Nikon LV100POL |
| Method | Crossed polarizer, silicone oil added |

NMR methods are shown in Table 29.

TABLE 29
NMR methods.

| Instrument | Bruker Avance-AV 400M (for ¹H-NMR) | |
|---|---|---|
| Frequency | 400 | MHz |
| Probe | 5 mm PABBO BB/19F-1H/D Z-GRD | |
| | Z108618/0406 (for ¹H-NMR) | |
| Number of scan | 8 | |
| Temperature | 297.6 | K |
| Relaxation delay | 1 | second |

IC methods are shown in Table 30.

TABLE 30
IC methods.

| Instrument | Metrohm 940 professional IC | |
|---|---|---|
| Sample center | 889 IC | |
| Detector | Conductivity detector | |
| Eluent (anion) | 3.2 mmol/L Na2CO3 + 1.0 mmol/L NaHCO3 | |
| Eluent (cation) | 2.5 mmol/L MSA | |
| Suppressor solutions | 0.5% H2SO4 | |
| Column: | Anion A SUPP 5-150 or Cation Column C4-150 | |
| Column temperature: | 30° | C. |
| Flow rate: | 0.7 mL/min (anion) or 0.9 mL/min (cation) | |
| Diluent: | Acetonitrile:water = 60:40 (v/v) | |
| Injection volume: | 20 | pL |

HPLC methods are shown in Table 31.

TABLE 31
HPLC methods.

| Instrument | SHIMADZU LC-20AD | | |
|---|---|---|---|
| Wavelength | 242 nm | | |
| Column | 4.6*150 mm, 3-Micron | | |
| Detector | UV | | |
| Column temperature | 30° C. | | |
| Flow rate | 1.0 mL/min | | |
| Mobile phase A | 90% Water, 10% Acetonitrile, 0.1% TFA | | |
| Mobile phase B | 10% Water, 90% Acetonitrile, 0.075% TFA | | |
| Diluent | H2O/Acetonitrile (v/v = 40%:60%) | | |
| Injection volume | 20 pL | | |
| | | Mobile | |
| | | Phase | Mobile |
| | Time | A (%) | Phase B |
| Gradient | 0 | 70 | 30 |
| | 2 | 70 | 30 |
| | 12 | 55 | 45 |
| | 17.5 | 0 | 100 |
| | 21 | 0 | 100 |
| | 23 | 70 | 30 |
| | 25 | 70 | 30 |

PSD methods are shown in Table 32.

TABLE 32
PSD methods.

| Instrument | SYMPATEC | |
|---|---|---|
| Method | Dry Dispersion | |
| Measuring Range | R3: <175 pm (R1 <35 pm; R5 <875 pm) | |
| Background test | 10 | s |
| Time base | 10 | ms |
| Trigger Conditions | Fast test | |
| Start | c.opt >= 1% | |
| Valid | 2% <= c.opt <= 30% | |
| Stop | 1 s c.opt <= 2% or 2 s real time | |
| Trigger termination | 60 | s |
| Dispersing System | RODOS-OASISDRY | |
| Dispersing medium | Air | |
| Injector | 4 | mm |
| Dispersing method | 0.5 bar 65% 1.5 mm OASISDRY, | |
| | feed rate = 65.00% | |
| Sampler | VIBRI | |
| Cleaning time | 10 | s |
| Vacuum Cleaner | Nilfisk | |
| Delay | 2 | s |

X-ray crystallographic analysis was performed on Rigaku Oxford Diffraction XtaLAB Synergy-S four-circle diffractometer equipped with a HyPix-6000HE area detector. The cryogenic system used was the Oxford Cryostream 800. Radiation type was CuKα: λ=1.54184 Å, 50 W. Micro focus source with multilayer mirror (μ-CMF) was used. Distance from the crystal to the CCD detector: d=35 mm. Tube Voltage: 50 kV; Tube Current: 1 mA.

For X-ray crystallographic analysis of Form III, a total of 44488 reflections were collected in the 2θ range from 4.450 to 133.184°. The limiting indices were: −8≤h≤8, −47≤k≤47, −9 111; which yielded 8833 unique reflections (Rᵢₙₜ=0.0609). The flack parameter was 0.065(6). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 553, compared with 8833 data. All reflections were included in the refinement. The goodness of fit on F² was 1.120 with a final R value for [I>=2σ (I)] of R₁=0.0651 and wR₂=0.1650 and a final R value for all data of R₁=0.0659 and wR₂=0.1654. The largest differential peak and hole were 0.74 and −0.51 Å⁻³, respectively.

For X-ray crystallographic analysis of Form I, a total of 37887 reflections were collected in the 2θ range from 5.170 to 133.182°. The limiting indices were: −8≤h≤8, −40≤k≤40, −11≤1≤9; which yielded 8061 unique reflections (Rᵢₙₜ=0.0639). The flack parameter was 0.021(8). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 552, compared with 8061 data. All reflections were included in the refinement. The goodness of fit on F² was 1.059 with a final R value for [I>2σ (I)] of R₁=0.0679 and wR₂=0.1634 and a final R value for all data of R₁=0.0720 and wR₂=0.1660. The largest differential peak and hole were 0.80 and −0.53 Å⁻³, respectively.

Crystals were prepared by dissolving 10 mg volixibat potassium salt in 1.5 mL ethanol with 3% water content and storing the solution in a 4 mL vial. 2.0 mL heptane were dribbled slowly into the vial so that ethanol and heptane formed discrete layers. The vial was sealed and the solution was allowed to spread slowly at room temperature. Crystals were observed on the second day.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A process of preparing a compound of Formula (I), 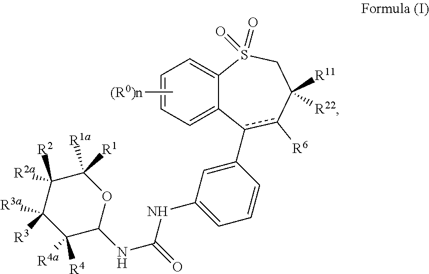
or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
one of R¹ and R^{1a} is an optionally substituted (C₁₋₆)alkylene-OSO₃H or —C(O)H and the other of R¹ and R^{1a} is H, (C₁₋₆)alkyl, NH₂, NH(C₁₋₆)alkyl, or N((C₁₋₆)alkyl)₂;
one of R² and R^{2a} is an optionally substituted (C₁₋₆)alkylene-OH, OH, or OSO₃H, and the other of R² and R^{2a} is H, (C₁₋₆)alkyl, NH₂, NH(C₁₋₆)alkyl, or N((C₁₋₆)alkyl)₂;
one of R³ and R^{3a} is an optionally substituted (C₁₋₆)alkylene-O—R^{3P} or —O—R^{3P}, wherein R^{3P} is a first alcohol protecting group, and the other of R³ and R^{3a} is H, (C₁₋₆)alkyl, NH₂, NH(C₁₋₆)alkyl, or N((C₁₋₆)alkyl)₂;
one of R⁴ and R^{4a} is an optionally substituted (C₁₋₆)alkylene-OH, OH, or OSO₃H, and the other of R⁴ and R^{4a} is H, (C₁₋₆)alkyl, NH₂, NH(C₁₋₆)alkyl, or N((C₁₋₆)alkyl)₂;
each of R¹¹ and R²² is independently an optionally substituted (C₁₋₆)alkyl;
n is an integer of 0-4;
R⁶ is H or OH;
the process comprising:
contacting the compound of Formula (I-26), 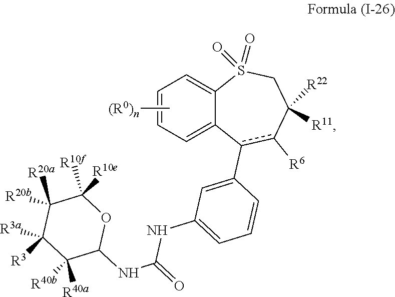
or a pharmaceutically acceptable hydrate or solvate thereof, wherein:
R⁰, R¹¹, R²², R³, R^{3a}, R⁶, and n are defined above;
one of R^{20a} and R^{20b} is an optionally substituted (C₁₋₆)alkylene-O—R^{2P}, O—R^{2P}, or OSO₃⁻(pyridine)⁺, wherein R^{2P} is a second alcohol protecting group, and the other of R^{20a} and R^{20b} is H, (C₁₋₆)alkyl, NH₂, NH(C₁₋₆)alkyl, or N((C₁₋₆)alkyl)₂;
one of R^{40a} and R^{40b} is an optionally substituted (C₁₋₆)alkylene-O—R^{3P}, O—R^{3P}, or OSO₃⁻(pyridine)⁺, wherein R^{3P} is a third alcohol protecting group, and the other of R^{40a} and R^{40b} is H, (C₁₋₆)alkyl, NH₂, NH(C₁₋₆)alkyl, or N((C₁₋₆)alkyl)₂;
one of R^{10e} and R^{10f} is C(O)H or an optionally substituted (C₁₋₆)alkylene-OSO₃⁻(pyridine)⁺ and the other of R^{10e} and R^{10f} is H, (C₁₋₆)alkyl, NH₂, NH(C₁₋₆)alkyl, or N((C₁₋₆)alkyl)₂;
with a base in an organic solvent comprising dichloromethane to produce a compound of Formula (I).

2.-15. (canceled)

16. The process of claim 1, wherein the compound of Formula (I-26) has the structure selected from the group consisting of Formula (I-AA-26), Formula (I-AB-26), Formula (I-BA-26), Formula (I-BB-26), Formula (I-CA-26), Formula (I-CB-26), Formula (I-DA-26), and Formula (I-DB-26), 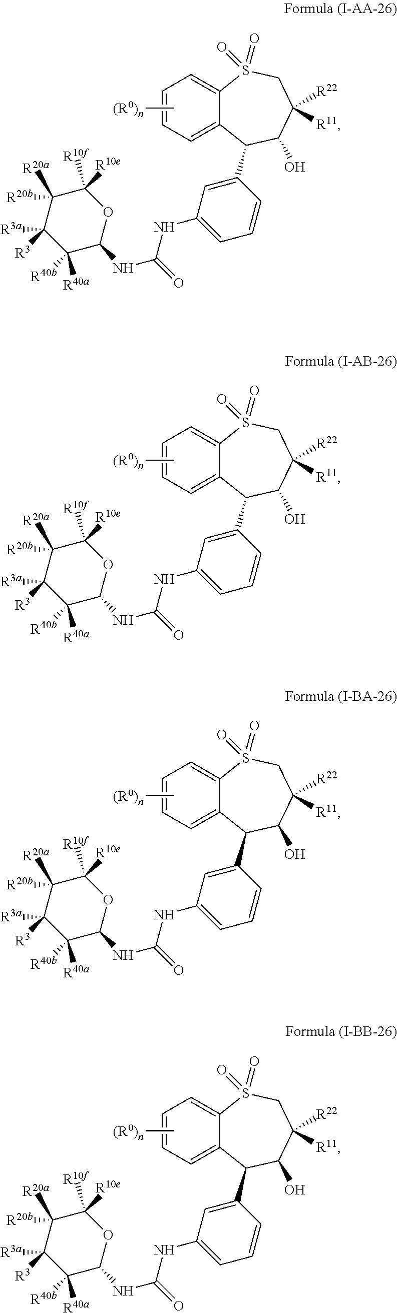 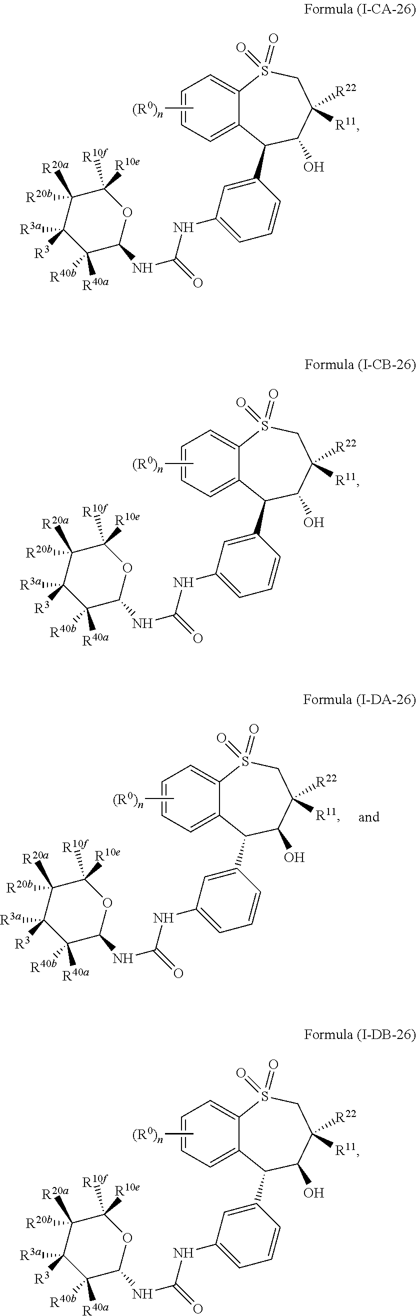
or a pharmaceutically acceptable hydrate or solvate thereof.

17. (canceled)

18. (canceled)

19. The process of claim 1, wherein the compound of Formula (I) has the structure selected from the group consisting of Formula (I-AA), Formula (I-AB), Formula (I-BA), Formula (I-BB), Formula (I-CA), Formula (I-CB), Formula (I-DA), and Formula (I-DB), 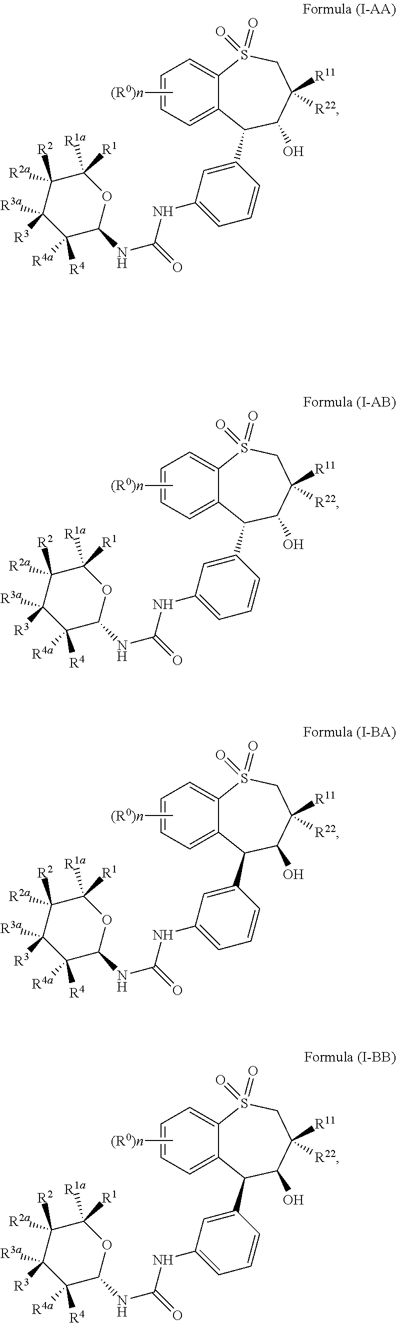 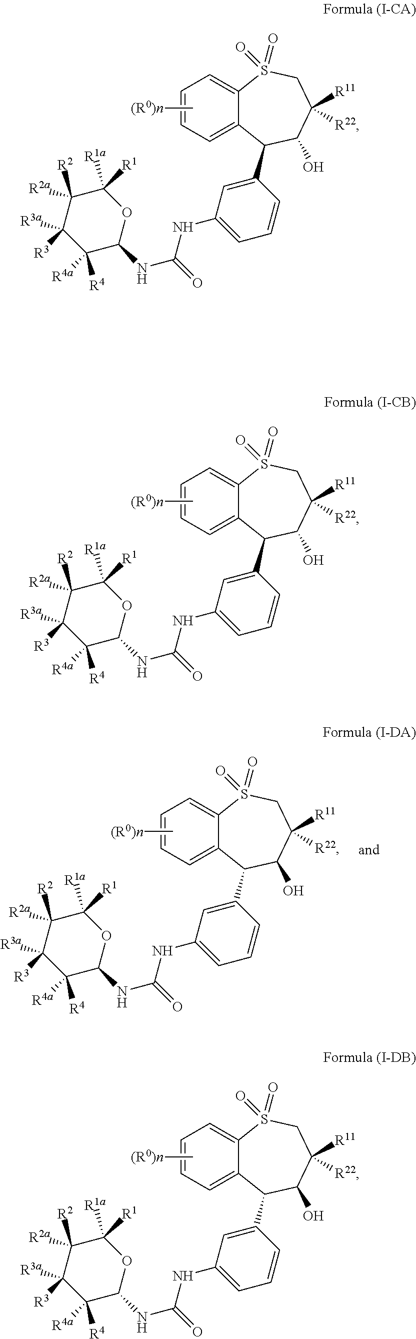
or a pharmaceutically acceptable salt, hydrate, or solvate thereof.

20.-23. (canceled)

24. The process of claim 1 further comprising the step of preparing a compound of Formula (I-26), 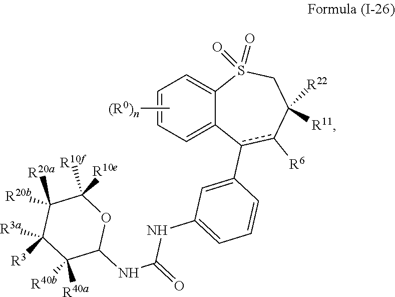
or a pharmaceutically acceptable hydrate or solvate thereof, the process comprising: contacting the compound of Formula (I-25), 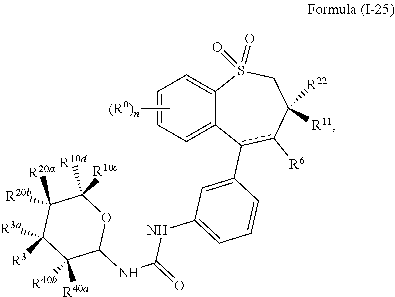
or a pharmaceutically acceptable hydrate or solvate thereof, wherein:
R⁰, R¹¹, R²², R^{10e}, R^{10f}, R^{20a}, R^{20b}, R³, R^{3a}, R^{40a}, R^{40b}, R⁶, and n are defined above;
one of R^{10c} and R^{10d} is C(O)H or an optionally substituted (C₁₋₆)alkylene-OH or —OH and the other of R^{10c} and R^{10d} is H, (C₁₋₆)alkyl, NH₂, NH(C₁₋₆)alkyl, or N((C₁₋₆)alkyl)₂;
with a sulfur trioxide-pyridine complex to produce a compound of Formula (I-26).

25.-36. (canceled)

37. The process of claim 24, wherein the compound of Formula (I-25) has the structure selected from the group consisting of Formula (I-AA-25), Formula (I-AB-25), Formula (I-BA-25), Formula (I-BB-25), Formula (I-CA-25), Formula (I-CB-25), Formula (I-DA-25), and Formula (I-DB-25), 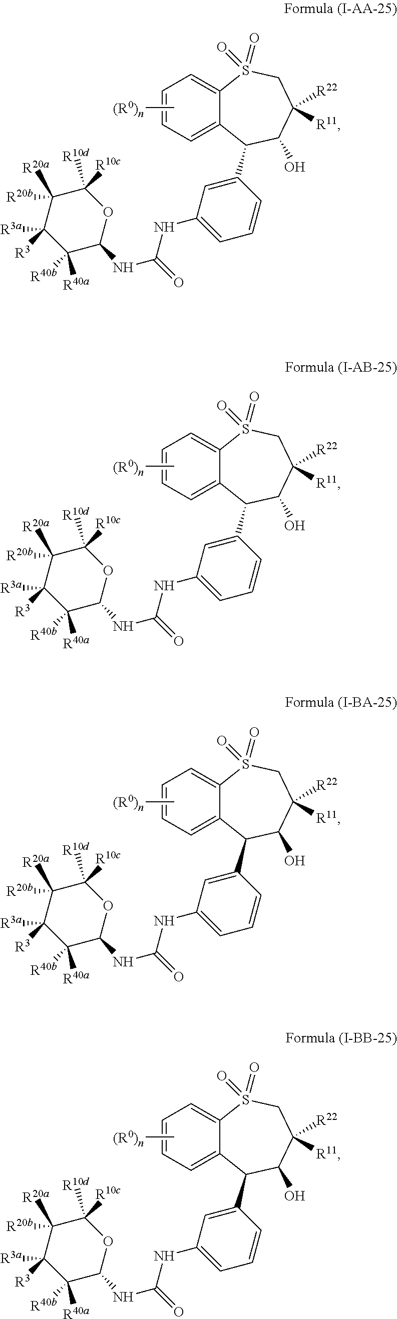 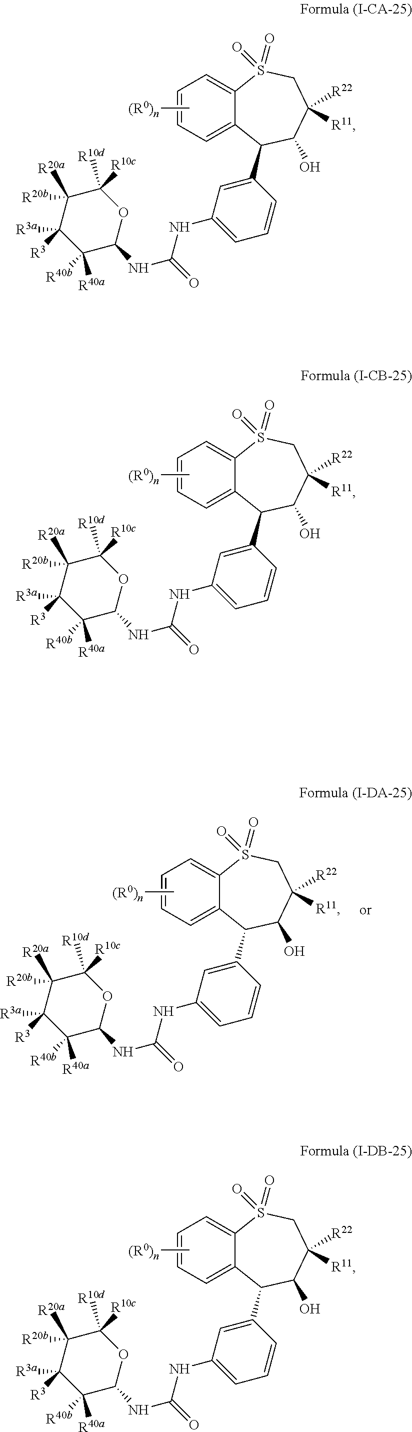
or a pharmaceutically acceptable hydrate or solvate thereof.

38.-39. (canceled)

40. The process of claim 24 further comprising the step of preparing a compound of Formula (I-25), or a pharmaceutically acceptable hydrate or solvate thereof, the process comprising: contacting the compound of Formula (I-20), 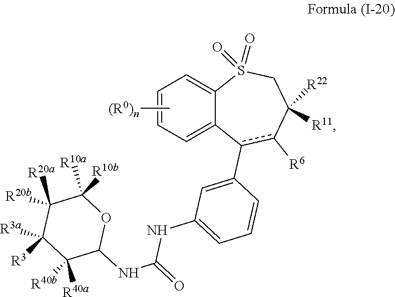
or a pharmaceutically acceptable salt, hydrate, or solvate thereof, wherein:
R⁰, R¹¹, R²², R^{20a}, R^{20b}, R³, R^{3a}, R^{40a}, R^{40b}, R⁶, and n are defined above;
one of R^{10a} and R^{10b} is C(O)H or an optionally substituted (C₁₋₆)alkylene-O—R^{4P} or O—R^{4P}, wherein R^{4P} is the fourth alcohol protecting group, and the other of R^{10a} and R^{10b} is H, (C₁-6)alkyl, NH₂, NH(C₁₋₆)alkyl, or N((C₁₋₆)alkyl)₂;
with a Sn-based catalyst to produce a compound of Formula (I-25).

41.-50. (canceled)

51. The process of claim 40, wherein the compound of Formula (I-20) has the structure selected from the group consisting of Formula (I-AA-20), Formula (I-AB-20), Formula (I-BA-20), Formula (I-BB-20), Formula (I-CA-20), Formula (I-CB-20), Formula (I-DA-20), and Formula (I-DB-20), 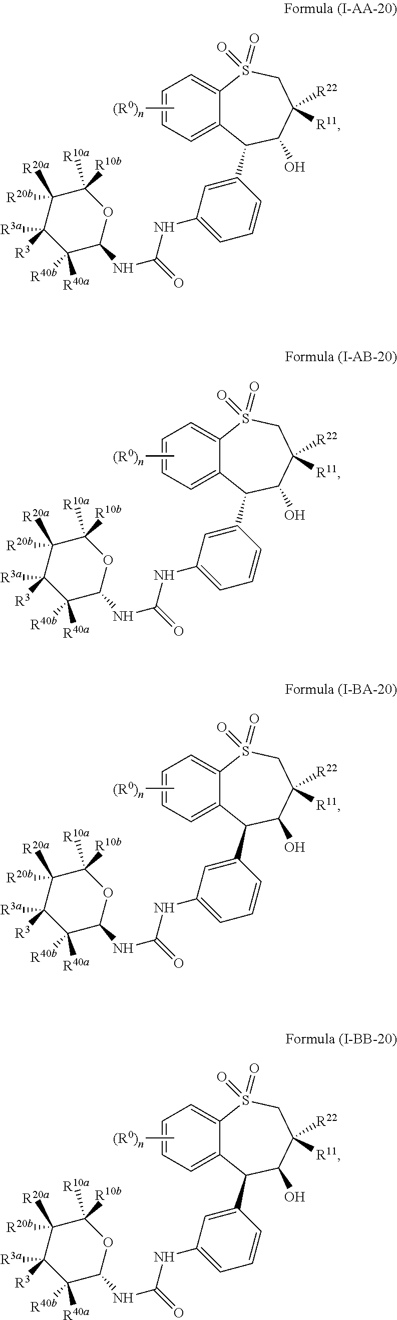 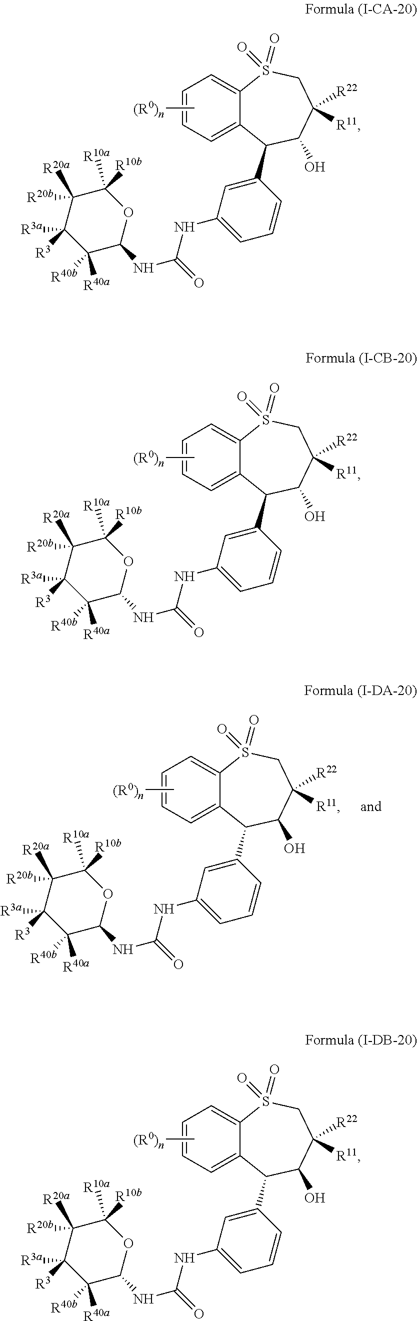
or a pharmaceutically acceptable salt, hydrate, or solvate thereof.

52. (canceled)

53. (canceled)

54. A process of preparing a compound of Formula (II-15), 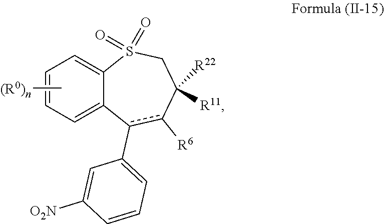
or a pharmaceutically acceptable salt, hydrate, or solvate thereof, the process comprising:
isolating the compound of Formula (II-15) from the mixture represented by Formula (II-15/115), 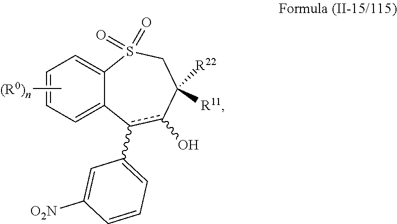
or a pharmaceutically acceptable salt, hydrate, or solvate thereof, wherein:
each R⁰ is independently halo, —OH, —SH, —CN, —N₃, —OH, —SH, —NH₂, —COOH, —O—R⁰⁰, —NHR⁰⁰, —NR⁰⁰R⁰⁰, —S—R⁰⁰, —OC(O)—R⁰⁰, —(C₁₋₆)alkylene-OH, —(C₁₋₆)alkylene-SH, —(C₁₋₆)alkylene-NH₂, wherein each R⁰⁰ is independently (C₁₋₆)alkyl;
each of R²² and R¹¹ is independently an optionally substituted (C₁₋₆)alkyl;
R⁶ is OH; and
n is an integer of 0-4;
and the process further comprising oxidizing and subsequently reducing the compound of Formula (II-15) or the mixture represented by Formula (II-15/115), forming a mixture represented by Formula (II-15/115).

55. (canceled)

56. The process of claim 54, wherein the compound of Formula (II-15) has the structure selected from the group consisting of: 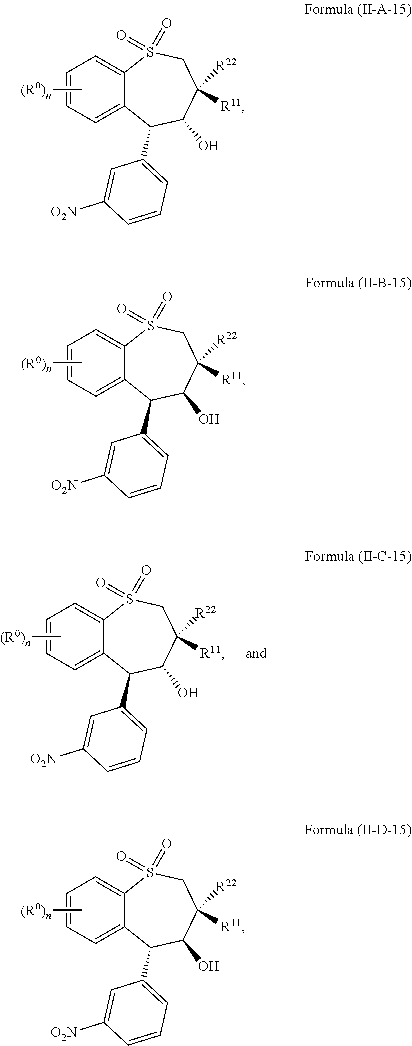
or a pharmaceutically acceptable salt, hydrate, or solvate thereof.

57.-311. (canceled)

312. A compound prepared by the process of claim 1.

313. A pharmaceutical composition comprising a therapeutically effective amount of the compound of claim 1.

314.-316. (canceled)

317. The pharmaceutical composition of claim 313, wherein the compound is in an amount of about 20 mg to about 80 mg.

318.-322. (canceled)

323. A method of treating a cholestatic liver disease in a subject in need thereof, comprising administering to the subject a therapeutically effective amount of the compound of claim 312.

324. (canceled)

325. (canceled)

326. The method of claim 323, wherein the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, PFIC type 4, PFIC type 5, PFIC type 6, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

327.-339. (canceled)

340. A pharmaceutical composition comprising a therapeutically effective amount of the compound of claim 312 and one or more excipients selected from the group consisting of:
(i) a diluent or a filler,
(ii) a disintegrant,
(iii) a channeling agent,
(iv) a glidant, and
(v) a lubricant.

341. A kit for treating cholestatic liver disease in a subject in need thereof, wherein the kit comprises at least one unit dosage of a therapeutically effective amount of the compound of claim 312.

342.-350. (canceled)
